SEQUENCE LISTING (27 sequences)

SEQ ID NO: 1 - DNA, 20 bp, Artificial
  synthetic
tggcgtcgtgattagtgatg

SEQ ID NO: 2 - DNA, 21 bp, Artificial
  synthetic
aacaccctttccaaatcctca

SEQ ID NO: 3 - DNA, 21 bp, Artificial
  synthetic
catgcaaatcacaatcacagg

SEQ ID NO: 4 - DNA, 20 bp, Artificial
  synthetic
ttgtggcttttgctgttgac

SEQ ID NO: 5 - DNA, 20 bp, Artificial
  synthetic
gagtgggtgcgacaagtttc

SEQ ID NO: 6 - DNA, 20 bp, Artificial
  synthetic
tgttctcttcccggaatgtc

SEQ ID NO: 7 - DNA, 22 bp, Artificial
  synthetic
cattgtgatgagcgtgttctgg

SEQ ID NO: 8 - DNA, 22 bp, Artificial
  synthetic
aactccccggttaggaccctta

SEQ ID NO: 9 - DNA, 21 bp, Artificial
  synthetic
ggaagtgtcagatgatcccca

SEQ ID NO: 10 - DNA, 20 bp, Artificial
  synthetic
ccgtttgcctcgtggataat

SEQ ID NO: 11 - DNA, 22 bp, Artificial
  synthetic
acagaggaggagttccgcacta

SEQ ID NO: 12 - DNA, 21 bp, Artificial
  synthetic
gcttgcttcatcttgttgcca

SEQ ID NO: 13 - DNA, 19 bp, Artificial
  synthetic
caaggccaatgacctggac

SEQ ID NO: 14 - DNA, 20 bp, Artificial
  synthetic
agctccctggacttttgcag

SEQ ID NO: 15 - DNA, 20 bp, Artificial
  synthetic
cttcgacaacctgatgcagc

SEQ ID NO: 16 - DNA, 20 bp, Artificial
  synthetic
tacttggagtctgtgccacc

SEQ ID NO: 17 - DNA, 19 bp, Artificial
  synthetic
gctgccctgatgaaccaac

SEQ ID NO: 18 - DNA, 21 bp, Artificial
  synthetic
gcaggtggttctttgaatcgg

SEQ ID NO: 19 - DNA, 20 bp, Artificial
  synthetic
gattcggaggaacctgagtg

SEQ ID NO: 20 - DNA, 19 bp, Artificial
  synthetic
cggaagcaggatcttctgg

SEQ ID NO: 21 - DNA, 21 bp, Artificial
  synthetic
agtggagaatggcacacccta

SEQ ID NO: 22 - DNA, 19 bp, Artificial
  synthetic
aagagccattgtcacccca

SEQ ID NO: 23 - DNA, 21 bp, Artificial
  synthetic
gatcccagctctcctcaatac

SEQ ID NO: 24 - DNA, 19 bp, Artificial
  synthetic
gccatttttgcaccgtgtg

SEQ ID NO: 25 - DNA, 20 bp, Artificial
  synthetic
tgaagtgtttgcccctggac

SEQ ID NO: 26 - DNA, 20 bp, Artificial
  synthetic
aggcaccatgatcatcgctg

SEQ ID NO: 27 - DNA, 25 bp, Artificial
  synthetic
gaaaggagacgaagguucaacauca

(19) United States
(12) Patent Application Publication
(10) Pub. No.: US 2022/0315548 A1
(43) Pub. Date: Oct. 6, 2022

(54) SMALL MOLECULE AGONISTS OF MUCOLIPIN 1 AND USES THEREOF

(71) Applicants: The Regents of the University of Michigan, Ann Arbor, MI (US); National Center for Advancing Translational Sciences, Rockville, MD (US)

(72) Inventors: Haoxing Xu, Ann Arbor, MI (US); Lu Yu, Ann Arbor, MI (US); Juan Jose Marugan, Gaithersburg, MD (US); Raul Rolando Calvo, Germantown, MD (US); Natalia Julia Martinez, Rockville, MD (US); Noel Terrence Southall, Potomac, MD (US); Marc Ferrer, Potomac, MD (US); Xin Hu, Frederick, MD (US)

(21) Appl. No.: 17/638,929

(22) PCT Filed: Aug. 28, 2020
(86) PCT No.: PCT/US2020/048482

§ 371 Date: Feb. 28, 2022

Related U.S. Application Data
(60) Provisional application No. 62/894,289, filed on Aug. 30, 2019.

(51) Int. Cl. C07D 291/08; C07D 211/26; C07C 311/21; C07D 207/323; C07D 295/135; C07D 207/09; C07D 223/04; C07D 401/04; C07D 471/08; C07D 401/12; C07D 235/08; A61P 21/06

(52) U.S. Cl. CPC C07D 291/08; C07D 211/26; C07C 311/21; C07D 207/323; C07D 295/135; C07D 207/09; C07D 223/04; C07D 401/04; C07D 471/08; C07D 401/12; C07D 235/08; A61P 21/06; C07C 2601/14

(57) ABSTRACT

This invention is in the field of medicinal chemistry. In particular, the invention relates to a new class of small-molecules having a phenyl-sulfonic amide (or similar) structure which function as agonists of mucolipin 1 (ML1), and their use as therapeutics for the treatment of Duchenne muscular dystrophy (DMD) and related disorders.

### STATEMENT REGARDING FEDERALLY SPONSORED RESEARCH OR DEVELOPMENT

This invention was made with government support under MH096595, NS062792, and NS091928 awarded by the National Institutes of Health. The government has certain rights in the invention.

### FIELD OF THE INVENTION

This invention is in the field of medicinal chemistry. In particular, the invention relates to a new class of small-molecules having a phenyl-sulfonic amide (or similar) structure which function as agonists of mucolipin 1 (ML1), and their use as therapeutics for the treatment of Duchenne muscular dystrophy (DMD) and related disorders.

### INTRODUCTION

An example of a condition caused in part by a genetic mutation is Duchenne muscular dystrophy (DMD). DMD is a progressive neuromuscular disease caused by mutations in the X-linked dystrophin gene (DMD), which encodes the protein dystrophin (see, Hoffman, E. P., et al.; Cell 1987, 51 (6), 919-28). DMD is the most common muscular dystrophy, affecting approximately 1 in 3500 male births. DMD patients experience progressive weakness and degeneration in skeletal muscle, including the diaphragm, cardiac muscle, and some smooth muscle (see, Wallace, G. Q., et al. Annu Rev Physiol 2009, 71, 37-57).

Symptoms of DMD usually appear in male children before age five, but may appear in infancy. Progressive proximal muscle weakness of the legs and pelvis from loss of muscle mass is observed first, which spreads to the arms, neck, and other areas. Early signs may include pseudohypertrophy, low endurance, and difficulties in standing. As the condition progresses, muscle tissue experiences wasting and eventually undergoes fibrosis. By age 10, braces are usually required to aid in walking, with most patients becoming wheelchair dependent by age 12. Later symptoms may include abnormal bone development that lead to skeletal deformities, including curvature of the spine. Due to progressive deterioration of muscle, loss of movement occurs eventually leading to paralysis. The average life expectancy for patients afflicted with DMD is around 25 years.

There is no current cure for DMD. Current treatments include steroids, physical therapy, orthopedic appliances, and respiratory support, however none are directed at the underlying mechanistic defect (see, Bushby, K.; et al.; Lancet Neurol 2010, 9 (2), 177-89; Bushby, K, et al.; Lancet Neurol 2010, 9 (1), 77-93). While these interventions have improved the lives of patients, DMD remains a lethal disease.

Accordingly, a treatment directed to the underlying mechanistic defect with DMD is needed.

### SUMMARY OF THE INVENTION

DMD is a devastating disease caused by mutations in dystrophin that compromise sarcolemma integrity, leaving muscles susceptible to contraction-induced damage. Currently, there is no treatment for DMD. Mutations in transient receptor potential mucolipin 1 (ML1), a lysosomal Ca²⁺ channel required for lysosomal exocytosis, produce a DMD-like phenotype.

Experiments conducted during the course of developing embodiments for the present invention demonstrated that transgenic overexpression or pharmacological activation of ML1 in vivo facilitates sarcolemma repair and alleviates the dystrophic phenotypes in both skeletal and cardiac muscles of mdx mice (a mouse model of DMD). Hallmark dystrophic features of DMD, including myofiber necrosis, central nucleation, fibrosis, elevated serum creatine kinase (CK) levels, reduced muscle force, impaired motor ability, and dilated cardiomyopathies, were all ameliorated by increasing ML1 activity. Considering that lysosome insufficiency occurs in DMD muscles, upregulation of ML1 restores lysosome function via activation of transcriptional factor EB (TFEB), leading to diminished muscle damage. Hence, manipulating lysosome function by targeting lysosomal Ca²⁺ channels may represent a promising approach to treat DMD and related muscle diseases. Hence, such experiments indicate that manipulating lysosome function by targeting lysosomal Ca²⁺ channels represents a promising approach to treating DMD and related muscle diseases. In addition, experiments conducted during the course of developing embodiments for the present invention designed and synthesized ML1 agonists having a phenyl-sulfonic amide (or similar) structure.

As such, the present invention relates to compounds having a phenyl-sulfonic amide (or similar) and their use to treat conditions, disorders and diseases associated with ML1 that regulate lysosomal calcium channel functioning in cells. The invention also discloses pharmaceutical compositions comprising the compounds and uses thereof to treat diseases and conditions associated with ML1, in particular DMD and other muscular dystrophy disorders related to ML1 activity (e.g., Becker's Muscular Dystrophy (BMD), Limb Girdle Muscular Dystrophy (LGMD), distal muscular dystrophy, facioscapulohumeral dystrophy, myotonic muscular dystrophy, Emery-Dreifuss muscular dystrophy, oculopharyngeal muscular dystrophy, and Congenital Muscular Dystrophy (CMD) (e.g., Laminin-α2-deficient CMD (MDC1A), Ullrich CMG (UCMDs 1, 2 and 3), Walker-Warburg syndrome (WWS), Muscle-eye-brain disease (MEB), Fukuyama CMD (FCMD), CMD plus secondary laminin deficiency 1 (MDC1B), CMD plus secondary laminin deficiency 2 (MDC1C), CMD with mental retardation and pachygyria (MDC1D), and Rigid spine with muscular dystrophy Type 1 (RSMD1)).

Accordingly, this invention relates to a new class of small-molecules having a phenyl-sulfonic amide (or similar) structure which function as agonists of ML1 protein, and their use as therapeutics for the treatment of disorders related to diminished ML1 activity (e.g., DMD and other muscular dystrophy disorders related to ML1 activity).

Certain phenyl-sulfonic amide (or similar) compounds of the present invention may exist as stereoisomers including optical isomers. The invention includes all stereoisomers, both as pure individual stereoisomer preparations and enriched preparations of each, and both the racemic mixtures of such stereoisomers as well as the individual diastereomers and enantiomers that may be separated according to methods that are well known to those of skill in the art.

In a particular embodiment, compounds encompassed within the following formulas are provided:

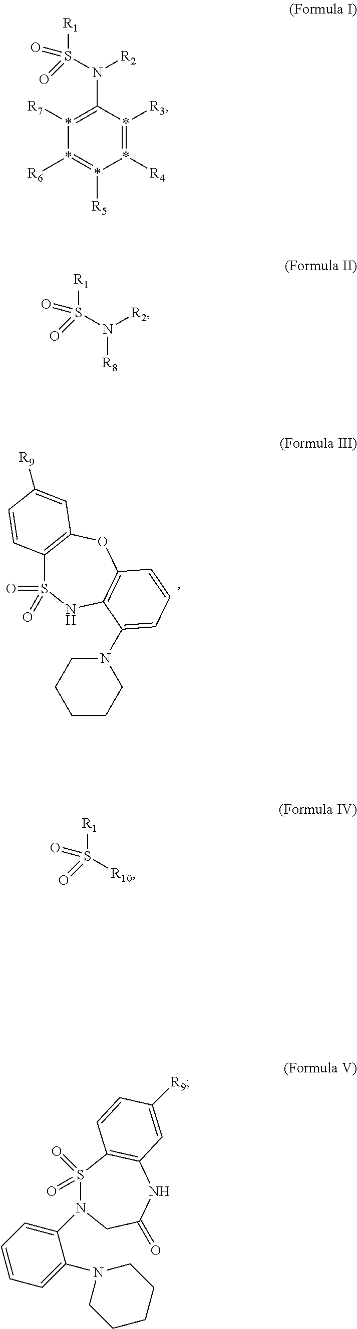

acceptable salts, solvates, and/or prodrugs thereof.

Formula I is not limited to a particular chemical moiety for R1, R2, R3, R4, R5, R6, R7 R8, R9, R10, and *. In some embodiments, the particular chemical moiety for R1, R2, R3, R4, R5, R6, R7 R8, R9, R10, and * independently include any chemical moiety that permits the resulting compound to have one or more of the following properties:

1) a capability to improve aberrant membrane repair capability related to diminished ML1 activity;

2) a capability to improve aberrant lysosomal exocytosis activity related to diminished ML1 activity;

3) a capability to improve aberrant lysosomal Ca²⁺ release channel activity related to diminished ML1 activity (required for lysosomal exocytosis);

4) a capability to repair damaged sarcolemma related to diminished ML1 activity;

5) a capability to improve aberrant Ca²⁺ dependent delivery of lysosomal membranes to damaged sarcolemma related to diminished ML1 activity;

6) a capability to improve aberrant lysosomal activity related to diminished ML1 activity;

7) a capability to repair muscle damage related to diminished ML1 activity;

8) a capability to ameliorate myofiber necrosis related to diminished ML1 activity;

9) a capability to ameliorate central nucleation related to diminished ML1 activity;

10) a capability to ameliorate fibrosis related to diminished ML1 activity;

11) a capability to ameliorate elevated serum creatine kinase (CK) levels related to diminished ML1 activity;

12) a capability to ameliorate reduced muscle force related to diminished ML1 activity;

13) a capability to ameliorate impaired motor ability related to diminished ML1 activity; and

14) a capability to restore lysosome function via activation of transcriptional factor EB (TFEB).

In some embodiments, each “*” is independently either carbon or nitrogen.

In some embodiments, at least one “*” is nitrogen, and the remainder are carbon.

In some embodiments, R1 is selected from hydrogen,

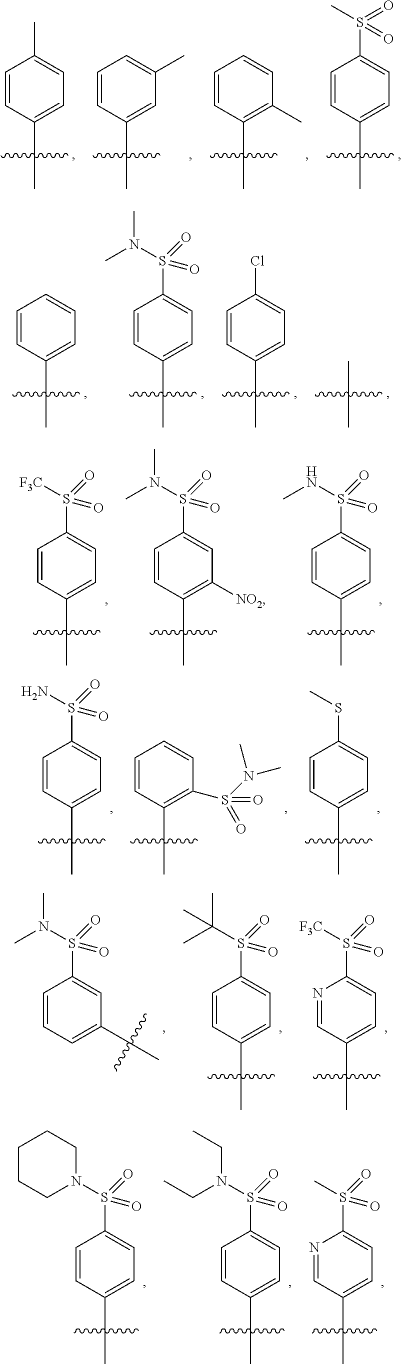

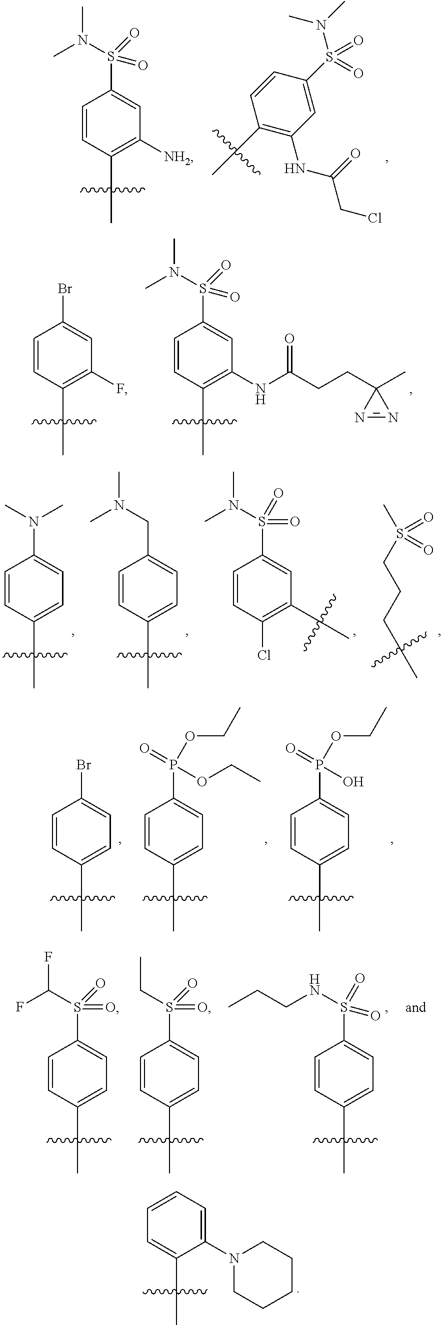

In some embodiments, R2 is selected from hydrogen,

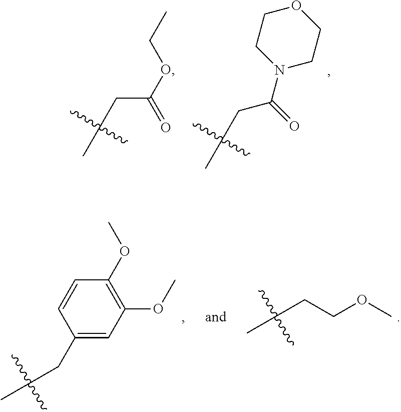

In some embodiments, R3, R4, R5, R6 and R7 are independently selected from hydrogen, Chlorine, Fluorine, CH₃,

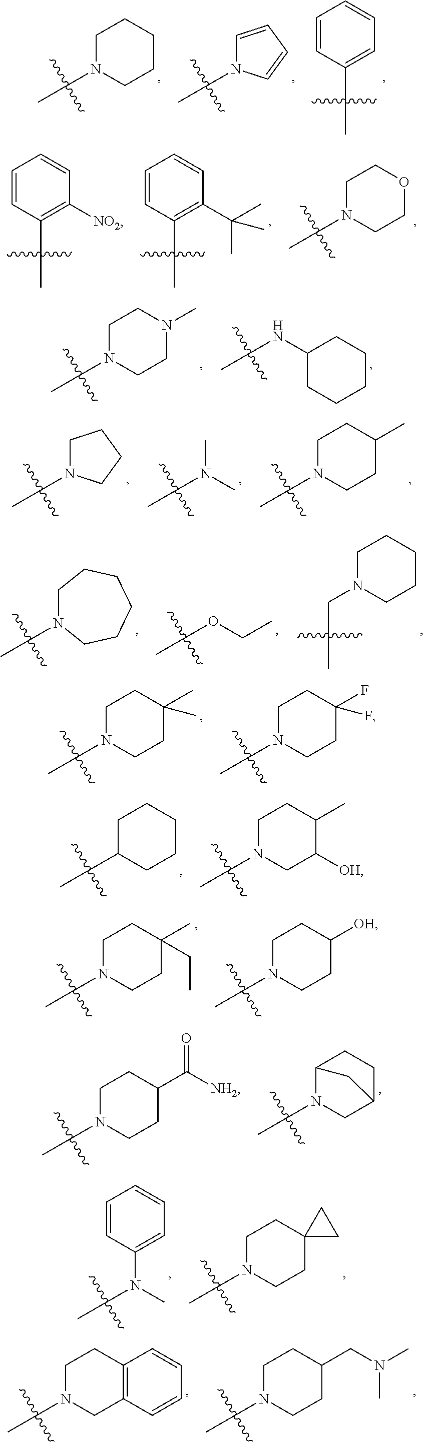

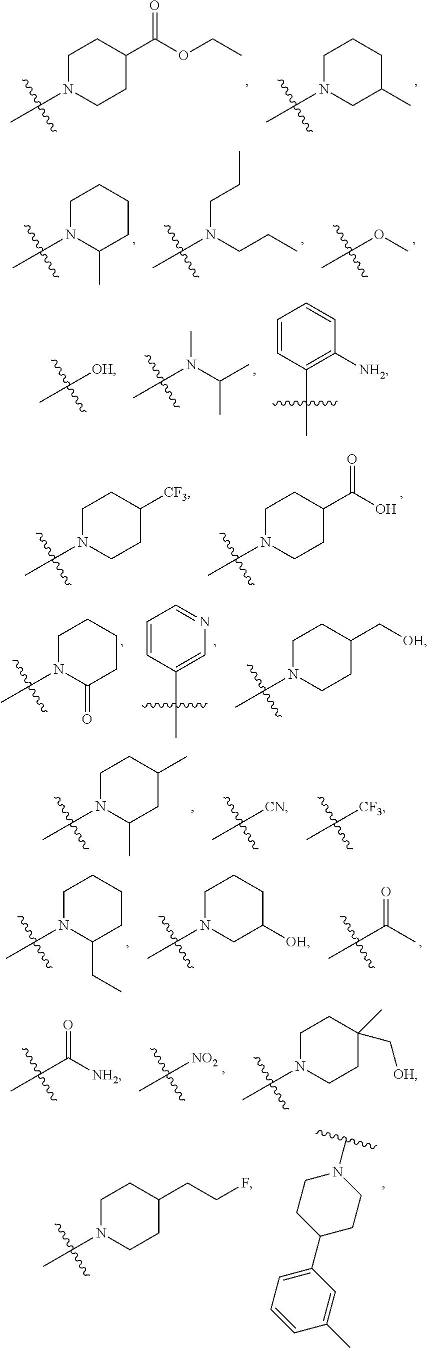

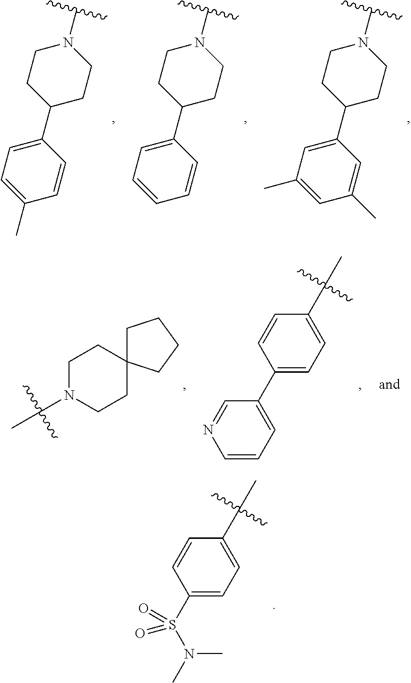 [SMILES: *N1CCC(c2ccc(C)cc2)CC1 ; *N1CCC(c2ccccc2)CC1 ; *N1CCC(c2cc(C)cc(C)c2)CC1 ; *N1CCC2(CCCC2)CC1 ; *c1ccc(-c2cccnc2)cc1 ; *c1ccc(S(=O)(=O)N(C)C)cc1]

In some embodiments, at least two of R3, R4, R5, R6 and R7 are hydrogen.

In some embodiments, at least three of R3, R4, R5, R6 and R7 are hydrogen.

In some embodiments, at least four of R3, R4, R5, R6 and R7 are hydrogen.

In some embodiments, R8 is selected from hydrogen,

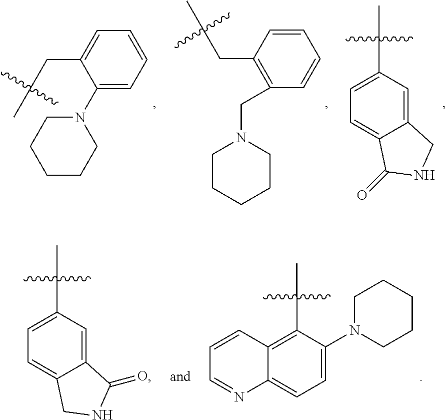

In some embodiments, R9 is selected from hydrogen,

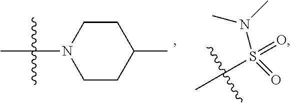

### Bromine,

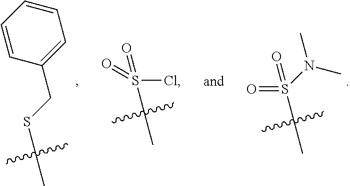

In some embodiments, R10 is selected from hydrogen,

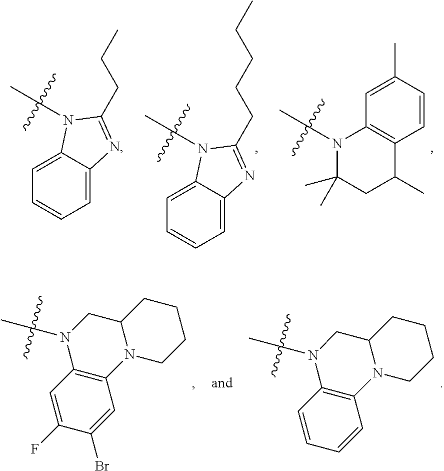

In some embodiments, the compound is one or more of the compounds recited in Examples 1-177.

The invention further provides processes for preparing any of the compounds of the present invention.

The invention also provides pharmaceutical compositions comprising the compounds of the invention in a pharmaceutically acceptable carrier. Indeed, the present invention also provides pharmaceutical compositions comprising one or more of the compounds of the invention, and at least one additive or excipient, e.g., fillers, diluents, binders, disintegrants, buffers, colorants, emulsifiers, flavor-improving agents, gellants, glidants, preservatives, solubilizers, stabilizers, suspending agents, sweeteners, tonicity agents, wetting agents, emulsifiers, dispersing agents, swelling agents, retardants, lubricants, absorbents, and viscosity-increasing agents. The compositions may be presented in capsules, granules, powders, solutions, sachets, suspensions, or tablet dosage form.

The compounds of the invention are useful for the treatment, amelioration, or prevention of disorders associated with diminished ML1 activity (e.g., DMD and other muscular dystrophy disorders related to ML1 activity (e.g., Becker's Muscular Dystrophy (BMD), Limb Girdle Muscular Dystrophy (LGMD), distal muscular dystrophy, facioscapulohumeral dystrophy, myotonic muscular dystrophy, Emery-Dreifuss muscular dystrophy, oculopharyngeal muscular dystrophy, and Congenital Muscular Dystrophy (CMD) (e.g., Laminin-α2-deficient CMD (MDC1A), Ullrich CMG (UCMDs 1, 2 and 3), Walker-Warburg syndrome (WWS), Muscle-eye-brain disease (MEB), Fukuyama CMD (FCMD), CMD plus secondary laminin deficiency 1 (MDC1B), CMD plus secondary laminin deficiency 2 (MDC1C), CMD with mental retardation and pachygyria (MDC1D), and Rigid spine with muscular dystrophy Type 1 (RSMD1)).

In certain embodiments, the compounds can be used to treat, ameliorate, or prevent symptoms related to diminished ML1 activity (e.g., aberrant membrane repair capability related to diminished ML1 activity; aberrant lysosomal exocytosis activity related to diminished ML1 activity; aberrant lysosomal Ca²⁺ release channel activity related to diminished ML1 activity (required for lysosomal exocytosis); damaged sarcolemma related to diminished ML1 activity; aberrant Ca²⁺ dependent delivery of lysosomal membranes to damaged sarcolemma related to diminished ML1 activity; aberrant lysosomal activity related to diminished ML1 activity; muscle damage related to diminished ML1 activity; myofiber necrosis related to diminished ML1 activity; central nucleation related to diminished ML1 activity; fibrosis related to diminished ML1 activity; elevated serum creatine kinase (CK) levels related to diminished ML1 activity; reduced muscle force related to diminished ML1 activity; impaired motor ability related to diminished ML1 activity). In some embodiments, the symptoms are being experienced by a subject (e.g., mammalian subject) (e.g., human subject). In some embodiments, the subject is a human patient suspect of having or having DMD and/or a muscular dystrophy disorder related to ML1 activity (e.g., Becker's Muscular Dystrophy (BMD), Limb Girdle Muscular Dystrophy (LGMD), distal muscular dystrophy, facioscapulohumeral dystrophy, myotonic muscular dystrophy, Emery-Dreifuss muscular dystrophy, oculopharyngeal muscular dystrophy, and Congenital Muscular Dystrophy (CMD) (e.g., Laminin-α2-deficient CMD (MDC1A), Ullrich CMG (UCMDs 1, 2 and 3), Walker-Warburg syndrome (WWS), Muscle-eye-brain disease (MEB), Fukuyama CMD (FCMD), CMD plus secondary laminin deficiency 1 (MDC1B), CMD plus secondary laminin deficiency 2 (MDC1C), CMD with mental retardation and pachygyria (MDC1D), and Rigid spine with muscular dystrophy Type 1 (RSMD1)).

The invention also provides kits comprising a compound of the invention and instructions for administering the compound to an animal. The kits may optionally contain other therapeutic agents, e.g., agents useful in treating DMD and other muscular dystrophy disorders related to ML1 activity.

### BRIEF DESCRIPTION OF THE DRAWINGS

FIG. 1A-K. Muscle-specific transgenic overexpression of ML1. (A) PCR genotyping of the mdx mutation, GCaMP3-ML1 transgene, and MCK-Cre. (B) Western blotting with anti-ML1 antibody in brain and various skeletal muscle tissues, including GAS, TA, and DIA from WT, ML1 ROSA-lSl;MCK Cre (ML1^{MCK}), mdx, and mdx; ML1^{MCK} mice (see FIG. 2B for the source files). GAPDH served as the loading control. (C) Immunofluorescence analysis of TA, GAS, and DIA cryo-sections from various transgenic mice. Scale bar=10 μm. (D) Immunofluorescence analysis of primary myotubes isolated from ML1^{MCK} mice. Scale bar=10 μm. (E) Whole-endolysosome ML1 currents (I_{ML1}) were activated by ML-SA1 (20 μM), a synthetic agonist of ML1, in primary myotubes harvested from WT, ML1^{MCK}, mdx, and mdx; ML1^{MCK} mice. Currents were stimulated with a ramp voltage protocol from −120 to +60 mV. Holding potential=0 mV. (F) I_{ML1} current densities of myotubes from E. Each open circle represents one cell/patch. (G, H) ML-SA5-induced lysosomal Ca²⁺ release, measured with GCaMP3 imaging, in primary myotubes isolated from ML1^{MCK} mice. GPN (glycyl-L-phenylalanine 2-naphthylamide), a dipeptide causing osmotic lysis of lysosomes, was used as a negative (depleting lysosomal Ca²⁺ stores) control. (I) Representative images showing H&E staining of GAS isolated from 2-month-old ML1^{-/-} and ML1^{-/-};ML1^{MCK} mice. Arrows label necrotic areas and asterisks show central-nucleated myofibers. Scale bar=100 μm. (J, K) Quantification of necrosis (J) and central-nucleation (K) in GAS sections from 2-month-old ML1^{-/-} and ML1^{-/-};ML1^{MCK} mice. All data are means±s.e.m; *p<0.05, **p<0.01, ***p<0.001.

FIG. 2A-E. Muscle-specific transgenic overexpression of ML1. (A) The GCaMP3-ML1 transgene was inserted into the ROSA26 locus with a loxSTOPlox cassette. The transgenic mice were crossed with MCK-Cre mice to achieve muscle-specific overexpression of ML1. (B) Source files for FIG. 1B showing western blotting with anti-ML1 antibody in brain and various skeletal muscle tissues, including GAS, TA, and DIA from WT, ML1 ROSA-lSl;MCK Cre (ML1^{MCK}), mdx, and mdx; ML1^{MCK} mice. (C) Western blotting analysis of GCaMP3-ML1 expression in ML1^{MCK} DIA and cardiac muscle with anti-GFP antibody. (D, E) Quantification of lysosomal Ca²⁺ release from ML1^{MCK} and mdx;ML1^{MCK} myotubes (see FIG. 1G, H). All data are means±s.e.m.; **p<0.01; ****p<0.0001.

FIG. 3A-H. Transgenic overexpression of ML1 reduces muscle pathologies in young mdx mice. (A) H&E staining of TA sections from WT, ML1^{MCK}, mdx, and mdx;ML1^{MCK} mice at the age of 1 month (mo.), before (rest) and after treadmill exercise. Both male and female mice were randomly assigned into different groups. Arrows label necrotic areas and asterisks show central-nucleated myofibers. Scale bar=500 μm or 50 μm (zoom-in images). (B) Percentage of necrotic area in TA muscles from various transgenic mice. Each datum (n indicates the number of the muscle) represents the averaged result from at least five representative images randomly selected from at least three sections. Statistical analyses were performed by experimenters who were blind to animal genotypes. (C) Percentage of central-nucleated fibers in TA muscles from different transgenic mice. (D) Serum CK levels in 1-month old WT, ML1^{MCK}, mdx, and mdx;ML1^{MCK} mice before and after treadmill exercise. (E) Specific force test of GAS from multiple 1-mo.-old mice (F) Body weight measurements of WT, utrophin^{-/-};mdx (utrn^{-/-};mdx), and utrn;mdx;ML1^{MCK} male mice at the age of 1 month. (G) The effect of ML1 overexpression on histology of TA muscles isolated from 2-month-old utrn; mdx mice. Scale=50 μm. (H) Quantification on central-nucleation of muscle histology from G. All data are means s.e.m; *p<0.05, **p<0.01, and ***p<0.001.

FIG. 4A-G. Muscle-specific transgenic overexpression of ML1 reduces GAS and DIA pathologies in mdx mice. (A, B) H&E staining of GAS from 1-month-old mdx and mdx;ML1^{MCK} mice under low (A) and high (B) magnification. Scale=500 m (A) or 50 μm (B). Both male and female mice were used. (C, D) Quantification of necrosis (C) and central-nucleation (D) of representative H&E stained images as shown in A and B. Each datum (n indicates the number of the muscle) represents the averaged result from at least three representative images randomly selected from at least three sections. Statistical analyses were performed by experimenters who were blind to animal genotypes. (E) H&E staining of DIA. Scale=50 μm. (F, G) Quantification of necrotic and central-nucleated fibers as shown in E. All data are means s.e.m.; *p<0.05, **p<0.01.

FIG. 5A-J. Transgenic ML1 overexpression ameliorates myopathies in aged mdx mice. (A) H&E staining of DIA isolated from mdx and mdx;ML1^{MCK} mice at the age of 1 mo., 4 mos. and 10 mos.. Both male and female mice were used in this experiment. Scale bar=50 μm. (B) Age-dependent progressive fibrosis in DIA muscles isolated from mdx and mdx;ML1^{MCK} mice. (C) Trichrome collagen staining of DIA from 4-mo.-old mice. Scale bar=100 μm. (D) Quantification of results (n indicates the number of the animal) averaged from multiple randomly selected images as shown in C. (E, F) Thickness of interventricular septum (IVS) was measured by echocardiography (see FIG. 6A) at the end diastole (E) and end systole (F) from 13-15-month-old WT, mdx, mdx;ML1^{MCK} male mice. The echocardiographer was blind to the mouse genotype. (G) Calculated left ventricle mass in 13-15 months old WT, mdx, mdx;ML1^{MCK} male mice. (H) Peak velocity of E wave measured by left ventricular Pulse wave Doppler (see FIG. 6B) in 13-15 months old WT, mdx, mdx;ML1^{MCK} male mice. (I) The ratios between peak velocity of E and A waves (see FIG. 6B) in WT, mdx, mdx;ML1^{MCK} male mice. (J) Quantification for the percentage of fibrotic area in WT, mdx, and mdx;ML1^{MCK} mice at the age of 13-15 months (see FIG. 6C). All data are means±s.e.m.; *p<0.05, **p<0.01, and *** p<0.001.

FIG. 6A-C. Diagrams of echocardiography. (A) A diagram demonstrating how the left ventricle echocardiograph was performed and quantified. IVS: interventricular septum; d: diastole; s: systole; LVID: left ventricular internal diameter; LVPW: left ventricular posterior wall. (B) Mitral valve E and A waves were measured by left ventricular Pulse wave (PW) Doppler. (C) H&E staining of cardiac muscle sections from 15 months old mdx and mdx;ML1^{MCK} mice. Only male mdx mice were used for echocardiography, but both male and female mice were included in the histology analysis. Scale=500 μm (low magnification) or 50 μm (high magnification).

FIG. 7A-I. Small-molecule ML1 agonists ameliorate muscular dystrophies in mdx mice. (A) Structure of ML-SA5. (B) ML-SA1 and ML-SA5 dose-dependently activated whole-endolysosomal ML1 currents in DMD myoblasts. (C) H&E staining of TA from 1-month-old mdx mice that received daily i.p. injection of ML-SA5 (2 mg/kg) for 14 d starting at P14. Arrows and asterisks indicate necrotic areas and central-nucleated fibers, respectively. Scale bar=500 or 50 μm (zoom-in). (D) Percentages of necrotic area in ML-SA5-injected mice. Each datum (n indicates the number of the muscle; ≥4) represents the averaged result from at least three representative images randomly selected from at least three sections. Statistical analyses were performed by experimenters who were blind to treatment conditions. (E) Percentage of centrally-nucleated fibers in ML-SA5-injected mice. (F) Serum CK levels in ML-SA5-treated mdx mice at the age of 1 mo. before and after treadmill exercise. (G, H) Effect of ML-SA5 i.p. injection on TA from 2-month-old ML1 KO mice. Injection starts from P14. (I) Treadmill exhaustion time of mdx mice treated with ML-SA5 vs. vehicle control. N, number of tested animals. The experimenter was blind to the treatment conditions. In the experiments shown in this figure, both male and female mice were randomly assigned into different treatment groups. All data are means±s.e.m.; *p<0.05, **p<0.01, and *** p<0.001.

FIG. 8. Pharmacokinetics of ML-SA5. Plasma concentrations of ML-SA5 at different time points (0, 0.5, 1, 2, 4, and 8h) following i.p. injection (5 mg/kg) into mdx mice (N=6 animals).

FIG. 9A-J. Toxicity analysis of ML-SA5. (A) Effects of i.p. injection of ML-SA5 and rapamycin on animal body weight. N indicates the number of tested animals in each group. Growth rate=(weight after injection−weight before injection)/weight before injection. (B-E) Complete blood count for white blood cells (B), hemoglobin (C), mean corpuscular volume (D), and platelets (E). Blood was collected from PBS-, vehicle-, and ML-SA5 (2 mg/kg)-injected mdx mice at the age of 2 mos.. Daily injection started from P14. Both male and female mice were randomly assigned into different treatment groups. (F) Liver histology of vehicle- and ML-SA5-injected mdx mice. (G, H) Liver biochemistry measuring the levels of serum aspartate aminotransferase (AST, G) and alanine aminotransferase (ALT, H). Serum was collected from mdx mice at the age of 2 mos.. (I) Kidney histology of vehicle- and ML-SA5-injected mdx mice. (J) Effects of ML-SA5 (2 mg/kg) i.p. injection on muscle histology in WT mice. All data are mean±s.e.m.; ***p<0.001.

FIG. 10A-F. ML1 agonist injection (i.p.) ameliorates GAS and DIA pathologies in mdx mice. (A) H&E staining of GAS from mice given daily i.p. injection of ML-SA5 (2 mg/kg) for 14 d. Scale bar=50 μm. (B, C) Quantification of necrotic (B) and centrally-nucleated fibers (C) in ML-SA5-injected GAS. (D-F) H&E staining of DIA from ML-SA5-injected mdx mice. Male and female mice were randomly assigned to different treatment groups in experiments shown in this figure. Scale bar=50 μm. All data are mean±s.e.m.; *p<0.05, and **p<0.01.

FIG. 11A-E. Inhibition of ML1 activity exacerbates myopathology in mdx mice. (A) ML-SA5 (0.5 μM)-activated whole-endolysosome ML1 currents were sensitive to the synthetic inhibitors of ML1, ML-SI3 (10, 30 μM), and ML-SI6 (1 μM) in DMD myoblasts. (B) H&E staining of TA from 1-month-old mdx mice given daily i.p. injection of ML-SI6 (2 mg/kg) for 14 d. Scale=500 μm or 20 μm (zoom in images). (C) Quantification of necrotic area of sections in B. (D, E) EB dye uptake in TA from ML-S16-injected mdx mice. Scale=100 μm. Male and female mice were randomly assigned to different treatment groups in experiments shown in this figure. All data are means s.e.m.; *p<0.05.

FIG. 12A-I. ML1 activation facilitates sarcolemma repair to reduce muscle damage in mdx mice. (A) Representative images of EB dye uptake in GAS isolated from WT, mdx, and mdx;ML1^{MCK} mice at the age of 1 mo., before (rest) and after treadmill exercise. Scale bar=10 μm. (B) Quantification of EB-positive fibers from panel A. Each datum (n, number of the muscle) represents the averaged result from at least five representative images selected from at least three sections. (C) EB dye uptake in GAS isolated from ML-SA5-treated mdx mice. Scale bar=10 μm. (D) Quantification of EB dye uptake in GAS from ML-SA5-treated mdx mice. (E, F) EB dye uptake in cardiac muscles isolated from 2-month-old, ML-SA5 (2 mg/kg)-injected mdx mice that were stimulated with β-isoproterenol to cause cardiac damage. Injection of ML-SA5 started from P14. Scale=500 μm. (G) Muscle force deficit of mechanically-stretched GAS from 1-mo.-old mice. (H) Representative images of laser damage-induced FM dye accumulation in isolated FDB fibers. Arrows highlight damage sites. Scale=20 μm. (I) Time-dependent laser damage-induced FM dye accumulation in FDB fibers isolated from WT, mdx, and mdx;ML1^{MCK} mice at the age of 1 mo.. N indicates the number of the FDB fibers for each genotype. In all the experiments shown in this figure, both male and female mice were randomly assigned into different experimental groups. All data are means s.e.m.; *p<0.05, **p<0.01, and ***p<0.001.

FIG. 13A-D. Transgenic overexpression or pharmacological activation of ML1 facilitates sarcolemma repair in mdx mice. (A, B) Transgenic overexpression of ML1 decreased EB dye uptake in DIA from mdx mice at the age of 1 mo.. Scale=100 μm. Both male and female mice were used. (C, D) ML-SA5 (2, 5 mg/kg) treatment decreased EB-positive fibers before and after treadmill exercise. Scale=10 μm. All data are means±s.e.m.; *p<0.05, **p<0.01, and ***p<0.001.

FIG. 14A-I. ML1 agonist activates TFEB to correct lysosomal insufficiency in mdx muscles. (A) Lamp1 immunofluorescence staining of GAS from WT, ML1^{MCK}, mdx, and mdx;ML1^{MCK} mice at the age of 1 mo.. Scale bar=100 μm. Both male and female mice were used in the experiments shown in this figure. (B) Quantitative analyses of images in A. For each datum representing each muscle, at least five representative images from at least three sections were analyzed. (C) Western blotting analysis of Lamp1 protein expression in GAS and DIA from 1-month-old mice. (D) Quantitation of western blotting results in panel C. (E) TFEB immunolabeling in GAS from ML-SA5 treated mice at the age of 1 mo.. (F) Quantitative analysis of nuclear vs. total TFEB ratio from G. N indicates muscle fibers in randomly selected images from at least four muscles in each group. (G) Lamp1 immunostaining in GAS from ML-SA5 (2 mg/kg)-treated mdx mice at the age of 1 mo.. Scale bar=100 μm. (H) Quantification of Lamp1 immunolabeling in GAS from ML-SA5-treated mdx mice. For each datum representing each muscle, at least five representative images from at least three sections were analyzed. (I) Genetic or pharmacological upregulation of ML1 ameliorates myopathies in vivo through TFEB-dependent lysosome biogenesis, Ca²⁺-dependent lysosomal exocytosis, and sarcolemma repair. Muscle damage may generate a yet-to-be-defined signal (e.g., reactive oxygen species (18)) to activate ML1 channels on lysosome membranes. Subsequent lysosomal Ca²⁺ release triggers Ca²⁺-dependent lysosomal exocytosis and nuclear translocation of TFEB, which then activates transcription of a unique set of genes related to lysosome biogenesis. Subsequently, lysosome function is boosted and sarcolemma repair may be facilitated to reduce muscle damage in DMD cells in vitro and in vivo. All data are means±s.e.m.; *p<0.05, and **p<0.01.

FIG. 15A-E. ML1 activation alleviates lysosomal and autophagic insufficiency in mdx mice. (A) Lamp1 expression was high in CD11b-positive macrophages. Scale bar=100 μm. (B, C) TFEB immunolabeling in GAS from WT, ML1^{MCK}, mdx, and mdx;ML1^{MCK} mice. Scale bar=m. Each datum represents one muscle fiber in randomly selected images from at least four animals in each group. (D) Treatment of 2 mg/kg ML-SA5 for 14 d decreased TFEB intensity (see FIG. 6G). (E) Western blotting analysis of LC3-II expression levels in GAS from various transgenic mice. All data are means s.e.m.; ****p<0.0001.

FIG. 16A-K. ML1 agonist activates TFEB/TFE3 and lysosomal biogenesis in DMD myoblasts. (A) Dystrophin immunolabeling in myotubes differentiated from immortalized WT and DMD human myoblasts. Scale bar=10 μm. (B) Western blotting analysis of dystrophin expression in WT and DMD myoblasts. (C) Lamp1 immunostaining in WT and DMD cells after ML-SA5 (0.5 μM) treatment. Scale bar=10 μm. (D) TFEB immunolabeling of WT and DMD myoblasts upon ML-SA5 (0.5 μM) and ML-SI3 (20 μM) treatment. Scale=10 μm. (E) Quantification of TFEB fluorescence intensity in nucleus divided by that in cytosol. (F, G) Images (F) and quantification (G) for TFE3 immunolabeling of WT and DMD myoblasts upon ML-SA5 (0.5 μM) and ML-SI3 (20 μM) treatment. Scale=10 μm. (H) mRNA expression levels of TFEB target genes determined by qPCR. HPRT was used as a control. (I-K) Lamp1 and TFEB protein levels in WT and DMD myoblasts were increased and decreased, respectively, upon ML-SA5 (0.5 μM) treatment for 12 h and 24 h. All data are means±s.e.m.; *p<0.05, **p<0.01, and ****p<0.0001.

FIG. 17A-E. Sarcolemmal Ca²⁺ entry is not required for ML-SA-induced TFEB nuclear translocation. (A) GCaMP3 Ca²⁺ imaging studies of isolated FDB single fibers from ML1^{MCK} mice. ML-SA1 (30 μM) induced Ca²⁺ increases in both sub-sarcolemmal regions (red) and cytoplasm (blue). Note that Lamp1-positive lysosomes are known to accumulate in the sub-sarcolemmal regions as well. Scale=50 μm. (B) ML-SA5-induced Ca²⁺ responses in mdx;ML1^{MCK} FDB single fibers in 0 vs. 2 mM Ca²⁺ Tyrode's solutions. (C) Quantification for ML-SA5-induced Ca²⁺ increase in 0 vs. 2 mM Ca²⁺ solutions in mdx;ML1^{MCK} FDB fibers. (D) ML-SA5 induced TFEB nuclear translocation in the presence of 3 mM La³⁺, a membrane-impermeable TRPML blocker (45). Scale bar=10 μm. (E) Quantification for TFEB fluorescence intensity from D. All data are means±s.e.m.; ****p<0.0001.

FIG. 18A-B. Membrane damage and activation of ML1 trigger lysosomal exocytosis in DMD myotubes. (A, B) Lamp1 surface immunolabeling of DMD differentiated myotubes (A) and myoblasts (B) after ML-SA5 (0.5 μM) pretreatment and SLO (1 μg/mL, 30 min) challenge. Surface expression of Lamp1 was assayed with an anti-Lamp1 antibody that recognizes a luminal epitope in non-permeabilized cells. Scale=10 μm.

FIG. 19A-H. ML1 agonist prevents cell death in DMD myoblasts via TFEB. (A) Flow cytometric analysis of the viability of cultured muscle cells, assayed by PI staining following SLO toxin-induced membrane damage. Human DMD myoblasts were pre-treated with ML-SA5 (0.5 μM) and ML-SI3 (20 μM) for 7 h before being subjected to SLO toxin (0.5-1 μg/ml, 10 min) challenge. (B) Quantification of five independent repeats as shown in A. (C, D) PI-staining-based flow cytometry of the viability of cultured WT myoblasts pretreated with ML-SI3 (20 μM) upon membrane damage by SLO toxin. (E) TFEB-specific siRNA decreased TFEB protein expression in DMD cells. (F-H) TFEB knockdown by siRNA blocked ML-SA5 effects on cell survival. Rescue percentage is calculated by the difference between the percentages of DMSO- and ML-SA5-treated cell death induced by SLO challenge divided by the percentage of DMSO-treated, SLO-induced cell death. All data are means±s.e.m.; *p<0.05, and **p<0.01.

FIG. 20A-B. Lysosomal exocytosis is required for ML-SA5-induced sarcolemma repair and cell survival. (A, B) Flow cytometric analysis of DMD myoblasts transfected with a Syt-VII dominant-negative construct to block lysosomal exocytosis. DMD differentiated myotubes were pre-treated with ML-SA5 (0.5 μM) and challenged with SLO (1 μg/mL, 30 min). Data are means±s.e.m.; **p<0.01.

FIG. 21A-E. Requirement of continuous agonist administration in achieving muscle protective effects. (A, B) The effects of ML-SA5 washout on TFEB nuclear translocation. Scale=10 μm. (C) H&E staining of TA muscle sections from mdx mice that were treated with ML-SA5 at P14 for one week followed by drug withdrawal for another week. Mice were sacrificed at the age of 1 mo.. (D, E) Quantifications of necrotic area (D) and central-nucleation (E) in the muscle sections shown in C. All data are means±s.e.m.; ****p<0.0001.

### DETAILED DESCRIPTION OF THE INVENTION

Duchenne muscular dystrophy (DMD), an X-linked inherited muscle disease (see, Mercuri, E. & Muntoni, F. Muscular dystrophies. Lancet 381, 845-860 (2013)), is caused by loss-of-function mutations affecting dystrophin, a large cytoplasmic protein that connects the cytoskeleton with extracellular matrix proteins via the muscle membrane (sarcolemma) (see, Davies, K. E. & Nowak, K. J. Nat Rev Mol Cell Biol 7, 762-773 (2006); Rahimov, F. & Kunkel, L. M. J Cell Biol 201, 499-510 (2013)). The fragile sarcolemma that makes human DMD muscles prone to contraction-induced muscle damage has been mimicked in the striated muscles of the mdx mouse, a murine model of DMD (see, Allen, D. G., Whitehead, N. P. & Froehner, S. C. Physiol Rev 96, 253-305 (2016); Campbell, K. P. Cell 80, 675-679 (1995); Stedman, H. H. et al. Nature 352, 536-539 (1991)). Although gene therapy approaches, including CRISPR technology (see, Amoasii, L. et al. Sci Transl Med 9 (2017)), have provided hope for a potential treatment approach for some specific variants of DMD, the existence of thousands of DMD-causing dystrophin mutations remains a major challenge (see, Mercuri, E. & Muntoni, F. Lancet 381, 845-860 (2013)). To develop a therapeutic approach that would be more broadly applicable to all forms of DMD, it is necessary to understand the common pathological mechanisms underlying the condition (see, Allen, D. G., Whitehead, N. P. & Froehner, S. C. Physiol Rev 96, 253-305 (2016)).

Muscle cells are especially sensitive to membrane damage, and recent studies have identified impairment of membrane repair capability as an important cause of muscular dystrophies (see, Bansal, D. et al. Defective membrane repair in dysferlin-deficient muscular dystrophy. Nature 423, 168-172 (2003); Cai, C. et al. Nat Cell Biol 11, 56-64 (2009); McNeil, P. Nat Cell Biol 11, 7-9 (2009)). For instance, it was found recently that mice lacking transient receptor potential mucolipin 1 (ML1), a lysosomal Ca²⁺ release channel required for lysosomal exocytosis (see, Dong, X. P. et al. Nat Commun 1, 38 (2010); Shen, D. et al. Nat Commun 3, 731 (2012)), display early-onset, progressive dystrophies similar to DMD (see, Cheng, X. et al. Nat Med 20, 1187-1192 (2014)). When ML1 is pharmacologically inhibited or genetically inactivated, membrane resealing is impaired in skeletal muscle (see, Cheng, X. et al. Nat Med 20, 1187-1192 (2014)). Hence, lysosomes may provide a major source of membranes for repairing damaged sarcolemma, and ML1 is essential for Ca²⁺-dependent delivery of lysosomal membranes (i.e., lysosomal exocytosis) to damaged sites (see, Cheng, X. et al. Nat Med 20, 1187-1192 (2014)). Studies of other muscle diseases have also connected lysosomal dysfunction with muscle pathogenesis (see, Spampanato, C. et al. EMBO Mol Med 5, 691-706 (2013)).

Experiments conducted during the course of developing embodiments for the present invention examined whether upregulation of ML1, by genetic or pharmacological methods, is sufficient to increase lysosomal functions and sarcolemma repair. If so, ML1 may be a putative target for amelioration of muscle damage in vivo. Such experiments demonstrated that transgenic overexpression or pharmacological activation of ML1 in vivo facilitates sarcolemma repair and alleviates the dystrophic phenotype in both skeletal and cardiac muscles of mdx mice (a mouse model of DMD) (see, Clarke, M. S., Khakee, R. & McNeil, P. L. J Cell Sci 106 (Pt 1), 121-133 (1993)). Hallmark dystrophic features of DMD, including myofiber necrosis, central nucleation, fibrosis, elevated serum creatine kinase (CK) levels, reduced muscle force, and impaired motor ability, were all ameliorated by increasing ML1 activity. Considering that lysosome insufficiency occurs in DMD muscle, upregulation of ML1 restores lysosome function via activation of transcriptional factor EB (TFEB), leading to diminished muscle damage. Hence, such experiments indicate that manipulating lysosome function by targeting lysosomal Ca²⁺ channels represents a promising approach to treating DMD and related muscle diseases. In addition, experiments conducted during the course of developing embodiments for the present invention designed and synthesized ML1 agonists having a phenyl-sulfonic amide (or similar) structure.

As such, the present invention addresses the need for effective therapies for DMD and related muscular dystrophy disorders related to diminished ML1 activity by providing potent and selective ML1 agonists able to increase ML1 activity and ameliorate symptoms related to DMD and related disorders.

Accordingly, this invention relates to a new class of small-molecules having a phenyl-sulfonic amide (or similar) structure which function as agonists of ML1, and their use as therapeutics for the treatment of DMD and related disorders.

In a particular embodiment, compounds encompassed within the following formulas are provided:

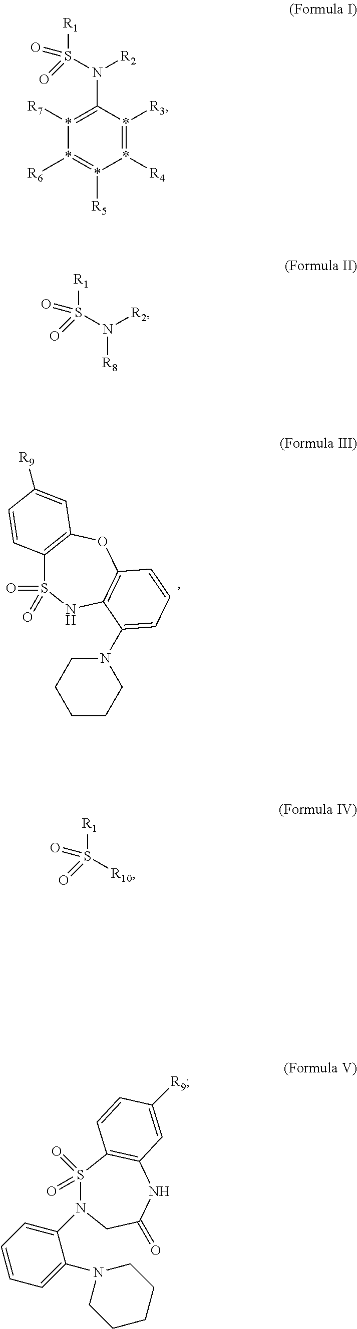

including pharmaceutically acceptable salts, solvates, and/or prodrugs thereof.

Formula I is not limited to a particular chemical moiety for R1, R2, R3, R4, R5, R6, R7 R8, R9, R10, and *. In some embodiments, the particular chemical moiety for R1, R2, R3, R4, R5, R6, R7 R8, R9, R10, and * independently include any chemical moiety that permits the resulting compound to have one or more of the following properties:

1) a capability to improve aberrant membrane repair capability related to diminished ML1 activity;

2) a capability to improve aberrant lysosomal exocytosis activity related to diminished ML1 activity;

3) a capability to improve aberrant lysosomal Ca²⁺ release channel activity related to diminished ML1 activity (required for lysosomal exocytosis);

4) a capability to repair damaged sarcolemma related to diminished ML1 activity;

5) a capability to improve aberrant Ca²⁺ dependent delivery of lysosomal membranes to damaged sarcolemma related to diminished ML1 activity;

6) a capability to improve aberrant lysosomal activity related to diminished ML1 activity;

7) a capability to repair muscle damage related to diminished ML1 activity;

8) a capability to ameliorate myofiber necrosis related to diminished ML1 activity;

9) a capability to ameliorate central nucleation related to diminished ML1 activity;

10) a capability to ameliorate fibrosis related to diminished ML1 activity;

11) a capability to ameliorate elevated serum creatine kinase (CK) levels related to diminished ML1 activity;

12) a capability to ameliorate reduced muscle force related to diminished ML1 activity;

13) a capability to ameliorate impaired motor ability related to diminished ML1 activity; and

14) a capability to restore lysosome function via activation of transcriptional factor EB (TFEB).

In some embodiments, each “*” is independently either carbon or nitrogen.

In some embodiments, at least one “*” is nitrogen, and the remainder are carbon.

In some embodiments, R1 is selected from hydrogen

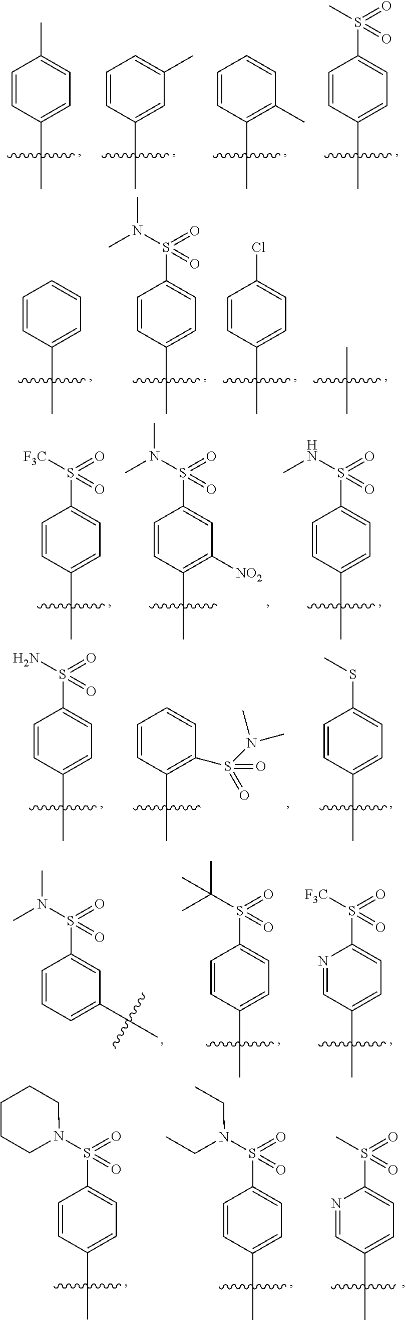

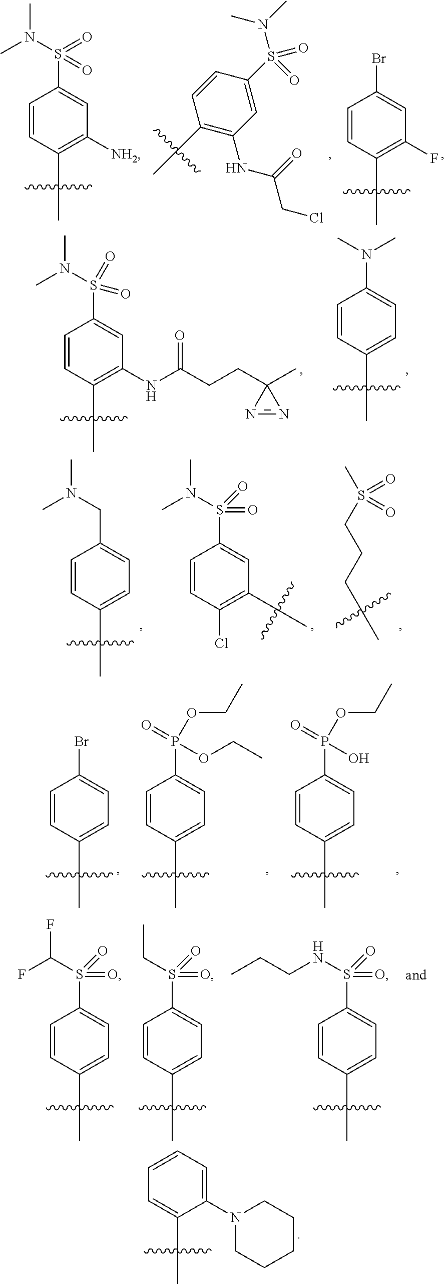

In some embodiments, R2 is selected from hydrogen,

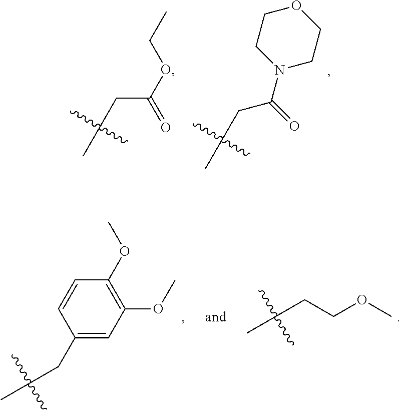

In some embodiments, R3, R4, R5 R6 and R7 are independently selected from hydrogen, Chlorine, Fluorine, CH₃,

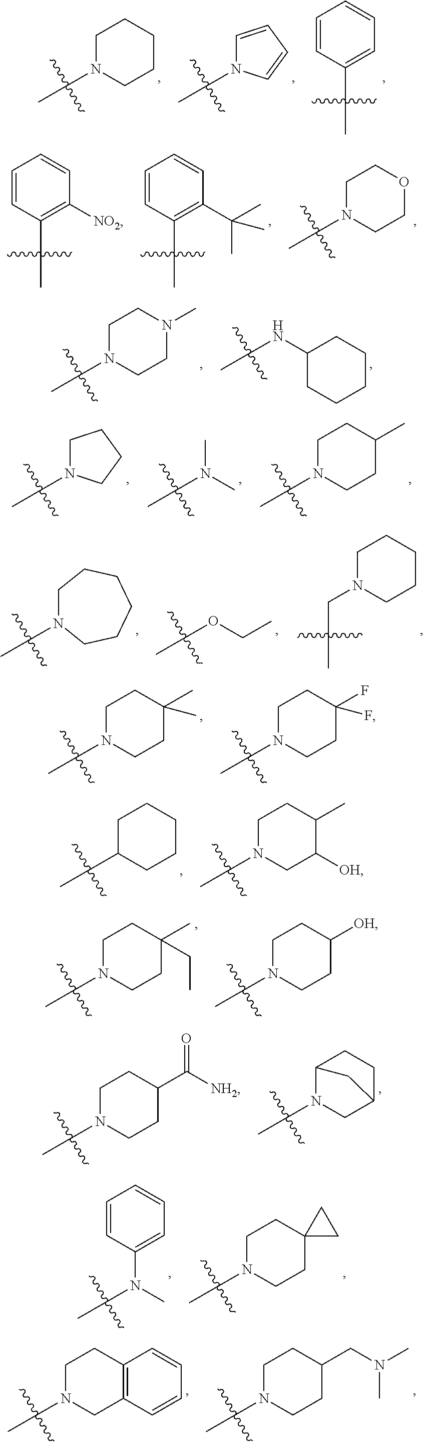

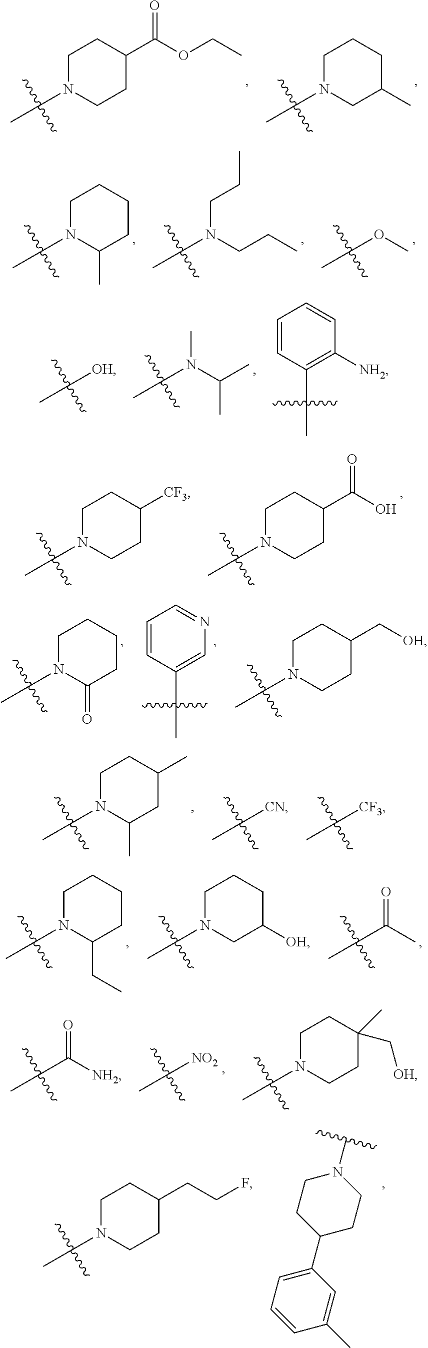

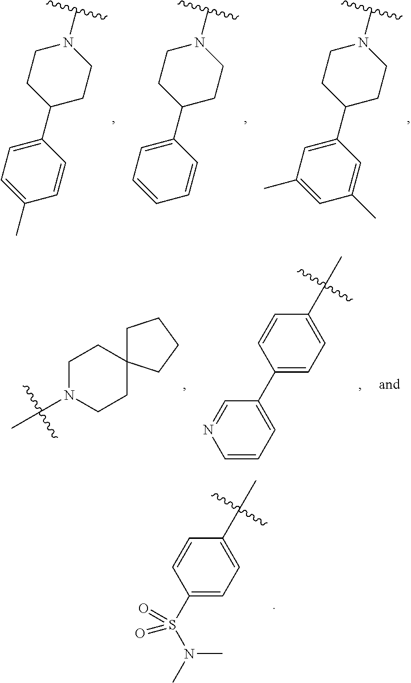 [SMILES: *N1CCC(c2ccc(C)cc2)CC1 ; *N1CCC(c2ccccc2)CC1 ; *N1CCC(c2cc(C)cc(C)c2)CC1 ; *N1CCC2(CCCC2)CC1 ; *c1ccc(-c2cccnc2)cc1 ; *c1ccc(S(=O)(=O)N(C)C)cc1]

In some embodiments, at least two of R3, R4, R5, R6 and R7 are hydrogen.

In some embodiments, at least three of R3, R4, R5, R6 and R7 are hydrogen.

In some embodiments, at least four of R3, R4, R5, R6 and R7 are hydrogen.

In some embodiments, R8 is selected from hydrogen,

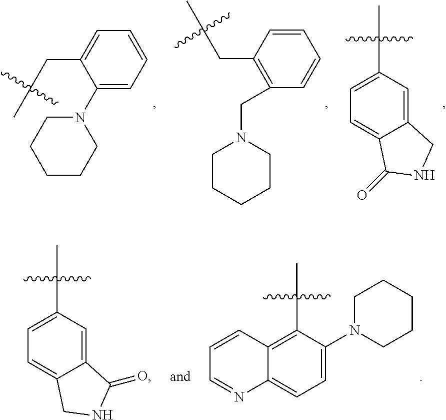

In some embodiments, R9 is selected from hydrogen,

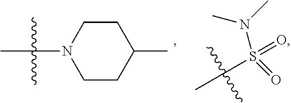

### Bromine,

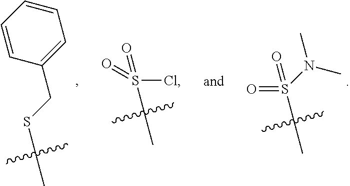

In some embodiments, R10 is selected from hydrogen,

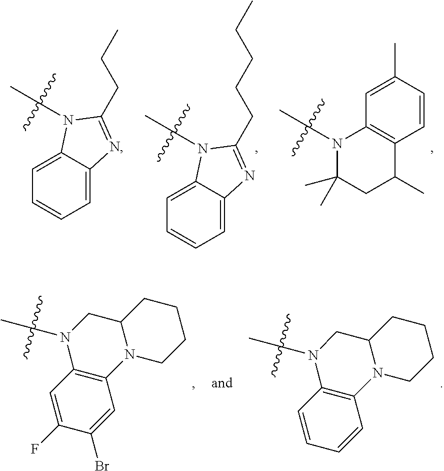

In some embodiments, the compound is one or more of the compounds recited in Examples 1-177.

The invention further provides processes for preparing any of the compounds of the present invention.

In some embodiments, the compositions and methods of the present invention are used to treat diseased cells, tissues, organs, or pathological conditions and/or disease states in an animal (e.g., a mammalian patient including, but not limited to, humans and veterinary animals). In this regard, various diseases and pathologies are amenable to treatment or prophylaxis using the present methods and compositions. A non-limiting exemplary list of these diseases and conditions includes, but is not limited to, conditions characterized with diminished ML1 activity (e.g., DMD and related muscular dystrophy disorders (e.g., Becker's Muscular Dystrophy (BMD), Limb Girdle Muscular Dystrophy (LGMD), distal muscular dystrophy, facioscapulohumeral dystrophy, myotonic muscular dystrophy, Emery-Dreifuss muscular dystrophy, oculopharyngeal muscular dystrophy, and Congenital Muscular Dystrophy (CMD) (e.g., Laminin-α2-deficient CMD (MDC1A), Ullrich CMG (UCMDs 1, 2 and 3), Walker-Warburg syndrome (WWS), Muscle-eye-brain disease (MEB), Fukuyama CMD (FCMD), CMD plus secondary laminin deficiency 1 (MDC1B), CMD plus secondary laminin deficiency 2 (MDC1C), CMD with mental retardation and pachygyria (MDC1D), and Rigid spine with muscular dystrophy Type 1 (RSMD1)).

In certain embodiments, the compounds can be used to treat, ameliorate, or prevent symptoms related to diminished ML1 activity (e.g., aberrant membrane repair capability related to diminished ML1 activity; aberrant lysosomal exocytosis activity related to diminished ML1 activity; aberrant lysosomal Ca²⁺ release channel activity related to diminished ML1 activity (required for lysosomal exocytosis); damaged sarcolemma related to diminished ML1 activity; aberrant Ca²⁺ dependent delivery of lysosomal membranes to damaged sarcolemma related to diminished ML1 activity; aberrant lysosomal activity related to diminished ML1 activity; muscle damage related to diminished ML1 activity; myofiber necrosis related to diminished ML1 activity; central nucleation related to diminished ML1 activity; fibrosis related to diminished ML1 activity; elevated serum creatine kinase (CK) levels related to diminished ML1 activity; reduced muscle force related to diminished ML1 activity; impaired motor ability related to diminished ML1 activity). In some embodiments, the symptoms are being experienced by a subject (e.g., mammalian subject) (e.g., human subject). In some embodiments, the subject is a human patient suspect of having or having DMD and/or a muscular dystrophy disorder related to ML1 activity (e.g., Becker's Muscular Dystrophy (BMD), Limb Girdle Muscular Dystrophy (LGMD), distal muscular dystrophy, facioscapulohumeral dystrophy, myotonic muscular dystrophy, Emery-Dreifuss muscular dystrophy, oculopharyngeal muscular dystrophy, and Congenital Muscular Dystrophy (CMD) (e.g., Laminin-α2-deficient CMD (MDC1A), Ullrich CMG (UCMDs 1, 2 and 3), Walker-Warburg syndrome (WWS), Muscle-eye-brain disease (MEB), Fukuyama CMD (FCMD), CMD plus secondary laminin deficiency 1 (MDC1B), CMD plus secondary laminin deficiency 2 (MDC1C), CMD with mental retardation and pachygyria (MDC1D), and Rigid spine with muscular dystrophy Type 1 (RSMD1)).

Some embodiments of the present invention provide methods for administering an effective amount of a compound of the invention and at least one additional therapeutic agent (including, but not limited to, any agent useful in treating DMD and related muscular dystrophy disorders). Examples of such additional therapeutic agents include, but are not limited to, corticosteroids (e.g., prednisone, deflazacort), P2 agonists, and ataluren.

Compositions within the scope of this invention include all compositions wherein the compounds of the present invention are contained in an amount which is effective to achieve its intended purpose. While individual needs vary, determination of optimal ranges of effective amounts of each component is within the skill of the art. Typically, the compounds may be administered to mammals, e.g. humans, orally at a dose of 0.0025 to 50 mg/kg, or an equivalent amount of the pharmaceutically acceptable salt thereof, per day of the body weight of the mammal being treated for disorders responsive to induction of apoptosis. In one embodiment, about 0.01 to about 25 mg/kg is orally administered to treat, ameliorate, or prevent such disorders. For intramuscular injection, the dose is generally about one-half of the oral dose. For example, a suitable intramuscular dose would be about 0.0025 to about 25 mg/kg, or from about 0.01 to about 5 mg/kg.

The unit oral dose may comprise from about 0.01 to about 1000 mg, for example, about 0.1 to about 100 mg of the compound. The unit dose may be administered one or more times daily as one or more tablets or capsules each containing from about 0.1 to about 10 mg, conveniently about 0.25 to 50 mg of the compound or its solvates.

In a topical formulation, the compound may be present at a concentration of about 0.01 to 100 mg per gram of carrier. In a one embodiment, the compound is present at a concentration of about 0.07-1.0 mg/ml, for example, about 0.1-0.5 mg/ml, and in one embodiment, about 0.4 mg/ml.

In addition to administering the compound as a raw chemical, the compounds of the invention may be administered as part of a pharmaceutical preparation containing suitable pharmaceutically acceptable carriers comprising excipients and auxiliaries which facilitate processing of the compounds into preparations which can be used pharmaceutically. The preparations, particularly those preparations which can be administered orally or topically and which can be used for one type of administration, such as tablets, dragees, slow release lozenges and capsules, mouth rinses and mouth washes, gels, liquid suspensions, hair rinses, hair gels, shampoos and also preparations which can be administered rectally, such as suppositories, as well as suitable solutions for administration by intravenous infusion, injection, topically or orally, contain from about 0.01 to 99 percent, in one embodiment from about 0.25 to 75 percent of active compound(s), together with the excipient.

The pharmaceutical compositions of the invention may be administered to any patient which may experience the beneficial effects of the compounds of the invention. Foremost among such patients are mammals, e.g., humans, although the invention is not intended to be so limited. Other patients include veterinary animals (cows, sheep, pigs, horses, dogs, cats and the like).

The compounds and pharmaceutical compositions thereof may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner which is itself known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are in one embodiment dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this invention are formulated in one embodiment as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The carriers may be those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762; each herein incorporated by reference in its entirety.

Ointments may be formulated by mixing a solution of the active ingredient in a vegetable oil such as almond oil with warm soft paraffin and allowing the mixture to cool. A typical example of such an ointment is one which includes about 30% almond oil and about 70% white soft paraffin by weight. Lotions may be conveniently prepared by dissolving the active ingredient, in a suitable high molecular weight alcohol such as propylene glycol or polyethylene glycol.

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

### EXAMPLES

The following examples are illustrative, but not limiting, of the compounds, compositions, and methods of the present invention. Other suitable modifications and adaptations of the variety of conditions and parameters normally encountered in clinical therapy and which are obvious to those skilled in the art are within the spirit and scope of the invention.

### Example 1

### 4-methyl-N-(2-(piperidin-1-yl)phenyl)benzenesulfonamide

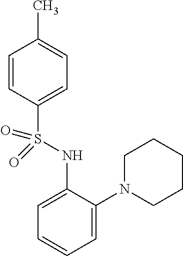 [SMILES: Cc1ccc(S(=O)(=O)Nc2ccccc2N2CCCCC2)cc1]

A solution of 2-(piperidin-1-yl)aniline (0.176 g, 1 mmol) in pyridine (2 ml) was treated with 4-methylbenzene-1-sulfonyl chloride (0.191 g, 1.000 mmol). The solution was microwaved at 115° C. for 1 hr. To the solution was added toluene (2 ml). The solution was concentrated. To the residue was added MeOH (1 ml). The crude product was purified by reversed-phase chromatography (4-100% CH₃CN over 15 min) to give desired product (0.155 g, 50% yield). (LCMS, ESI pos.) Calculated for C₁₈H₂₂N₂O₂S: 331.1 (M+H), Measured: 331.2. ¹H NMR (400 MHz, DMSO-d6) δ 8.57 (s, 1H), 7.68-7.61 (m, 2H), 7.35-7.23 (m, 3H), 7.17-6.98 (m, 3H), 2.46-2.38 (m, 4H), 2.31 (s, 3H), 1.57 (p, J=5.4 Hz, 4H), 1.44 (q, J=6.1 Hz, 2H).

### Example 2

### 3-methyl-N-(2-(piperidin-1-yl)phenyl)benzenesulfonamide

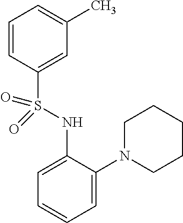 [SMILES: Cc1cccc(S(=O)(=O)Nc2ccccc2N2CCCCC2)c1]

A solution of 2-(piperidin-1-yl)aniline (0.176 g, 1 mmol) in pyridine (2 ml) was treated with 3-methylbenzene-1-sulfonyl chloride (0.191 g, 1.000 mmol). The solution was microwaved at 115° C. for 1 hr. To the solution was added toluene (2 ml). The solution was concentrated. To the residue was added MeOH (1 ml). The crude product was purified by reversed-phase chromatography (4-100% CH₃CN over 15 min) to give desired product (0.155 g, 50% yield). (LCMS, ESI pos.) Calculated for C₁₈H₂₂N₂O₂S: 331.1 (M+H), Measured: 331.1. ¹H NMR (400 MHz, DMSO-d6) δ 8.67 (s, 1H), 7.66 (tt, J=1.7, 0.8 Hz, 1H), 7.60-7.54 (m, 1H), 7.52-7.30 (m, 3H), 7.24-7.14 (m, 1H), 7.10 (dd, J=6.0, 3.5 Hz, 2H), 2.52-2.42 (m, 3H), 1.63 (p, J=5.5 Hz, 4H), 1.51 (q, J=6.2 Hz, 2H).

### Example 3

### 2-methyl-N-(2-(piperidin-1-yl)phenyl)benzenesulfonamide

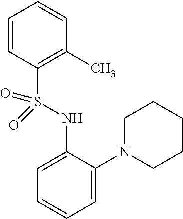 [SMILES: Cc1ccccc1S(=O)(=O)Nc1ccccc1N1CCCCC1]

A solution of 2-(piperidin-1-yl)aniline (0.176 g, 1 mmol) in pyridine (Volume: 2 ml) was treated with 2-methylbenzene-1-sulfonyl chloride (0.191 g, 1.000 mmol). The solution was microwaved at 115° C. for 1 hr. To the solution was added toluene (2 ml). The solution was concentrated. To the residue was added MeOH (1 ml). The solution was filtered. The solid was dried in vacuo to give desired product (0.040 g, 12% yield). (LCMS, ESI pos.) Calculated for C₁₈H₂₂N₂O₂S: 331.1 (M+H), Measured: 331.1. ¹H NMR (400 MHz, DMSO-d6) δ 8.72 (s, 1H), 7.88 (dd, J=7.9, 1.4 Hz, 1H), 7.55 (td, J=7.5, 1.4 Hz, 1H), 7.45-7.33 (m, 2H), 7.30-7.24 (m, 1H), 7.23-7.18 (m, 1H), 7.08-7.03 (m, 2H), 2.62 (d, J=7.3 Hz, 8H), 1.65 (p, J=5.5 Hz, 5H), 1.53 (q, J=6.5 Hz, 2H).

### Example 4

### 4-(methylsulfonyl)-N-(2-(piperidin-1-yl)phenyl)benzenesulfonamide

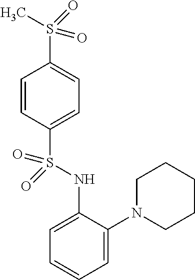 [SMILES: CS(=O)(=O)c1ccc(S(=O)(=O)Nc2ccccc2N2CCCCC2)cc1]

A solution of 2-(piperidin-1-yl)aniline (0.176 g, 1 mmol) in pyridine (Volume: 2 ml) was treated with 4-(methylsulfonyl)benzene-1-sulfonyl chloride (0.255 g, 1.000 mmol). The solution was microwaved at 115° C. for 1 hr. To the solution was added toluene (2 ml). The solution was concentrated. To the residue was added MeOH (1 ml). The crude product was purified by reversed-phase chromatography (4-100% CH₃CN over 15 min) to give desired product (0.002 g, 2% yield). (LCMS, ESI pos.) Calculated for C₁₈H₂₂N₂O₄S₂: 395.1 (M+H), Measured: 395.1. ¹H NMR (400 MHz, DMSO-d6) δ 9.18 (s, 1H), 8.19-8.11 (m, 2H), 8.10-8.03 (m, 2H), 7.29 (dd, J=7.5, 1.2 Hz, 1H), 7.20-7.13 (m, 2H), 7.09 (ddd, J=7.8, 5.7, 3.2 Hz, 1H), 3.31 (s, 3H), 2.52-2.47 (m, 4H), 1.63-1.41 (m, 6H).

### Example 5

### N-(2-(piperidin-1-yl)phenyl)benzenesulfonamide

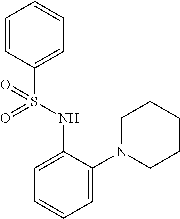 [SMILES: O=S(=O)(Nc1ccccc1N1CCCCC1)c1ccccc1]

A solution of 2-(piperidin-1-yl)aniline (0.176 g, 1 mmol) in pyridine (Volume: 2 ml) was treated with benzenesulfonyl chloride (0.128 ml, 1.000 mmol). The solution was microwaved at 115° C. for 1 hr. To the solution was added toluene (2 ml). The solution was concentrated. To the residue was added MeOH (1 ml). The solution was filtered. The solid was dried in vacuo to give desired product (0.0042 g, 2%). (LCMS, ESI pos.) Calculated for C₁₇H₂₀N₂O₂S: 317.1 (M+H), Measured: 317.1. ¹H NMR NMR (400 MHz, DMSO-d6) δ 8.71 (s, 1H), 7.85-7.79 (m, 2H), 7.70-7.63 (m, 1H), 7.62-7.55 (m, 2H), 7.41-7.33 (m, 1H), 7.20-7.13 (m, 1H), 7.12-7.05 (m, 2H), 2.51-2.41 (m, 5H), 1.65-1.56 (m, 5H), 1.49 (td, J=7.1, 6.6, 3.4 Hz, 2H).

### Example 6

### N¹,N¹-dimethyl-N⁴-(2-(piperidin-1-yl)phenyl)benzene-1,4-disulfonamide

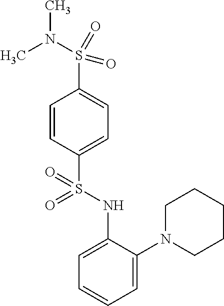 [SMILES: CN(C)S(=O)(=O)c1ccc(S(=O)(=O)Nc2ccccc2N2CCCCC2)cc1]

A solution of 2-(piperidin-1-yl)aniline (0.176 g, 1 mmol) in pyridine (Volume: 2 ml) was treated with 4-(N,N-dimethylsulfamoyl)benzene-1-sulfonyl chloride (0.284 g, 1.000 mmol). The solution was microwaved at 115° C. for 1 hr. To the solution was added toluene (2 ml). The solution was concentrated. To the residue was added MeOH (1 ml). The crude product was purified by reversed-phase chromatography (4-100% CH₃CN over 15 min) to give desired product (0.210 g, 50% yield). (LCMS, ESI pos.) Calculated for C₁₉H₂₅N₃O₄S₂: 424.1 (M+H), Measured: 424.1. ¹H NMR (400 MHz, DMSO-d6) δ 9.16 (s, 1H), 8.07-8.01 (m, 2H), 7.98-7.93 (m, 2H), 7.31 (d, J=7.8 Hz, 1H), 7.22-7.06 (m, 3H), 2.65 (s, 6H), 2.49 (d, J=6.3 Hz, 4H), 1.56 (t, J=5.5 Hz, 4H), 1.47 (d, J=5.3 Hz, 2H).

### Example 7

### 4-chloro-N-(2-(piperidin-1-yl)phenyl)benzenesulfonamide

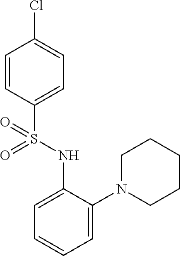 [SMILES: O=S(=O)(Nc1ccccc1N1CCCCC1)c1ccc(Cl)cc1]

A solution of 2-(piperidin-1-yl)aniline (0.176 g, 1 mmol) in pyridine (Volume: 2 ml) was treated with 4-chlorobenzene-1-sulfonyl chloride (0.211 g, 1.000 mmol). The solution was microwaved at 115° C. for 1 hr. To the solution was added toluene (2 ml). The solution was concentrated. To the residue was added MeOH (1 ml). The solution was filtered. The solid was dried in vacuo to give desired product (0.0042 g, 2%). (LCMS, ESI pos.) Calculated for C₁₇H₁₉ClN₂O₂S: 351.1, 353.3 (M+H), Measured: 351.1, 353.1. ¹H NMR NMR (400 MHz, DMSO-d6) δ 8.85 (s, 1H), 7.79-7.73 (m, 2H), 7.64-7.58 (m, 2H), 7.27-7.21 (m, 1H), 7.13-6.97 (m, 3H), 2.45 (t, J=5.3 Hz, 4H), 1.54 (p, J=5.8 Hz, 4H), 1.43 (td, J=6.4, 3.5 Hz, 2H).

### Example 8

### N-(2-(piperidin-1-yl)phenyl)methanesulfonamide

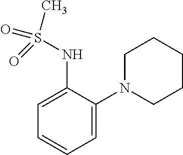 [SMILES: CS(=O)(=O)Nc1ccccc1N1CCCCC1]

A solution of 2-(piperidin-1-yl)aniline (0.176 g, 1 mmol) in pyridine (Volume: 2 ml) was treated with methanesulfonyl chloride (0.078 ml, 1.000 mmol). The solution was microwaved at 115° C. for 1 hr. To the solution was added toluene (2 ml). The solution was concentrated. To the residue was added MeOH (1 ml). The crude product was purified by reversed-phase chromatography (4-100% CH₃CN over 15 min) to give desired product (0.209 g, 82% yield). (LCMS, ESI pos.) Calculated for C₁₂H₁₈N₂O₂S: 255.1 (M+H), Measured: 255.1. ¹H NMR (400 MHz, DMSO-d6) δ 8.30 (s, 1H), 7.42-7.34 (m, 1H), 7.28 (s, 1H), 7.16 (t, J=4.7 Hz, 2H), 3.17 (s, 3H), 2.83 (s, 4H), 1.73 (p, J=5.5 Hz, 4H), 1.62-1.51 (m, 2H).

### Example 9

### N¹-(2-(1H-pyrrol-1-yl)phenyl)-N⁴,N⁴-dimethylbenzene-1,4-disulfonamide

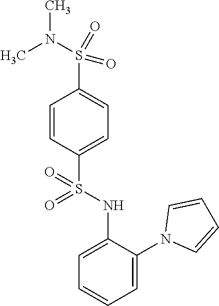 [SMILES: CN(C)S(=O)(=O)c1ccc(S(=O)(=O)Nc2ccccc2-n2cccc2)cc1]

A solution of 2-(1H-pyrrol-1-yl)aniline (0.079 g, 0.500 mmol) in pyridine (Volume: 1 ml) was treated with 4-(N,N-dimethylsulfamoyl)benzene-1-sulfonyl chloride (0.142 g, 0.5 mmol). The solution was microwaved at 115° C. for 1 hr. To the solution was added toluene (2 ml). The solution was concentrated. To the residue was added MeOH (1 ml). The crude product was purified by reversed-phase chromatography (4-100% CH₃CN over 15 min) to give desired product (0.0567 g, 14% yield). (LCMS, ESI pos.) Calculated for C₁₈H₁₉N₃O₄S₂: 406.1 (M+H), Measured: 406.2. ¹H NMR (400 MHz, DMSO-d6) δ 10.12 (s, 1H), 7.89-7.83 (m, 2H), 7.81-7.75 (m, 2H), 7.46-7.29 (m, 3H), 7.22-7.16 (m, 1H), 6.85 (td, J=2.2, 0.7 Hz, 2H), 6.15 (td, J=2.2, 0.7 Hz, 2H), 2.70 (d, J=0.7 Hz, 6H).

### Example 10

### N¹-([1,1′-biphenyl]-2-yl)-N⁴,N⁴-dimethylbenzene-1,4-disulfonamide

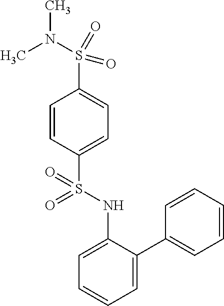 [SMILES: CN(C)S(=O)(=O)c1ccc(S(=O)(=O)Nc2ccccc2-c2ccccc2)cc1]

A solution of [1,1′-biphenyl]-2-amine (0.085 g, 0.500 mmol) in pyridine (Volume: 1 ml) was treated with 4-(N,N-dimethylsulfamoyl)benzene-1-sulfonyl chloride (0.142 g, 0.5 mmol). The solution was microwaved at 115° C. for 1 hr. To the solution was added toluene (2 ml). The solution was concentrated. To the residue was added MeOH (1 ml). The crude product was purified by reversed-phase chromatography (4-100% CH₃CN over 15 min) to give desired product (0.0405 g, 20% yield). (LCMS, ESI pos.) Calculated for C₂₀H₂₀N₂O₄S₂: 417.1 (M+H), Measured: 417.7. ¹H NMR (400 MHz, DMSO-d6) δ 9.94 (s, 1H), 7.86-7.80 (m, 2H), 7.78-7.72 (m, 2H), 7.40-7.28 (m, 6H), 7.26-7.21 (m, 2H), 7.14-7.09 (m, 1H), 2.68 (d, J=0.7 Hz, 6H).

### Example 11

N¹,N¹-dimethyl-N⁴-(2-nitrophenyl)benzene-1,4-disulfonamide

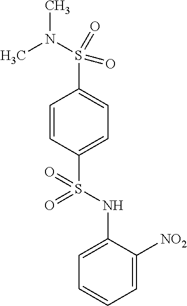 [SMILES: CN(C)S(=O)(=O)c1ccc(S(=O)(=O)Nc2ccccc2[N+](=O)[O-])cc1]

A solution of 2-nitroaniline (0.069 g, 0.500 mmol) in pyridine (Volume: 1 ml) was treated with 4-(N,N-dimethylsulfamoyl)benzene-1-sulfonyl chloride (0.142 g, 0.5 mmol). The solution was microwaved at 115° C. for 1 hr. To the solution was added toluene (2 ml). The solution was concentrated. To the residue was added MeOH (1 ml). The crude product was purified by reversed-phase chromatography (4-100% CH₃CN over 15 min) to give desired product (0.003 g, 3% yield). (LCMS, ESI pos.) Calculated for C₁₄H₁₅N₃O₆S₂: 386.0 (M+H), Measured: 385.8. ¹H NMR (400 MHz, DMSO-d6) δ 10.64 (s, 1H), 7.95 (d, J=0.8 Hz, 5H), 7.68 (t, J=7.9 Hz, 1H), 7.47 (t, J=7.8 Hz, 1H), 7.33 (dd, J=8.2, 1.3 Hz, 1H), 2.67 (d, J=0.7 Hz, 6H).

### Example 12

N¹-(2-(tert-butyl)phenyl)-N⁴,N⁴-dimethylbenzene-1,4-disulfonamide

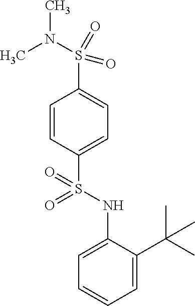 [SMILES: CN(C)S(=O)(=O)c1ccc(S(=O)(=O)Nc2ccccc2C(C)(C)C)cc1]

A solution of 2-(tert-butyl)aniline (0.078 ml, 0.500 mmol) in pyridine (Volume: 1 ml) was treated with 4-(N,N-dimethylsulfamoyl)benzene-1-sulfonyl chloride (0.142 g, 0.5 mmol). The solution was microwaved at 115° C. for 1 hr. To the solution was added toluene (2 ml). The solution was concentrated. To the residue was added MeOH (1 ml). The crude product was purified by reversed-phase chromatography (4-100% CH₃CN over 15 min) to give desired product (0.0909 g, 46% yield). (LCMS, ESI pos.) Calculated for C₁₈H₂₄N₂O₄S₂: 397.1 (M+H), Measured: 397.1. ¹H NMR (400 MHz, DMSO-d6) δ 9.67 (s, 1H), 8.20-7.98 (m, 4H), 7.48 (d, J=8.0 Hz, 1H), 7.26 (t, J=7.7 Hz, 1H), 7.07 (t, J=7.5 Hz, 1H), 6.46 (d, J=7.8 Hz, 1H), 2.71 (d, J=0.6 Hz, 6H), 1.46 (d, J=0.7 Hz, 9H).

### Example 13

### N-(2-(piperidin-1-yl)phenyl)-4-((trifluoromethyl)sulfonyl)benzenesulfonamide

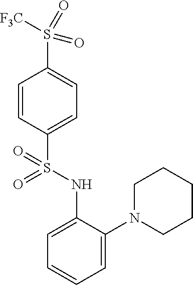 [SMILES: O=S(=O)(Nc1ccccc1N1CCCCC1)c1ccc(S(=O)(=O)C(F)(F)F)cc1]

A solution of 2-(piperidin-1-yl)aniline (0.088 g, 0.500 mmol) in pyridine (Volume: 1 ml) was treated with 4-((trifluoromethyl)sulfonyl)benzene-1-sulfonyl chloride (0.154 g, 0.5 mmol). The solution was microwaved at 115° C. for 1 hr. To the solution was added toluene (2 ml). The solution was concentrated. To the residue was added MeOH (1 ml). The crude product was purified by reversed-phase chromatography (4-100% CH₃CN over 15 min) to give desired product (0.128 g, 57% yield). (LCMS, ESI pos.) Calculated for C₁₈H₁₉F₃N₂O₄S₂: 449.1 (M+H), Measured: 448.7. ¹H NMR (400 MHz, DMSO-d6) δ 9.49 (s, 1H), 8.39 (d, J=8.4 Hz, 2H), 8.21-8.14 (m, 2H), 7.31 (dd, J=7.9, 1.5 Hz, 1H), 7.24-7.07 (m, 3H), 2.47 (t, J=4.8 Hz, 4H), 1.45 (p, J=6.5, 5.7 Hz, 6H).

### Example 14

### N⁴,N⁴-dimethyl-2-nitro-N¹-(2-(piperidin-1-yl)phenyl)benzene-1,4-disulfonamide

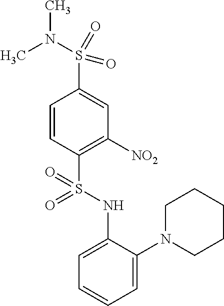 [SMILES: CN(C)S(=O)(=O)c1ccc(S(=O)(=O)Nc2ccccc2N2CCCCC2)c([N+](=O)[O-])c1]

A solution of 2-(piperidin-1-yl)aniline (0.088 g, 0.5 mmol) in pyridine (Volume: 1 ml) was treated with 4-(N,N-dimethylsulfamoyl)-2-nitrobenzene-1-sulfonyl chloride (0.164 g, 0.500 mmol). The resulting solution was microwaved at 115° C. for 1 hr. The solution was treated with toluene (1 ml) and concentrated. The crude product was purified by reversed-phase chromatography (4-100% CH₃CN over 15 min) to give desired product (0.002 g, 2% yield). (LCMS, ESI pos.) Calculated for C₁₉H₂₄N₄₀₆S₂: 468.5 (M+H), Measured: 468.7. ¹H NMR (400 MHz, DMSO-d6) δ 9.56 (s, 1H), 8.46 (q, J=1.0 Hz, 1H), 8.19 (t, J=0.9 Hz, 2H), 7.35 (dd, J=7.7, 1.7 Hz, 1H), 7.27 (dd, J=7.8, 1.7 Hz, 1H), 7.24-7.09 (m, 2H), 2.74 (d, J=0.7 Hz, 6H), 2.68 (t, J=5.0 Hz, 4H), 1.58-1.42 (m, 6H).

### Example 15

### N-(2-morpholinophenyl)-4-((trifluoromethyl)sulfonyl)benzenesulfonamide

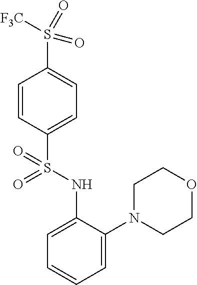 [SMILES: O=S(=O)(Nc1ccccc1N1CCOCC1)c1ccc(S(=O)(=O)C(F)(F)F)cc1]

A solution of 2-morpholinoaniline (0.089 g, 0.5 mmol) in pyridine (Volume: 1 ml) was treated with 4-((trifluoromethyl)sulfonyl)benzene-1-sulfonyl chloride (0.154 g, 0.500 mmol). The solution was microwaved at 115° C. for 1 hr. The solution was treated with toluene (1 ml) and concentrated. The crude product was purified by reversed-phase chromatography (4-100% CH₃CN over 15 min) to give desired product (0.058 g, 21% yield). (LCMS, ESI pos.) Calculated for C₁₇H₁₇F₃N₂O₅S₂: 450.5 (M+H), Measured: 450.7. ¹H NMR (400 MHz, DMSO-d6) δ 9.75 (s, 1H), 8.40 (d, J=8.3 Hz, 2H), 8.24-8.06 (m, 2H), 7.39-7.05 (m, 4H), 3.57-3.48 (m, 4H), 2.53-2.47 (m, 5H).

### Example 16

### N¹-methyl-N⁴-(2-(piperidin-1-yl)phenyl)benzene-1,4-disulfonamide

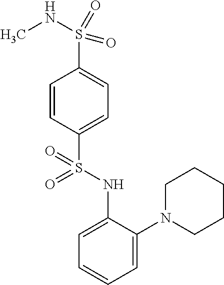 [SMILES: CNS(=O)(=O)c1ccc(S(=O)(=O)Nc2ccccc2N2CCCCC2)cc1]

A solution of 2-(piperidin-1-yl)aniline (0.088 g, 0.5 mmol) in pyridine (Volume: 1 ml) was treated with 4-(N-methylsulfamoyl)benzene-1-sulfonyl chloride (0.135 g, 0.500 mmol). The solution was microwaved at 115° C. for 1 hr. The solution was treated with toluene (3 ml). The solution was concentrated. The crude product was purified by reversed-phase chromatography (4-100% CH₃CN over 15 min) to give desired product (0.069 g, 17% yield). (LCMS, ESI pos.) Calculated for C₁₈H₂₃N₃O₄S₂: 410.5 (M+H), Measured: 410.1. ¹H NMR (400 MHz, DMSO-d6) δ 9.07 (s, 1H), 8.06-7.99 (m, 2H), 7.99-7.92 (m, 2H), 7.73 (q, J=4.9 Hz, 1H), 7.37-7.28 (m, 1H), 7.23-7.06 (m, 3H), 2.51-2.40 (m, 7H), 1.55 (q, J=5.4 Hz, 4H), 1.46 (d, J=9.3 Hz, 2H).

### Example 17

### N-(2-(piperidin-1-yl)phenyl)benzene-1,4-disulfonamide

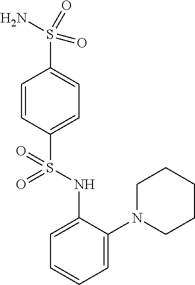 [SMILES: NS(=O)(=O)c1ccc(S(=O)(=O)Nc2ccccc2N2CCCCC2)cc1]

A solution of 2-(piperidin-1-yl)aniline (0.088 g, 0.5 mmol) in pyridine (Volume: 1 ml) was treated with 4-sulfamoylbenzene-1-sulfonyl chloride (0.128 g, 0.500 mmol). The resulting solution was microwaved at 115° C. for 1 hr. The solution was treated with toluene (3 ml). The solution was concentrated. The crude product was purified by reversed-phase chromatography (4-100% CH₃CN over 15 min) to give desired product (0.085 g, 22% yield). (LCMS, ESI pos.) Calculated for C₁₇H₂₁N₃O₄S₂: 396.5 (M+H), Measured: 396.1. ¹H NMR (400 MHz, DMSO-d6) δ 9.03 (s, 1H), 8.10-7.91 (m, 4H), 7.61 (s, 2H), 7.29 (dd, J=7.6, 1.7 Hz, 1H), 7.22-6.99 (m, 3H), 1.71-1.37 (m, 6H).

### Example 18

### N¹,N¹-dimethyl-N⁴-(2-(4-methylpiperazin-1-yl)phenyl)benzene-1,4-disulfonamide

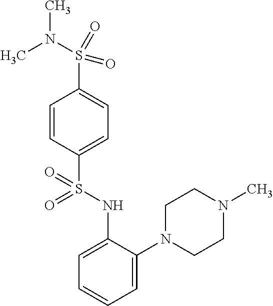

A solution of 2-(4-methylpiperazin-1-yl)aniline (0.096 g, 0.500 mmol) in pyridine (Volume: 1 ml) was treate with 4-(N,N-dimethylsulfamoyl)benzene-1-sulfonyl chloride (0.142 g, 0.5 mmol). The resulting solution was microwaved at 115° C. for 1 hr. The solution was treated with toluene (3 ml). The solution was concentrated. The crude product was purified by reversed-phase chromatography (4-100% CH₃CN over 15 min) to give desired product (0.005 g, 2% yield). (LCMS, ESI pos.) Calculated for C₁₉H₂₆N₄O₄S₂: 439.6 (M+H), Measured: 439.1. ¹H NMR (400 MHz, DMSO-d6) δ 9.52 (s, 2H), 8.07-8.00 (m, 2H), 8.04-7.94 (m, 2H), 7.34 (d, J=7.6 Hz, 1H), 7.21 (d, J=4.3 Hz, 3H), 2.71 (t, J=1.9 Hz, 2H), 2.67 (d, J=1.0 Hz, 6H), 2.51-2.46 (m, 1H), 2.37 (dt, J=3.3, 1.8 Hz, 1H).

### Example 19

### N¹-(2-(cyclohexylamino)phenyl)-N⁴,N⁴-dimethylbenzene-1,4-disulfonamide

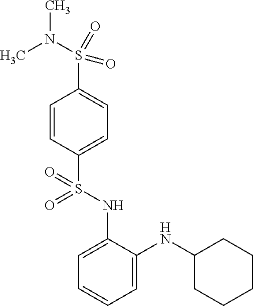 [SMILES: CN(C)S(=O)(=O)c1ccc(S(=O)(=O)Nc2ccccc2NC2CCCCC2)cc1]

A solution of N1-cyclohexylbenzene-1,2-diamine (0.095 g, 0.500 mmol) in pyridine (Volume: 1 ml) was treated with 4-(N,N-dimethylsulfamoyl)benzene-1-sulfonyl chloride (0.142 g, 0.5 mmol). The resulting solution was microwaved at 115° C. for 1 hr. The solution was treated with toluene (3 ml). The solution was concentrated. The crude product was purified by reversed-phase chromatography (4-100% CH₃CN over 15 min) to give desired product (0.080 g, 18% yield). (LCMS, ESI pos.) Calculated for C₂₀H₂₇N₃O₄S₂: 438.6 (M+H), Measured: 438.1. ¹H NMR (400 MHz, DMSO-d6) δ 8.01-7.82 (m, 4H), 7.05 (ddt, J=8.4, 7.3, 1.4 Hz, 1H), 6.83 (dt, J=7.8, 1.4 Hz, 1H), 6.62-6.52 (m, 1H), 6.48 (tt, J=7.5, 1.3 Hz, 1H), 3.15-3.04 (m, 1H), 2.67 (d, J=1.1 Hz, 6H), 1.72 (d, J=12.4 Hz, 2H), 1.67-1.50 (m, 4H), 1.24 (dq, J=35.9, 11.9 Hz, 3H), 0.97-0.81 (m, 2H).

### Example 20

### N¹,N¹-dimethyl-N⁴-(2-(pyrrolidin-1-yl)phenyl)benzene-1,4-disulfonamide

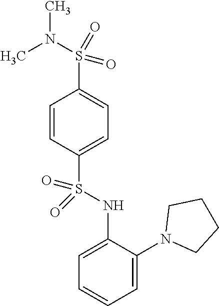 [SMILES: CN(C)S(=O)(=O)c1ccc(S(=O)(=O)Nc2ccccc2N2CCCC2)cc1]

A solution of 2-(pyrrolidin-1-yl)aniline (0.081 g, 0.500 mmol) in pyridine (Volume: 1 ml) was treated with 4-(N,N-dimethylsulfamoyl)benzene-1-sulfonyl chloride (0.142 g, 0.5 mmol). The resulting solution was microwaved at 115° C. for 1 hr. The solution was treated with toluene (3 ml). The solution was concentrated. The crude product was purified by reversed-phase chromatography (4-100% CH₃CN over 15 min) to give desired product (0.047 g, 10% yield). (LCMS, ESI pos.) Calculated for C₁₈H₂₃N₃O₄S₂: 410.5 (M+H), Measured: 410.1. ¹H NMR (400 MHz, DMSO-d6) δ 9.61 (s, 1H), 8.05-7.85 (m, 4H), 7.20-7.04 (m, 1H), 6.82 (d, J=8.3 Hz, 1H), 6.60 (dt, J=4.1, 1.4 Hz, 2H), 3.26-3.18 (m, 4H), 2.68 (d, J=1.1 Hz, 6H), 1.80 (q, J=4.8, 3.2 Hz, 4H).

### Example 21

N¹,N¹-dimethyl-N⁴-(4-(piperidin-1-yl)phenyl)benzene-1,4-disulfonamide

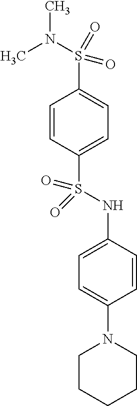 [SMILES: CN(C)S(=O)(=O)c1ccc(S(=O)(=O)Nc2ccc(N3CCCCC3)cc2)cc1]

A solution of 4-(piperidin-1-yl)aniline (0.088 g, 0.500 mmol) in pyridine (Volume: 1 ml) was treated with 4-(N,N-dimethylsulfamoyl)benzene-1-sulfonyl chloride (0.142 g, 0.5 mmol). The resulting solution was microwaved at 115° C. for 1 hr. The solution was treated with toluene (3 ml). The solution was concentrated. The crude product was purified by reversed-phase chromatography (4-100% CH₃CN over 15 min) to give desired product (0.14 g, 33% yield). (LCMS, ESI pos.) Calculated for C₁₉H₂₅N₃O₄S₂: 424.6 (M+H), Measured: 424.1. ¹H NMR (400 MHz, DMSO-d6) δ 10.34 (s, 1H), 8.06-7.87 (m, 4H), 7.03 (s, 3H), 3.39-3.06 (m, 4H), 2.66 (d, J=1.1 Hz, 5H), 1.63 (d, J=49.9 Hz, 6H).

### Example 22

### N¹,N¹-dimethyl-N²-(2-(piperidin-1-yl)phenyl)benzene-1,2-disulfonamide

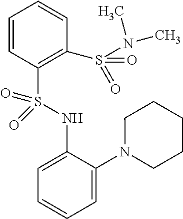 [SMILES: CN(C)S(=O)(=O)c1ccccc1S(=O)(=O)Nc1ccccc1N1CCCCC1]

A solution of 2-(piperidin-1-yl)aniline (0.088 g, 0.5 mmol) in pyridine (Volume: 1 ml) was treated with 2-(N,N-dimethylsulfamoyl)benzene-1-sulfonyl chloride (0.142 g, 0.500 mmol). The resulting solution was microwaved at 115° C. for 1 hr. The solution was treated with toluene (3 ml). The solution was concentrated. The residue was treated with MeOH (2 ml). The solution was filtered. The solid was dried in vacuo to give desired product (0.134, 32%). (LCMS, ESI pos.) Calculated for C₁₉H₂₅N₃O₄S₂: 424.5 (M+H), Measured: 424.1. ¹H NMR (400 MHz, DMSO-d6) δ 9.39 (s, 1H), 8.39-8.31 (m, 1H), 8.01-7.95 (m, 1H), 7.92-7.85 (m, 2H), 7.54-7.46 (m, 1H), 7.26-7.19 (m, 1H), 7.10-6.97 (m, 2H), 2.90 (d, J=0.7 Hz, 6H), 2.66 (t, J=5.3 Hz, 4H), 1.72 (dq, J=11.4, 5.8, 5.3 Hz, 4H), 1.54 (d, J=7.2 Hz, 2H).

### Example 23

### N¹-(3-chloro-2-(piperidin-1-yl)phenyl)-N⁴,N⁴-dimethylbenzene-1,4-disulfonamide

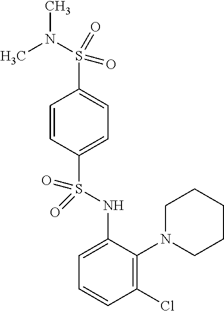 [SMILES: CN(C)S(=O)(=O)c1ccc(S(=O)(=O)Nc2cccc(Cl)c2N2CCCCC2)cc1]

A solution of 3-chloro-2-(piperidin-1-yl)aniline dihydrochloride (0.533 g, 1.879 mmol) in Pyridine (3 ml) was treated with 4-(N,N-dimethylsulfamoyl)benzenesulfonyl chloride (0.533 g, 1.879 mmol). The solution was microwaved at 120 deg for 1 hr. The solution was treated with toluene (3 ml) and concentrated. The residue was taken up in MeOH (2 ml). The solution was filtered. The solid was dried in vacuo to give desired product (0.53 g, 61%). (LCMS, ESI pos.) Calculated for C₁₉H₂₄ClN₃O₄S₂: 458.0, 460.0 (M+H), Measured: 457.7, 459.7. ¹H NMR (400 MHz, DMSO-d6) δ 9.25 (s, 1H), 8.05-7.93 (m, 4H), 7.31-7.13 (m, 3H), 3.17 (s, 2H), 2.65 (d, J=0.6 Hz, 6H), 2.35 (d, J=10.8 Hz, 2H), 1.57 (s, 5H), 1.35 (s, 1H).

### Example 24

### N¹-(2-(dimethylamino)phenyl)-N⁴,N⁴-dimethylbenzene-1,4-disulfonamide

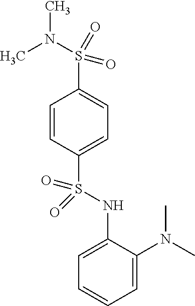

A solution of N¹,N¹-dimethylbenzene-1,2-diamine (0.065 ml, 0.5 mmol) in pyridine (Volume: 1 ml) was treated with 4-(N,N-dimethylsulfamoyl)benzene-1-sulfonyl chloride (0.142 g, 0.500 mmol). The solution was stirred at room temperature for a few minutes then microwaved at 115° C. for 1 hr. The solution was treated with toluene (3 ml) and concentrated. The crude product was purified by reversed-phase chromatography (4-100% CH₃CN over 15 min) to give desired product (0.060 g, 16% yield). (LCMS, ESI pos.) Calculated for C₁₆H₂₁N₃O₄S₂: 384.5 (M+H), Measured: 384.5. ¹H NMR (400 MHz, DMSO-d6) δ 8.13-8.01 (m, 2H), 8.01-7.93 (m, 2H), 7.23 (d, J=23.7 Hz, 2H), 7.06 (d, J=7.7 Hz, 2H), 2.67 (d, J=1.2 Hz, 6H).

### Example 25

### N¹,N¹-dimethyl-N⁴-phenylbenzene-1,4-disulfonamide

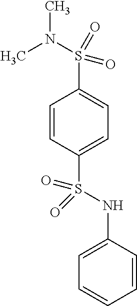 [SMILES: CN(C)S(=O)(=O)c1ccc(S(=O)(=O)Nc2ccccc2)cc1]

A solution of aniline (0.046 ml, 0.500 mmol) in pyridine (Volume: 1 ml) was treated with 4-(N,N-dimethylsulfamoyl)benzene-1-sulfonyl chloride (0.142 g, 0.5 mmol). The solution was stirred at room temperature for a few minutes then microwaved at 115° C. for 1 hr. The solution was treated with toluene (3 ml) and concentrated. The crude product was purified by reversed-phase chromatography (4-100% CH₃CN over 15 min) to give desired product (0.072 g, 21% yield). (LCMS, ESI pos.) Calculated for C₁₄H₁₆N₂O₄S₂: 341.4 (M+H), Measured: 341.0. ¹H NMR (400 MHz, DMSO-d6) δ 10.59-10.46 (m, 1H), 8.07-7.86 (m, 4H), 7.36-7.22 (m, 2H), 7.18-6.96 (m, 3H), 2.65 (d, J=1.0 Hz, 6H).

### Example 26

### N¹,N¹-dimethyl-N⁴-(2-(4-methylpiperidin-1-yl)phenyl)benzene-1,4-disulfonamide

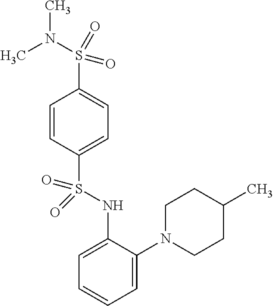

A solution of 2-(4-methylpiperidin-1-yl)aniline (1 g, 5.26 mmol) in Pyridine (10.51 ml) was treated with 4-(N,N-dimethylsulfamoyl)benzenesulfonyl chloride (1.491 g, 5.26 mmol). The solution was microwaved at 120 deg for 1 hr. The solution was treated with toluene (3 ml) and concentrated. The residue was taken up in MeOH (2 ml). The solution was filtered. The solid was dried in vacuo to give desired product (1.6 g, 68%). (LCMS, ESI pos.) Calculated for C₂₀H₂₇N₃O₄S₂: 438.6 (M+H), Measured: 438.1. ¹H NMR (400 MHz, DMSO-d6) δ 9.14 (s, 1H), 8.04 (d, J=8.4 Hz, 2H), 7.96 (d, J=8.4 Hz, 2H), 7.37-7.24 (m, 1H), 7.21-7.02 (m, 3H), 2.66 (s, 6H), 2.47 (td, J=11.5, 2.4 Hz, 2H), 1.61-1.49 (m, 2H), 1.41 (dq, J=10.7, 6.4, 5.2 Hz, 1H), 1.26 (qd, J=11.6, 4.1 Hz, 2H), 0.96 (d, J=6.3 Hz, 3H).

### Example 27

### N¹,N¹-dimethyl-N⁴-(3-(piperidin-1-yl)phenyl)benzene-1,4-disulfonamide

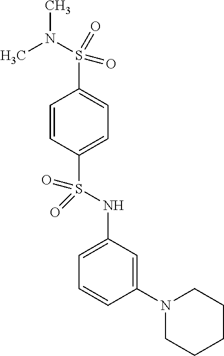 [SMILES: CN(C)S(=O)(=O)c1ccc(S(=O)(=O)Nc2cccc(N3CCCCC3)c2)cc1]

A solution of 3-(piperidin-1-yl)aniline (0.088 g, 0.500 mmol) in pyridine (Volume: 1.000 ml) was treated with 4-(N,N-dimethylsulfamoyl)benzene-1-sulfonyl chloride (0.142 g, 0.5 mmol). The resulting solution was microwaved at 115° C. for 1 hr. The reaction solution was treated with toluene (3 ml) and concentrated. The crude product was purified by reversed-phase chromatography (4-100% CH₃CN over 15 min) to give desired product (0.12 g, 28% yield). (LCMS, ESI pos.) Calculated for C₁₉H₂₅N₃O₄S₂: 424.6 (M+H), Measured: 424.1. ¹H NMR (400 MHz, DMSO-d6) δ 10.45 (s, 1H), 8.04-7.98 (m, 2H), 7.99-7.92 (m, 2H), 7.18 (t, J=8.1 Hz, 1H), 6.83 (d, J=25.4 Hz, 2H), 6.66 (s, 1H), 3.20-3.09 (m, 3H), 2.65 (s, 6H), 1.61 (d, J=32.8 Hz, 6H).

### Example 28

### N¹-(2-(azepan-1-yl)phenyl)-N⁴,N⁴-dimethylbenzene-1,4-disulfonamide

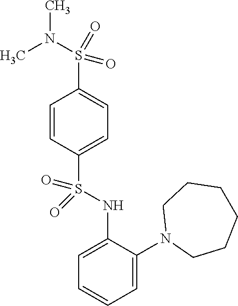 [SMILES: CN(C)S(=O)(=O)c1ccc(S(=O)(=O)Nc2ccccc2N2CCCCCC2)cc1]

A solution of 2-(azepan-1-yl)aniline (0.095 g, 0.500 mmol) in pyridine (Volume: 1 ml) was treated with 4-(N,N-dimethylsulfamoyl)benzene-1-sulfonyl chloride (0.142 g, 0.5 mmol). The solution was microwaved at 115° C. for 1 hr. The solution was treated with toluene (3 ml) and concentrated. The residue was taken up in MeOH (2 ml). The solution was filtered. The solid was dried in vacuo to give desired product (0.053, 12%). (LCMS, ESI pos.) Calculated for C₂₀H₂₇N₃O₄S₂: 438.6 (M+H), Measured: 438.1. ¹H NMR (400 MHz, DMSO-d6) δ 9.17 (s, 1H), 8.07-8.00 (m, 2H), 8.00-7.93 (m, 2H), 7.17-7.05 (m, 3H), 6.94 (dddd, J=9.0, 5.9, 3.1, 1.0 Hz, 1H), 2.90 (t, J=5.1 Hz, 4H), 2.66 (d, J=1.0 Hz, 6H), 1.63 (s, 8H).

### Example 29

### Ethyl N-((4-(N,N-dimethylsulfamoyl)phenyl)sulfonyl)-N-(2-(piperidin-1-yl)phenyl)glycinate

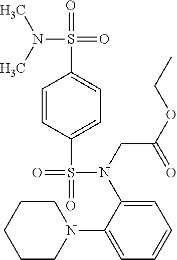 [SMILES: CCOC(=O)CN(c1ccccc1N1CCCCC1)S(=O)(=O)c1ccc(S(=O)(=O)N(C)C)cc1]

A solution of 2-(piperidin-1-yl)aniline (0.088 g, 0.5 mmol) in DMF (Volume: 1 ml) was treated with DIEA (0.1 ml) followed by 4-(N,N-dimethylsulfamoyl)benzene-1-sulfonyl chloride (0.142 g, 0.500 mmol). The resulting solution was stirred at room temperature for 1 hr. After 1 hr the reaction solution was treated with DIEA (0.118 ml) followed by ethyl 2-bromoacetate (0.055 ml, 0.500 mmol). The reaction solution was microwaved at 125° C. for 2 hrs. The reaction solution was treated with toluene (3 ml) and concentrated. The crude product was purified by reversed-phase chromatography (4-100% CH₃CN over 15 min) to give desired product (0.089 g, 17% yield). (LCMS, ESI pos.) Calculated for C₂₃H₃₁N₃O₆S₂: 510.6 (M+H), Measured: 510.1. ¹H NMR (400 MHz, DMSO-d6) δ 8.16 (d, J=8.2 Hz, 2H), 8.05-7.98 (m, 2H), 7.33 (dd, J=18.0, 8.3 Hz, 2H), 7.20 (d, J=7.9 Hz, 1H), 7.10 (t, J=7.6 Hz, 1H), 4.78 (s, 2H), 3.91 (q, J=7.1 Hz, 2H), 2.78 (s, 4H), 2.71 (d, J=1.0 Hz, 6H), 1.56 (s, 6H), 1.02 (t, J=7.1 Hz, 3H).

### Example 30

### N¹-(2-ethoxyphenyl)-N⁴,N⁴-dimethyl-N¹-(2-morpholino-2-oxoethyl)benzene-1,4-disulfonamide

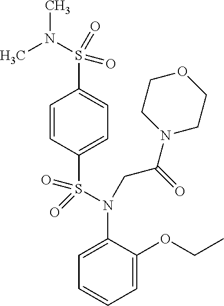 [SMILES: CCOc1ccccc1N(CC(=O)N1CCOCC1)S(=O)(=O)c1ccc(S(=O)(=O)N(C)C)cc1]

A solution of 2-ethoxyaniline (0.065 ml, 0.5 mmol) and DIEA (0.1 ml) in DMF (Volume: 1 ml) was treated with 4-(N,N-dimethylsulfamoyl)benzene-1-sulfonyl chloride (0.142 g, 0.500 mmol). The resulting solution was stirred at room temperature for 1 hr. The solution was treated with DIEA (0.118 ml) followed by 2-bromo-1-morpholinoethanone (0.104 g, 0.500 mmol). The solution was microwaved at 125° C. for 2 hrs. The reaction solution was treated with toluene (3 ml) and concentrated. The crude product was purified by reversed-phase chromatography (4-100% CH₃CN over 15 min) to give desired product (0.018 g, 4% yield). (LCMS, ESI pos.) Calculated for C₂₂H₂₉N₃O₇S₂: 512.6 (M+H), Measured: 512.1. ¹H NMR (400 MHz, DMSO-d6) δ 7.91 (qd, J=8.9, 8.4, 1.3 Hz, 4H), 7.52 (dt, J=7.7, 1.4 Hz, 1H), 7.33 (ddt, J=9.1, 7.9, 1.5 Hz, 1H), 6.97 (ddt, J=9.2, 7.9, 1.4 Hz, 2H), 4.54 (s, 2H), 3.68 (q, J=6.9 Hz, 2H), 3.57 (d, J=14.5 Hz, 4H), 3.43 (d, J=26.2 Hz, 4H), 2.70 (d, J=1.2 Hz, 6H), 0.93 (td, J=6.9, 1.2 Hz, 3H).

### Example 31

### N¹-(2-ethoxyphenyl)-N⁴,N⁴-dimethylbenzene-1,4-disulfonamide

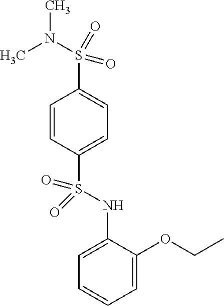 [SMILES: CCOc1ccccc1NS(=O)(=O)c1ccc(S(=O)(=O)N(C)C)cc1]

A solution of 2-ethoxyaniline (0.065 ml, 0.5 mmol) in pyridine (Volume: 1 ml) was treated with 4-(N,N-dimethylsulfamoyl)benzene-1-sulfonyl chloride (0.142 g, 0.500 mmol). The resulting solution was microwaved at 115° C. for 1 hr. The reaction solution was concentrated. Added toluene (3 ml). The crude product was purified by reversed-phase chromatography (4-100% CH₃CN over 15 min) to give desired product (0.084 g, 22% yield). (LCMS, ESI pos.) Calculated for C₁₆H₂₀N₂O₅S₂: 385.5 (M+H), Measured: 385.5. ¹H NMR (400 MHz, DMSO-d6) δ 9.77 (s, 1H), 8.01-7.83 (m, 4H), 7.29 (dt, J=7.8, 1.4 Hz, 1H), 7.19 (tt, J=8.0, 1.4 Hz, 1H), 7.02-6.83 (m, 2H), 3.71 (qd, J=7.0, 1.0 Hz, 2H), 2.67 (d, J=1.1 Hz, 6H), 1.06 (td, J=7.0, 1.1 Hz, 3H).

### Example 32

### N-(2-ethoxyphenyl)-4-(methylsulfonyl)-N-(2-morpholino-2-oxoethyl)benzenesulfonamide

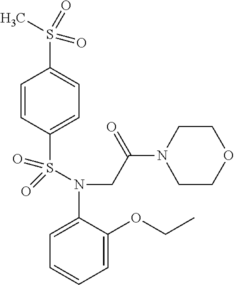 [SMILES: CCOc1ccccc1N(CC(=O)N1CCOCC1)S(=O)(=O)c1ccc(S(C)(=O)=O)cc1]

A solution of 2-ethoxyaniline (0.065 ml, 0.5 mmol) in DMF (Volume: 1 ml) was treated with DIEA (0.1 ml) followed by 4-(methylsulfonyl)benzene-1-sulfonyl chloride (0.127 g, 0.500 mmol). The resulting solution was stirred at room temperature for 1 hr. To the solution was added 2-bromo-1-morpholinoethanone (0.104 g, 0.5 mmol). The solution was microwaved at 125° C. for 2 hrs. The reaction solution was concentrated. Added toluene (3 ml). The crude product was purified by reversed-phase chromatography (4-100% CH₃CN over 15 min) to give desired product (0.006 g, 1% yield). (LCMS, ESI pos.) Calculated for C₂₁H₂₆N₂O₇S₂: 483.0 (M+H), Measured: 482.7. ¹H NMR (400 MHz, DMSO-d6) δ 8.16-8.09 (m, 2H), 7.93-7.87 (m, 2H), 7.52 (dt, J=7.8, 1.5 Hz, 1H), 7.33 (ddt, J=9.0, 7.4, 1.5 Hz, 1H), 7.01-6.91 (m, 2H), 4.54 (s, 2H), 3.73-3.37 (m, 11H), 3.33 (d, J=1.2 Hz, 3H), 0.87 (td, J=7.0, 1.2 Hz, 3H).

### Example 33

### N¹-(3,4-dimethoxybenzyl)-N⁴,N⁴-dimethyl-N¹-(2-(piperidin-1-yl)phenyl)benzene-1,4-disulfonamide

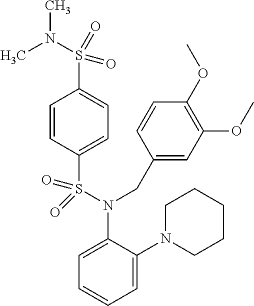

A solution of 2-(piperidin-1-yl)aniline (0.088 g, 0.5 mmol) and DIEA (0.218 ml, 1.250 mmol) in DMF (Volume: 1 ml) was treated with 4-(N,N-dimethylsulfamoyl)benzene-1-sulfonyl chloride (0.142 g, 0.500 mmol). The solution was stirred at room temperature for 1 hr. To the solution was added 4-(bromomethyl)-1,2-dimethoxybenzene (0.116 g, 0.500 mmol). The solution was microwaved at 125° C. for 2 hrs. The crude product was purified by reversed-phase chromatography (4-100% CH₃CN over 15 min) to give desired product (0.006 g, 1% yield). (LCMS, ESI pos.) Calculated for C₂₈H₃₅N₃O₆S₂: 574.7 (M+H), Measured: 574.1. ¹H NMR (400 MHz, DMSO-d6) δ 8.20 (d, J=8.1 Hz, 2H), 8.01 (d, J=8.2 Hz, 2H), 7.28 (t, J=7.5 Hz, 1H), 7.20 (d, J=7.9 Hz, 1H), 6.98 (dt, J=15.2, 7.8 Hz, 2H), 6.70 (d, J=8.2 Hz, 1H), 6.44 (d, J=8.1 Hz, 1H), 6.34 (s, 1H), 4.97 (s, 2H), 3.66 (d, J=1.2 Hz, 4H), 3.49 (s, 3H), 2.72 (d, J=1.2 Hz, 6H), 1.58 (d, J=27.1 Hz, 7H).

### Example 34

### N¹,N¹-dimethyl-N4-(2-(piperidin-1-yl)benzyl)benzene-1,4-disulfonamide

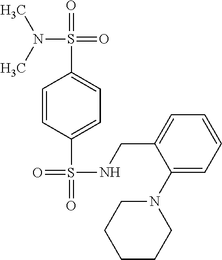 [SMILES: CN(C)S(=O)(=O)c1ccc(S(=O)(=O)NCc2ccccc2N2CCCCC2)cc1]

A solution of (2-(piperidin-1-yl)phenyl)methanamine (0.095 g, 0.500 mmol) in pyridine (Volume: 1 ml) was treated with 4-(N,N-dimethylsulfamoyl)benzene-1-sulfonyl chloride (0.142 g, 0.5 mmol). The solution was microwaved at 115° C. for 1 hr. The crude product was purified by reversed-phase chromatography (4-100% CH₃CN over 15 min) to give desired product (0.103 g, 24% yield). (LCMS, ESI pos.) Calculated for C₂₀H₂₇N₃O₄S₂: 438.6 (M+H), Measured: 438.1. ¹H NMR (400 MHz, DMSO-d6) δ 8.34-8.26 (m, 1H), 8.06-7.99 (m, 2H), 7.97-7.91 (m, 2H), 7.32 (dd, J=7.7, 1.7 Hz, 1H), 7.20 (t, J=7.6 Hz, 1H), 7.02 (q, J=8.6, 7.4 Hz, 2H), 4.14 (d, J=5.8 Hz, 2H), 2.68 (d, J=1.1 Hz, 9H), 1.53 (d, J=19.2 Hz, 6H).

### Example 35

### 4-(methylsulfonyl)-N-(2-(piperidin-1-yl)benzyl)benzenesulfonamide

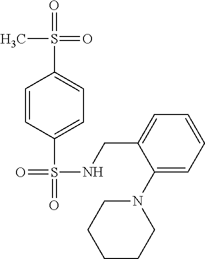 [SMILES: CS(=O)(=O)c1ccc(S(=O)(=O)NCc2ccccc2N2CCCCC2)cc1]

A solution of (2-(piperidin-1-yl)phenyl)methanamine (0.095 g, 0.5 mmol) in pyridine (Volume: 1 ml) was treated with 4-(methylsulfonyl)benzene-1-sulfonyl chloride (0.127 g, 0.500 mmol). The solution was microwaved at 115° C. for 1 hr. The crude product was purified by reversed-phase chromatography (4-100% CH₃CN over 15 min) to give desired product (0.046 g, 11% yield). (LCMS, ESI pos.) Calculated for C₁₉H₂₄N₂O₄S₂: 409.5 (M+H), Measured: 409.1. ¹H NMR (400 MHz, DMSO-d6) δ 8.29 (t, J=6.1 Hz, 1H), 8.16-8.10 (m, 2H), 8.04 (dd, J=8.4, 1.2 Hz, 2H), 7.32 (dd, J=7.7, 1.7 Hz, 1H), 7.21 (t, J=7.6 Hz, 1H), 7.09-6.97 (m, 2H), 4.13 (d, J=5.9 Hz, 2H), 3.32 (d, J=1.1 Hz, 3H), 2.72 (d, J=5.0 Hz, 4H), 1.55 (s, 5H).

### Example 36

### Ethyl N-((4-(N,N-dimethylsulfamoyl)phenyl)sulfonyl)-N-(2-(piperidin-1-yl)benzyl)glycinate

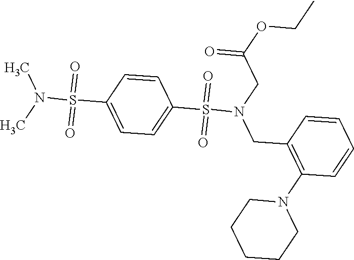 [SMILES: CCOC(=O)CN(Cc1ccccc1N1CCCCC1)S(=O)(=O)c1ccc(S(=O)(=O)N(C)C)cc1]

A solution of (2-(piperidin-1-yl)phenyl)methanamine (0.095 g, 0.500 mmol) and DIEA (0.087 ml, 0.500 mmol) in DMF (Volume: 1 ml) was treated with 4-(N,N-dimethylsulfamoyl)benzene-1-sulfonyl chloride (0.142 g, 0.5 mmol). The solution was stirred at room temperature for 1 hr. To the solution was added ethyl 2-bromoacetate (0.084 g, 0.500 mmol). The solution was microwaved at 125° C. for 2 hrs. The crude product was purified by reversed-phase chromatography (4-100% CH₃CN over 15 min) to give desired product (0.010 g, 2% yield). (LCMS, ESI pos.) Calculated for C₂₄H₃₃N₃O₆S₂: 524.7 (M+H), Measured: 524.1. ¹H NMR (400 MHz, DMSO-d6) δ 8.12 (dd, J=8.2, 1.2 Hz, 2H), 7.97 (dd, J=7.6, 1.5 Hz, 2H), 7.33-7.22 (m, 2H), 7.15 (d, J=8.0 Hz, 1H), 7.06 (t, J=7.4 Hz, 1H), 4.56 (s, 2H), 4.05 (s, 2H), 4.01-3.92 (m, 2H), 2.70 (d, J=0.9 Hz, 11H), 1.53 (d, J=26.5 Hz, 7H), 1.10 (td, J=7.1, 0.9 Hz, 3H).

### Example 37

### N¹-(2-methoxyethyl)-N⁴,N⁴-dimethyl-N1-(2-(piperidin-1-yl)benzyl)benzene-1,4-disulfonamide

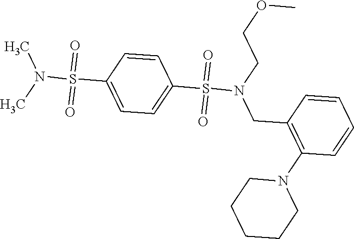 [SMILES: COCCN(Cc1ccccc1N1CCCCC1)S(=O)(=O)c1ccc(S(=O)(=O)N(C)C)cc1]

A solution of (2-(piperidin-1-yl)phenyl)methanamine (0.095 g, 0.5 mmol) and N-ethyl-N-isopropylpropan-2-amine (0.218 ml, 1.250 mmol) in DMF (Volume: 1 ml) was treated with N-ethyl-N-isopropylpropan-2-amine (0.218 ml, 1.250 mmol). The solution was stirred at room temperature for 1 hr. To the solution was added 1-bromo-2-methoxyethane (0.047 ml, 0.500 mmol). The solution was microwaved at 125° C. for 2 hrs. The crude product was purified by reversed-phase chromatography (4-100% CH₃CN over 15 min) to give desired product (0.070 g, 14% yield). (LCMS, ESI pos.) Calculated for C₂₃H₃₃N₃O₅S₂: 496.0 (M+H), Measured: 495.8. ¹H NMR (400 MHz, DMSO-d6) δ 8.11-8.04 (m, 2H), 7.97-7.91 (m, 2H), 7.30-7.20 (m, 2H), 7.11 (d, J=7.9 Hz, 1H), 7.04 (t, J=7.5 Hz, 1H), 4.46 (s, 2H), 3.29-3.15 (m, 4H), 2.96 (d, J=0.9 Hz, 3H), 2.71 (s, 4H), 2.65 (d, J=1.0 Hz, 6H), 1.53 (d, J=37.4 Hz, 6H).

### Example 38

### N¹-(2-fluoro-6-(piperidin-1-yl)phenyl)-N⁴,N⁴-dimethylbenzene-1,4-disulfonamide

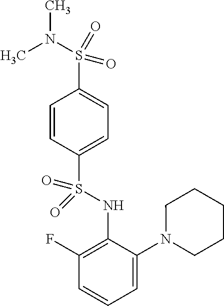 [SMILES: CN(C)S(=O)(=O)c1ccc(S(=O)(=O)Nc2c(F)cccc2N2CCCCC2)cc1]

A solution of 2-fluoro-6-(piperidin-1-yl)aniline (0.097 g, 0.500 mmol) in pyridine (Volume: 1 ml) was treated with 4-(N,N-dimethylsulfamoyl)benzene-1-sulfonyl chloride (0.142 g, 0.5 mmol). The solution was microwaved at 115° C. for 1 hr. The crude product was purified by reversed-phase chromatography (4-100% CH₃CN over 15 min) to give desired product (0.091 g, 20% yield). (LCMS, ESI pos.) Calculated for C₁₉H₂₄FN₃O₄S₂: 442.5 (M+H), Measured: 442.1. ¹H NMR (400 MHz, DMSO-d6) δ 9.51 (s, 1H), 8.06-7.84 (m, 4H), 7.22 (tdd, J=8.0, 6.6, 1.1 Hz, 1H), 6.84 (td, J=8.5, 1.1 Hz, 2H), 2.73 (t, J=4.8 Hz, 4H), 2.65 (d, J=1.0 Hz, 6H), 1.38-1.21 (m, 7H).

### Example 39

### N¹,N¹-dimethyl-N⁴-(2-(piperidin-1-ylmethyl)phenyl)benzene-1,4-disulfonamide

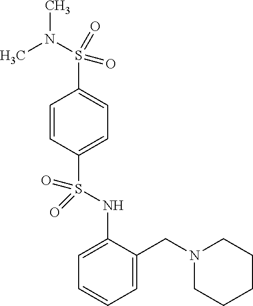 [SMILES: CN(C)S(=O)(=O)c1ccc(S(=O)(=O)Nc2ccccc2CN2CCCCC2)cc1]

A solution of 2-(piperidin-1-ylmethyl)aniline, H2SO4 (0.144 g, 0.500 mmol) in pyridine (Volume: 1 ml) was treated with 4-(N,N-dimethylsulfamoyl)benzene-1-sulfonyl chloride (0.142 g, 0.5 mmol). The solution was microwaved at 115° C. for 1 hr. The crude product was purified by reversed-phase chromatography (4-100% CH₃CN over 15 min) to give desired product (0.037 g, 9% yield). (LCMS, ESI pos.) Calculated for C₂₀H₂₇N₃O₄S₂: 438.6 (M+H), Measured: 438.1. ¹H NMR (400 MHz, DMSO-d6) δ 10.23 (s, 1H), 9.10 (s, 1H), 8.08-7.83 (m, 4H), 7.66 (s, 1H), 7.52-7.26 (m, 2H), 6.79 (s, 1H), 4.38 (s, 2H), 3.31 (d, J=12.2 Hz, 2H), 2.97 (s, 2H), 2.68 (d, J=1.1 Hz, 6H), 1.94-1.29 (m, 7H).

### Example 40

### N¹,N¹-dimethyl-N⁴-(3-(piperidin-1-yl)pyridin-2-yl)benzene-1,4-disulfonamide

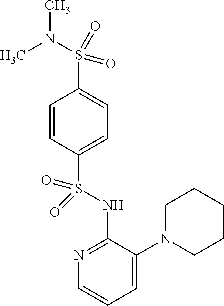 [SMILES: CN(C)S(=O)(=O)c1ccc(S(=O)(=O)Nc2ncccc2N2CCCCC2)cc1]

A solution of 3-(piperidin-1-yl)pyridin-2-amine (0.089 g, 0.500 mmol) in pyridine (Volume: 1 ml) was treated with 4-(N,N-dimethylsulfamoyl)benzene-1-sulfonyl chloride (0.142 g, 0.5 mmol). The solution was microwaved at 115° C. for 1 hr. The crude product was purified by reversed-phase chromatography (4-100% CH₃CN over 15 min) to give desired product (0.032 g, 8% yield). (LCMS, ESI pos.) Calculated for C₁₈H₂₄N₄O₄S₂: 425.5 (M+H), Measured: 425.1. ¹H NMR (400 MHz, DMSO-d6) δ 12.54 (s, 1H), 8.18 (s, 2H), 7.95 (d, J=8.0 Hz, 2H), 7.33 (s, 1H), 6.93 (d, J=39.6 Hz, 1H), 2.92 (s, 4H), 2.67 (d, J=1.0 Hz, 6H), 1.59 (d, J=32.7 Hz, 7H).

### Example 41

### N¹,N¹-dimethyl-N⁴-(4-(piperidin-1-yl)pyridin-3-yl)benzene-1,4-disulfonamide

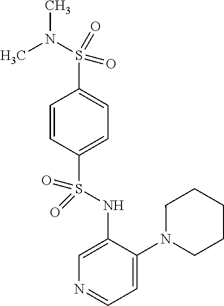 [SMILES: CN(C)S(=O)(=O)c1ccc(S(=O)(=O)Nc2cnccc2N2CCCCC2)cc1]

A solution of 4-(piperidin-1-yl)pyridin-3-amine (0.089 g, 0.500 mmol) in pyridine (Volume: 1 ml) was treated with 4-(N,N-dimethylsulfamoyl)benzene-1-sulfonyl chloride (0.142 g, 0.5 mmol). The solution was microwaved at 115° C. for 1 hr. The crude product was purified by reversed-phase chromatography (4-100% CH₃CN over 15 min) to give desired product (0.025 g, 6% yield). (LCMS, ESI pos.) Calculated for C₁₈H₂₄N₄O₄S₂: 425.5 (M+H), Measured: 425.1. ¹H NMR (400 MHz, DMSO-d6) δ 8.11 (dd, J=7.0, 1.3 Hz, 1H), 7.92 (qd, J=8.9, 1.0 Hz, 4H), 7.56 (d, J=1.1 Hz, 1H), 7.18 (d, J=7.1 Hz, 1H), 3.58 (t, J=4.8 Hz, 4H), 2.64 (d, J=1.0 Hz, 6H), 1.56 (d, J=8.9 Hz, 7H).

### Example 42

### N¹,N¹-dimethyl-N⁴-(3-(piperidin-1-yl)pyridin-4-yl)benzene-1,4-disulfonamide

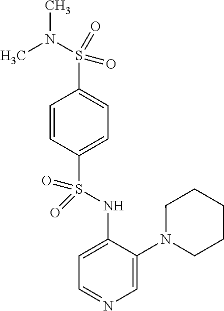 [SMILES: CN(C)S(=O)(=O)c1ccc(S(=O)(=O)Nc2ccncc2N2CCCCC2)cc1]

A solution of 3-(piperidin-1-yl)pyridin-4-amine (0.089 g, 0.500 mmol) in pyridine (Volume: 1 ml) was treated with 4-(N,N-dimethylsulfamoyl)benzene-1-sulfonyl chloride (0.142 g, 0.5 mmol). The solution was microwaved at 115° C. for 1 hr. The crude product was purified by reversed-phase chromatography (4-100% CH₃CN over 15 min) to give desired product (0.013 g, 3% yield). (LCMS, ESI pos.) Calculated for C₁₈H₂₄N₄O₄S₂: 425.5 (M+H), Measured: 425.1. ¹H NMR (400 MHz, DMSO-d6) δ 8.13-8.07 (m, 2H), 7.94-7.84 (m, 4H), 7.69 (s, 1H), 7.28 (dd, J=6.7, 1.1 Hz, 1H), 3.04 (s, 4H), 2.67 (d, J=1.0 Hz, 6H), 1.60 (d, J=31.4 Hz, 7H).

### Example 43

### N¹,N¹-dimethyl-N⁴-(2-(piperidin-1-yl)pyridin-3-yl)benzene-1,4-disulfonamide

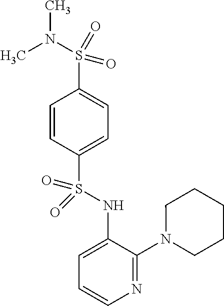 [SMILES: CN(C)S(=O)(=O)c1ccc(S(=O)(=O)Nc2cccnc2N2CCCCC2)cc1]

A solution of 2-(piperidin-1-yl)pyridin-3-amine (0.089 g, 0.500 mmol) in pyridine (Volume: 1 ml) was treated with 4-(N,N-dimethylsulfamoyl)benzene-1-sulfonyl chloride (0.142 g, 0.5 mmol). The solution was microwaved at 115° C. for 1 hr. The crude product was purified by reversed-phase chromatography (4-100% CH₃CN over 15 min) to give desired product (0.113 g, 27% yield). (LCMS, ESI pos.) Calculated for C₁₈H₂₄N₄O₄S₂: 425.5 (M+H), Measured: 425.1. ¹H NMR (400 MHz, DMSO-d6) δ 8.14-8.08 (m, 1H), 8.05-7.96 (m, 4H), 7.35 (ddd, J=7.8, 1.9, 0.9 Hz, 1H), 6.93 (ddd, J=7.9, 4.9, 1.1 Hz, 1H), 3.02 (d, J=5.2 Hz, 4H), 2.69 (d, J=1.0 Hz, 6H), 1.51 (d, J=2.7 Hz, 6H).

### Example 44

### N¹,N¹,N⁴-trimethyl-N4-(2-(piperidin-1-yl)phenyl)benzene-1,4-disulfonamide

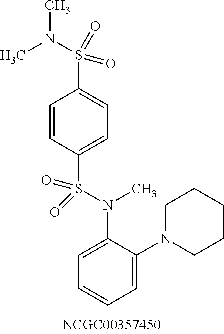 [SMILES: CN(C)S(=O)(=O)c1ccc(S(=O)(=O)N(C)c2ccccc2N2CCCCC2)cc1]

A solution of 2-(piperidin-1-yl)aniline (0.088 g, 0.5 mmol) in pyridine (Volume: 1 ml) was treated with DIEA (0.105 ml, 0.600 mmol). To the solution was added 4-(N,N-dimethylsulfamoyl)benzene-1-sulfonyl chloride (0.142 g, 0.500 mmol). The solution was stirred at room temperature for 1 hr. METHYL IODIDE (0.031 ml, 0.500 mmol) was added to the solution. The solution was microwaved at 115° C. for 2 hrs. The crude product was purified by reversed-phase chromatography (4-100% CH₃CN over 15 min) to give desired product (0.061 g, 14% yield). (LCMS, ESI pos.) Calculated for C₂₀H₂₇N₃O₄S₂: 437.6 (M+H), Measured: 437.8. ¹H NMR (400 MHz, DMSO-d6) δ 8.16-7.91 (m, 4H), 7.33 (d, J=8.1 Hz, 1H), 7.20 (s, 1H), 7.03 (s, 1H), 6.91 (d, J=7.8 Hz, 1H), 3.27 (d, J=1.4 Hz, 3H), 2.91 (d, J=9.2 Hz, 4H), 2.72 (d, J=1.6 Hz, 7H), 1.57 (d, J=16.7 Hz, 6H).

### Example 45

### 4-(methylsulfonyl)-N-(3-(piperidin-1-yl)phenyl)benzenesulfonamide

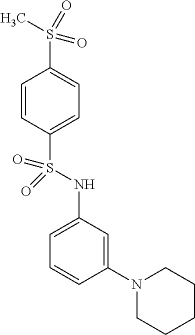 [SMILES: CS(=O)(=O)c1ccc(S(=O)(=O)Nc2cccc(N3CCCCC3)c2)cc1]

A solution of 3-(piperidin-1-yl)aniline (0.088 g, 0.500 mmol) in pyridine (Volume: 1 ml) was treated with 4-(methylsulfonyl)benzene-1-sulfonyl chloride (0.127 g, 0.5 mmol). The solution was microwaved at 115° C. for 1 hr. The crude product was purified by reversed-phase chromatography (4-100% CH₃CN over 15 min) to give desired product (0.069 g, 18% yield). (LCMS, ESI pos.) Calculated for C₁₈H₂₂N₂O₄S₂: 394.5 (M+H), Measured: 394.8. ¹H NMR (400 MHz, DMSO-d6) δ 10.57-10.39 (m, 1H), 8.21-8.09 (m, 2H), 8.09-7.96 (m, 2H), 7.16 (t, J=8.1 Hz, 1H), 6.73 (d, J=63.4 Hz, 3H), 3.31 (d, J=1.0 Hz, 3H), 3.15 (d, J=6.4 Hz, 4H), 1.61 (d, J=24.4 Hz, 6H).

### Example 46

### N-(2-(4-methylpiperazin-1-yl)phenyl)-4-(methylsulfonyl)benzenesulfonamide

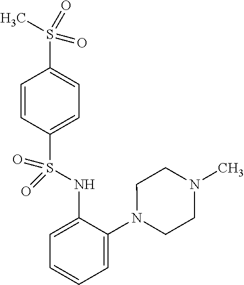 [SMILES: CN1CCN(c2ccccc2NS(=O)(=O)c2ccc(S(C)(=O)=O)cc2)CC1]

A solution of 2-(4-methylpiperazin-1-yl)aniline (0.096 g, 0.500 mmol) in pyridine (Volume: 1 ml) was treated with 2-(4-methylpiperazin-1-yl)aniline (0.096 g, 0.500 mmol). The solution was microwaved at 115° C. for 1 hr. The crude product was purified by reversed-phase chromatography (4-100% CH₃CN over 15 min) to give desired product (0.116 g, 28% yield). (LCMS, ESI pos.) Calculated for C₁₈H₂₃N₃O₄S₂: 410.1 (M+H), Measured: 409.8. ¹H NMR (400 MHz, DMSO-d6) δ 9.60 (s, 1H), 8.20-8.12 (m, 2H), 8.08-8.01 (m, 2H), 7.37 (d, J=7.3 Hz, 1H), 7.25-7.15 (m, 2H), 3.38 (s, 4H), 3.33 (s, 3H), 2.87 (s, 3H), 2.67 (s, 2H).

### Example 47

### N¹-(2-(4,4-dimethylpiperidin-1-yl)phenyl)-N⁴,N⁴-dimethylbenzene-1,4-disulfonamide

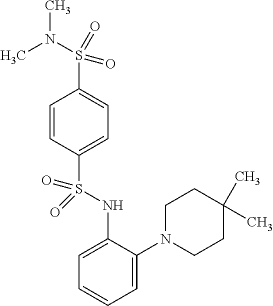

A mixture of 2-(4,4-dimethylpiperidin-1-yl)aniline (0.20 g, 1.0 mmol) and potassium carbonate (0.14 g, 1.0 mmol) in DMF (1.0 ml) was treated with 4-(N,N-dimethylsulfamoyl)benzene-1-sulfonyl chloride (0.28 g, 1.0 mmol). The solution was microwaved at 155° C. for 1 hr. The solution was cooled to room temperature. The solution was filtered to give a solid (2.0 mg, 1% yield). (LCMS, ESI pos.) Calculated for C₂₁H₂₉N₃O₄S₂: 452.6 (M+H), Measured: 452.1. ¹H NMR (400 MHz, DMSO-d6) δ 9.12 (s, 1H), 8.07-8.00 (m, 2H), 7.99-7.92 (m, 2H), 7.31 (dt, J=7.8, 1.3 Hz, 1H), 7.23 (d, J=8.0 Hz, 1H), 7.15 (dd, J=8.4, 6.8 Hz, 1H), 7.12-7.05 (m, 1H), 2.65 (d, J=1.0 Hz, 6H), 2.50 (d, J=5.7 Hz, 4H), 1.37 (t, J=5.5 Hz, 4H), 0.96 (d, J=1.0 Hz, 6H).

### Example 48

### 3-(methylsulfonyl)-N-(4-(piperidin-1-yl)phenyl)benzenesulfonamide

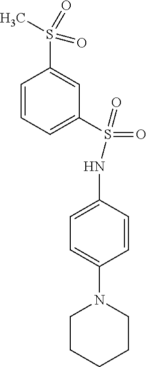 [SMILES: CS(=O)(=O)c1cccc(S(=O)(=O)Nc2ccc(N3CCCCC3)cc2)c1]

A solution of 4-(piperidin-1-yl)aniline (0.088 g, 0.500 mmol) in pyridine (Volume: 1 ml) was treated with 3-(methylsulfonyl)benzene-1-sulfonyl chloride (0.127 g, 0.5 mmol). The solution was microwaved at 115° C. for 1 hr. The crude product was purified by reversed-phase chromatography (4-100% CH₃CN over 15 min) to give desired product (0.126 g, 32% yield). (LCMS, ESI pos.) Calculated for C₁₈H₂₂N₂O₄S₂: 395.5 (M+H), Measured: 395.1. ¹H NMR (400 MHz, DMSO-d6) δ 10.38 (s, 1H), 8.22 (dd, J=7.4, 1.2 Hz, 2H), 8.06 (d, J=7.9 Hz, 1H), 7.97-7.77 (m, 1H), 7.11 (d, J=34.2 Hz, 4H), 3.30 (s, 3H), 1.65 (d, J=52.3 Hz, 6H).

### Example 49

### 3-(methylsulfonyl)-N-(2-(piperidin-1-yl)phenyl)benzenesulfonamide

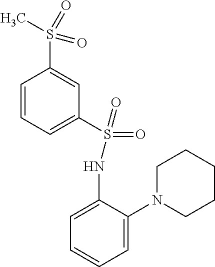 [SMILES: CS(=O)(=O)c1cccc(S(=O)(=O)Nc2ccccc2N2CCCCC2)c1]

A solution of 2-(piperidin-1-yl)aniline (0.088 g, 0.500 mmol) in pyridine (Volume: 1 ml) was treated with 3-(methylsulfonyl)benzene-1-sulfonyl chloride (0.127 g, 0.5 mmol). The solution was microwaved at 115° C. for 1 hr. The crude product was purified by reversed-phase chromatography (4-100% CH₃CN over 15 min) to give desired product (0.072 g, 18% yield). (LCMS, ESI pos.) Calculated for C₁₈H₂₂N₂O₄S₂: 395.5 (M+H), Measured: 395.1. ¹H NMR (400 MHz, DMSO-d6) δ 9.17 (s, 1H), 8.34-8.17 (m, 2H), 8.14 (ddd, J=7.9, 1.9, 1.1 Hz, 1H), 7.90 (td, J=7.8, 0.5 Hz, 1H), 7.35-7.00 (m, 4H), 3.29 (s, 3H), 1.65-1.40 (m, 7H).

### Example 5

### N-(2-(4-methylpiperidin-1-yl)phenyl)-3-(methylsulfonyl)benzenesulfonamide

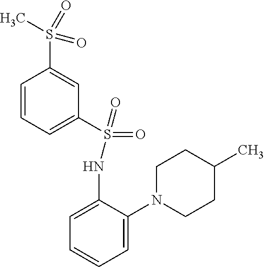 [SMILES: CC1CCN(c2ccccc2NS(=O)(=O)c2cccc(S(C)(=O)=O)c2)CC1]

A solution of 2-(4-methylpiperidin-1-yl)aniline (0.095 g, 0.500 mmol) in pyridine (Volume: 1 ml) was treated with 3-(methylsulfonyl)benzene-1-sulfonyl chloride (0.127 g, 0.5 mmol). The solution was microwaved at 115° C. for 1 hr. The crude product was purified by reversed-phase chromatography (4-100% CH₃CN over 15 min) to give desired product (0.073 g, 18% yield). (LCMS, ESI pos.) Calculated for C₁₉H₂₄N₂O₄S₂: 409.5 (M+H), Measured: 409.1. ¹H NMR (400 MHz, DMSO-d6) δ 9.17 (s, 1H), 8.33-8.18 (m, 2H), 8.14 (ddd, J=7.9, 1.9, 1.1 Hz, 1H), 7.90 (td, J=7.8, 0.5 Hz, 1H), 7.33-6.97 (m, 4H), 3.29 (s, 3H), 2.63 (d, J=11.3 Hz, 2H), 2.48 (d, J=11.5 Hz, 2H), 1.56 (d, J=12.4 Hz, 2H), 1.34-1.10 (m, 2H), 0.95 (d, J=6.4 Hz, 3H).

### Example 51

### N-(2-(4-methylpiperazin-1-yl)phenyl)-3-(methylsulfonyl)benzenesulfonamide

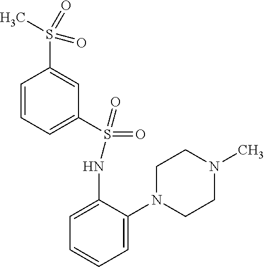 [SMILES: CN1CCN(c2ccccc2NS(=O)(=O)c2cccc(S(C)(=O)=O)c2)CC1]

A solution of 2-(4-methylpiperazin-1-yl)aniline (0.096 g, 0.500 mmol) in pyridine (Volume: 1 ml) was treated with 3-(methylsulfonyl)benzene-1-sulfonyl chloride (0.127 g, 0.5 mmol). The solution was microwaved at 115° C. for 1 hr. The crude product was purified by reversed-phase chromatography (4-100% CH₃CN over 15 min) to give desired product (0.077 g, 19% yield). (LCMS, ESI pos.) Calculated for C₁₈H₂₃N₃O₄S₂: 410.5 (M+H), Measured: 410.1. ¹H NMR (400 MHz, DMSO-d6) δ 9.74 (s, 1H), 9.58 (s, 1H), 8.35-8.18 (m, 2H), 8.11 (ddd, J=7.9, 1.9, 1.1 Hz, 1H), 7.90 (td, J=7.8, 0.5 Hz, 1H), 7.44-7.31 (m, 1H), 7.29-7.09 (m, 3H), 3.41 (d, J=11.9 Hz, 2H), 3.33 (s, 3H), 3.13 (s, 2H), 2.87 (s, 5H), 2.70 (d, J=12.7 Hz, 2H).

### Example 52

### 3-(methylsulfonyl)-N-(3-(piperidin-1-yl)phenyl)benzenesulfonamide

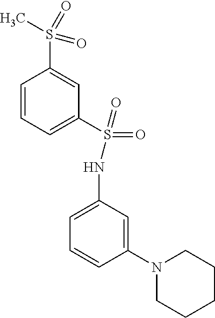 [SMILES: CS(=O)(=O)c1cccc(S(=O)(=O)Nc2cccc(N3CCCCC3)c2)c1]

A solution of 3-(piperidin-1-yl)aniline (0.088 g, 0.500 mmol) in pyridine (Volume: 1 ml) was treated with 3-(methylsulfonyl)benzene-1-sulfonyl chloride (0.127 g, 0.5 mmol). The solution was microwaved at 115° C. for 1 hr. The crude product was purified by reversed-phase chromatography (4-100% CH₃CN over 15 min) to give desired product (0.117 g, 30% yield). (LCMS, ESI pos.) Calculated for C₁₈H₂₂N₂O₄S₂: 395.5 (M+H), Measured: 395.1. ¹H NMR (400 MHz, DMSO-d6) δ 10.34 (s, 1H), 8.30-8.08 (m, 2H), 8.03 (ddd, J=7.9, 1.9, 1.1 Hz, 1H), 7.83 (td, J=7.8, 0.5 Hz, 1H), 7.07 (d, J=9.0 Hz, 1H), 6.64 (d, J=74.0 Hz, 3H), 3.24 (s, 3H), 3.07 (d, J=6.7 Hz, 4H), 1.54 (d, J=24.1 Hz, 6H).

### Example 53

### N-(3-chloro-2-(piperidin-1-yl)phenyl)-3-(methylsulfonyl)benzenesulfonamide

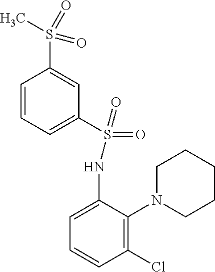 [SMILES: CS(=O)(=O)c1cccc(S(=O)(=O)Nc2cccc(Cl)c2N2CCCCC2)c1]

A solution of 3-chloro-2-(piperidin-1-yl)aniline, 2HCl (0.142 g, 0.500 mmol) in pyridine (Volume: 1 ml) was treated with 3-(methylsulfonyl)benzene-1-sulfonyl chloride (0.127 g, 0.5 mmol). The solution was microwaved at 115° C. for 1 hr. The crude product was purified by reversed-phase chromatography (4-100% CH₃CN over 15 min) to give desired product (0.092 g, 21% yield). (LCMS, ESI pos.) Calculated for C₁₈H₂₁ClN₂O₄S₂: 428.9 (M+H), Measured: 429.1, 431.0. ¹H NMR (400 MHz, DMSO-d6) δ 9.27 (s, 1H), 8.34-8.19 (m, 2H), 8.10 (ddd, J=7.9, 1.9, 1.1 Hz, 1H), 7.91 (td, J=7.8, 0.6 Hz, 1H), 7.33-7.09 (m, 3H), 3.30 (s, 3H), 3.15 (d, J=19.1 Hz, 2H), 2.43 (s, 2H), 1.58 (d, J=5.9 Hz, 5H), 1.36 (s, 1H).

### Example 54

### N¹-(2-(4,4-difluoropiperidin-1-yl)phenyl)-N⁴,N⁴-dimethylbenzene-1,4-disulfonamide

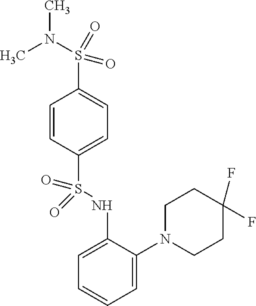 [SMILES: CN(C)S(=O)(=O)c1ccc(S(=O)(=O)Nc2ccccc2N2CCC(F)(F)CC2)cc1]

A solution of 2-(4,4-difluoropiperidin-1-yl)aniline (0.212 g, 1.000 mmol) in DMF (Volume: 2 ml) was treated with POTASSIUM CARBONATE (0.138 g, 1 mmol). To the solution was added 4-(N,N-dimethylsulfamoyl)benzene-1-sulfonyl chloride (0.284 g, 1.000 mmol). The solution was stirred at room temperature for 1 hr then microwaved at 155° C. for 2 hrs. The crude product was purified by reversed-phase chromatography (4-100% CH₃CN over 15 min) to give desired product (0.024 g, 5% yield). (LCMS, ESI pos.) Calculated for C₁₉H₂₃F₂N₃O₄S₂: 460.5 (M+H), Measured: 460.1. ¹H NMR (400 MHz, DMSO-d6) δ 9.55 (s, 1H), 8.08-8.01 (m, 2H), 8.01-7.94 (m, 2H), 7.39-7.30 (m, 1H), 7.26-7.05 (m, 3H), 2.69-2.60 (m, 11H), 2.05 (tt, J=13.3, 5.6 Hz, 4H).

### Example 55

### 4-(methylthio)-N-(2-(piperidin-1-yl)phenyl)benzenesulfonamide

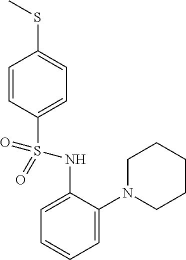 [SMILES: CSc1ccc(S(=O)(=O)Nc2ccccc2N2CCCCC2)cc1]

A solution of 2-(piperidin-1-yl)aniline (0.088 g, 0.500 mmol) in pyridine (Volume: 1 ml) was treated with 4-(methylthio)benzene-1-sulfonyl chloride (0.111 g, 0.5 mmol). The solution was microwaved at 115° C. for 1 hr. The solution was cooled to room temperature. The solution was filtered to give a solid (0.074 g, 20% yield). (LCMS, ESI pos.) Calculated for C₁₈H₂₂N₂O₂S₂: 363.5 (M+H), Measured: 363.1. ¹H NMR (400 MHz, DMSO-d6) δ 8.67 (s, 1H), 7.78-7.62 (m, 2H), 7.48-7.37 (m, 2H), 7.37-7.27 (m, 1H), 7.22-7.12 (m, 1H), 7.13-7.02 (m, 2H), 2.53 (s, 3H), 2.52-2.48 (m, 5H), 1.63 (dq, J=10.8, 5.0 Hz, 5H), 1.51 (q, J=6.0 Hz, 2H).

### Example 56

### N¹-(2-cyclohexylphenyl)-N⁴,N⁴-dimethylbenzene-1,4-disulfonamide

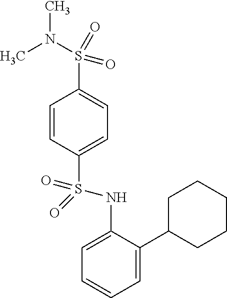 [SMILES: CN(C)S(=O)(=O)c1ccc(S(=O)(=O)Nc2ccccc2C2CCCCC2)cc1]

A solution of 2-cyclohexylaniline, HCl (0.106 g, 0.500 mmol) in pyridine (Volume: 1 ml) was treated with 4-(N,N-dimethylsulfamoyl)benzene-1-sulfonyl chloride (0.142 g, 0.5 mmol). The solution was microwaved at 115° C. for 1 hr. The crude product was purified by reversed-phase chromatography (4-100% CH₃CN over 15 min) to give desired product (0.080 g, 19% yield). (LCMS, ESI pos.) Calculated for C₂₀H₂₆N₂O₄S₂: 423.6 (M+H), Measured: 423.1. ¹H NMR (400 MHz, DMSO-d6) δ 10.05 (s, 1H), 8.00-7.93 (m, 2H), 7.93-7.84 (m, 2H), 7.27-7.21 (m, 2H), 7.19-7.12 (m, 1H), 7.09 (dt, J=7.9, 1.1 Hz, 1H), 2.68 (s, 6H), 1.64 (d, J=7.1 Hz, 3H), 1.16 (s, 7H).

### Example 57 (a)

### trans-4-methyl-1-(2-nitrophenyl)piperidin-3-ol

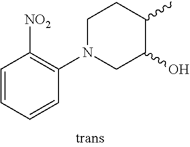 [SMILES: CC1CCN(c2ccccc2[N+](=O)[O-])CC1O]

A solution of 4-methylpiperidin-3-ol (0.115 g, 1 mmol) and POTASSIUM CARBONATE (0.138 g, 1.000 mmol) in DMF (Volume: 1 ml) was treated with 1-fluoro-2-nitrobenzene (0.106 ml, 1.000 mmol). The solution was stirred at room temperature for a few minutes. Microwaved at 155° C. for 2 hrs. The reaction solution was filtered through Celite. The filtrate was used as is for the next step.

### Example 57 (b)

### Trans-1-(2-aminophenyl)-4-methylpiperidin-3-ol

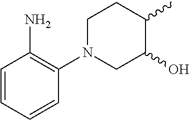

The solution of 4-methyl-1-(2-nitrophenyl)piperidin-3-ol (0.236 g, 1 mmol) in MeOH was subjected to H-cube conditions: 0.8 ml/min, 100% H2, 70 bar, 70° C. The solution was concentrated. Used as is for the next step.

### Example 57 (c) trans-N1-(2-(3-hydroxy-4-methylpiperidin-1-yl)phenyl)-N4,N₄-dimethylbenzene-1,4-disulfonamide

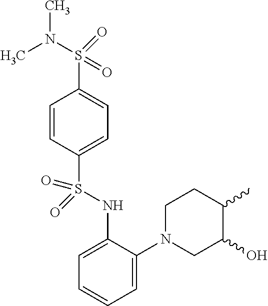 [SMILES: CC1CCN(c2ccccc2NS(=O)(=O)c2ccc(S(=O)(=O)N(C)C)cc2)CC1O]

A solution of 1-(2-aminophenyl)-4-methylpiperidin-3-ol (0.206 g, 1.000 mmol) and potassium carbonate (0.138 g, 1.000 mmol) in DMF (Volume: 2 ml) was treated with 4-(N,N-dimethylsulfamoyl)benzene-1-sulfonyl chloride (0.284 g, 1 mmol). The solution was microwaved at 155° C. for 1 hr. The crude product was purified by reversed-phase chromatography (4-100% CH₃CN over 15 min) to give desired product (0.041 g, 11% yield). (LCMS, ESI pos.) Calculated for C₂₀H₂₇N₃O₅S₂: 454.6 (M+H), Measured: 454.1. ¹H NMR (400 MHz, DMSO-d6) δ 9.22 (s, 1H), 8.11-8.03 (m, 2H), 8.00-7.92 (m, 2H), 7.33-7.25 (m, 1H), 7.18-7.11 (m, 2H), 7.12-7.02 (m, 1H), 3.30-3.18 (m, 2H), 2.77 (ddd, J=10.8, 4.4, 1.6 Hz, 1H), 2.66 (s, 7H), 2.45-2.34 (m, 1H), 2.27 (dd, J=10.8, 9.2 Hz, 1H), 1.67-1.56 (m, 1H), 1.33 (dd, J=13.6, 5.7 Hz, 2H), 1.02 (d, J=6.0 Hz, 3H).

### Example 58

### N¹-(2-(4-ethyl-4-methylpiperidin-1-yl)phenyl)-N⁴,N⁴-dimethylbenzene-1,4-disulfonamide

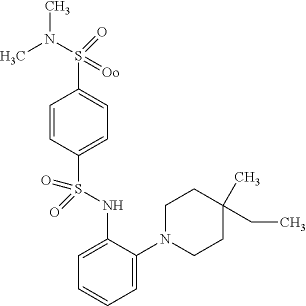

A solution of 2-(4-ethyl-4-methylpiperidin-1-yl)aniline (0.218 g, 1.000 mmol; prepared using the procedure from Example xxx (a) and (b) with 4-ethyl-4-methylpiperidine (0.25 g, 2.0 mmol)) and POTASSIUM CARBONATE (0.138 g, 1.000 mmol) in DMF (Volume: 2 ml) was treated with 4-(N,N-dimethylsulfamoyl)benzene-1-sulfonyl chloride (0.284 g, 1 mmol). The solution was microwaved at 155° C. for 1 hr. The crude product was purified by reversed-phase chromatography (4-100% CH₃CN over 15 min) to give desired product (0.057 g, 12% yield). (LCMS, ESI pos.) Calculated for C₂₂H₃₁N₃O₄S₂: 466.6 (M+H), Measured: 466.1. ¹H NMR (400 MHz, DMSO-d6) δ 9.14 (s, 1H), 8.11-7.99 (m, 2H), 7.99-7.89 (m, 2H), 7.31 (dd, J=7.9, 1.6 Hz, 1H), 7.23 (dd, J=7.9, 1.6 Hz, 1H), 7.16 (td, J=7.6, 1.7 Hz, 1H), 7.09 (td, J=7.6, 1.6 Hz, 1H), 2.65 (s, 6H), 2.48 (h, J=4.6, 4.2 Hz, 2H), 1.47-1.25 (m, 7H), 0.90 (s, 3H), 0.84 (t, J=7.5 Hz, 3H).

### Example 59

### N¹-(2-(4-hydroxypiperidin-1-yl)phenyl)-N⁴,N⁴-dimethylbenzene-1,4-disulfonamide

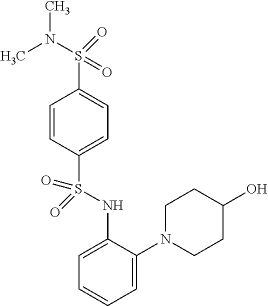 [SMILES: CN(C)S(=O)(=O)c1ccc(S(=O)(=O)Nc2ccccc2N2CCC(O)CC2)cc1]

A solution of 1-(2-aminophenyl)piperidin-4-ol (0.096 g, 0.500 mmol) in pyridine (Volume: 1 ml) was treated with 4-(N,N-dimethylsulfamoyl)benzene-1-sulfonyl chloride (0.142 g, 0.5 mmol). The solution was microwaved at 115° C. for 1 hr. The crude product was purified by reversed-phase chromatography (4-100% CH₃CN over 15 min) to give desired product (0.103 g, 23% yield). (LCMS, ESI pos.) Calculated for C₁₉H₂₅N₃O₅S₂: 440.6 (M+H), Measured: 440.1. ¹H NMR (400 MHz, DMSO-d6) δ 9.20 (s, 1H), 8.07-8.01 (m, 2H), 7.99-7.93 (m, 2H), 7.34-7.28 (m, 1H), 7.21-7.05 (m, 3H), 3.57 (tt, J=8.4, 4.0 Hz, 1H), 3.21 (s, 1H), 2.66 (s, 7H), 2.62 (dd, J=11.9, 5.8 Hz, 1H), 2.44 (t, J=9.9 Hz, 2H), 1.73 (dd, J=13.4, 4.2 Hz, 2H), 1.52 (dtd, J=12.6, 9.2, 3.7 Hz, 2H).

### Example 60

### 1-(2-((4-(N,N-dimethylsulfamoyl)phenyl)sulfonamido)phenyl)piperidine-4-carboxamide

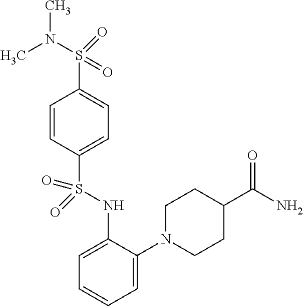 [SMILES: CN(C)S(=O)(=O)c1ccc(S(=O)(=O)Nc2ccccc2N2CCC(C(N)=O)CC2)cc1]

A solution of 1-(2-aminophenyl)piperidine-4-carboxamide (0.110 g, 0.500 mmol) in pyridine (Volume: 1 ml) was treated with 4-(N,N-dimethylsulfamoyl)benzene-1-sulfonyl chloride (0.142 g, 0.5 mmol). The solution was microwaved at 115° C. for 1 hr. The crude product was purified by reversed-phase chromatography (4-100% CH₃CN over 15 min) to give desired product (0.024 g, 5% yield). (LCMS, ESI pos.) Calculated for C₂₀H₂₆N₄₀₅S₂: 467.5 (M+H), Measured: 467.1. ¹H NMR (400 MHz, DMSO-d6) δ 9.24 (s, 1H), 8.14-8.01 (m, 2H), 8.00-7.89 (m, 2H), 7.38-7.23 (m, 2H), 7.23-7.00 (m, 3H), 6.78 (s, 1H), 3.21 (d, J=5.1 Hz, 1H), 2.66 (s, 6H), 2.59 (d, J=11.3 Hz, 1H), 2.49 (td, J=11.5, 2.6 Hz, 2H), 2.16 (tt, J=11.4, 4.0 Hz, 1H), 1.78 (qd, J=12.0, 4.0 Hz, 2H), 1.66 (dd, J=13.1, 3.6 Hz, 2H).

### Example 61

### N¹-(2-(2-azabicyclo[2.2.1]heptan-2-yl)phenyl)-N⁴,N⁴-dimethylbenzene-1,4-disulfonamide

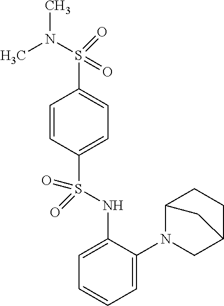 [SMILES: CN(C)S(=O)(=O)c1ccc(S(=O)(=O)Nc2ccccc2N2CC3CCC2C3)cc1]

A solution of 2-((1S,4R)-2-azabicyclo[2.2.1]heptan-2-yl)aniline (0.094 g, 0.500 mmol) in pyridine (Volume: 1 ml) was treated with 4-(N,N-dimethylsulfamoyl)benzene-1-sulfonyl chloride (0.142 g, 0.5 mmol). The resulting solution was microwaved at 115° C. for 1 hr. The crude product was purified by reversed-phase chromatography (4-100% CH₃CN over 15 min) to give desired product (0.046 g, 10% yield). (LCMS, ESI pos.) Calculated for C₂₀H₂₅N₃O₄S₂: 436.6 (M+H), Measured: 436.1. ¹H NMR (400 MHz, DMSO-d6) δ 9.47 (s, 1H), 8.06-7.82 (m, 4H), 7.07 (ddd, J=8.3, 6.0, 2.9 Hz, 1H), 6.77-6.64 (m, 1H), 6.58-6.40 (m, 2H), 4.09 (s, 1H), 3.72-3.55 (m, 1H), 2.75 (d, J=8.8 Hz, 1H), 2.69 (s, 6H), 2.50 (s, 1H), 1.73 (t, J=8.6 Hz, 1H), 1.64-1.57 (m, 2H), 1.49 (d, J=9.4 Hz, 1H), 1.40 (d, J=9.2 Hz, 1H), 1.26 (d, J=11.2 Hz, 1H).

### Example 62

### N¹,N¹-dimethyl-N⁴-(2-(methyl(phenyl)amino)phenyl)benzene-1,4-disulfonamide

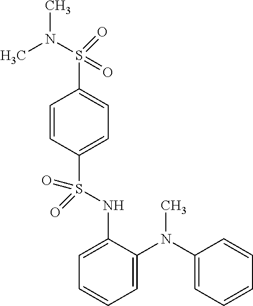 [SMILES: CN(c1ccccc1)c1ccccc1NS(=O)(=O)c1ccc(S(=O)(=O)N(C)C)cc1]

A solution of N1-methyl-N1-phenylbenzene-1,2-diamine (0.099 g, 0.5 mmol) in pyridine (Volume: 1 ml) was treated with 4-(N,N-dimethylsulfamoyl)benzene-1-sulfonyl chloride (0.142 g, 0.500 mmol). The solution was microwaved at 115° C. for 1 hr. The solution was cooled to room temperature. The solution was filtered to give a solid (0.103 g, 23% yield). (LCMS, ESI pos.) Calculated for C₂₁H₂₃N₃O₄S₂: 446.5 (M+H), Measured: 446.1. ¹H NMR (400 MHz, DMSO-d6) δ 9.92 (s, 1H), 8.06-7.97 (m, 2H), 7.94-7.86 (m, 2H), 7.42 (dd, J=8.0, 1.6 Hz, 1H), 7.32-7.16 (m, 2H), 7.15-7.05 (m, 3H), 6.72 (tt, J=7.4, 1.1 Hz, 1H), 6.39 (dt, J=7.9, 1.0 Hz, 2H), 2.88 (s, 3H), 2.66 (s, 6H).

### Example 63

### N¹,N¹-dimethyl-N⁴-(2-(piperidin-1-ylmethyl)benzyl)benzene-1,4-disulfonamide

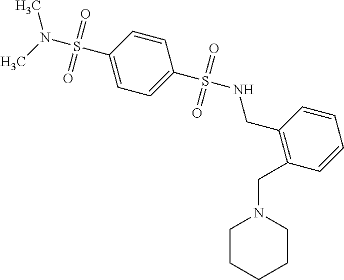 [SMILES: CN(C)S(=O)(=O)c1ccc(S(=O)(=O)NCc2ccccc2CN2CCCCC2)cc1]

A solution of (2-(piperidin-1-ylmethyl)phenyl)methanamine (0.102 g, 0.500 mmol) in pyridine (Volume: 1 ml) was treated with 4-(N,N-dimethylsulfamoyl)benzene-1-sulfonyl chloride (0.142 g, 0.5 mmol). The resulting solution was microwaved at 115° C. for 1 hr. The crude product was purified by reversed-phase chromatography (4-100% CH₃CN over 15 min) to give desired product (0.045 g, 10% yield). (LCMS, ESI pos.) Calculated for C₂₁H₂₉N₃O₄S₂: 452.6 (M+H), Measured: 452.1. ¹H NMR (400 MHz, DMSO-d6) δ 9.08 (s, 1H), 8.50 (t, J=6.1 Hz, 1H), 8.01-7.94 (m, 2H), 7.93-7.86 (m, 2H), 7.48-7.40 (m, 1H), 7.37-7.27 (m, 2H), 4.29 (d, J=5.4 Hz, 2H), 4.22 (d, J=6.1 Hz, 2H), 3.26 (d, J=12.2 Hz, 2H), 2.93 (q, J=10.9 Hz, 2H), 2.64 (s, 6H), 1.84-1.49 (m, 5H), 1.35 (dd, J=16.9, 7.9 Hz, 1H).

### Example 64

### N¹-(2-(6-azaspiro[2.5]octan-6-yl)phenyl)-N⁴,N⁴-dimethylbenzene-1,4-disulfonamide

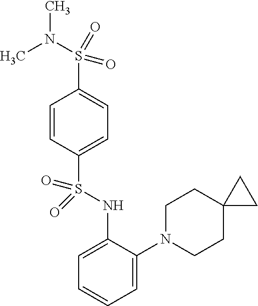 [SMILES: CN(C)S(=O)(=O)c1ccc(S(=O)(=O)Nc2ccccc2N2CCC3(CC2)CC3)cc1]

2-(6-azaspiro[2.5]octan-6-yl)aniline (0.202 g, 1.000 mmol; prepared as in Example xxx (a),(b) using 6-azaspiro[2.5]octane (0.22 g, 2.0 mmol)) was taken up in DMF (Volume: 2 ml) and POTASSIUM CARBONATE (0.138 g, 1.000 mmol) was added. To the resulting solution was added 4-(N,N-dimethylsulfamoyl)benzene-1-sulfonyl chloride (0.284 g, 1 mmol). The solution was microwaved at 155° C. for 2 hrs. The crude product was purified by reversed-phase chromatography (4-100% CH₃CN over 15 min) to give desired product (0.042 g, 10% yield). (LCMS, ESI pos.) Calculated for C₂₁H₂₇N₃O₄S₂: 450.6 (M+H), Measured: 450.1. ¹H NMR (400 MHz, DMSO-d6) δ 9.13 (s, 1H), 8.07-7.92 (m, 2H), 7.94-7.83 (m, 2H), 7.33-7.21 (m, 1H), 7.18-6.93 (m, 3H), 2.58 (s, 6H), 2.50 (d, J=5.3 Hz, 4H), 1.32 (s, 4H), 0.25 (s, 4H).

### Example 65

### N¹-(3-chloro-2-(piperidin-1-yl)phenyl)-N³,N³-dimethylbenzene-1,3-disulfonamide

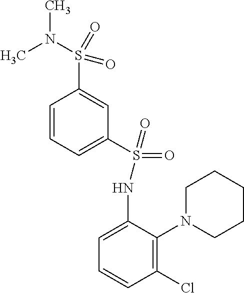 [SMILES: CN(C)S(=O)(=O)c1cccc(S(=O)(=O)Nc2cccc(Cl)c2N2CCCCC2)c1]

A solution of 3-chloro-2-(piperidin-1-yl)aniline, 2HCl (0.142 g, 0.500 mmol) in pyridine (Volume: 1 ml) was treated with 3-(N,N-dimethylsulfamoyl)benzene-1-sulfonyl chloride (0.142 g, 0.5 mmol). The solution was microwaved at 115° C. for 1 hr. The crude product was purified by reversed-phase chromatography (4-100% CH₃CN over 15 min) to give desired product (0.040 g, 10% yield). (LCMS, ESI pos.) Calculated for C₁₉H₂₄ClN₃O₄S₂: 458.0 (M+H), Measured: 458.0, 460.1. ¹H NMR (400 MHz, DMSO-d6) δ 9.25 (s, 1H), 8.18 (ddd, J=7.8, 1.9, 1.1 Hz, 1H), 8.07 (dd, J=7.9, 1.7 Hz, 1H), 8.02-7.85 (m, 2H), 7.33 (dd, J=7.3, 2.3 Hz, 1H), 7.20 (d, J=8.0 Hz, 2H), 3.18 (d, J=20.9 Hz, 2H), 2.42-2.23 (m, 2H), 1.57 (s, 5H), 1.35 (s, 1H).

### Example 66

### N¹,N¹-dimethyl-N³-(2-(4-methylpiperidin-1-yl)phenyl)benzene-1,3-disulfonamide

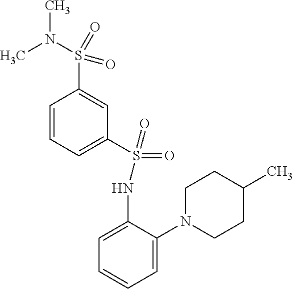

A solution of 2-(4-methylpiperidin-1-yl)aniline (0.095 g, 0.500 mmol) in pyridine (Volume: 1 ml) was treated with 3-(N,N-dimethylsulfamoyl)benzene-1-sulfonyl chloride (0.142 g, 0.5 mmol). The solution was microwaved at 115° C. for 1 hr. The crude product was purified by reversed-phase chromatography (4-100% CH₃CN over 15 min) to give desired product (0.046 g, 11% yield). (LCMS, ESI pos.) Calculated for C₂₀H₂₇N₃O₄S₂: 438.6 (M+H), Measured: 438.1. ¹H NMR (400 MHz, DMSO-d6) δ 9.07 (s, 1H), 8.12 (ddd, J=7.8, 1.9, 1.1 Hz, 1H), 7.96 (ddd, J=7.8, 1.8, 1.1 Hz, 1H), 7.92 (td, J=1.9, 0.6 Hz, 1H), 7.85 (td, J=7.8, 0.6 Hz, 1H), 7.31-7.23 (m, 1H), 7.14-6.98 (m, 3H), 2.45 (s, 6H), 2.44-2.39 (m, 4H), 1.48 (d, J=12.2 Hz, 2H), 1.34 (dd, J=7.4, 3.8 Hz, 0H), 1.19 (dtd, J=24.1, 12.8, 12.0, 5.6 Hz, 2H), 0.89 (d, J=6.4 Hz, 3H).

### Example 67

### N¹,N¹-dimethyl-N³-(3-(piperidin-1-yl)phenyl)benzene-1,3-disulfonamide

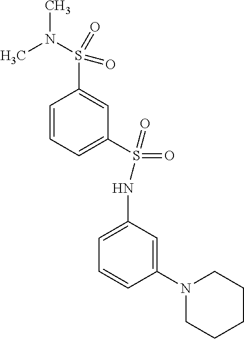 [SMILES: CN(C)S(=O)(=O)c1cccc(S(=O)(=O)Nc2cccc(N3CCCCC3)c2)c1]

A solution of 3-(piperidin-1-yl)aniline (0.088 g, 0.500 mmol) in pyridine (Volume: 1 ml) was treated with 3-(N,N-dimethylsulfamoyl)benzene-1-sulfonyl chloride (0.142 g, 0.5 mmol). The solution was microwaved at 115° C. for 1 hr. The crude product was purified by reversed-phase chromatography (4-100% CH₃CN over 15 min) to give desired product (0.122 g, 29% yield). (LCMS, ESI pos.) Calculated for C₁₉H₂₅N₃O₄S₂: 424.6 (M+H), Measured: 424.1. ¹H NMR (400 MHz, DMSO-d6) δ 10.31 (s, 1H), 8.08 (ddd, J=7.8, 1.8, 1.1 Hz, 1H), 8.06-7.96 (m, 2H), 7.88 (dd, J=8.1, 7.5 Hz, 1H), 7.08 (t, J=8.0 Hz, 1H), 6.69 (s, 2H), 6.54 (d, J=7.7 Hz, 1H), 3.07 (s, 4H).

### Example 68

### N¹-(3-chloro-2-(4-methylpiperidin-1-yl)phenyl)-N³,N³-dimethylbenzene-1,3-disulfonamide

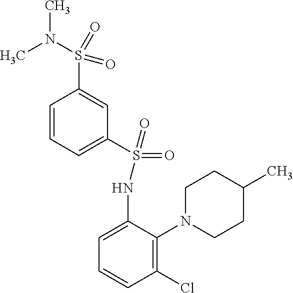

A solution of 3-chloro-2-(4-methylpiperidin-1-yl)aniline (0.112 g, 0.500 mmol) in pyridine (Volume: 1 ml) was treated with 4-(N,N-dimethylsulfamoyl)benzene-1-sulfonyl chloride (0.142 g, 0.5 mmol). The solution was microwaved at 115° C. for 1 hr. The solution was cooled to room temperature. The solution was filtered to give a solid (0.091 g, 19% yield). (LCMS, ESI pos.) Calculated for C₂₀H₂₆ClN₃O₄S₂: 472.0 (M+H), Measured: 472.1. ¹H NMR (400 MHz, DMSO-d6) δ 9.07 (s, 1H), 8.20 (s, 1H), 8.07 (ddd, J=7.8, 1.8, 1.1 Hz, 1H), 7.93 (td, J=7.9, 0.5 Hz, 2H), 7.52 (s, 1H), 7.23 (d, J=30.0 Hz, 2H), 3.35 (s, 2H), 3.21 (s, 2H), 2.53-2.44 (m, 6H), 2.09 (s, 1H), 1.52 (s, 2H), 1.29 (qd, J=11.3, 4.0 Hz, 2H), 0.98 (d, J=6.1 Hz, 3H).

### Example 69

### N¹-(2-(3,4-dihydroisoquinolin-2(1H)-yl)phenyl)-N⁴,N⁴-dimethylbenzene-1,4-disulfonamide

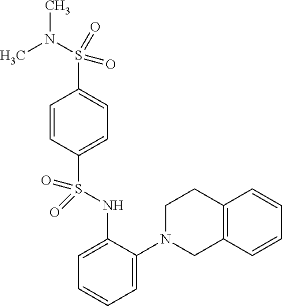 [SMILES: CN(C)S(=O)(=O)c1ccc(S(=O)(=O)Nc2ccccc2N2CCc3ccccc3C2)cc1]

A solution of 2-(3,4-dihydroisoquinolin-2(1H)-yl)aniline (0.112 g, 0.500 mmol) in pyridine (Volume: 1 ml) was treated with 4-(N,N-dimethylsulfamoyl)benzene-1-sulfonyl chloride (0.142 g, 0.5 mmol). The solution was microwaved at 115° C. for 1 hr. The solution was cooled to room temperature. The solution was filtered to give a solid (0.123 g, 26% yield). (LCMS, ESI pos.) Calculated for C₂₃H₂₅N₃O₄S₂: 472.6 (M+H), Measured: 472.1. ¹H NMR (400 MHz, DMSO-d6) δ 9.57 (s, 1H), 8.10-8.02 (m, 2H), 7.96-7.89 (m, 2H), 7.26 (ddd, J=7.9, 3.7, 1.5 Hz, 2H), 7.22-7.15 (m, 4H), 7.14-7.00 (m, 2H), 3.90 (s, 2H), 2.96 (t, J=5.6 Hz, 2H), 2.88 (t, J=5.9 Hz, 2H), 2.66 (s, 6H).

### Example 70

### N¹-(3-chloro-2-(4-methylpiperidin-1-yl)phenyl)-N⁴,N⁴-dimethylbenzene-1,4-disulfonamide

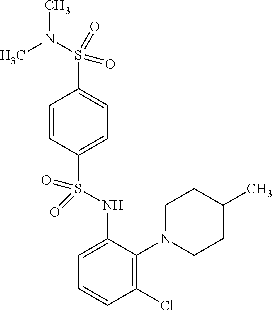

A solution of 3-chloro-2-(4-methylpiperidin-1-yl)aniline (0.112 g, 0.500 mmol) in pyridine (Volume: 1 ml) was treated with 4-(N,N-dimethylsulfamoyl)benzene-1-sulfonyl chloride (0.142 g, 0.5 mmol). The solution was microwaved at 115° C. for 1 hr. The solution was cooled to room temperature. The solution was filtered to give a solid (0.105 g, 23% yield). (LCMS, ESI pos.) Calculated for C₂₀H₂₆ClN₃O₄S₂: 472.0 (M+H), Measured: 472.1. ¹H NMR (400 MHz, DMSO-d6) δ 8.99 (s, 1H), 7.93 (q, J=8.5 Hz, 4H), 7.38 (d, J=17.8 Hz, 1H), 7.15 (s, 2H), 3.28 (s, 4H), 2.59 (s, 6H), 2.11 (s, 1H), 1.47 (d, J=10.9 Hz, 2H), 1.22 (qd, J=11.5, 4.0 Hz, 2H), 0.92 (d, J=6.2 Hz, 3H).

### Example 71

### N¹-(2-(4-((dimethylamino)methyl)piperidin-1-yl)phenyl)-N⁴,N⁴-dimethylbenzene-1,4-disulfonamide

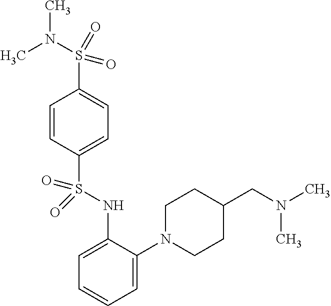

A solution of 2-(4-((dimethylamino)methyl)piperidin-1-yl)aniline (0.117 g, 0.500 mmol) in pyridine (Volume: 1 ml) was treated with 4-(N,N-dimethylsulfamoyl)benzene-1-sulfonyl chloride (0.142 g, 0.5 mmol). The solution was microwaved at 115° C. for 1 hr. The crude product was purified by reversed-phase chromatography (4-100% CH₃CN over 15 min) to give desired product (0.074 g, 15% yield). (LCMS, ESI pos.) Calculated for C₂₂H₃₂N₄O₄S₂: 481.6 (M+H), Measured: 481.1. ¹H NMR (400 MHz, DMSO-d6) δ 9.26 (d, J=21.2 Hz, 3H), 8.07-8.01 (m, 2H), 7.99-7.93 (m, 2H), 7.31-7.26 (m, 1H), 7.22-7.07 (m, 4H), 3.21 (s, 2H), 3.03 (t, J=6.4 Hz, 3H), 2.84 (d, J=4.8 Hz, 7H), 2.67 (s, 11H), 1.83 (ddt, J=11.2, 7.7, 3.8 Hz, 1H), 1.67 (d, J=12.2 Hz, 2H), 1.41-1.24 (m, 3H).

### Example 72

### Ethyl 1-(2-((4-(N,N-dimethylsulfamoyl)phenyl)sulfonamido)phenyl)piperidine-4-carboxylate

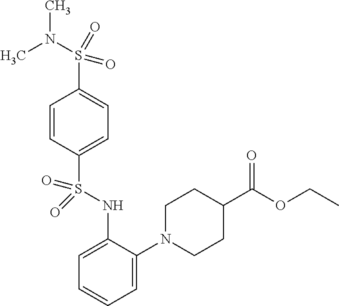 [SMILES: CCOC(=O)C1CCN(c2ccccc2NS(=O)(=O)c2ccc(S(=O)(=O)N(C)C)cc2)CC1]

A solution of ethyl 1-(2-aminophenyl)piperidine-4-carboxylate (0.124 g, 0.500 mmol) in pyridine (Volume: 1 ml) was treated with 4-(N,N-dimethylsulfamoyl)benzene-1-sulfonyl chloride (0.142 g, 0.5 mmol). The solution was microwaved at 115° C. for 1 hr. The crude product was purified by reversed-phase chromatography (4-100% CH₃CN over 15 min) to give desired product (0.029 g, 6% yield). (LCMS, ESI pos.) Calculated for C₂₂H₂₉N₃O₆S₂: 496.6 (M+H), Measured: 496.1. ¹H NMR (400 MHz, DMSO-d6) δ 9.32 (s, 1H), 8.12-8.02 (m, 2H), 8.01-7.90 (m, 2H), 7.38-7.25 (m, 1H), 7.20-7.00 (m, 3H), 4.13 (q, J=7.1 Hz, 2H), 3.45 (s, 4H), 2.66 (s, 6H), 2.44-2.29 (m, 1H), 1.78 (dd, J=7.8, 3.8 Hz, 4H), 1.24 (t, J=7.1 Hz, 3H).

### Example 73

### 4-(tert-butylsulfonyl)-N-(2-(piperidin-1-yl)phenyl)benzenesulfonamide

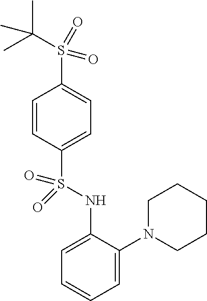 [SMILES: CC(C)(C)S(=O)(=O)c1ccc(S(=O)(=O)Nc2ccccc2N2CCCCC2)cc1]

A solution of 2-(piperidin-1-yl)aniline (0.088 g, 0.5 mmol) in pyridine (Volume: 1 ml) was treated with 4-(tert-butylsulfonyl)benzene-1-sulfonyl chloride (0.148 g, 0.500 mmol). The solution was microwaved at 115° C. for 1 hr. The solution was cooled to room temperature. The solution was filtered to give a solid (0.083 g, 19% yield). (LCMS, ESI pos.) Calculated for C₂₁H₂₈N₂O₄S₂: 437.6 (M+H), Measured: 437.1. ¹H NMR (400 MHz, DMSO-d6) δ 9.17 (s, 1H), 8.12-7.96 (m, 4H), 7.33 (dt, J=7.7, 1.1 Hz, 1H), 7.25-7.03 (m, 3H), 2.48 (t, J=5.2 Hz, 4H), 1.60-1.51 (m, 4H), 1.46 (q, J=5.9 Hz, 2H), 1.26 (s, 9H).

### Example 74

### 6-(methylsulfonyl)-N-(2-(piperidin-1-yl)phenyl)pyridine-3-sulfonamide

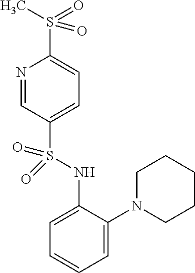 [SMILES: CS(=O)(=O)c1ccc(S(=O)(=O)Nc2ccccc2N2CCCCC2)cn1]

A solution of 2-(piperidin-1-yl)aniline (0.088 g, 0.500 mmol) in pyridine (Volume: 1 ml) was treated with 6-(methylsulfonyl)pyridine-3-sulfonyl chloride (0.128 g, 0.5 mmol). The solution was microwaved at 115° C. for 1 hr. The crude product was purified by reversed-phase chromatography (4-100% CH₃CN over 15 min) to give desired product (0.075 g, 19% yield). (LCMS, ESI pos.) Calculated for C₁₇H₂₁N₃O₄S₂: 396.5 (M+H), Measured: 396.1. ¹H NMR (400 MHz, DMSO-d6) δ 9.59 (s, 1H), 9.11 (dd, J=2.3, 0.9 Hz, 1H), 8.48 (dd, J=8.2, 2.3 Hz, 1H), 8.26 (dd, J=8.2, 0.8 Hz, 1H), 7.29 (dd, J=7.9, 1.5 Hz, 1H), 7.25-7.14 (m, 2H), 7.11 (ddd, J=7.9, 7.1, 1.8 Hz, 1H), 3.37 (s, 3H), 1.57-1.37 (m, 5H).

### Example 75

### N¹,N¹-dimethyl-N⁴-(2-(3-methylpiperidin-1-yl)phenyl)benzene-1,4-disulfonamide

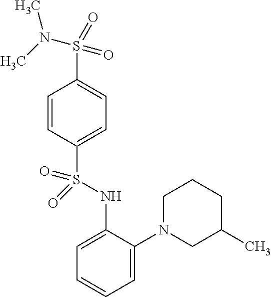

A solution of 2-(3-methylpiperidin-1-yl)aniline (0.190 g, 1.000 mmol) in pyridine (Volume: 2 ml) was treated with 4-(N,N-dimethylsulfamoyl)benzene-1-sulfonyl chloride (0.284 g, 1 mmol). The solution was microwaved at 115° C. for 1 hr. The crude product was purified by reversed-phase chromatography (4-100% CH₃CN over 15 min) to give desired product (0.065 g, 15% yield). (LCMS, ESI pos.) Calculated for C₂₀H₂₇N₃O₄S₂: 438.6 (M+H), Measured: 438.1. ¹H NMR (400 MHz, DMSO-d6) δ 9.12 (s, 1H), 8.06-7.98 (m, 2H), 7.99-7.91 (m, 2H), 7.33 (dt, J=7.7, 1.1 Hz, 1H), 7.21-7.14 (m, 2H), 7.11 (dt, J=7.8, 4.4 Hz, 1H), 2.66 (s, 6H), 2.51-2.34 (m, 3H), 2.11 (t, J=10.5 Hz, 1H), 1.70 (d, J=12.0 Hz, 2H), 1.56 (d, J=8.4 Hz, 2H), 1.02-0.85 (m, 1H), 0.81 (d, J=6.5 Hz, 3H).

### Example 76

### N-(2-(piperidin-1-yl)phenyl)-4-(piperidin-1-ylsulfonyl)benzenesulfonamide

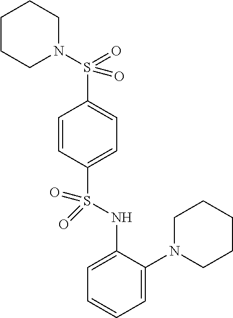 [SMILES: O=S(=O)(Nc1ccccc1N1CCCCC1)c1ccc(S(=O)(=O)N2CCCCC2)cc1]

A solution of 4-(piperidin-1-ylsulfonyl)benzene-1-sulfonyl chloride (0.324 g, 1 mmol) in pyridine (Volume: 2 ml) was treated with 2-(piperidin-1-yl)aniline (0.176 g, 1.000 mmol). The solution was microwaved at 115° C. for 1 hr. The solution was cooled to room temperature. The solution was filtered to give a solid (0.228 g, 49% yield). (LCMS, ESI pos.) Calculated for C₂₂H₂₉N₃O₄S₂: 464.6 (M+H), Measured: 464.1. ¹H NMR (400 MHz, DMSO-d6) δ 9.05 (s, 1H), 8.03-7.92 (m, 2H), 7.91-7.80 (m, 2H), 7.34-6.93 (m, 4H), 2.92-2.80 (m, 4H), 2.45-2.38 (m, 4H), 1.49 (hept, J=5.0 Hz, 9H), 1.41 (q, J=5.8 Hz, 2H), 1.36-1.26 (m, 2H).

### Example 77

### N¹,N¹-dimethyl-N³-(2-(piperidin-1-yl)phenyl)benzene-1,3-disulfonamide

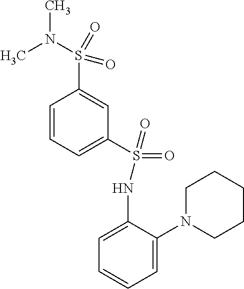 [SMILES: CN(C)S(=O)(=O)c1cccc(S(=O)(=O)Nc2ccccc2N2CCCCC2)c1]

A solution of 2-(piperidin-1-yl)aniline (0.176 g, 1.000 mmol) in pyridine (Volume: 2 ml) was treated with 3-(N,N-dimethylsulfamoyl)benzene-1-sulfonyl chloride (0.284 g, 1 mmol). The solution was microwaved at 115° C. The solution was cooled to room temperature. The solution was filtered to give a solid (0.092 g, 22% yield). (LCMS, ESI pos.) Calculated for C₁₉H₂₅N₃O₄S₂: 424.6 (M+H), Measured: 424.1. ¹H NMR (400 MHz, DMSO-d6) δ 9.14 (s, 1H), 8.19 (ddd, J=7.8, 1.9, 1.1 Hz, 1H), 8.03 (ddd, J=7.8, 1.8, 1.1 Hz, 1H), 7.98 (td, J=1.8, 0.5 Hz, 1H), 7.91 (td, J=7.8, 0.5 Hz, 1H), 7.34 (dd, J=7.3, 1.5 Hz, 1H), 7.19-7.06 (m, 3H), 2.78-2.62 (m, OH), 2.52 (s, 6H), 2.49-2.44 (m, 4H), 1.56 (d, J=5.6 Hz, 4H), 1.47 (d, J=5.7 Hz, 2H).

### Example 78

### N¹,N¹-dimethyl-N⁴-(2-(2-methylpiperidin-1-yl)phenyl)benzene-1,4-disulfonamide

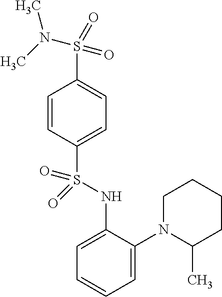 [SMILES: CC1CCCCN1c1ccccc1NS(=O)(=O)c1ccc(S(=O)(=O)N(C)C)cc1]

A solution of 2-(2-methylpiperidin-1-yl)aniline (0.095 g, 0.500 mmol) in pyridine (Volume: 1 ml) was treated with 4-(N,N-dimethylsulfamoyl)benzene-1-sulfonyl chloride (0.142 g, 0.5 mmol). The solution was microwaved at 115° C. for 1 hr. The solution was cooled to room temperature. The solution was filtered to give a solid (0.005 g, 1% yield). (LCMS, ESI pos.) Calculated for C₂₀H₂₇N₃O₄S₂: 438.6 (M+H), Measured: 438.1. ¹H NMR (400 MHz, DMSO-d6) δ 9.04 (s, 1H), 8.14-8.01 (m, 2H), 7.99-7.86 (m, 2H), 7.54 (dd, J=8.0, 1.6 Hz, 1H), 7.26 (dd, J=7.7, 1.8 Hz, 1H), 7.20 (td, J=7.7, 1.8 Hz, 1H), 7.15 (td, J=7.5, 1.7 Hz, 1H), 2.80 (t, J=6.1 Hz, 1H), 2.63 (s, 6H), 2.37 (td, J=11.3, 2.7 Hz, 1H), 1.98 (d, J=11.4 Hz, 1H), 1.77-1.29 (m, 6H), 0.49 (d, J=6.1 Hz, 3H).

### Example 79

### N¹-(2-(dipropylamino)phenyl)-N⁴,N⁴-dimethylbenzene-1,4-disulfonamide

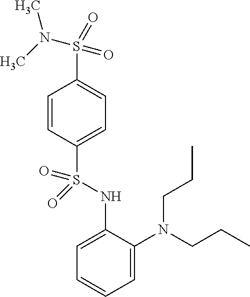 [SMILES: CCCN(CCC)c1ccccc1NS(=O)(=O)c1ccc(S(=O)(=O)N(C)C)cc1]

A solution of N1,N1-dipropylbenzene-1,2-diamine (0.192 g, 1 mmol) in pyridine (Volume: 2.000 ml) was treated with 4-(N,N-dimethylsulfamoyl)benzene-1-sulfonyl chloride (0.284 g, 1.000 mmol). The solution was microwaved at 115° C. for 1 hr. The crude product was purified by reversed-phase chromatography (4-100% CH₃CN over 15 min) to give desired product (0.030 g, 7% yield). (LCMS, ESI pos.) Calculated for C₂₀H₂₉N₃O₄S₂: 440.6 (M+H), Measured: 440.1. ¹H NMR (400 MHz, DMSO-d6) δ 9.04 (s, 1H), 8.13-8.07 (m, 2H), 8.00-7.93 (m, 2H), 7.40 (d, J=10.6 Hz, 1H), 7.26 (d, J=9.4 Hz, 1H), 7.13 (dd, J=6.8, 2.6 Hz, 2H), 2.82-2.67 (m, 1H), 2.66 (s, 7H), 1.17 (q, J=7.6 Hz, 4H), 0.74 (t, J=7.4 Hz, 7H).

### Example 80

### N,N-diethyl-4-((4-methylpiperidin-1-yl)sulfonyl)benzenesulfonamide

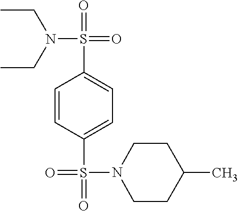 [SMILES: CCN(CC)S(=O)(=O)c1ccc(S(=O)(=O)N2CCC(C)CC2)cc1]

A solution of 4-methylpiperidine (0.539 ml, 1.000 mmol) in DMF (Volume: 1.5 ml) was treated with 4-(N,N-diethylsulfamoyl)benzene-1-sulfonyl chloride (0.156 g, 0.5 mmol). The resulting solution was microwaved at 130° C. for 1 hr. The crude product was purified by reversed-phase chromatography (4-100% CH₃CN over 15 min) to give desired product (0.107 g, 29% yield). (LCMS, ESI pos.) Calculated for C₁₆H₂₆N₂O₄S₂: 375.5 (M+H), Measured: 375.1. ¹H NMR (400 MHz, DMSO-d6) δ 8.13-8.01 (m, 2H), 8.02-7.89 (m, 2H), 3.67 (dt, J=12.0, 3.1 Hz, 2H), 3.25 (q, J=7.1 Hz, 4H), 2.32 (td, J=12.0, 2.6 Hz, 2H), 1.76-1.60 (m, 2H), 1.46-1.26 (m, 1H), 1.15 (td, J=12.5, 4.1 Hz, 2H), 1.08 (t, J=7.1 Hz, 6H), 0.88 (d, J=6.5 Hz, 3H).

### Example 81

### N¹,N¹-diethyl-N⁴-(2-(4-methylpiperidin-1-yl)phenyl)benzene-1,4-disulfonamide

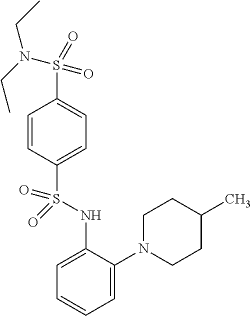 [SMILES: CCN(CC)S(=O)(=O)c1ccc(S(=O)(=O)Nc2ccccc2N2CCC(C)CC2)cc1]

A solution of 2-(4-methylpiperidin-1-yl)aniline (0.190 g, 1 mmol) in pyridine (Volume: 2 ml) was treated with 4-(N,N-diethylsulfamoyl)benzene-1-sulfonyl chloride (0.312 g, 1.000 mmol). The solution was microwaved at 115° C. for 1 hr. The crude product was purified by reversed-phase chromatography (4-100% CH₃CN over 15 min) to give desired product (0.045 g, 10% yield). (LCMS, ESI pos.) Calculated for C₂₂H₃₁N₃O₄S₂: 466.6 (M+H), Measured: 466.2. ¹H NMR (400 MHz, DMSO-d6) δ 9.09 (s, 1H), 8.00 (s, 4H), 7.32-7.28 (m, 1H), 7.17-7.12 (m, 2H), 7.12-7.05 (m, 1H), 3.20 (q, J=7.1 Hz, 4H), 2.46 (td, J=11.4, 2.3 Hz, 2H), 1.55 (d, J=11.9 Hz, 2H), 1.41 (ddd, J=10.8, 6.7, 3.6 Hz, 0H), 1.27 (qd, J=11.6, 4.0 Hz, 2H), 1.05 (t, J=7.1 Hz, 6H), 0.96 (d, J=6.4 Hz, 3H).

### Example 82

### N¹,N¹-dimethyl-N⁴-(3-methyl-2-(4-methylpiperidin-1-yl)phenyl)benzene-1,4-disulfonamide

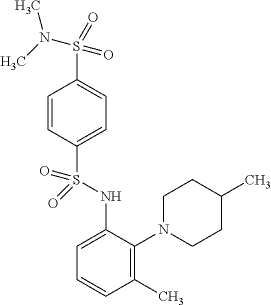

A solution of 3-methyl-2-(4-methylpiperidin-1-yl)aniline (0.204 g, 1 mmol) in pyridine (Volume: 2 ml) was treated with 4-(N,N-dimethylsulfamoyl)benzene-1-sulfonyl chloride (0.284 g, 1.000 mmol). The resulting solution was microwaved at 115° C. for 1 hr. The crude product was purified by reversed-phase chromatography (4-100% CH₃CN over 15 min) to give desired product (0.001 g, 1% yield). (LCMS, ESI pos.) Calculated for C₂₁H₂₉N₃O₄S₂: 452.6 (M+H), Measured: 452.2. ¹H NMR (400 MHz, DMSO-d6) δ 8.90 (s, 1H), 8.08-7.90 (m, 4H), 7.33 (d, J=8.0 Hz, 1H), 7.10 (s, 1H), 6.91 (d, J=7.5 Hz, 1H), 3.04 (t, J=11.4 Hz, 2H), 2.64 (s, 6H), 2.27 (s, 4H), 1.59 (d, J=12.6 Hz, 2H), 1.45 (s, 1H), 1.25 (qd, J=11.7, 4.1 Hz, 2H), 1.00 (d, J=6.5 Hz, 3H).

### Example 83

### N¹-(4-methoxy-2-(4-methylpiperidin-1-yl)phenyl)-N⁴,N⁴-dimethylbenzene-1,4-disulfonamide

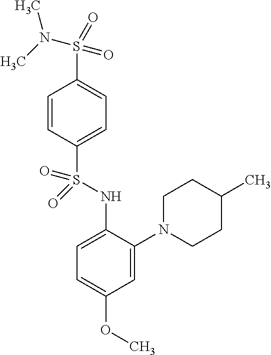

A solution of 4-methoxy-2-(4-methylpiperidin-1-yl)aniline (0.220 g, 1 mmol) in pyridine (Volume: 2 ml) was treated with 4-(N,N-dimethylsulfamoyl)benzene-1-sulfonyl chloride (0.284 g, 1.000 mmol). The solution was microwaved at 115° C. for 1 hr. The solution was cooled to room temperature. The solution was filtered to give a solid (0.001 g, 1% yield). (LCMS, ESI pos.) Calculated for C₂₁H₂₉N₃O₅S₂: 468.6 (M+H), Measured: 468.2. ¹H NMR (400 MHz, DMSO-d6) δ 9.08 (s, 1H), 8.47-8.41 (m, OH), 8.19-8.12 (m, OH), 7.94 (s, 4H), 7.16 (d, J=8.7 Hz, 1H), 6.65 (dd, J=8.8, 2.8 Hz, 1H), 6.61 (d, J=2.8 Hz, 1H), 3.73 (s, 3H), 2.74 (s, 1H), 2.67 (s, 6H), 2.65-2.58 (m, 2H), 2.47-2.34 (m, 2H), 1.48 (d, J=12.5 Hz, 2H), 1.15-1.00 (m, 2H), 0.92 (d, J=6.5 Hz, 3H).

### Example 84

### N1-(3-hydroxy-2-(4-methylpiperidin-1-yl)phenyl)-N⁴,N⁴-dimethylbenzene-1,4-disulfonamide

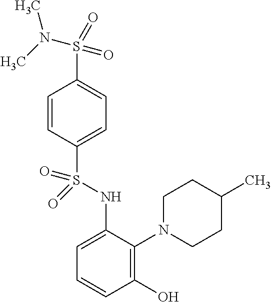

A solution of 3-amino-2-(4-methylpiperidin-1-yl)phenol (0.206 g, 1 mmol) and pyridine (Volume: 2 ml) was treated with 4-(N,N-dimethylsulfamoyl)benzene-1-sulfonyl chloride (0.284 g, 1.000 mmol). The solution was microwaved at 115° C. for 1 hr. The solution was cooled to room temperature. The solution was filtered to give a solid (0.019 g, 4% yield). (LCMS, ESI pos.) Calculated for C₂₀H₂₇N₃O₅S₂: 454.6 (M+H), Measured: 454.1. ¹H NMR (400 MHz, DMSO-d6) δ 9.64 (s, 1H), 8.92 (s, 1H), 8.05-7.84 (m, 4H), 7.00 (q, J=9.3, 8.6 Hz, 2H), 6.60 (d, J=8.3 Hz, 1H), 3.17 (d, J=30.5 Hz, 2H), 2.63 (s, 6H), 2.00 (d, J=42.0 Hz, 2H), 1.54 (d, J=12.2 Hz, 2H), 1.33-1.12 (m, 2H), 0.97 (d, J=6.4 Hz, 3H).

### Example 85

### 4-(tert-butylsulfonyl)-N-(3-(piperidin-1-yl)phenyl)benzenesulfonamide

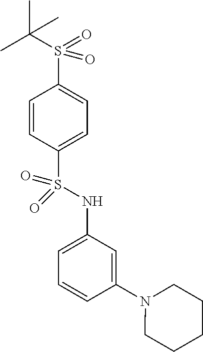 [SMILES: CC(C)(C)S(=O)(=O)c1ccc(S(=O)(=O)Nc2cccc(N3CCCCC3)c2)cc1]

A solution of 3-(piperidin-1-yl)aniline (0.088 g, 0.500 mmol) in pyridine (Volume: 1.000 ml) was treated with 4-(tert-butylsulfonyl)benzene-1-sulfonyl chloride (0.148 g, 0.5 mmol). The solution was microwaved at 115° C. for 1 hr. The crude product was purified by reversed-phase chromatography (4-100% CH₃CN over 15 min) to give desired product (0.197 g, 45% yield). (LCMS, ESI pos.) Calculated for C₂₁H₂₈N₂O₄S₂: 437.6 (M+H), Measured: 437.1. ¹H NMR (400 MHz, DMSO-d6) δ 10.40 (s, 1H), 8.08-7.96 (m, 4H), 7.12 (d, J=8.2 Hz, 1H), 6.87-6.49 (m, 3H), 3.10 (s, 4H), 1.58 (d, J=20.1 Hz, 6H), 1.25 (s, 9H).

### Example 86

### 4-(tert-butylsulfonyl)-N-(2-(4-methylpiperazin-1-yl)phenyl)benzenesulfonamide

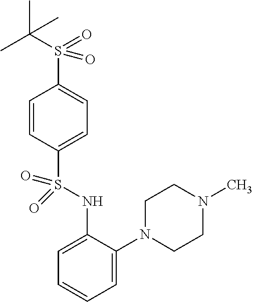 [SMILES: CN1CCN(c2ccccc2NS(=O)(=O)c2ccc(S(=O)(=O)C(C)(C)C)cc2)CC1]

A solution of 2-(4-methylpiperazin-1-yl)aniline (0.096 g, 0.500 mmol) in pyridine (Volume: 1 ml) was treated with 4-(tert-butylsulfonyl)benzene-1-sulfonyl chloride (0.148 g, 0.5 mmol). The solution was microwaved at 115° C. for 1 hr. The crude product was purified by reversed-phase chromatography (4-100% CH₃CN over 15 min) to give desired product (0.079 g, 18% yield). (LCMS, ESI pos.) Calculated for C₂₁H₂₉N₃O₄S₂: 452.6 (M+H), Measured: 452.2. ¹H NMR (400 MHz, DMSO-d6) δ 9.66 (s, 1H), 8.07 (s, 4H), 7.33 (dt, J=7.5, 1.3 Hz, 1H), 7.26-7.10 (m, 3H), 3.18 (s, 2H), 2.89 (s, 5H), 2.75-2.64 (m, 2H), 1.27 (s, 9H).

### Example 87

### N¹,N¹-diethyl-N⁴-(2-(piperidin-1-yl)phenyl)benzene-1,4-disulfonamide

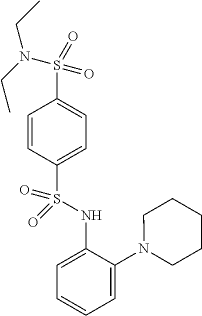 [SMILES: CCN(CC)S(=O)(=O)c1ccc(S(=O)(=O)Nc2ccccc2N2CCCCC2)cc1]

A solution of 2-(piperidin-1-yl)aniline (0.088 g, 0.500 mmol) pyridine (Volume: 1.000 ml) was treated with 4-(N,N-diethylsulfamoyl)benzene-1-sulfonyl chloride (0.156 g, 0.5 mmol). The solution was microwaved at 115° C. for 1 hr. The solution was cooled to room temperature. The solution was filtered to give a solid (0.152 g, 34% yield). (LCMS, ESI pos.) Calculated for C₂₁H₂₉N₃O₄S₂: 452.6 (M+H), Measured: 452.2. ¹H NMR (400 MHz, DMSO-d6) δ 9.10 (s, 1H), 7.99 (s, 4H), 7.36-7.24 (m, 1H), 7.21-7.00 (m, 3H), 3.20 (q, J=7.1 Hz, 4H), 2.48 (t, J=5.1 Hz, 4H), 1.56 (h, J=5.2 Hz, 5H), 1.52-1.43 (m, 2H), 1.05 (t, J=7.1 Hz, 6H).

### Example 88

### N¹,N¹-diethyl-N⁴-(3-(piperidin-1-yl)phenyl)benzene-1,4-disulfonamide

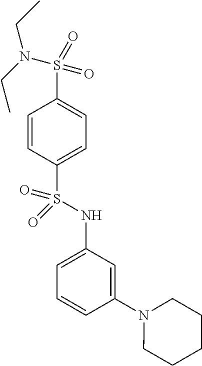 [SMILES: CCN(CC)S(=O)(=O)c1ccc(S(=O)(=O)Nc2cccc(N3CCCCC3)c2)cc1]

A solution of 3-(piperidin-1-yl)aniline (0.088 g, 0.500 mmol) in pyridine (Volume: 1.000 ml) was treated with 4-(N,N-diethylsulfamoyl)benzene-1-sulfonyl chloride (0.156 g, 0.5 mmol). The solution was microwaved at 115° C. for 1 hr. The crude product was purified by reversed-phase chromatography (4-100% CH₃CN over 15 min) to give desired product (0.163 g, 36% yield). (LCMS, ESI pos.) Calculated for C₂₁H₂₉N₃O₄S₂: 452.6 (M+H), Measured: 452.2. ¹H NMR (400 MHz, DMSO-d6) δ 10.40 (s, 1H), 8.08-7.88 (m, 4H), 7.12 (d, J=8.5 Hz, 1H), 6.91-6.45 (m, 3H), 3.20 (q, J=7.1 Hz, 4H), 3.12 (s, 4H), 1.60 (d, J=21.8 Hz, 6H), 1.03 (t, J=7.1 Hz, 6H).

### Example 89

### N¹-(2-(isopropyl(methyl)amino)phenyl)-N⁴,N⁴-dimethylbenzene-1,4-disulfonamide

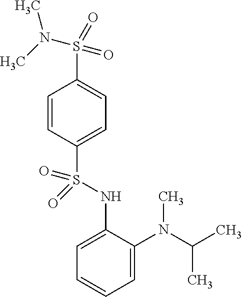 [SMILES: CC(C)N(C)c1ccccc1NS(=O)(=O)c1ccc(S(=O)(=O)N(C)C)cc1]

A solution of N1-isopropyl-N1-methylbenzene-1,2-diamine, 2HCl (0.119 g, 0.500 mmol) in pyridine (Volume: 1 ml) was treated with 4-(N,N-dimethylsulfamoyl)benzene-1-sulfonyl chloride (0.142 g, 0.5 mmol). The solution was microwaved at 115° C. for 1 hr. The crude product was purified by reversed-phase chromatography (4-100% CH₃CN over 15 min) to give desired product (0.12 g, 29% yield). (LCMS, ESI pos.) Calculated for C₁₈H₂₅N₃O₄S₂: 412.5 (M+H), Measured: 412.1. ¹H NMR (400 MHz, DMSO-d6) δ 8.08 (d, J=8.4 Hz, 2H), 8.00-7.94 (m, 2H), 7.37-7.05 (m, 4H), 2.65 (s, 6H), 0.93 (d, J=6.1 Hz, 6H).

### Example 90

### N¹,N¹-dimethyl-N⁴-(1-oxoisoindolin-5-yl)benzene-1,4-disulfonamide

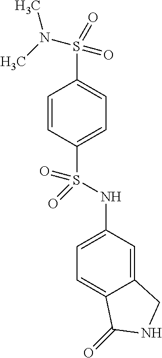 [SMILES: CN(C)S(=O)(=O)c1ccc(S(=O)(=O)Nc2ccc3c(c2)CNC3=O)cc1]

A solution of 5-aminoisoindolin-1-one (0.074 g, 0.500 mmol) in pyridine (Volume: 1 ml) was treated with 4-(N,N-dimethylsulfamoyl)benzene-1-sulfonyl chloride (0.142 g, 0.5 mmol). The solution was microwaved at 115° C. for 1 hr. The solution was cooled to room temperature. The solution was filtered to give a solid (0.128 g, 32% yield). (LCMS, ESI pos.) Calculated for C₁₆H₁₇N₃O₅S₂: 396.5 (M+H), Measured: 396.0. ¹H NMR (400 MHz, DMSO-d6) δ 10.96 (s, 1H), 8.45 (s, 1H), 8.10-8.03 (m, 2H), 8.00-7.93 (m, 2H), 7.58 (dd, J=8.2, 0.6 Hz, 1H), 7.34 (dq, J=1.6, 0.8 Hz, 1H), 7.21 (dd, J=8.2, 1.9 Hz, 1H), 4.32 (s, 2H), 2.65 (s, 6H).

### Example 91

### N¹-(2-(3,4-dihydroisoquinolin-2(1H)-yl)phenyl)-N³,N³-dimethylbenzene-1,3-disulfonamide

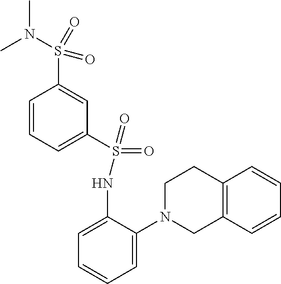 [SMILES: CN(C)S(=O)(=O)c1cccc(S(=O)(=O)Nc2ccccc2N2CCc3ccccc3C2)c1]

A solution of 2-(3,4-dihydroisoquinolin-2(1H)-yl)aniline (0.112 g, 0.500 mmol) in pyridine (Volume: 1 ml) was treated with 3-(N,N-dimethylsulfamoyl)benzene-1-sulfonyl chloride (0.142 g, 0.5 mmol). The solution was microwaved at 115° C. for 1 hr. The crude product was purified by reversed-phase chromatography (4-100% CH₃CN over 15 min) to give desired product (0.003 g, 1% yield). (LCMS, ESI pos.) Calculated for C₂₃H₂₅N₃O₄S₂: 472.6 (M+H), Measured: 472.1. ¹H NMR (400 MHz, DMSO-d6) δ 9.61 (s, 1H), 8.18 (ddd, J=7.9, 1.9, 1.1 Hz, 1H), 8.07-7.97 (m, 2H), 7.85 (td, J=7.8, 0.5 Hz, 1H), 7.32-6.98 (m, 8H), 3.86 (s, 2H), 2.93 (d, J=5.3 Hz, 2H), 2.87 (t, J=5.7 Hz, 2H), 2.54 (s, 5H).

### Example 92

### 4-(tert-butylsulfonyl)-N-(2-(4-methylpiperidin-1-yl)phenyl)benzenesulfonamide

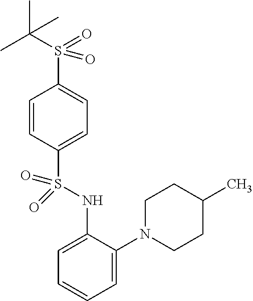 [SMILES: CC1CCN(c2ccccc2NS(=O)(=O)c2ccc(S(=O)(=O)C(C)(C)C)cc2)CC1]

A solution of 2-(4-methylpiperidin-1-yl)aniline (0.095 g, 0.500 mmol) in pyridine (Volume: 1 ml) was treated with 4-(tert-butylsulfonyl)benzene-1-sulfonyl chloride (0.148 g, 0.5 mmol). The solution was microwaved at 115° C. for 1 hr. The solution was cooled to room temperature. The solution was filtered to give a solid (0.0012 g, 1% yield). (LCMS, ESI pos.) Calculated for C₂₂H₃₀N₂O₄S₂: 450.6 (M+H), Measured: 451.2. ¹H NMR (400 MHz, DMSO-d6) δ 9.19 (s, 1H), 8.09-7.99 (m, 4H), 7.30 (dd, J=7.5, 1.2 Hz, 1H), 7.13 (d, J=4.1 Hz, 2H), 7.11-7.03 (m, 1H), 2.57 (s, 2H), 2.46 (td, J=11.5, 2.4 Hz, 2H), 1.54 (dd, J=12.7, 3.4 Hz, 3H), 1.40 (ddd, J=10.9, 6.7, 3.6 Hz, 1H), 1.26 (s, 11H), 0.96 (d, J=6.4 Hz, 3H).

### Example 93

### N-(2-(4-methylpiperazin-1-yl)phenyl)-4-((trifluoromethyl)sulfonyl)benzenesulfonamide

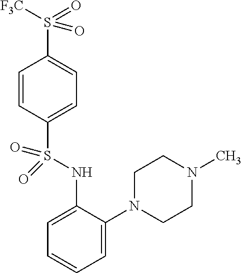 [SMILES: CN1CCN(c2ccccc2NS(=O)(=O)c2ccc(S(=O)(=O)C(F)(F)F)cc2)CC1]

A solution of 2-(4-methylpiperazin-1-yl)aniline (0.096 g, 0.500 mmol) in pyridine (Volume: 1.0 ml) was treated with 4-((trifluoromethyl)sulfonyl)benzene-1-sulfonyl chloride (0.154 g, 0.5 mmol). The solution was microwaved at 115° C. for 1 hr. The crude product was purified by reversed-phase chromatography (4-100% CH₃CN over 15 min) to give desired product (0.001 g, 1% yield). (LCMS, ESI pos.) Calculated for C₁₈H₂₀F₃N₃O₄S₂: 463.5 (M+H), Measured: 464.1.

### Example 94

### N¹,N¹-diethyl-N⁴-(2-(4-methylpiperazin-1-yl)phenyl)benzene-1,4-disulfonamide

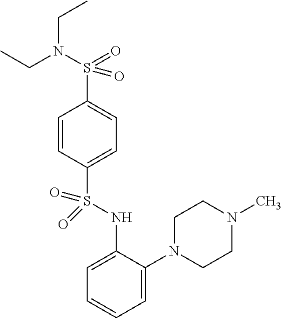 [SMILES: CCN(CC)S(=O)(=O)c1ccc(S(=O)(=O)Nc2ccccc2N2CCN(C)CC2)cc1]

A solution of 2-(4-methylpiperazin-1-yl)aniline (0.096 g, 0.500 mmol) in pyridine (Volume: 1 ml) was treated with 4-(N,N-diethylsulfamoyl)benzene-1-sulfonyl chloride (0.156 g, 0.5 mmol). The solution was microwaved at 115° C. for 1 hr. The crude product was purified by reversed-phase chromatography (4-100% CH₃CN over 15 min) to give desired product (0.019 g, 7% yield). (LCMS, ESI pos.) Calculated for C₂₁H₃₀N₄O₄S₂: 466.6 (M+H), Measured: 467.2. ¹H NMR (400 MHz, DMSO-d6) δ 9.24 (s, 1H), 8.07-7.91 (m, 4H), 7.26 (dt, J=7.8, 1.1 Hz, 1H), 7.18-7.15 (m, 2H), 7.08 (dt, J=7.9, 4.5 Hz, 1H), 3.21 (q, J=7.1 Hz, 4H), 2.58 (q, J=4.0, 3.3 Hz, 4H), 2.38 (d, J=8.5 Hz, 4H), 2.24 (s, 3H), 1.05 (t, J=7.1 Hz, 6H).

### Example 95

### N¹,N¹-dimethyl-N³-(2-(3-methylpiperidin-1-yl)phenyl)benzene-1,3-disulfonamide

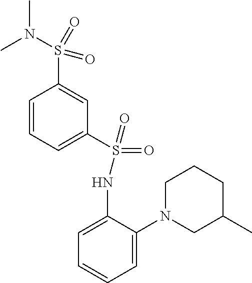

A solution of 2-(3-methylpiperidin-1-yl)aniline (0.095 g, 0.500 mmol) in pyridine (Volume: 1 ml) was treated with 3-(N,N-dimethylsulfamoyl)benzene-1-sulfonyl chloride (0.142 g, 0.5 mmol). The solution was microwaved at 115° C. for 1 hr. The crude product was purified by reversed-phase chromatography (4-100% CH₃CN over 15 min) to give desired product (0.024 g, 11% yield). (LCMS, ESI pos.) Calculated for C₂₀H₂₇N₃O₄S₂: 438.6 (M+H), Measured: 438.1. ¹H NMR (400 MHz, DMSO-d6) δ 9.08 (s, 1H), 8.11 (ddd, J=7.8, 1.9, 1.1 Hz, 1H), 7.98 (ddd, J=7.8, 1.8, 1.1 Hz, 1H), 7.92-7.81 (m, 2H), 7.31 (dd, J=6.8, 1.7 Hz, 1H), 7.14-7.01 (m, 3H), 2.44 (s, 6H), 2.42-2.24 (m, 3H), 2.02 (t, J=10.5 Hz, 1H), 1.70-1.46 (m, 2H), 0.86 (td, J=11.6, 5.6 Hz, 1H), 0.74 (d, J=6.5 Hz, 3H).

### Example 96

### N1,N1-dimethyl-N3-(2-(2-methylpiperidin-1-yl)phenyl)benzene-1,3-disulfonamide

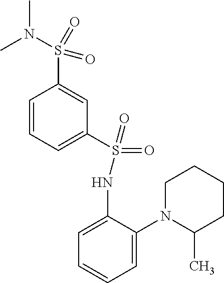 [SMILES: CC1CCCCN1c1ccccc1NS(=O)(=O)c1cccc(S(=O)(=O)N(C)C)c1]

A solution of 2-(2-methylpiperidin-1-yl)aniline (0.095 g, 0.500 mmol) in pyridine (Volume: 1 ml) was treated with 3-(N,N-dimethylsulfamoyl)benzene-1-sulfonyl chloride (0.142 g, 0.5 mmol). The solution was microwaved at 115° C. for 1 hr. The solution was cooled to room temperature. The solution was filtered to give a solid (0.084 g, 38% yield). (LCMS, ESI pos.) Calculated for C₂₀H₂₇N₃O₄S₂: 438.6 (M+H), Measured: 438.1. ¹H NMR (400 MHz, DMSO-d6) δ 9.06 (s, 1H), 8.25 (ddd, J=7.8, 1.9, 1.1 Hz, 1H), 8.02 (dt, J=7.8, 1.4 Hz, 1H), 7.98 (td, J=1.8, 0.5 Hz, 1H), 7.90 (t, J=7.8 Hz, 1H), 7.57 (dd, J=8.2, 1.5 Hz, 1H), 7.29-7.07 (m, 3H), 2.80 (s, 1H), 2.46 (s, 6H), 2.41-2.27 (m, 1H), 1.88 (d, J=11.4 Hz, 1H), 1.77-1.63 (m, 2H), 1.61-1.28 (m, 2H), 0.49 (d, J=6.1 Hz, 3H).

### Example 97

### N¹,N¹-diethyl-N⁴-(3-(4-methylpiperidin-1-yl)phenyl)benzene-1,4-disulfonamide

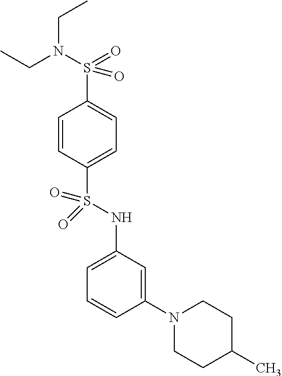 [SMILES: CCN(CC)S(=O)(=O)c1ccc(S(=O)(=O)Nc2cccc(N3CCC(C)CC3)c2)cc1]

A solution of 3-(4-methylpiperidin-1-yl)aniline (0.095 g, 0.500 mmol) in pyridine (Volume: 1 ml) was treated with 4-(N,N-diethylsulfamoyl)benzene-1-sulfonyl chloride (0.156 g, 0.5 mmol). The solution was microwaved at 115° C. for 1 hr. The crude product was purified by reversed-phase chromatography (4-100% CH₃CN over 15 min) to give desired product (0.019 g, 9% yield). (LCMS, ESI pos.) Calculated for C₂₂H₃₁N₃O₄S₂: 466.6 (M+H), Measured: 466.1. ¹H NMR (400 MHz, DMSO-d6) δ 10.28 (s, 1H), 8.04-7.82 (m, 4H), 7.04 (t, J=8.2 Hz, 1H), 6.79-6.35 (m, 3H), 3.46 (d, J=12.4 Hz, 1H), 3.14 (q, J=7.1 Hz, 4H), 2.71-2.53 (m, 2H), 1.63 (d, J=13.0 Hz, 2H), 1.48 (s, 1H), 1.14 (d, J=12.4 Hz, 2H), 0.97 (t, J=7.1 Hz, 6H), 0.89 (d, J=6.5 Hz, 3H).

### Example 98

### N¹,N¹-dimethyl-N⁴-(3-oxoisoindolin-5-yl)benzene-1,4-disulfonamide

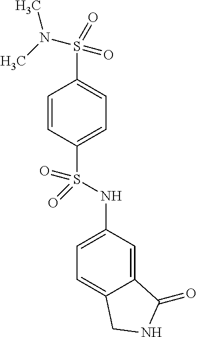 [SMILES: CN(C)S(=O)(=O)c1ccc(S(=O)(=O)Nc2ccc3c(c2)C(=O)NC3)cc1]

A solution of 6-aminoisoindolin-1-one (0.074 g, 0.500 mmol) in pyridine (Volume: 1 ml) was treated with 4-(N,N-dimethylsulfamoyl)benzene-1-sulfonyl chloride (0.142 g, 0.5 mmol). The solution was microwaved at 115° C. for 1 hr. The solution was cooled to room temperature. The solution was filtered to give a solid (0.137 g, 69% yield). (LCMS, ESI pos.) Calculated for C₁₆H₁₇N₃O₅S₂: 396.5 (M+H), Measured: 396.0. ¹H NMR (400 MHz, DMSO-d6) δ 10.73 (s, 1H), 8.62 (s, 1H), 8.09-7.86 (m, 4H), 7.51 (dq, J=7.9, 0.8 Hz, 1H), 7.40-7.29 (m, 2H), 4.31 (d, J=1.0 Hz, 2H), 2.64 (s, 6H).

### Example 99

### 6-(methylsulfonyl)-N-(4-(piperidin-1-yl)pyridine-3-yl)pyridine-3-sulfonamide

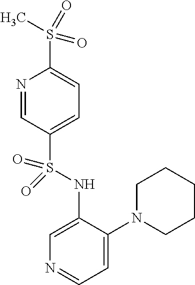 [SMILES: CS(=O)(=O)c1ccc(S(=O)(=O)Nc2cnccc2N2CCCCC2)cn1]

A solution of 4-(piperidin-1-yl)pyridin-3-amine (0.044 g, 0.250 mmol) in pyridine (Volume: 1 ml) was treated with 6-(methylsulfonyl)pyridine-3-sulfonyl chloride (0.064 g, 0.25 mmol). The solution was microwaved at 115° C. for 1 hr. The crude product was purified by reversed-phase chromatography (4-100% CH₃CN over 15 min) to give desired product (0.0035 g, 2% yield). (LCMS, ESI pos.) Calculated for C₁₆H₂₀N₄O₄S₂: 397.5 (M+H), Measured: 397.1. ¹H NMR (400 MHz, DMSO-d6) δ 9.02 (dd, J=2.2, 0.8 Hz, 1H), 8.40 (dd, J=8.2, 2.2 Hz, 1H), 8.23 (d, J=8.2 Hz, 1H), 8.09 (d, J=6.9 Hz, 1H), 7.88 (d, J=1.2 Hz, 1H), 7.16 (d, J=6.9 Hz, 1H), 3.64 (t, J=5.2 Hz, 6H), 3.37 (s, 4H), 1.69-1.53 (m, 8H).

### Example 100

### N¹,N¹-dimethyl-N³-(4-(piperidin-1-yl)pyridin-3-yl)benzene-1,3-disulfonamide

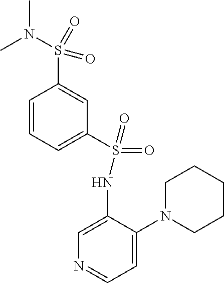 [SMILES: CN(C)S(=O)(=O)c1cccc(S(=O)(=O)Nc2cnccc2N2CCCCC2)c1]

A solution of 4-(piperidin-1-yl)pyridin-3-amine (0.044 g, 0.250 mmol) in pyridine (Volume: 1 ml) was treated with 3-(N,N-dimethylsulfamoyl)benzene-1-sulfonyl chloride (0.071 g, 0.25 mmol). The solution was microwaved at 115° C. for 1 hr. The crude product was purified by reversed-phase chromatography (4-100% CH₃CN over 15 min) to give desired product (0.0019 g, 1% yield). (LCMS, ESI pos.) Calculated for C₁₈H₂₄N₄O₄S₂: 425.5 (M+H), Measured: 425.1. ¹H NMR (400 MHz, DMSO-d6) δ 8.13 (d, J=6.9 Hz, 1H), 8.08 (d, J=7.8 Hz, 1H), 8.04 (ddd, J=7.9, 1.9, 1.1 Hz, 1H), 7.94 (t, J=1.7 Hz, 1H), 7.91 (d, J=7.8 Hz, 1H), 7.61 (s, 1H), 7.19 (d, J=7.0 Hz, 1H), 3.62 (s, 4H), 2.65 (s, 6H), 1.70-1.54 (m, 7H).

### Example 101

### N-(2-(4-methylpiperidin-1-yl)phenyl)-6-(methylsulfonyl)pyridine-3-sulfonamide

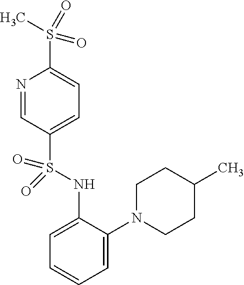 [SMILES: CC1CCN(c2ccccc2NS(=O)(=O)c2ccc(S(C)(=O)=O)nc2)CC1]

A solution of 2-(4-methylpiperidin-1-yl)aniline (0.095 g, 0.500 mmol) in pyridine (Volume: 1 ml) was treated with 6-(methylsulfonyl)pyridine-3-sulfonyl chloride (0.128 g, 0.5 mmol). The solution was microwaved at 115° C. for 1 hr. The crude product was purified by reversed-phase chromatography (4-100% CH₃CN over 15 min) to give desired product (0.0023 g, 1% yield). (LCMS, ESI pos.) Calculated for C₁₈H₂₃N₃O₄S₂: 410.5 (M+H), Measured: 410.1. ¹H NMR (400 MHz, DMSO-d6) δ 9.61 (s, 1H), 9.11 (dd, J=2.3, 0.8 Hz, 1H), 8.49 (dd, J=8.2, 2.2 Hz, 1H), 8.27 (dd, J=8.2, 0.8 Hz, 1H), 7.32-7.03 (m, 4H), 3.37 (s, 3H), 2.73-2.59 (m, 3H), 1.61-1.47 (m, 2H), 1.20-1.04 (m, 2H), 0.94 (d, J=6.5 Hz, 3H).

### Example 102

### N-(3-(4-methylpiperidin-1-yl)phenyl)-6-(methylsulfonyl)pyridine-3-sulfonamide

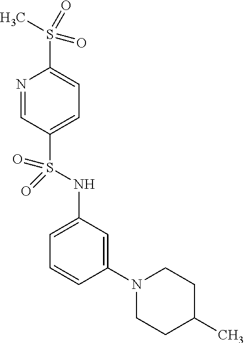 [SMILES: CC1CCN(c2cccc(NS(=O)(=O)c3ccc(S(C)(=O)=O)nc3)c2)CC1]

A solution of 3-(4-methylpiperidin-1-yl)aniline (0.095 g, 0.500 mmol) in pyridine (Volume: 1 ml) was treated with 6-(methylsulfonyl)pyridine-3-sulfonyl chloride (0.128 g, 0.5 mmol). The solution was microwaved at 115° C. for 1 hr. The solution was cooled to room temperature. The solution was filtered to give a solid (0.081 g, 40% yield). (LCMS, ESI pos.) Calculated for C₁₈H₂₃N₃O₄S₂: 410.5 (M+H), Measured: 410.1. ¹H NMR (400 MHz, DMSO-d6) δ 10.54 (s, 1H), 9.06 (dd, J=2.3, 0.8 Hz, 1H), 8.47 (dd, J=8.3, 2.3 Hz, 1H), 8.27 (dd, J=8.2, 0.8 Hz, 1H), 7.16-7.00 (m, 1H), 6.71 (ddd, J=8.5, 2.5, 0.8 Hz, 1H), 6.64 (t, J=2.2 Hz, 1H), 6.53 (ddd, J=7.9, 2.0, 0.8 Hz, 1H), 3.55 (dt, J=13.0, 3.1 Hz, 2H), 3.37 (s, 3H), 2.63 (td, J=12.3, 2.6 Hz, 2H), 1.72-1.63 (m, 2H), 1.51 (ttt, J=10.2, 6.6, 3.5 Hz, 1H), 1.24-1.10 (m, 2H), 0.94 (d, J=6.5 Hz, 3H).

### Example 103

### N-(4-(piperidin-1-yl)pyridin-3-yl)-4-((trifluoromethyl)sulfonyl)benzenesulfonamide

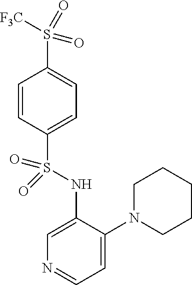 [SMILES: O=S(=O)(Nc1cnccc1N1CCCCC1)c1ccc(S(=O)(=O)C(F)(F)F)cc1]

A solution of 4-(piperidin-1-yl)pyridin-3-amine (0.044 g, 0.250 mmol) in pyridine (Volume: 1 ml) was treated with 4-((trifluoromethyl)sulfonyl)benzene-1-sulfonyl chloride (0.077 g, 0.25 mmol). The solution was microwaved at 115° C. for 1 hr. The crude product was purified by reversed-phase chromatography (4-100% CH₃CN over 15 min) to give desired product (0.001 g, 1% yield). (LCMS, ESI pos.) Calculated for C₁₇H₁₈F₃N₃O₄S₂: 450.5 (M+H), Measured: 450.1.

### Example 104

### 4-(tert-butylsulfonyl)-N-(4-(piperidin-1-yl)pyridin-3-yl)benzenesulfonamide

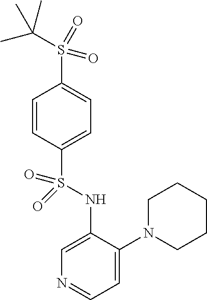 [SMILES: CC(C)(C)S(=O)(=O)c1ccc(S(=O)(=O)Nc2cnccc2N2CCCCC2)cc1]

A solution of 4-(piperidin-1-yl)pyridin-3-amine (0.044 g, 0.250 mmol) in pyridine (Volume: 1 ml) was treated with 4-(tert-butylsulfonyl)benzene-1-sulfonyl chloride (0.074 g, 0.25 mmol). The solution was microwaved at 115° C. for 1 hr. The crude product was purified by reversed-phase chromatography (4-100% CH₃CN over 15 min) to give desired product (0.005 g, 2.3% yield). (LCMS, ESI pos.) Calculated for C₂₀H₂₇N₃O₄S₂: 438.6 (M+H), Measured: 438.2. ¹H NMR (400 MHz, DMSO-d6) δ 8.18-8.13 (m, 1H), 8.10-8.05 (m, 2H), 8.00-7.95 (m, 2H), 7.63 (d, J=1.2 Hz, 1H), 7.23 (d, J=7.1 Hz, 1H), 3.65 (d, J=5.8 Hz, 5H), 1.62 (s, 5H), 1.30 (s, 9H).

### Example 105

### N-(2-chloro-6-(4-methylpiperidin-1-yl)phenyl)-6-(methylsulfonyl)pyridine-3-sulfonamide

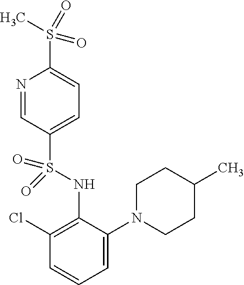 [SMILES: CC1CCN(c2cccc(Cl)c2NS(=O)(=O)c2ccc(S(C)(=O)=O)nc2)CC1]

A solution of 3-chloro-2-(4-methylpiperidin-1-yl)aniline (0.112 g, 0.500 mmol) in pyridine (Volume: 1 ml) was treated with 6-(methylsulfonyl)pyridine-3-sulfonyl chloride (0.128 g, 0.5 mmol). The solution was microwaved at 115° C. for 1 hr. The crude product was purified by reversed-phase chromatography (4-100% CH₃CN over 15 min) to give desired product (0.001 g, 1% yield). (LCMS, ESI pos.) Calculated for C₁₈H₂₂C₁N₃O₄S₂: 444.0 (M+H), Measured: 444.1. ¹H NMR (400 MHz, DMSO-d6) δ 10.51 (s, 1H), 9.29 (d, J=2.4 Hz, 1H), 8.74 (dd, J=2.4, 0.7 Hz, 1H), 8.07 (dddd, J=7.6, 5.8, 1.6, 0.7 Hz, 2H), 8.00 (s, 2H), 7.81 (ddd, J=8.4, 6.9, 1.4 Hz, 1H), 7.69 (ddd, J=8.1, 6.9, 1.1 Hz, 1H).

### Example 106

### N¹-(2-fluorophenyl)-N⁴,N⁴-dimethyl-2-nitrobenzene-1,4-disulfonamide

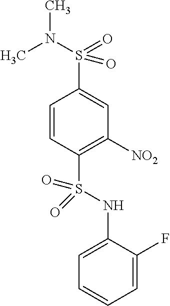 [SMILES: CN(C)S(=O)(=O)c1ccc(S(=O)(=O)Nc2ccccc2F)c([N+](=O)[O-])c1]

A solution of 2-fluoroaniline (0.048 ml, 0.500 mmol) in pyridine (Volume: 2 ml) was treated with 4-(N,N-dimethylsulfamoyl)-2-nitrobenzene-1-sulfonyl chloride (0.164 g, 0.5 mmol). The solution was microwaved at 115° C. for 1 hr. The crude product was purified by reversed-phase chromatography (4-100% CH₃CN over 15 min) to give desired product (0.009 g, 4.5% yield). (LCMS, ESI pos.) Calculated for C₁₄H₁₄FN₃O₆S₂: 403.4 (M+H), Measured: 403.9. ¹H NMR (400 MHz, DMSO-d6) δ 10.85 (s, 1H), 8.44 (d, J=1.7 Hz, 1H), 8.23 (dd, J=8.3, 1.8 Hz, 1H), 8.17 (d, J=8.3 Hz, 1H), 7.41-7.31 (m, 1H), 7.31-7.18 (m, 3H), 2.74 (s, 6H).

### Example 107

### N¹-(2-fluoro-6-(piperidin-1-yl)phenyl)-N⁴,N⁴-dimethyl-2-nitrobenzene-1,4-disulfonamide

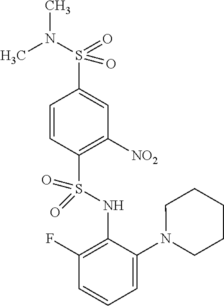 [SMILES: CN(C)S(=O)(=O)c1ccc(S(=O)(=O)Nc2c(F)cccc2N2CCCCC2)c([N+](=O)[O-])c1]

A solution of 2-fluoro-6-(piperidin-1-yl)aniline (0.194 g, 1.000 mmol) in pyridine (Volume: 1 ml) was treated with 4-(N,N-dimethylsulfamoyl)-2-nitrobenzene-1-sulfonyl chloride (0.164 g, 0.5 mmol). The resulting solution was microwaved at 120° C. for 1 hr. The crude product was purified by reversed-phase chromatography (4-100% CH₃CN over 15 min) to give desired product (0.012 g, 5% yield). (LCMS, ESI pos.) Calculated for C₁₉H₂₃FN₄O₆S₂: 487.5 (M+H), Measured: 487.1. ¹H NMR (400 MHz, DMSO-d6) δ 9.94 (s, 1H), 8.47 (d, J=1.7 Hz, 1H), 8.22 (dd, J=8.3, 1.8 Hz, 1H), 8.12 (d, J=8.2 Hz, 1H), 7.35 (td, J=8.3, 6.6 Hz, 1H), 7.07-6.82 (m, 2H), 2.77 (s, 11H), 1.28 (d, J=5.4 Hz, 3H), 1.16 (s, 4H).

### Example 108

### N,N-dimethyl-4-((2-propyl-1H-benzo[d]96midazole-1-yl)sulfonyl)benzenesulfonamide

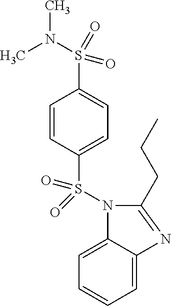 [SMILES: CCCc1nc2ccccc2n1S(=O)(=O)c1ccc(S(=O)(=O)N(C)C)cc1]

A solution of 2-propyl-1H-benzo[d]imidazole (0.080 g, 0.5 mmol) in pyridine (Volume: 1.000 ml) was treated with 4-(N,N-dimethylsulfamoyl)benzene-1-sulfonyl chloride (0.142 g, 0.500 mmol). The solution was microwaved at 120° C. for 1 hr. The solution was cooled to room temperature. The solution was filtered to give a solid (0.001 g, 1% yield). (LCMS, ESI pos.) Calculated for C₁₈H₂₁N₃O₄S₂: 408.5 (M+H), Measured: 408.1. ¹H NMR (400 MHz, DMSO-d6) δ 8.35-8.29 (m, 2H), 8.04-7.97 (m, 3H), 7.75-7.71 (m, 1H), 7.48-7.38 (m, 2H), 3.18 (dd, J=7.7, 7.1 Hz, 2H), 2.66 (s, 6H), 1.96-1.82 (m, 2H), 1.02 (t, J=7.4 Hz, 3H).

### Example 109

### 2-amino-N⁴,N⁴-dimethyl-N1-(2-(piperidin-1-yl)phenyl)benzene-1,4-disulfonamide

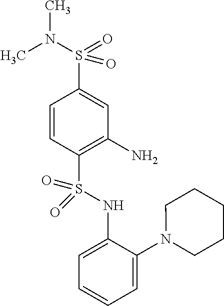 [SMILES: CN(C)S(=O)(=O)c1ccc(S(=O)(=O)Nc2ccccc2N2CCCCC2)c(N)c1]

A solution of N⁴,N⁴-dimethyl-2-nitro-N1-(2-(piperidin-1-yl)phenyl)benzene-1,4-disulfonamide (0.5 g, 1.067 mmol) in MeOH (Volume: 107 ml) (a small amount of DMF was added to get the compound in solution) was subjected to H-cube conditions: 0.8 ml/min, 40 barr, 40° C., 100% H2. The solution was concentrated and dried in vacuo to give the desired compound (0.024 g, 11%). (LCMS, ESI pos.) Calculated for C₁₉H₂₆N₄O₄S₂: 439.6 (M+H), Measured: 439.1. ¹H NMR (400 MHz, DMSO-d6) δ 9.04 (s, 1H), 7.99 (s, 1H), 7.67 (d, J=8.4 Hz, 1H), 7.32-7.27 (m, 1H), 7.26-7.23 (m, 1H), 7.21-7.16 (m, 1H), 7.13-7.02 (m, 2H), 6.83 (dd, J=8.4, 1.8 Hz, 1H), 6.64 (s, 2H), 2.93 (d, J=0.5 Hz, 3H), 2.77 (d, J=0.7 Hz, 3H), 2.62 (s, 6H), 2.61-2.56 (m, 4H), 1.70 (dq, J=11.1, 5.2 Hz, 4H), 1.52 (d, J=5.4 Hz, 2H).

### Example 110

### N¹-(5-hydroxy-2-(4-methylpiperidin-1-yl)phenyl)-N⁴,N⁴-dimethylbenzene-1,4-disulfonamide

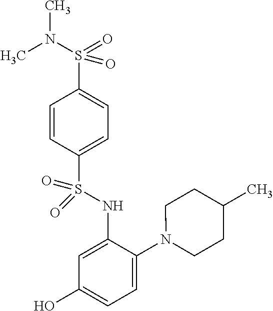

A solution of 3-amino-4-(4-methylpiperidin-1-yl)phenol (0.206 g, 1 mmol) in pyridine (Volume: 2 ml) was treated with 4-(N,N-dimethylsulfamoyl)benzene-1-sulfonyl chloride (0.284 g, 1.000 mmol). The solution was microwaved at 130° C. for 1 hr. The crude product was purified by reversed-phase chromatography (4-100% CH₃CN over 15 min) to give desired product (0.026 g, 11% yield). (LCMS, ESI pos.) Calculated for C₂₀H₂₇N₃O₅S₂: 454.6 (M+H), Measured: 454.1. ¹H NMR (400 MHz, DMSO-d6) δ 9.50 (s, 1H), 8.96 (s, 1H), 8.04-8.00 (m, 2H), 7.97 (d, J=8.6 Hz, 2H), 7.03 (s, 1H), 6.88 (s, 1H), 6.54 (s, 1H), 2.64 (s, 6H), 2.25 (s, 2H), 1.64-1.18 (m, 5H), 0.96 (d, J=6.0 Hz, 3H).

### Example 111

### N,N-dimethyl-4-oxo-2-(2-(piperidin-1-yl)phenyl)-2,3,4,5-tetrahydrobenzo[f][1,2,5]thiadiazepine-7-sulfonamide 1,1-dioxide

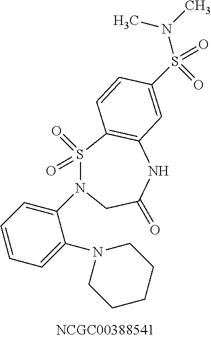 [SMILES: CN(C)S(=O)(=O)c1ccc2c(c1)NC(=O)CN(c1ccccc1N1CCCCC1)S2(=O)=O]

To a solution of 2-amino-N⁴,N⁴-dimethyl-N¹-(2-(piperidin-1-yl)phenyl)benzene-1,4-disulfonamide (Example 109) (0.05 g, 0.114 mmol) in THF (Volume: 0.456 ml) was added Et₃N (0.048 ml, 0.342 mmol). To the resulting solution it was added dropwise (VIOLENT reaction!) 2-bromoacetyl chloride (0.013 ml, 0.125 mmol). The solution was microwaved at 90° C. for 1 hr. The crude product was purified by reversed-phase chromatography (4-100% CH₃CN over 15 min) to give desired product (0.001 g, 1% yield). (LCMS, ESI pos.) Calculated for C₂₁H₂₆N₄₀₅S₂: 479.6 (M+H), Measured: 479.1.

### Example 112

### N,N-dimethyl-4-((2-pentyl-1H-benzo[d]imidazol-1-yl)sulfonyl)benzenesulfonamide

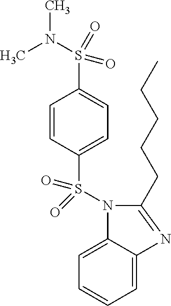 [SMILES: CCCCCc1nc2ccccc2n1S(=O)(=O)c1ccc(S(=O)(=O)N(C)C)cc1]

A solution of 2-pentyl-1H-benzo[d]imidazole (0.094 g, 0.500 mmol) in pyridine (Volume: 1.000 ml) was treated with 4-(N,N-dimethylsulfamoyl)benzene-1-sulfonyl chloride (0.142 g, 0.5 mmol). The resulting solution was microwaved at 115° C. for 1 hr. The solution was cooled to room temperature. The solution was filtered to give a solid (0.155 g, 71% yield). (LCMS, ESI pos.) Calculated for C₂₀H₂₅N₃O₄S₂: 436.6 (M+H), Measured: 436.1. ¹H NMR (400 MHz, DMSO-d6) δ 8.35-8.29 (m, 2H), 8.05-7.97 (m, 3H), 7.75-7.70 (m, 1H), 7.49-7.38 (m, 2H), 3.39 (s, 2H), 3.18 (dd, J=8.0, 7.1 Hz, 2H), 2.66 (s, 6H), 1.85 (p, J=7.5 Hz, 2H), 1.46-1.28 (m, 2H), 0.96-0.85 (m, 2H).

### Example 113

### N¹-(2-aminophenyl)-N⁴,N⁴-dimethylbenzene-1,4-disulfonamide

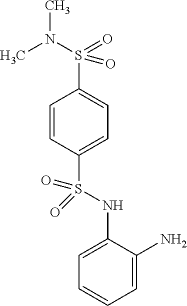 [SMILES: CN(C)S(=O)(=O)c1ccc(S(=O)(=O)Nc2ccccc2N)cc1]

A solution of 1-(2-aminophenyl)piperidine-2,6-dione (0.1 g, 0.490 mmol) in pyridine (Volume: 0.490 ml) was treated with 4-(N,N-dimethylsulfamoyl)benzene-1-sulfonyl chloride (0.139 g, 0.490 mmol). The solution was microwaved at 120° C. for 1 hr. The crude product was purified by reversed-phase chromatography (4-100% CH₃CN over 15 min) to give desired product (0.052 g, 24% yield). (LCMS, ESI pos.) Calculated for C₁₄H₁₇N₃O₄S₂: 356.4 (M+H), Measured: 356.1. ¹H NMR (400 MHz, DMSO-d6) δ 7.93 (s, 4H), 6.97 (ddd, J=8.0, 7.2, 1.6 Hz, 1H), 6.69 (ddd, J=9.8, 8.0, 1.4 Hz, 2H), 6.49 (ddd, J=7.9, 7.2, 1.5 Hz, 1H), 2.65 (s, 6H).

### Example 114

### 2-chloro-N-(5-(N,N-dimethylsulfamoyl)-2-(N-(2-(piperidin-1-yl)phenyl)sulfamoyl)phenyl)acetamide

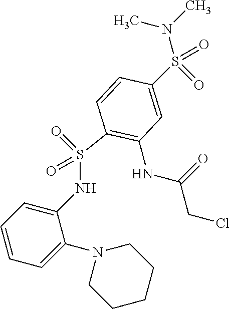 [SMILES: CN(C)S(=O)(=O)c1ccc(S(=O)(=O)Nc2ccccc2N2CCCCC2)c(NC(=O)CCl)c1]

A solution of 2-amino-N⁴,N⁴-dimethyl-N¹-(2-(piperidin-1-yl)phenyl)benzene-1,4-disulfonamide (Example 109) (0.2 g, 0.391 mmol) and Et₃N (0.163 ml, 1.173 mmol) in THF (Volume: 1.564 ml) was treated with 2-bromoacetyl chloride (0.045 ml, 0.430 mmol) (added dropwise but with no cooling of the reaction solution). The resulting brown solution was allowed to cool to room temperature. The solution was microwaved at 90° C. for 1 hr. The crude product was purified by reversed-phase chromatography (4-100% CH₃CN over 15 min) to give desired product (0.001 g, 1% yield). (LCMS, ESI pos.) Calculated for C₂₁H₂₇ClN₄O₅S₂: 515.0 (M+H), Measured: 515.1. ¹H NMR (400 MHz, DMSO-d6) δ 10.09 (s, 1H), 9.65 (s, 1H), 8.66 (d, J=1.8 Hz, 1H), 7.98 (d, J=8.3 Hz, 1H), 7.65 (dd, J=8.3, 1.9 Hz, 1H), 7.32 (dd, J=7.9, 1.5 Hz, 1H), 7.24-7.06 (m, 3H), 4.43 (s, OH), 4.41 (s, 2H), 4.20 (t, J=6.3 Hz, 0H), 3.71 (t, J=6.4 Hz, 0H), 3.61 (t, J=6.6 Hz, 0H), 2.68 (s, 6H), 1.54-1.34 (m, 7H).

### Example 115

### Resolution of enantiomers of (+/−)N¹,N¹-dimethyl-N4-(2-(3-methylpiperidin-1-yl)phenyl)benzene-1,4-disulfonamide

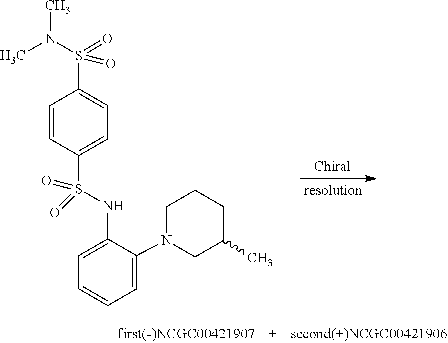

(+/−)N¹,N¹-dimethyl-N4-(2-(3-methylpiperidin-1-yl)phenyl)benzene-1,4-disulfonamide was resolved into the corresponding enantiomers by normal phase chromatography using a CHIRALPAK®AD® column (5×50 cm, 20 mm) on an Agilent 1200 HPLC with a Methanol/Diethylamine (100:0.05) isocratic mobile phase eluting at 40 mL/min and detecting at 230 nm. Separation of the racemic mixture provided NCGC00421907_1st_neg with ee greater than 98%, and NCGC00421906_2nd_pos with ee greater than 98%. The individual fractions were collected separately, concentrated and dried in vacuo to give the desired compounds: (−)NCGC00421907 (0.0038 g; 1% yield). Calculated for C₂₀H₂₇N₃O₄S₂: 438.6 (M+H), Measured: 438.1. ¹H NMR (400 MHz, DMSO-d6) δ 9.15-8.88 (m, 1H), 8.01 (d, J=8.5 Hz, 2H), 7.95 (d, J=8.5 Hz, 2H), 7.33 (d, J=7.7 Hz, 1H), 7.15 (d, J=4.0 Hz, 2H), 7.13-7.06 (m, 1H), 2.66 (s, 6H), 2.51-2.32 (m, 3H), 2.09 (t, J=10.5 Hz, 1H), 1.75-1.61 (m, 2H), 1.56 (dd, J=7.5, 3.8 Hz, 2H), 0.93 (q, J=10.1, 9.0 Hz, 1H), 0.81 (d, J=6.4 Hz, 3H). (+)NCGC00421906 (0.0048 g, 1% yield). Calculated for C₂₀H₂₇N₃O₄S₂: 438.6 (M+H), Measured: 438.1. ¹H NMR (400 MHz, DMSO-d6) (400 MHz, DMSO-d6) δ 9.02 (s, 1H), 8.01 (d, J=8.5 Hz, 2H), 7.95 (d, J=8.5 Hz, 2H), 7.36-7.31 (m, 1H), 7.18-7.13 (m, 2H), 7.11 (ddd, J=7.6, 5.2, 3.7 Hz, 1H), 2.66 (s, 6H), 2.52-2.31 (m, 3H), 2.09 (t, J=10.5 Hz, 1H), 1.68 (dddd, J=13.8, 10.4, 6.8, 3.5 Hz, 2H), 1.56 (dd, J=7.4, 3.7 Hz, 2H), 1.00-0.85 (m, 1H), 0.81 (d, J=6.4 Hz, 3H).

### Example 116

### Resolution of enantiomers of (+/−)N¹,N¹-dimethyl-N4-(2-(2-methylpiperidin-1-yl)phenyl)benzene-1,4-disulfonamide

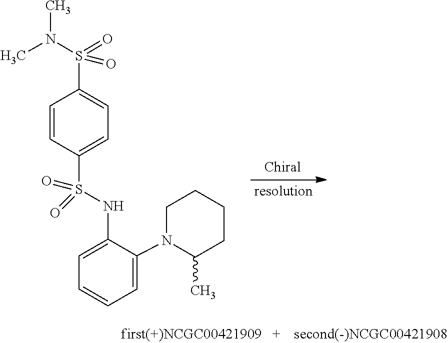 [SMILES: CC1CCCCN1c1ccccc1NS(=O)(=O)c1ccc(S(=O)(=O)N(C)C)cc1]

(+/−)N¹,N¹-dimethyl-N⁴-(2-(2-methylpiperidin-1-yl)phenyl)benzene-1,4-disulfonamide was resolved into the corresponding enantiomers by normal phase chromatography using a CHIRALPAK®AS® column (5×50 cm, 20 mm) on an Agilent 1200 HPLC with a Hexane/Ethanol (30:70) isocratic mobile phase eluting at 40 mL/min and detecting at 230 nm. Separation of the racemic mixture provided NCGC00421909_1st_pos with ee 86%, and NCGC00421908_2nd_neg with ee greater than 98%. The individual fractions were collected separately, concentrated and dried in vacuo to give the desired compounds: (+)NCGC00421909 (0.0033 g; 1% yield). Calculated for C₂₀H₂₇N₃O₄S₂: 438.6 (M+H), Measured: 438.2. ¹H NMR (400 MHz, DMSO-d6) δ 9.05 (s, 1H), 8.07 (d, J=8.2 Hz, 2H), 7.92 (d, J=8.1 Hz, 2H), 7.52 (s, 1H), 7.20 (d, J=26.9 Hz, 4H), 2.62 (s, 6H), 1.77-1.63 (m, 2H), 1.63-1.25 (m, 4H), 0.50 (d, J=6.1 Hz, 3H).
(−)NCGC00421908 (0.0015 g, 1% yield). Calculated for C₂₀H₂₇N₃O₄S₂: 438.6 (M+H), Measured: 438.1. ¹H NMR (400 MHz, DMSO-d6) (400 MHz, DMSO-d₆) δ 9.04 (s, 1H), 8.07 (d, J=8.2 Hz, 2H), 7.92 (d, J=8.1 Hz, 2H), 7.52 (s, 1H), 7.41-6.88 (m, 5H), 2.62 (s, 6H), 1.83-1.15 (m, 9H), 0.50 (d, J=6.1 Hz, 3H).

### Example 117

### 4-bromo-2-fluoro-N-(2-hydroxy-6-(piperidin-1-yl)phenyl)benzenesulfonamide

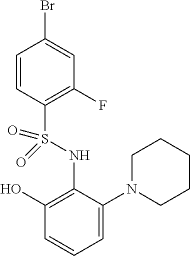 [SMILES: O=S(=O)(Nc1c(O)cccc1N1CCCCC1)c1ccc(Br)cc1F]

A solution of 2-amino-3-(piperidin-1-yl)phenol (0.096 g, 0.5 mmol) in pyridine (Volume: 1.000 ml) was treated with 4-bromo-2-fluorobenzene-1-sulfonyl chloride (0.137 g, 0.500 mmol). The solution was microwaved at 120° C. for 1 hr. The crude product was purified by reversed-phase chromatography (4-100% CH₃CN over 15 min) to give desired product (0.018 g, 8% yield). (LCMS, ESI pos.) Calculated for C₁₇H₁₈BrFN₂O₃S: 429.3 (M+H), Measured: 429.0. ¹H NMR (400 MHz, DMSO-d6) δ 9.30 (d, J=53.3 Hz, 1H), 8.90 (s, 1H), 7.83 (dd, J=9.6, 1.8 Hz, 1H), 7.64 (t, J=8.0 Hz, 1H), 7.56 (dd, J=8.4, 1.9 Hz, 1H), 7.02 (s, 1H), 6.51 (s, 2H), 3.05-2.62 (m, 4H), 1.44 (s, 7H).

### Example 118

### 2-bromo-7-(piperidin-1-yl)-6H-dibenzo[b,f][1,4,5]oxathiazepine 5,5-dioxide

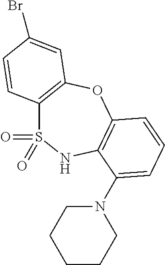 [SMILES: O=S1(=O)Nc2c(cccc2N2CCCCC2)Oc2cc(Br)ccc21]

A solution of 2-amino-3-(piperidin-1-yl)phenol (0.1 g, 0.520 mmol) in DMF (Volume: 2.001 ml) was treated with pyridine (0.042 ml, 0.520 mmol) followed by 4-bromo-2-fluorobenzene-1-sulfonyl chloride (0.142 g, 0.520 mmol). The solution was microwaved at 120° C. for 1 hr. To the solution was added POTASSIUM CARBONATE (0.144 g, 1.040 mmol). The solution was microwaved at 160° C. for 2 hrs. The crude product was purified by reversed-phase chromatography (4-100% CH₃CN over 15 min) to give desired product (0.013 g, 6.4% yield). (LCMS, ESI pos.) Calculated for C₁₇H₁₇BrN₂O₃S: 409.3 (M+H), Measured: 409.0, 411.0. ¹H NMR (400 MHz, DMSO-d6) δ 7.84 (d, J=1.9 Hz, 1H), 7.71 (d, J=8.4 Hz, 1H), 7.63 (dd, J=8.4, 1.9 Hz, 1H), 7.24 (t, J=8.1 Hz, 1H), 7.08 (dd, J=8.2, 1.4 Hz, 1H), 6.98 (dd, J=8.2, 1.4 Hz, 1H), 3.07-3.01 (m, 4H), 1.76-1.66 (m, 4H), 1.59 (q, J=5.8 Hz, 2H).

### Example 119 (a)

### 1-(2-nitrophenyl)-4-(trifluoromethyl)piperidine

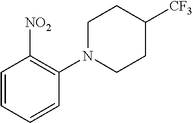 [SMILES: O=[N+]([O-])c1ccccc1N1CCC(C(F)(F)F)CC1]

A solution of 4-(trifluoromethyl)piperidine (0.306 g, 2 mmol) in DMF (Volume: 2.000 ml) was treated with POTASSIUM CARBONATE (0.276 g, 2.000 mmol) (grounded using a mortar and pestle) followed by addition of 1-fluoro-2-nitrobenzene (0.211 ml, 2.000 mmol). The resulting heterogeneous solution was microwaved at 155° C. for 2 hrs. The reaction was cooled to room temperature. The solution was filtered through Celite and charcoal. The filtrate was used as is for the next step.

### Example 119 (b)

### 2-(4-(trifluoromethyl)piperidin-1-yl)aniline

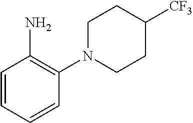 [SMILES: Nc1ccccc1N1CCC(C(F)(F)F)CC1]

The solution of 1-(2-nitrophenyl)-4-(trifluoromethyl)piperidine (0.548 g, 2 mmol) in EtOH (Volume: 100 ml) was subjected to H-cube conditions: 0.8 ml/min, 100% H₂, 40 barr, 40° C. The solution was concentrated and dried in vacuo. The residue was used as is for the next step.

### Example 119 (c)

### N¹,N¹-dimethyl-N⁴-(2-(4-(trifluoromethyl)piperidin-1-yl)phenyl)benzene-1,4-disulfonamide

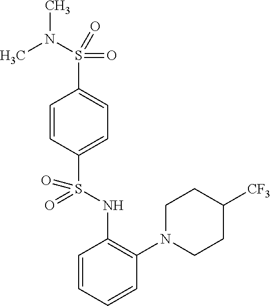 [SMILES: CN(C)S(=O)(=O)c1ccc(S(=O)(=O)Nc2ccccc2N2CCC(C(F)(F)F)CC2)cc1]

A solution of 2-(4-(trifluoromethyl)piperidin-1-yl)aniline (0.5 g, 2.047 mmol) and DIEA (0.715 ml, 4.09 mmol) in DMF (Volume: 4.09 ml) was treated with 4-(N,N-dimethylsulfamoyl)benzene-1-sulfonyl chloride (0.581 g, 2.047 mmol). The solution was microwaved at 120° C. for 1 hr. The crude product was purified by reversed-phase chromatography (4-100% CH₃CN over 15 min) to give desired product (0.182 g, 74% yield). (LCMS, ESI pos.) Calculated for C₂₀H₂₄F₃N₃O₄S₂: 492.5 (M+H), Measured: 492.1. ¹H NMR (400 MHz, DMSO-d6) δ 9.42 (s, 1H), 8.07-8.01 (m, 2H), 8.00-7.94 (m, 2H), 7.34-7.29 (m, 1H), 7.20-7.07 (m, 3H), 4.11 (q, J=5.2 Hz, 0H), 3.21 (d, J=5.3 Hz, 1H), 2.66 (s, 8H), 2.60-2.57 (m, 1H), 1.69 (dp, J=12.1, 8.0, 6.8 Hz, 4H).

### Example 120 (a)

### 2-(benzylthio)-7-(piperidin-1-yl)-6H-dibenzo[b,f][1,4,5]oxathiazepine 5,5-dioxide

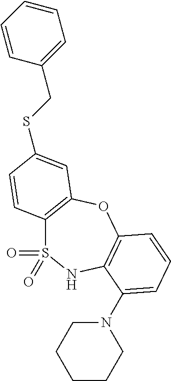 [SMILES: O=S1(=O)Nc2c(cccc2N2CCCCC2)Oc2cc(SCc3ccccc3)ccc21]

POTASSIUM CARBONATE (0.061 g, 0.443 mmol) in xylene (2.0 ml) was degassed and filled with N2 (3×). The solution was cooled using an ice/water bath. To the solution was added dropwise phenylmethanethiol (0.104 ml, 0.886 mmol). The resulting system was allowed to warm up to room temperature and was stirred for 1 hr. (SOLUTION A). Separately a mixture of XANTPHOS (0.045 g, 0.078 mmol) and Pd2(dba)3 (0.065 g, 0.071 mmol) was treated with a solution of 2-bromo-7-(piperidin-1-yl)-6H-dibenzo[b,f][1,4,5]oxathiazepine 5,5-dioxide (0.29 g, 0.709 mmol) in xylene (10 ml). The solution was evacuated and fed with N2 (3×). The solution was stirred under N2 for 20 minutes. The solution was added to SOLUTION A. The system was evacuated and fed with N2 (3×). The solution was microwaved at 165° C. for 2 hrs. The desired product was purified using a 40 g silica gel column. Elution was done with a gradient (10-80% EtOAc in hexanes). Desired fractions were combined, concentrated and dried in vacuo to give the desired product (0.26 g, 81% yield). ¹H NMR (400 MHz, Chloroform-d) δ 7.69 (d, J=8.2 Hz, 1H), 7.38-7.23 (m, 6H), 7.20 (d, J=1.7 Hz, 1H), 7.13 (dd, J=8.2, 1.8 Hz, 1H), 7.05 (dd, J=8.0, 1.7 Hz, 1H), 7.02-6.91 (m, 2H), 4.20 (s, 2H), 2.81-2.72 (m, 4H), 1.71 (dq, J=10.9, 5.2 Hz, 5H), 1.58 (q, J=6.1, 5.6 Hz, 2H).

### Example 120 (b)

### 7-(piperidin-1-yl)-6H-dibenzo[b,f][1,4,5]oxathiazepine-2-sulfonyl chloride 5,5-dioxide

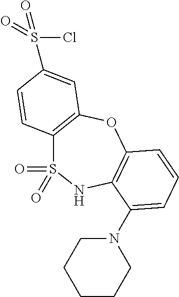 [SMILES: O=S(=O)(Cl)c1ccc2c(c1)Oc1cccc(N3CCCCC3)c1NS2(=O)=O]

2-(benzylthio)-7-(piperidin-1-yl)-6H-dibenzo[b,f][1,4,5]oxathiazepine 5,5-dioxide (0.065 g, 0.144 mmol) was taken up in CH3CN/CH3COOH/H2O 40:1.5:1.0 (Volume: 1.436 ml) (Note: a precipitate formed from the solution upon standing). The solution was cooled using an ice/water bath. To the solution was added portionwise over a period of 2 min 1,3-dichloro-5,5-dimethylimidazolidine-2,4-dione (0.057 g, 0.287 mmol). The solution was stirred in the cold. Used as is for the next step.

### Example 120 (c)

### N,N-dimethyl-7-(piperidin-1-yl)-6H-dibenzo[b,f][1,4,5]oxathiazepine-2-sulfonamide 5,5-dioxide

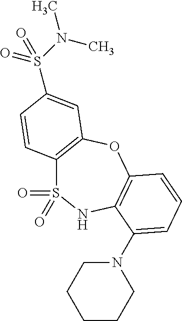 [SMILES: CN(C)S(=O)(=O)c1ccc2c(c1)Oc1cccc(N3CCCCC3)c1NS2(=O)=O]

7-(piperidin-1-yl)-6H-dibenzo[b,f][1,4,5]oxathiazepine-2-sulfonyl chloride 5,5-dioxide (0.062 g, 0.144 mmol) was treated with dimethylamine (5 ml, 10 mmol). The solution was stirred at room temperature. The crude product was purified using reversed-phase chromatography (0-100% CH3CN over 15 min). Desired fractions were combined and concentrated to give desired compound (0.0047 g, 2% yield). (LCMS, ESI pos.) Calculated for C₁₉H₂₃N₃O₅S₂: 438.5 (M+H), Measured: 438.1. ¹H NMR (400 MHz, DMSO-d6) δ 10.18 (s, 1H), 8.03 (d, J=8.2 Hz, 1H), 7.85 (d, J=1.7 Hz, 1H), 7.75 (dd, J=8.2, 1.8 Hz, 1H), 7.24 (t, J=8.1 Hz, 1H), 7.18 (dd, J=8.1, 1.5 Hz, 1H), 7.01 (dd, J=8.0, 1.5 Hz, 1H), 3.11-3.04 (m, 4H), 2.72 (s, 6H), 2.59 (t, J=5.6 Hz, 0H), 1.73 (p, J=5.4 Hz, 5H), 1.60 (q, J=5.8 Hz, 2H).

### Example 121

### 1-(2-((4-(N,N-dimethylsulfamoyl)phenyl)sulfonamido)phenyl)piperidine-4-carboxylic acid

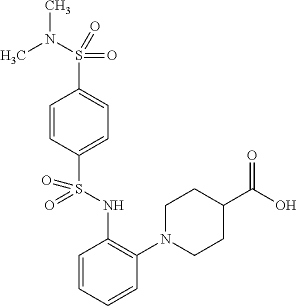 [SMILES: CN(C)S(=O)(=O)c1ccc(S(=O)(=O)Nc2ccccc2N2CCC(C(=O)O)CC2)cc1]

A heterogeneous solution of ethyl 1-(2-(4-(N,N-dimethylsulfamoyl)phenylsulfonamido)phenyl)piperidine-4-carboxylate (0.1024 g, 0.207 mmol) in EtOH/DMF 1.0/0.5 (Volume: 1.5 ml) was treated with SODIUM HYDROXIDE (0.248 ml, 0.248 mmol). The solution became homogeneous after the addition of the base. The solution was stirred at room temperature for 18 hrs. The residue was taken up in water and cooled using a ice/water bath. To the solution was added slowly 1N HCl to pH 6 (litmus). The solution was placed in the refrigerator. The solution was filtered. The solid was dried in vacuo to give the desired compound (0.027 g, 12% yield). (LCMS, ESI pos.) Calculated for C₂₀H₂₅N₃O₆S₂: 468.6 (M+H), Measured: 468.1. ¹H NMR (400 MHz, DMSO-d6) δ 12.23 (s, 1H), 9.32 (s, 1H), 8.07-8.02 (m, 2H), 7.99-7.94 (m, 2H), 7.31 (dt, J=7.7, 1.2 Hz, 1H), 7.17-7.13 (m, 2H), 7.13-7.06 (m, 1H), 2.66 (s, 6H), 2.28 (q, J=8.5, 7.1 Hz, 1H), 1.76 (td, J=10.1, 8.8, 4.0 Hz, 4H).

### Example 122

### N¹,N¹-dimethyl-N⁴-(3-methyl-2-(2-oxopiperidin-1-yl)phenyl)benzene-1,4-disulfonamide

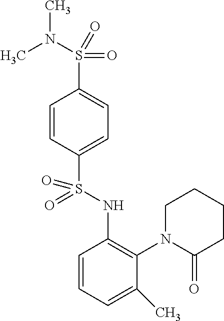 [SMILES: Cc1cccc(NS(=O)(=O)c2ccc(S(=O)(=O)N(C)C)cc2)c1N1CCCCC1=O]

A solution of 1-(2-amino-6-methylphenyl)piperidin-2-one (0.204 g, 1 mmol) in pyridine (Volume: 2.000 ml) was treated with 4-(N,N-dimethylsulfamoyl)benzene-1-sulfonyl chloride (0.284 g, 1.000 mmol). The solution was microwaved at 120° C. for 1 hr. The crude product was purified by reversed-phase chromatography (4-100% CH₃CN over 15 min) to give desired product (0.30 g, 68% yield). (LCMS, ESI pos.) Calculated for C₂₀H₂₅N₃O₅S₂: 452.6 (M+H), Measured: 452.1. ¹H NMR (400 MHz, DMSO-d6) δ 10.03 (s, 1H), 7.99-7.93 (m, 2H), 7.92-7.85 (m, 2H), 7.21 (td, J=7.9, 0.7 Hz, 1H), 7.12 (dd, J=7.9, 1.3 Hz, 1H), 6.97 (dd, J=8.0, 1.3 Hz, 1H), 3.53 (dt, J=11.3, 3.6 Hz, 1H), 3.23-3.13 (m, 1H), 2.66 (s, 6H), 2.39-2.31 (m, 2H), 1.94-1.77 (m, 4H), 1.70 (s, 3H).

### Example 123

### N¹,N¹-dimethyl-N⁴-(2-(pyridin-3-yl)phenyl)benzene-1,4-disulfonamide

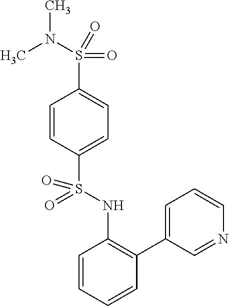 [SMILES: CN(C)S(=O)(=O)c1ccc(S(=O)(=O)Nc2ccccc2-c2cccnc2)cc1]

A solution of 2-(pyridin-3-yl)aniline (0.170 g, 1.000 mmol) in pyridine (Volume: 2.000 ml) was treated with 4-(N,N-dimethylsulfamoyl)benzene-1-sulfonyl chloride (0.284 g, 1 mmol). The solution was microwaved at 120° C. for 1 hr. The product was purified by reversed-phase chromatography (4-100% CH₃CN over 15 min) to give desired product (0.013 g, 6% yield). (LCMS, ESI pos.) Calculated for C₁₉H₁₉N₃O₄S₂: 418.5 (M+H), Measured: 418.1. ¹H NMR (400 MHz, DMSO-d6) δ 10.16 (s, 1H), 8.62 (dd, J=5.0, 1.6 Hz, 1H), 8.55 (d, J=2.2 Hz, 1H), 7.93-7.85 (m, 3H), 7.81-7.73 (m, 2H), 7.55 (dd, J=7.9, 5.0 Hz, 1H), 7.47-7.39 (m, 3H), 7.06 (dt, J=6.3, 1.7 Hz, 1H), 2.70 (s, 6H).

### Example 124 (a)

### 3-(3-methyl-3H-diazirin-3-yl)propanoyl chloride

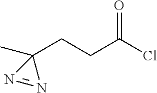

A solution of 3-(3-methyl-3H-diazirin-3-yl)propanoic acid (0.03 g, 0.234 mmol) in CH2Cl2 (Volume: 0.750 ml) was treated with OXALYL CHLORIDE (0.041 ml, 0.468 mmol). The solution was treated with DMF (0.181 μl, 2.341 μmol). The resulting bubbling solution was stirred at room temperature under N2. After 1 hr the solution was concentrated. Used as is for the next step.

### Example 124 (b)

### N-(5-(N,N-dimethylsulfamoyl)-2-(N-(2-(piperidin-1-yl)phenyl)sulfamoyl)phenyl)-3-(3-methyl-3H-diazirin-3-yl)propanamide

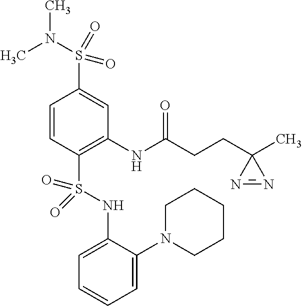

A solution of 2-amino-N⁴,N⁴-dimethyl-N¹-(2-(piperidin-1-yl)phenyl)benzene-1,4-disulfonamide (0.05 g, 0.114 mmol) in pyridine (0.5 ml) was added to 3-(3-methyl-3H-diazirin-3-yl)propanoyl chloride (0.034 g, 0.23 mmol). The solution was stirred at room temperature under N2. After 2 hrs, the reaction solution was treated with 1 N NaOH (1 ml). Stirring was continued for 48 hr. The reaction solution was treated with toluene. The solution was concentrated. The residue was taken up in DMF (2 ml), filtered. The filtrate was purified by reversed-phase chromatography (4-100% CH₃CN over 15 min) to give desired product (0.088 g, 3% yield). (LCMS, ESI pos.) Calculated for C₂₄H₃₂N₆O₅S₂: 549.7 (M+H), Measured: 549.1. ¹H NMR (400 MHz, DMSO-d6) δ 9.75 (s, 1H), 9.56 (s, 1H), 8.70 (d, J=1.8 Hz, 1H), 7.83 (d, J=8.4 Hz, 1H), 7.54 (dd, J=8.3, 1.9 Hz, 1H), 7.39 (dd, J=7.8, 1.6 Hz, 1H), 7.26-7.09 (m, 3H), 2.66 (s, 6H), 2.45 (t, J=5.1 Hz, 5H), 2.37-2.30 (m, 2H), 1.73-1.65 (m, 2H), 1.42 (d, J=23.8 Hz, 7H), 1.08 (s, 3H).

### Example 125

### 4-(dimethylamino)-N-(2-(piperidin-1-yl)phenyl)benzenesulfonamide

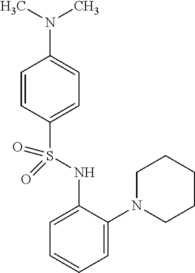 [SMILES: CN(C)c1ccc(S(=O)(=O)Nc2ccccc2N2CCCCC2)cc1]

A solution of 2-(piperidin-1-yl)aniline (0.176 g, 1 mmol) in pyridijne (Volume: 1.000 ml) was treated with 4-(dimethylamino)benzene-1-sulfonyl chloride (0.220 g, 1.000 mmol). The solution was microwaved at 115° C. for 1 hr. The solution was cooled to room temperature. The solution was filtered to give a solid (0.128 g, 36% yield). (LCMS, ESI pos.) Calculated for C₁₉H₂₅N₃₀₂S: 360.5 (M+H), Measured: 360.1. ¹H NMR (400 MHz, DMSO-d6) δ 8.25 (s, 1H), 7.60-7.55 (m, 2H), 7.43-7.38 (m, 1H), 7.17 (dd, J=7.7, 1.7 Hz, 1H), 7.11-7.00 (m, 2H), 6.76-6.70 (m, 2H), 2.98 (s, 6H), 2.52-2.46 (m, 4H), 1.65 (dq, J=10.9, 5.2 Hz, 5H), 1.57-1.47 (m, 2H).

### Example 126

### 4-((dimethylamino)methyl)-N-(2-(piperidin-1-yl)phenyl)benzenesulfonamide

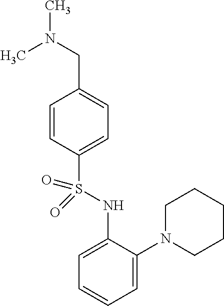 [SMILES: CN(C)Cc1ccc(S(=O)(=O)Nc2ccccc2N2CCCCC2)cc1]

A solution of 2-(piperidin-1-yl)aniline (0.176 g, 1.000 mmol) in pyridine (Volume: 1.000 ml) was treated with 4-((dimethylamino)methyl)benzene-1-sulfonyl chloride (0.234 g, 1 mmol). The solution was microwaved at 115° C. for 1 hr. The solution was cooled to room temperature. The solution was filtered to give a solid (0.073 g, 20% yield). (LCMS, ESI pos.) Calculated for C₂₀H₂₇N₃₀₂S: 374.5 (M+H), Measured: 374.1. ¹H NMR (400 MHz, DMSO-d6) δ 9.90 (s, 1H), 8.90 (s, 1H), 7.98-7.90 (m, 2H), 7.72-7.67 (m, 2H), 7.38-7.31 (m, 1H), 7.21-7.04 (m, 3H), 4.37 (d, J=4.2 Hz, 2H), 2.74 (d, J=3.6 Hz, 6H), 2.49 (t, J=5.2 Hz, 4H), 1.61 (q, J=5.8, 5.4 Hz, 4H), 1.50 (t, J=6.3 Hz, 2H).

### Example 127

### 4-chloro-N¹,N¹-dimethyl-N³-(2-(piperidin-1-yl)phenyl)benzene-1,3-disulfonamide

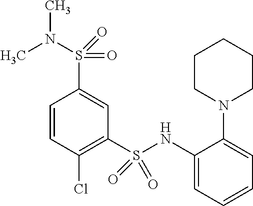 [SMILES: CN(C)S(=O)(=O)c1ccc(Cl)c(S(=O)(=O)Nc2ccccc2N2CCCCC2)c1]

A solution of 2-(piperidin-1-yl)aniline (0.176 g, 1.000 mmol) in pyridine (1.000 ml) was treated with 2-chloro-5-(N,N-dimethylsulfamoyl)benzenesulfonyl chloride (0.318 g, 1 mmol). The solution was microwaved at 115° C. for 1 hr. The solution was cooled to room temperature. The solution was filtered to give a solid (0.020 g, 4% yield). (LCMS, ESI pos.) Calculated for C₁₉H₂₄C₁N₃O₄S₂: 457.99 (M+H), Measured: 458.0. ¹H NMR (400 MHz, DMSO-d6) δ 8.16 (t, J=1.3 Hz, 1H), 8.00 (s, 2H), 7.25 (d, J=8.3 Hz, 1H), 7.08 (d, J=26.0 Hz, 2H), 2.70 (d, J=11.4 Hz, 4H), 2.58 (s, 6H), 1.62 (d, J=5.9 Hz, 4H), 1.52 (d, J=5.6 Hz, 2H).

### Example 128

### N¹-(2-(4-(hydroxymethyl)piperidin-1-yl)phenyl)-N⁴,N⁴-dimethylbenzene-1,4-disulfonamide

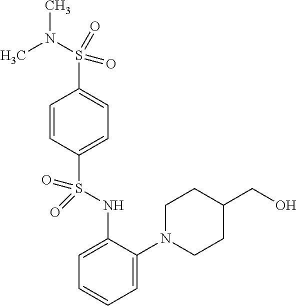 [SMILES: CN(C)S(=O)(=O)c1ccc(S(=O)(=O)Nc2ccccc2N2CCC(CO)CC2)cc1]

A solution of (1-(2-aminophenyl)piperidin-4-yl)methanol (0.206 g, 1 mmol) in pyridine (Volume: 1.000 ml) was treated with 4-(N,N-dimethylsulfamoyl)benzene-1-sulfonyl chloride (0.284 g, 1.000 mmol). The solution was microwaved at 115° C. for 1 hr. The crude product was purified by reversed-phase chromatography (4-100% CH₃CN over 15 min) to give desired product (0.020 g, 4% yield). (LCMS, ESI pos.) Calculated for C₂₀H₂₇N₃O₅S₂: 454.6 (M+H), Measured: 454.1. ¹H NMR (400 MHz, DMSO-d6) δ 9.17 (s, 1H), 8.08-8.00 (m, 2H), 7.99-7.92 (m, 2H), 7.34-7.28 (m, 1H), 7.21-7.05 (m, 3H), 3.31 (d, J=6.2 Hz, 2H), 2.65 (s, 6H), 2.47 (t, J=11.4 Hz, 2H), 1.67-1.56 (m, 2H), 1.48-1.34 (m, 1H), 1.28 (qd, J=11.7, 3.9 Hz, 2H).

### Example 129

### N¹-(2-(2,4-dimethylpiperidin-1-yl)phenyl)-N⁴,N⁴-dimethylbenzene-1,4-disulfonamide

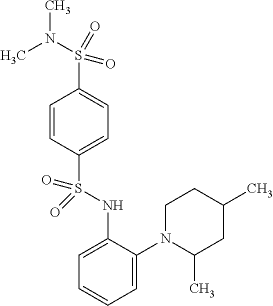

A solution of 2-(2,4-dimethylpiperidin-1-yl)aniline (0.204 g, 0.998 mmol) in pyridine (Volume: 2.001 ml) was treated with 4-(N,N-dimethylsulfamoyl)benzene-1-sulfonyl chloride (0.283 g, 0.998 mmol). The solution was microwaved at 115° C. for 1 hr. The solution was cooled to room temperature. The solution was filtered to give a solid (0.187 g, 41% yield). (LCMS, ESI pos.) Calculated for C₂₁H₂₉N₃O₄S₂: 452.6 (M+H), Measured: 452.1. ¹H NMR (400 MHz, DMSO-d6) δ 9.04 (s, 1H), 8.13-8.00 (m, 2H), 7.98-7.88 (m, 2H), 7.56 (dd, J=8.0, 1.5 Hz, 1H), 7.33-7.08 (m, 3H), 2.87-2.76 (m, 1H), 2.62 (s, 6H), 2.40 (td, J=11.7, 2.3 Hz, 1H), 1.92 (d, J=11.2 Hz, 1H), 1.71-1.41 (m, 3H), 1.27 (qd, J=12.2, 3.9 Hz, 1H), 1.17-1.05 (m, 1H), 0.96 (d, J=6.4 Hz, 3H), 0.47 (d, J=6.1 Hz, 3H).

### Example 130

### 3-(methylsulfonyl)-N-(2-(piperidin-1-yl)phenyl)propane-1-sulfonamide

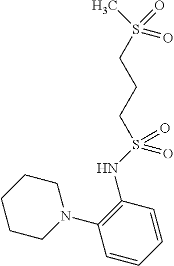 [SMILES: CS(=O)(=O)CCCS(=O)(=O)Nc1ccccc1N1CCCCC1]

2-(piperidin-1-yl)aniline (0.088 g, 0.500 mmol) in Pyridine (1 ml) was treated with 3-(methylsulfonyl)propane-1-sulfonyl chloride (0.110 g, 0.5 mmol) portion wise. The solution was microwaved at 115 deg C. for 1 hr. The crude product was purified by reversed-phase chromatography (4-100% CH₃CN over 15 min) to give desired product (0.0053 g, 2% yield). (LCMS, ESI pos.) Calculated for C₁₅H₂₄N₂O₄S₂: 361.5 (M+H), Measured: 361.1. ¹H NMR (400 MHz, DMSO-d6) δ 8.40 (s, 1H), 7.39-7.34 (m, 1H), 7.30-7.25 (m, 1H), 7.20-7.10 (m, 2H), 3.01 (d, J=0.7 Hz, 3H), 2.81 (t, J=5.3 Hz, 4H), 2.71 (p, J=1.9 Hz, 1H), 2.37 (p, J=1.9 Hz, 1H), 2.23-2.12 (m, 2H), 1.72 (p, J=5.7 Hz, 4H), 1.57 (d, J=5.7 Hz, 2H).

### Example 131 (a)

### 4-bromo-N-(2-(piperidin-1-yl)phenyl)benzenesulfonamide

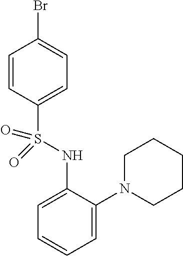 [SMILES: O=S(=O)(Nc1ccccc1N1CCCCC1)c1ccc(Br)cc1]

A solution of 2-(piperidin-1-yl)aniline (2.76 g, 15.65 mmol) in Pyridine (15.65 ml) was treated with 4-bromobenzenesulfonyl chloride (4 g, 15.65 mmol). The solution was stirred for 40 min. The reaction solution was microwaved at 115° C. for 1 hr. The solution was cooled to room temperature. The solution was filtered to give a solid (4.25 g, 69% yield). (LCMS, ESI pos.) Calculated for C₁₇H₁₉BrN₂O₂S: 395.3, 397.3 (M+H), Measured: 395.0, 397.0. ¹H NMR (400 MHz, DMSO-d6) δ 8.91 (s, 1H), 7.85-7.78 (m, 2H), 7.77-7.71 (m, 2H), 7.30 (dd, J=7.5, 1.9 Hz, 1H), 7.20-7.05 (m, 3H), 2.51 (d, J=5.4 Hz, 4H), 1.60 (dq, J=10.7, 5.1 Hz, 5H), 1.49 (q, J=5.4, 4.8 Hz, 2H).

### Example 131 (b)

### diethyl (4-(N-(2-(piperidin-1-yl)phenyl)sulfamoyl)phenyl)phosphonate

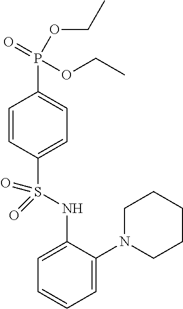 [SMILES: CCOP(=O)(OCC)c1ccc(S(=O)(=O)Nc2ccccc2N2CCCCC2)cc1]

A mixture of 4-bromo-N-(2-(piperidin-1-yl)phenyl)benzenesulfonamide (1.011 g, 2.56 mmol), cesium carbonate (0.909 g, 2.79 mmol) (grounded using a mortar and pestle), diethyl phosphite (0.3 ml, 2.324 mmol), and Pd(Ph3p)4 (0.134 g, 0.116 mmol) in THF (10.11 ml) was microwaved at 120° C. for 30 min. The reaction solution was cooled to room temperature. The solution was filtered through Celite. The filtrate was concentrated. The residue was purified by silica gel chromatography (EtOAc/hexanes 1:9). Desired fractions were concentrated and dried in vacuo to give the desired product (0.62 g, 59% yield). (LCMS, ESI pos.) Calculated for C₂₁H₂₉N₂O₅PS: 453.0 (M+H), Measured: 453.0. ¹H NMR (400 MHz, DMSO-d6) δ 9.01 (s, 1H), 7.98-7.86 (m, 4H), 7.33 (ddd, J=7.6, 1.5, 0.7 Hz, 1H), 7.19-7.05 (m, 3H), 4.14-3.97 (m, 4H), 2.50-2.40 (m, 4H), 1.61-1.40 (m, 7H), 1.25 (td, J=7.0, 0.4 Hz, 5H).

### Example 132

### N,N-dimethyl-4-((2,2,4,7-tetramethyl-3,4-dihydroquinolin-1(2H)-yl)sulfonyl)benzenesulfonamide

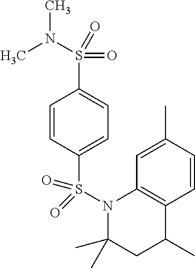

A solution of 2,2,4,7-tetramethyl-1,2,3,4-tetrahydroquinoline (0.095 g, 0.5 mmol) in Pyridine (1.000 ml) was treated with 2,2,4,7-tetramethyl-1,2,3,4-tetrahydroquinoline (0.095 g, 0.5 mmol). The solution was microwaved at 120 deg for 1 hr. The crude product was purified by reversed-phase chromatography (4-100% CH₃CN over 15 min) to give desired product (0.001 g, 1% yield). (LCMS, ESI pos.) Calculated for C₂₁H₂₈N₂O₄S₂: 437.6 (M+H), Measured: 437.0

### Example 133

### Ethyl hydrogen (4-(N-(2-(piperidin-1-yl)phenyl)sulfamoyl)phenyl)phosphonate

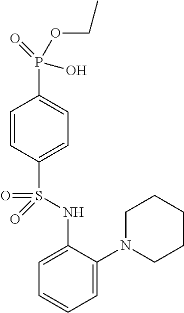 [SMILES: CCOP(=O)(O)c1ccc(S(=O)(=O)Nc2ccccc2N2CCCCC2)cc1]

A solution of diethyl (4-(N-(2-(piperidin-1-yl)phenyl)sulfamoyl)phenyl)phosphonate (0.62 g, 1.370 mmol) in Ethanol (6.85 ml) was treated with sodium hydroxide (8.22 ml, 16.44 mmol). The resulting clear yellow solution was microwaved at 80 deg for 2 hrs. The reaction solution was concentrated. The aqueous residue was cooled and acidified using 1N HCl (pH 1 litmus). The aqueous layer was extracted with CH₂Cl₂ (2×). The organic layers were combined, washed with brine, dried over MgSO₄, filtered and concentrated. Dried in vacuo to give desired product. (0.065 g, 15% yield). (LCMS, ESI pos.) Calculated for C₁₉H₂₅N₂O₅PS: 425.4 (M+H), Measured: 425.1. ¹H NMR (400 MHz, DMSO-d6) δ 8.94 (s, 1H), 7.95-7.82 (m, 4H), 7.33 (dd, J=7.2, 2.2 Hz, 1H), 7.19-7.05 (m, 3H), 3.93-3.82 (m, 2H), 2.50-2.41 (m, 4H), 1.64-1.40 (m, 7H), 1.27 (s, 1H), 1.17 (t, J=7.0 Hz, 3H).

### Example 134

### N¹-(5-cyano-2-(piperidin-1-yl)phenyl)-N⁴,N⁴-dimethylbenzene-1,4-disulfonamide

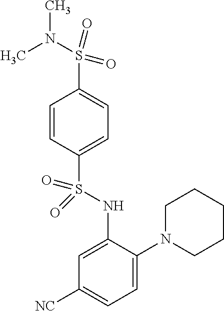 [SMILES: CN(C)S(=O)(=O)c1ccc(S(=O)(=O)Nc2cc(C#N)ccc2N2CCCCC2)cc1]

A solution of 3-amino-4-(piperidin-1-yl)benzonitrile (0.101 g, 0.500 mmol) in Pyridine (1.000 ml) was treated with 4-(N,N-dimethylsulfamoyl)benzenesulfonyl chloride (0.142 g, 0.5 mmol). The solution was microwaved at 120 deg for 1 hr. The crude product was purified by reversed-phase chromatography (4-100% CH₃CN over 15 min) to give desired product (0.0042 g, 1% yield). (LCMS, ESI pos.) Calculated for C₂₀H₂₄N₄O₄S₂: 449.6 (M+H), Measured: 449.1. ¹H NMR (400 MHz, DMSO-d6) δ 9.75 (s, 1H), 8.05 (d, J=8.6 Hz, 2H), 7.99 (d, J=8.6 Hz, 2H), 7.82 (d, J=5.0 Hz, 0H), 7.60 (d, J=8.4 Hz, 1H), 7.34 (d, J=2.0 Hz, 1H), 7.17 (d, J=8.4 Hz, 1H), 2.79 (t, J=4.9 Hz, 4H), 2.68 (s, 6H), 2.64 (d, J=6.7 Hz, 1H), 1.52 (s, 6H).

### Example 135

### N¹,N¹-dimethyl-N⁴-(2-(piperidin-1-yl)-5-(trifluoromethyl)phenyl)benzene-1,4-disulfonamide

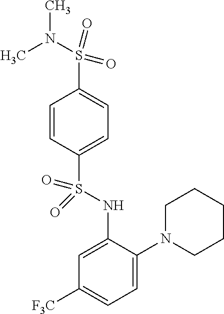 [SMILES: CN(C)S(=O)(=O)c1ccc(S(=O)(=O)Nc2cc(C(F)(F)F)ccc2N2CCCCC2)cc1]

A solution of 2-(piperidin-1-yl)-5-(trifluoromethyl)aniline (0.122 g, 0.500 mmol) in Pyridine (1.000 ml) was treated with Pyridine (1.000 ml). The solution was microwaved at 120 deg for 1 hr. The crude product was purified by reversed-phase chromatography (4-100% CH₃CN over 15 min) to give desired product (0.030 g, 6% yield). (LCMS, ESI pos.) Calculated for C₂₀H₂₄F₃N₃O₄S₂: 492.5 (M+H), Measured: 492.1. ¹H NMR (400 MHz, DMSO-d6) δ 9.72 (s, 1H), 8.06 (d, J=8.6 Hz, 2H), 8.00 (d, J=8.7 Hz, 2H), 7.93-7.85 (m, OH), 7.77-7.73 (m, OH), 7.51 (dd, J=8.6, 2.2 Hz, 1H), 7.45 (d, J=1.8 Hz, 0H), 7.34 (d, J=8.7 Hz, 0H), 7.32-7.24 (m, 2H), 2.72 (t, J=5.1 Hz, 5H), 2.66 (s, 6H), 2.63 (d, J=10.7 Hz, 2H), 1.56 (q, J=5.4 Hz, 5H), 1.53-1.46 (m, 3H).

### Example 136

### N¹-(2-(2-ethylpiperidin-1-yl)-5-methylphenyl)-N⁴,N⁴-dimethylbenzene-1,4-disulfonamide

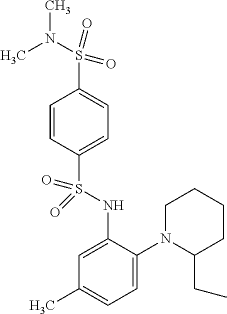 [SMILES: CCC1CCCCN1c1ccc(C)cc1NS(=O)(=O)c1ccc(S(=O)(=O)N(C)C)cc1]

A solution of 2-(2-ethylpiperidin-1-yl)-5-methylaniline (0.109 g, 0.5 mmol) in Pyridine (1.000 ml) was treated with 4-(N,N-dimethylsulfamoyl)benzenesulfonyl chloride (0.142 g, 0.500 mmol). The solution was microwaved at 120 deg for 1 hr. The solution was cooled to room temperature. The solution was filtered to give a solid (0.139 g, 30% yield). (LCMS, ESI pos.) Calculated for C₂₂H₃₁N₃O₄S₂: 466.6 (M+H), Measured: 466.2. ¹H NMR (400 MHz, DMSO-d6) δ 8.86 (s, 1H), 8.12-8.06 (m, 2H), 7.99-7.91 (m, 2H), 7.38 (d, J=1.9 Hz, 1H), 7.13 (d, J=8.0 Hz, 1H), 6.95 (dd, J=7.9, 1.9 Hz, 1H), 2.64 (s, 6H), 2.40-2.26 (m, 4H), 1.94 (d, J=11.3 Hz, 1H), 1.85-1.71 (m, 2H), 1.60-1.25 (m, 3H), 0.94-0.69 (m, 2H), 0.55 (t, J=7.5 Hz, 3H).

### Example 137

### N¹,N¹-dimethyl-N⁴-(5-methyl-2-(3-methylpiperidin-1-yl)phenyl)benzene-1,4-disulfonamide

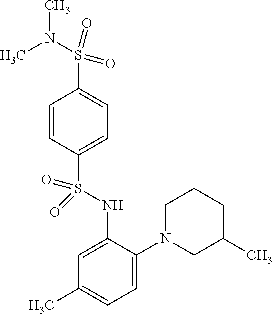

A solution of 5-methyl-2-(3-methylpiperidin-1-yl)aniline (0.102 g, 0.5 mmol) in Pyridine (1.000 ml) was treated with 4-(N,N-dimethylsulfamoyl)benzenesulfonyl chloride (0.142 g, 0.500 mmol). The solution was microwaved at 130deg for 1 hr. The crude product was purified by reversed-phase chromatography (4-100% CH₃CN over 15 min) to give desired product (0.071 g, 16% yield). (LCMS, ESI pos.) Calculated for C₂₁H₂₉N₃O₄S₂: 452.6 (M+H), Measured: 452.2. ¹H NMR (400 MHz, DMSO-d6) δ 9.01 (s, 1H), 8.04-7.98 (m, 2H), 7.97-7.91 (m, 2H), 7.22-7.15 (m, 1H), 7.07 (d, J=8.1 Hz, 1H), 6.98 (dd, J=7.7, 1.8 Hz, 1H), 2.65 (s, 6H), 2.47-2.30 (m, OH), 2.27 (s, 3H), 2.09 (d, J=13.3 Hz, 1H), 1.75-1.49 (m, 4H), 0.98-0.83 (m, 1H), 0.80 (d, J=6.5 Hz, 3H).

### Example 138

### N¹-(3-chloro-2-(4-hydroxypiperidin-1-yl)phenyl)-N⁴,N⁴-dimethylbenzene-1,4-disulfonamide

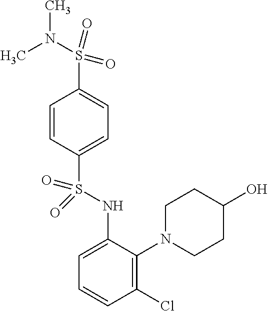 [SMILES: CN(C)S(=O)(=O)c1ccc(S(=O)(=O)Nc2cccc(Cl)c2N2CCC(O)CC2)cc1]

A solution of 1-(2-amino-6-chlorophenyl)piperidin-4-ol (0.113 g, 0.5 mmol) in Pyridine (1.000 ml) was treated with 4-(N,N-dimethylsulfamoyl)benzenesulfonyl chloride (0.142 g, 0.500 mmol). The solution was microwaved at 130deg for 1 hr. The crude product was purified by reversed-phase chromatography (4-100% CH₃CN over 15 min) to give desired product (0.001 g, 1% yield). (LCMS, ESI pos.) Calculated for C₁₉H₂₄ClN₃O₅S₂: 473.9 (M+H), Measured: 474.1. ¹H NMR (400 MHz, DMSO-d6) δ 9.18 (s, 1H), 8.08-7.92 (m, 4H), 7.38 (s, 1H), 7.23 (s, 2H), 7.09 (s, 1H), 3.28-3.11 (m, 1H), 2.66 (s, 6H), 2.28 (s, OH), 1.74 (s, 2H), 1.53 (d, J=11.0 Hz, 2H).

### Example 139

### N¹-(5-chloro-2-(3-hydroxypiperidin-1-yl)phenyl)-N⁴,N⁴-dimethylbenzene-1,4-disulfonamide

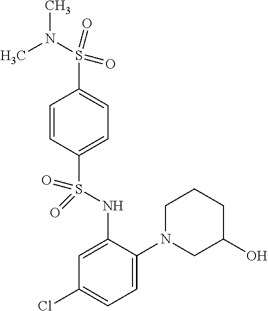 [SMILES: CN(C)S(=O)(=O)c1ccc(S(=O)(=O)Nc2cc(Cl)ccc2N2CCCC(O)C2)cc1]

A solution of 1-(2-amino-4-chlorophenyl)piperidin-3-ol (0.113 g, 0.5 mmol) in Pyridine (1.000 ml) was treated with 4-(N,N-dimethylsulfamoyl)benzenesulfonyl chloride (0.142 g, 0.500 mmol). The solution was microwaved at 130 deg for 1 hr. The crude product was purified by reversed-phase chromatography (4-100% CH₃CN over 15 min) to give desired product (0.122 g, 26% yield). (LCMS, ESI pos.) Calculated for C₁₉H₂₄ClN₃O₅S₂: 474.0 (M+H), Measured: 474.1. ¹H NMR (400 MHz, DMSO-d6) δ 9.61 (s, 1H), 8.11-8.06 (m, 2H), 8.02-7.97 (m, 2H), 7.37 (d, J=2.3 Hz, 1H), 7.18 (dd, J=8.6, 2.4 Hz, 1H), 7.13 (d, J=8.6 Hz, 1H), 3.76-3.68 (m, 1H), 2.76-2.68 (m, 1H), 2.65 (s, 6H), 2.49 (ddd, J=11.0, 7.5, 2.7 Hz, 1H), 2.34 (dt, J=14.6, 5.4 Hz, 2H), 1.83-1.59 (m, 2H), 1.53-1.37 (m, 3H).

### Example 140

### N¹-(2-(4-hydroxypiperidin-1-yl)-5-methylphenyl)-N⁴,N⁴-dimethylbenzene-1,4-disulfonamide

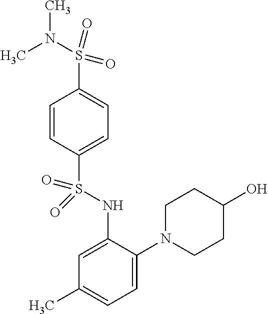 [SMILES: Cc1ccc(N2CCC(O)CC2)c(NS(=O)(=O)c2ccc(S(=O)(=O)N(C)C)cc2)c1]

A solution of 1-(2-amino-4-methylphenyl)piperidin-4-ol (0.103 g, 0.5 mmol) in Pyridine (1.000 ml) was treated with 4-(N,N-dimethylsulfamoyl)benzenesulfonyl chloride (0.142 g, 0.500 mmol). The solution was microwaved at 120deg for 1 hr. The crude product was purified by reversed-phase chromatography (4-100% CH₃CN over 15 min) to give desired product (0.0873 g, 19% yield). (LCMS, ESI pos.) Calculated for C₂₀H₂₇N₃O₅S₂: 454.6 (M+H), Measured: 454.2. ¹H NMR (400 MHz, DMSO-d6) δ 9.09 (s, 1H), 8.05-8.00 (m, 2H), 7.99-7.93 (m, 2H), 7.17 (s, 1H), 7.07 (d, J=8.1 Hz, 1H), 7.01-6.94 (m, 1H), 3.55 (dt, J=8.6, 4.5 Hz, 1H), 2.66 (s, 6H), 2.49 (d, J=5.0 Hz, 2H), 2.38 (t, J=10.1 Hz, 2H), 2.26 (s, 3H), 1.76-1.63 (m, 3H), 1.57-1.41 (m, 2H).

### Example 141

### N¹-(2,5-di(piperidin-1-yl)phenyl)-N⁴,N⁴-dimethylbenzene-1,4-disulfonamide

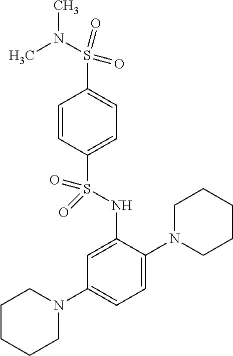 [SMILES: CN(C)S(=O)(=O)c1ccc(S(=O)(=O)Nc2cc(N3CCCCC3)ccc2N2CCCCC2)cc1]

A solution of 2,5-di(piperidin-1-yl)aniline (0.130 g, 0.5 mmol) in Pyridine (1.000 ml) was treated with 4-(N,N-dimethylsulfamoyl)benzenesulfonyl chloride (0.142 g, 0.500 mmol). The solution was microwaved at 120 deg for 1 hr. The crude product was purified by reversed-phase chromatography (4-100% CH₃CN over 15 min) to give desired product (0.18 g, 36% yield). (LCMS, ESI pos.) Calculated for C₂₄H₃₄N₄O₄S₂: 507.6 (M+H), Measured: 507.3. ¹H NMR (400 MHz, DMSO-d6) δ 8.03 (d, J=8.5 Hz, 2H), 7.97 (d, J=8.5 Hz, 2H), 7.09 (s, 2H), 3.13 (s, 4H), 2.65 (s, 6H), 2.49-2.32 (m, 4H), 1.56 (t, J=41.8 Hz, 11H).

### Example 142

### N¹-(2-fluoro-6-(4-hydroxypiperidin-1-yl)phenyl)-N⁴,N⁴-dimethylbenzene-1,4-disulfonamide

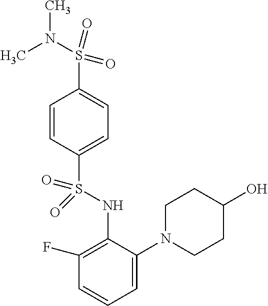 [SMILES: CN(C)S(=O)(=O)c1ccc(S(=O)(=O)Nc2c(F)cccc2N2CCC(O)CC2)cc1]

A solution of 1-(2-amino-3-fluorophenyl)piperidin-4-ol (0.105 g, 0.5 mmol) in Pyridine (1.000 ml) was treated with 4-(N,N-dimethylsulfamoyl)benzenesulfonyl chloride (0.142 g, 0.500 mmol). The solution was microwaved at 120 deg for 1 hr. The crude product was purified by reversed-phase chromatography (4-100% CH₃CN over 15 min) to give desired product (0.0521 g, 11% yield). (LCMS, ESI pos.) Calculated for C₁₉H₂₄FN₃O₄S₂: 458.5 (M+H), Measured: 458.1. ¹H NMR (400 MHz, DMSO-d6) δ 9.57 (s, 1H), 8.11-7.93 (m, 4H), 7.26 (td, J=8.3, 6.5 Hz, 1H), 6.96-6.81 (m, 2H), 3.51 (tt, J=8.3, 3.9 Hz, 1H), 3.07 (dt, J=10.8, 4.5 Hz, 2H), 2.62 (ddd, J=12.2, 9.5, 3.0 Hz, 3H), 1.66-1.56 (m, 2H), 1.31 (ddt, J=12.6, 8.9, 4.4 Hz, 2H).

### Example 143

### N¹-(5-chloro-2-(4-(hydroxymethyl)piperidin-1-yl)phenyl)-N⁴,N⁴-dimethylbenzene-1,4-disulfonamide

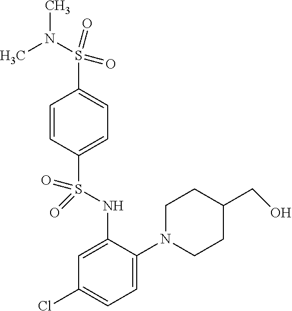 [SMILES: CN(C)S(=O)(=O)c1ccc(S(=O)(=O)Nc2cc(Cl)ccc2N2CCC(CO)CC2)cc1]

A solution of (1-(2-amino-4-chlorophenyl)piperidin-4-yl)methanol (0.120 g, 0.500 mmol) in Pyridine (1.000 ml) was treated with 4-(N,N-dimethylsulfamoyl)benzenesulfonyl chloride (0.142 g, 0.5 mmol). The solution was microwaved at 120 deg for 1 hr. The crude product was purified by reversed-phase chromatography (4-100% CH₃CN over 15 min) to give desired product (0.0055 g, 1% yield). (LCMS, ESI pos.) Calculated for C₂₀H₂₆ClN₃O₅S₂: 488.0 (M+H), Measured: 488.1. ¹H NMR (400 MHz, DMSO-d6) δ 9.49 (d, J=34.2 Hz, 1H), 8.10-8.03 (m, 2H), 7.98 (d, J=8.5 Hz, 2H), 7.30 (d, J=2.4 Hz, 0H), 7.28 (d, J=2.2 Hz, 1H), 7.23 (d, J=2.3 Hz, 0H), 7.22-7.18 (m, 1H), 7.18 (d, J=3.5 Hz, 0H), 3.30 (d, J=6.2 Hz, 2H), 2.66 (s, 6H), 2.45 (td, J=11.6, 2.5 Hz, 2H), 1.59 (dd, J=13.0, 3.3 Hz, 2H), 1.53-1.19 (m, 3H).

### Example 144

### N¹-(4,5-difluoro-2-(piperidin-1-yl)phenyl)-N⁴,N⁴-dimethylbenzene-1,4-disulfonamide

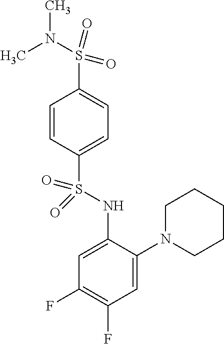 [SMILES: CN(C)S(=O)(=O)c1ccc(S(=O)(=O)Nc2cc(F)c(F)cc2N2CCCCC2)cc1]

A solution of 4,5-difluoro-2-(piperidin-1-yl)aniline (0.106 g, 0.500 mmol) in Pyridine (1.000 ml) was treated with 4-(N,N-dimethylsulfamoyl)benzenesulfonyl chloride (0.142 g, 0.5 mmol). The solution was microwaved at 120 deg for 1 hr. The crude product was purified by reversed-phase chromatography (4-100% CH₃CN over 15 min) to give desired product (0.001 g, 1% yield). (LCMS, ESI pos.) Calculated for C₁₉H₂₃F₂N₃O₄S₂: 460.5 (M+H), Measured: 460.1. ¹H NMR (400 MHz, DMSO-d6) δ 10.30 (s, 1H), 7.96 (s, 4H), 7.02-6.90 (m, 1H), 6.82 (dd, J=11.8, 8.5 Hz, 1H), 2.96 (t, J=5.3 Hz, 4H), 2.66 (s, 6H), 1.63 (d, J=5.9 Hz, 4H), 1.54 (d, J=5.5 Hz, 2H).

### Example 145

### N¹-(5-acetyl-2-(piperidin-1-yl)phenyl)-N⁴,N⁴-dimethylbenzene-1,4-disulfonamide

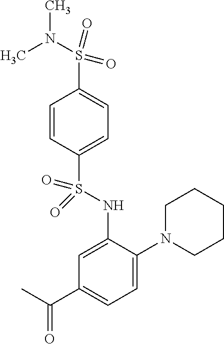 [SMILES: CC(=O)c1ccc(N2CCCCC2)c(NS(=O)(=O)c2ccc(S(=O)(=O)N(C)C)cc2)c1]

To a solution of 1-(3-amino-4-(piperidin-1-yl)phenyl)ethan-1-one (0.109 g, 0.500 mmol) in Pyridine (1.000 ml) was added 4-(N,N-dimethylsulfamoyl)benzenesulfonyl chloride (0.142 g, 0.5 mmol). The solution was microwaved at 120 deg for 1 hr. The crude product was purified by reversed-phase chromatography (4-100% CH₃CN over 15 min) to give desired product (0.001 g, 3% yield). (LCMS, ESI pos.) Calculated for C₂₁H₂₇N₃O₅S₂: 466.6 (M+H), Measured: 466.1. ¹H NMR (400 MHz, DMSO-d6) δ 9.58 (s, 1H), 8.08-7.96 (m, 4H), 7.85-7.74 (m, 1H), 7.53 (d, J=2.1 Hz, 1H), 7.14 (d, J=8.4 Hz, 1H), 5.79 (s, OH), 2.82 (t, J=5.1 Hz, 4H), 2.68 (s, 5H), 2.64 (s, 1H), 2.45 (s, 3H), 1.63-1.45 (m, 6H).

### Example 146

### N¹,N¹-dimethyl-N⁴-(5-methyl-2-(4-methylpiperidin-1-yl)phenyl)benzene-1,4-disulfonamide

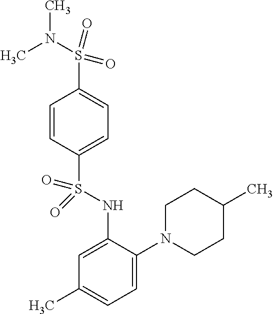

To a solution of 5-methyl-2-(4-methylpiperidin-1-yl)aniline (0.102 g, 0.500 mmol) in Pyridine (1.000 ml) was added 4-(N,N-dimethylsulfamoyl)benzenesulfonyl chloride (0.142 g, 0.5 mmol). The solution was microwaved at 120 deg for 1 hr. The crude product was purified by reversed-phase chromatography (4-100% CH₃CN over 15 min) to give desired product (0.131 g, 30% yield). (LCMS, ESI pos.) Calculated for C₂₁H₂₉N₃O₄S₂: 452.6 (M+H), Measured: 452.1. ¹H NMR (400 MHz, DMSO-d6) δ 9.02 (s, 1H), 8.06-7.99 (m, 2H), 7.95 (d, J=8.5 Hz, 2H), 7.17 (d, J=1.9 Hz, 1H), 7.05 (d, J=8.1 Hz, 1H), 6.96 (d, J=8.2 Hz, 1H), 2.65 (s, 6H), 2.43 (d, J=7.9 Hz, 4H), 2.26 (s, 3H), 1.52 (d, J=12.4 Hz, 2H), 1.24 (dt, J=12.3, 8.0 Hz, 2H), 0.95 (d, J=6.4 Hz, 3H).

### Example 147

### N¹-(5-chloro-2-(2-ethylpiperidin-1-yl)phenyl)-N⁴,N⁴-dimethylbenzene-1,4-disulfonamide

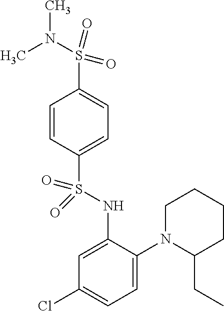 [SMILES: CCC1CCCCN1c1ccc(Cl)cc1NS(=O)(=O)c1ccc(S(=O)(=O)N(C)C)cc1]

To a solution of 5-chloro-2-(2-ethylpiperidin-1-yl)aniline (0.119 g, 0.500 mmol) in Pyridine (1.000 ml) was added 4-(N,N-dimethylsulfamoyl)benzenesulfonyl chloride (0.142 g, 0.5 mmol). The solution was microwaved at 120 deg for 1 hr. The crude product was purified by reversed-phase chromatography (4-100% CH₃CN over 15 min) to give desired product (0.126 g, 26% yield). (LCMS, ESI pos.) Calculated for C₂₁H₂₈ClN₃O₄S₂: 486.0, 488.0 (M+H), Measured: 486.1, 488.1. ¹H NMR (400 MHz, DMSO-d6) δ 9.11 (s, 1H), 8.04-7.97 (m, 2H), 7.89-7.84 (m, 2H), 7.41 (d, J=2.4 Hz, 1H), 7.18 (d, J=8.6 Hz, 1H), 7.09 (dd, J=8.6, 2.4 Hz, 1H), 2.54 (s, 7H), 2.24 (td, J=11.3, 2.8 Hz, 1H), 1.92 (d, J=11.4 Hz, 1H), 1.72-1.57 (m, 2H), 1.46 (t, J=11.8 Hz, 1H), 1.36-1.15 (m, 3H), 0.86-0.66 (m, 1H), 0.45 (t, J=7.4 Hz, 3H).

### Example 148

### N¹-(3-chloro-2-(2-ethylpiperidin-1-yl)phenyl)-N⁴,N⁴-dimethylbenzene-1,4-disulfonamide

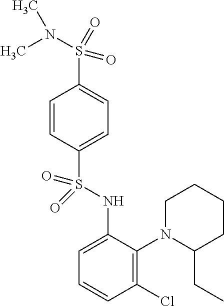 [SMILES: CCC1CCCCN1c1c(Cl)cccc1NS(=O)(=O)c1ccc(S(=O)(=O)N(C)C)cc1]

To a solution of 3-chloro-2-(2-ethylpiperidin-1-yl)aniline (0.119 g, 0.500 mmol) in Pyridine (1.000 ml) was added 4-(N,N-dimethylsulfamoyl)benzenesulfonyl chloride (0.142 g, 0.5 mmol). The solution was microwaved at 120 deg for 1 hr. The crude product was purified by reversed-phase chromatography (4-100% CH₃CN over 15 min) to give desired product (0.127 g, 26% yield). (LCMS, ESI pos.) Calculated for C₂₁H₂₈ClN₃O₄S₂: 486.0 (M+H), Measured: 486.1. ¹H NMR (400 MHz, DMSO-d6) δ 9.04 (d, J=10.8 Hz, 1H), 8.17-8.07 (m, 2H), 8.04-7.93 (m, 2H), 7.57 (dd, J=8.2, 1.4 Hz, 1H), 7.28 (t, J=8.1 Hz, 1H), 7.16 (dd, J=8.1, 1.4 Hz, 1H), 3.28 (q, J=10.1, 8.7 Hz, 1H), 3.10 (dt, J=11.6, 8.0 Hz, 1H), 2.63 (d, J=7.9 Hz, 6H), 1.92 (d, J=11.5 Hz, 1H), 1.83 (s, 2H), 1.51 (s, 2H), 1.35-1.23 (m, 1H), 1.14-1.02 (m, 1H), 0.93-0.80 (m, 1H), 0.68-0.57 (m, 3H).

### Example 149

### N¹-(5-chloro-2-(4-hydroxypiperidin-1-yl)phenyl)-N⁴,N⁴-dimethylbenzene-1,4-disulfonamide

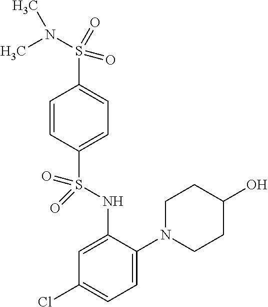 [SMILES: CN(C)S(=O)(=O)c1ccc(S(=O)(=O)Nc2cc(Cl)ccc2N2CCC(O)CC2)cc1]

A solution of 1-(2-amino-4-chlorophenyl)piperidin-4-ol (0.113 g, 0.500 mmol) in Pyridine (1.000 ml) was treated with 4-(N,N-dimethylsulfamoyl)benzenesulfonyl chloride (0.142 g, 0.5 mmol). The solution was microwaved at 120 deg for 1 hr. The crude product was purified by reversed-phase chromatography (4-100% CH₃CN over 15 min) to give desired product (0.085 g, 19% yield). (LCMS, ESI pos.) Calculated for C₁₉H₂₄ClN₃O₅S₂: 474.0 (M+H), Measured: 474.1. ¹H NMR (400 MHz, DMSO-d6) δ 9.51 (s, 1H), 8.09-8.04 (m, 2H), 8.02-7.96 (m, 2H), 7.28 (d, J=2.2 Hz, 1H), 7.20 (d, J=2.3 Hz, 1H), 7.19 (s, 1H), 3.56 (dt, J=8.6, 4.4 Hz, 1H), 2.67 (s, 6H), 2.65-2.56 (m, 2H), 2.41 (ddd, J=11.8, 9.4, 3.0 Hz, 2H), 1.70 (dq, J=12.8, 3.7 Hz, 2H), 1.51 (dtd, J=12.5, 9.0, 3.6 Hz, 2H).

### Example 150

### N¹-(3-fluoro-2-(piperidin-1-yl)phenyl)-N⁴,N⁴-dimethylbenzene-1,4-disulfonamide

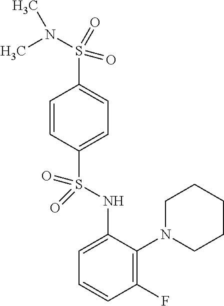 [SMILES: CN(C)S(=O)(=O)c1ccc(S(=O)(=O)Nc2cccc(F)c2N2CCCCC2)cc1]

A solution of 3-fluoro-2-(piperidin-1-yl)aniline (0.097 g, 0.500 mmol) in Pyridine (1.000 ml) was treated with 4-(N,N-dimethylsulfamoyl)benzenesulfonyl chloride (0.142 g, 0.5 mmol). The solution was microwaved at 120 deg for 1 hr. The crude product was purified by reversed-phase chromatography (4-100% CH₃CN over 15 min) to give desired product (0.090 g, 20% yield). (LCMS, ESI pos.) Calculated for C₁₉H₂₄FN₃O₄S₂: 442.5 (M+H), Measured: 442.1. ¹H NMR (400 MHz, DMSO-d6) δ 9.23 (s, 1H), 8.02 (d, J=8.6 Hz, 2H), 7.96 (d, J=8.6 Hz, 2H), 7.32-7.18 (m, 2H), 7.09-6.97 (m, 1H), 2.65 (s, 6H), 1.60-1.40 (m, 7H).

### Example 151

### N¹-(5-fluoro-2-(3-methylpiperidin-1-yl)phenyl)-N⁴,N⁴-dimethylbenzene-1,4-disulfonamide

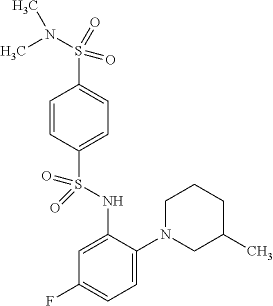

A solution of 5-fluoro-2-(3-methylpiperidin-1-yl)aniline hydrochloride (0.122 g, 0.500 mmol) in Pyridine (1.000 ml) was treated with 4-(N,N-dimethylsulfamoyl)benzenesulfonyl chloride (0.142 g, 0.5 mmol). The solution was microwaved at 120 deg for 1 hr. The crude product was purified by reversed-phase chromatography (4-100% CH₃CN over 15 min) to give desired product (0.067 g, 15% yield). (LCMS, ESI pos.) Calculated for C₁₉H₂₄FN₃O₄S₂: 456.6 (M+H), Measured: 456.1. ¹H NMR (400 MHz, DMSO-d6) δ 9.30 (s, 1H), 8.08-8.03 (m, 2H), 7.99-7.94 (m, 2H), 7.21 (ddd, J=13.2, 9.6, 4.5 Hz, 2H), 7.00 (td, J=8.5, 3.0 Hz, 1H), 2.66 (s, 6H), 2.41-2.26 (m, 3H), 2.07 (t, J=10.4 Hz, 1H), 1.80-1.49 (m, 4H), 0.90 (qd, J=12.0, 4.0 Hz, 1H), 0.79 (d, J=6.4 Hz, 3H).

### Example 152

### N¹-(5-chloro-2-(3-methylpiperidin-1-yl)phenyl)-N⁴,N⁴-dimethylbenzene-1,4-disulfonamide

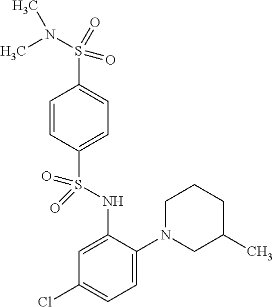

A solution of 5-chloro-2-(3-methylpiperidin-1-yl)aniline (0.112 g, 0.500 mmol) in Pyridine (1.000 ml) was treated with 4-(N,N-dimethylsulfamoyl)benzenesulfonyl chloride (0.142 g, 0.5 mmol). The solution was microwaved at 120 deg for 1 hr. The crude product was purified by reversed-phase chromatography (4-100% CH₃CN over 15 min) to give desired product (0.11 g, 24% yield). (LCMS, ESI pos.) Calculated for C₂₀H₂₆ClN₃O₄S₂: 472.0, 474.0 (M+H), Measured: 472.1, 474.1. ¹H NMR (400 MHz, DMSO-d6) δ 9.42 (s, 1H), 8.04 (d, J=8.6 Hz, 2H), 7.98 (d, J=8.6 Hz, 2H), 7.30 (d, J=2.4 Hz, 1H), 7.23 (dd, J=8.6, 2.4 Hz, 1H), 7.17 (d, J=8.6 Hz, 1H), 2.67 (s, 6H), 2.53-2.32 (m, 3H), 2.08 (t, J=10.6 Hz, 1H), 1.76-1.47 (m, 4H), 0.99-0.83 (m, 1H), 0.79 (d, J=6.5 Hz, 3H).

### Example 153

### N¹,N¹-dimethyl-N⁴-(6-(piperidin-1-yl)quinolin-5-yl)benzene-1,4-disulfonamide

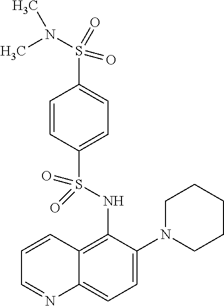 [SMILES: CN(C)S(=O)(=O)c1ccc(S(=O)(=O)Nc2c(N3CCCCC3)ccc3ncccc23)cc1]

A solution of 6-(piperidin-1-yl)quinolin-5-amine (0.114 g, 0.500 mmol) in Pyridine (1.000 ml) was treated with 4-(N,N-dimethylsulfamoyl)benzenesulfonyl chloride (0.142 g, 0.5 mmol). The solution was microwaved at 120 deg for 1 hr. The crude product was purified by reversed-phase chromatography (4-100% CH₃CN over 15 min) to give desired product (0.108 g, 23% yield). (LCMS, ESI pos.) Calculated for C₂₂H₂₆N₄O₄S₂: 475.0 (M+H), Measured: 475.1. ¹H NMR (400 MHz, DMSO-d6) δ 10.03 (s, 1H), 8.83 (dd, J=4.3, 1.5 Hz, 1H), 8.43-8.30 (m, 1H), 8.02 (dd, J=9.3, 0.8 Hz, 1H), 7.92 (q, J=8.7 Hz, 4H), 7.73 (d, J=9.3 Hz, 1H), 7.55 (dd, J=8.6, 4.3 Hz, 1H), 2.90 (t, J=5.0 Hz, 4H), 2.70 (s, 6H), 1.36 (dd, J=25.6, 6.4 Hz, 6H).

### Example 154

### 3-((4-(N,N-dimethylsulfamoyl)phenyl)sulfonamido)-4-(piperidin-1-yl)benzamide

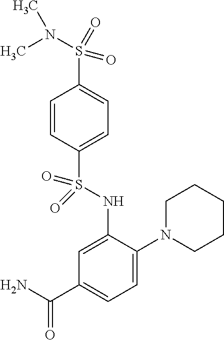 [SMILES: CN(C)S(=O)(=O)c1ccc(S(=O)(=O)Nc2cc(C(N)=O)ccc2N2CCCCC2)cc1]

To a solution of 3-amino-4-(piperidin-1-yl)benzamide (0.110 g, 0.500 mmol) in Pyridine (1.000 ml) was added 4-(N,N-dimethylsulfamoyl)benzenesulfonyl chloride (0.142 g, 0.5 mmol). Solution was microwaved at 120° C. for 1 hour. The crude product was purified by reversed-phase chromatography (4-100% CH₃CN over 15 min) to give desired product (0.043 g, 9% yield). (LCMS, ESI pos.) Calculated for C₂₀H₂₆N₄₀₅S₂: 467.6 (M+H), Measured: 467.1. ¹H NMR (400 MHz, DMSO-d6) δ 9.38 (s, 1H), 8.02 (d, J=8.6 Hz, 2H), 7.97 (d, J=8.6 Hz, 2H), 7.87 (s, 1H), 7.69 (d, J=7.2 Hz, 2H), 7.26 (s, 1H), 7.16-7.08 (m, 1H), 2.64 (d, J=5.5 Hz, 4H), 1.50 (dd, J=21.3, 6.2 Hz, 7H).

### Example 155

### N¹-(5-methoxy-2-(piperidin-1-yl)phenyl)-N⁴,N⁴-dimethylbenzene-1,4-disulfonamide

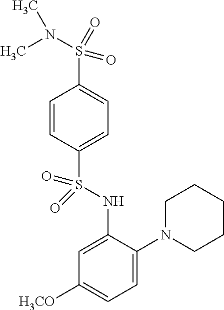 [SMILES: COc1ccc(N2CCCCC2)c(NS(=O)(=O)c2ccc(S(=O)(=O)N(C)C)cc2)c1]

To a solution of 5-methoxy-2-(piperidin-1-yl)aniline dihydrochloride (0.140 g, 0.5 mmol) in Pyridine (1.000 ml) was added 4-(N,N-dimethylsulfamoyl)benzenesulfonyl chloride (0.142 g, 0.5 mmol). Solution was microwaved at 120° C. for 1 hour. The crude product was purified by reversed-phase chromatography (4-100% CH₃CN over 15 min) to give desired product (0.044 g, 9% yield). (LCMS, ESI pos.) Calculated for C₂₀H₂₇N₃O₅S₂: 454.6 (M+H), Measured: 454.1. ¹H NMR (400 MHz, DMSO-d6) δ 8.08-8.02 (m, 2H), 7.96 (d, J=8.5 Hz, 2H), 7.14 (d, J=8.9 Hz, 1H), 6.96 (d, J=2.9 Hz, 1H), 6.73 (dd, J=8.8, 2.9 Hz, 1H), 3.74 (s, 3H), 2.74-2.68 (m, 3H), 2.65 (s, 6H), 2.36 (dt, J=6.3, 3.2 Hz, 6H), 1.55 (s, 4H).

### Example 156

### N¹,N¹-dimethyl-N4-(5-nitro-2-(piperidin-1-yl)phenyl)benzene-1,4-disulfonamide

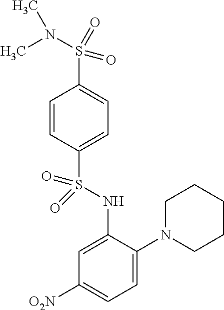 [SMILES: CN(C)S(=O)(=O)c1ccc(S(=O)(=O)Nc2cc([N+](=O)[O-])ccc2N2CCCCC2)cc1]

To a solution of 5-nitro-2-(piperidin-1-yl)aniline (0.111 g, 0.5 mmol) in Pyridine (1.000 ml) was added 4-(N,N-dimethylsulfamoyl)benzenesulfonyl chloride (0.142 g, 0.5 mmol). Solution was microwaved at 120° C. for 1 hour. The crude product was purified by reversed-phase chromatography (4-100% CH₃CN over 15 min) to give desired product (0.14 g, 31% yield). (LCMS, ESI pos.) Calculated for C₁₉H₂₄N₄₀₆S₂: 469.5 (M+H), Measured: 469.1. ¹H NMR (400 MHz, DMSO-d6) δ 9.93 (s, 1H), 8.06-7.98 (m, 5H), 7.82 (d, J=5.0 Hz, 1H), 7.61 (d, J=2.8 Hz, 1H), 7.18 (d, J=9.1 Hz, 1H), 3.05 (t, J=4.9 Hz, 4H), 2.67 (s, 6H), 1.67-1.51 (m, 6H).

### Example 157

### N¹,N¹-dimethyl-N4-(5-methyl-2-(piperidin-1-yl)phenyl)benzene-1,4-disulfonamide

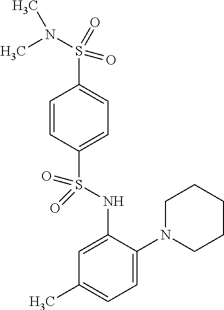 [SMILES: Cc1ccc(N2CCCCC2)c(NS(=O)(=O)c2ccc(S(=O)(=O)N(C)C)cc2)c1]

To a solution of 5-methyl-2-(piperidin-1-yl)aniline (0.095 g, 0.500 mmol) in Pyridine (1.000 ml) was added 4-(N,N-dimethylsulfamoyl)benzenesulfonyl chloride (0.142 g, 0.5 mmol). Solution was microwaved at 120° C. for 1 hour. The crude product was purified by reversed-phase chromatography (4-100% CH₃CN over 15 min) to give desired product (0.11 g, 25% yield). (LCMS, ESI pos.) Calculated for C₂₀H₂₇N₃O₄S₂: 438.6 (M+H), Measured: 438.1.

### Example 158

### N¹,N¹-dimethyl-N⁴-(4-methyl-2-(2-methylpiperidin-1-yl)phenyl)benzene-1,4-disulfonamide

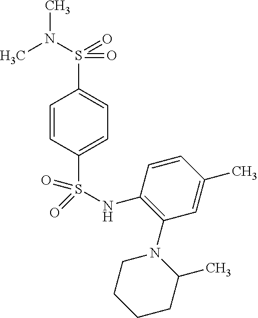

To a solution of 5-methyl-2-(2-methylpiperidin-1-yl)aniline (0.102 g, 0.500 mmol) in Pyridine (1.000 ml) was added 4-(N,N-dimethylsulfamoyl)benzenesulfonyl chloride (0.142 g, 0.5 mmol). The solution was microwaved at 120 deg for 1 hr. The solution was cooled to room temperature. The solution was filtered to give a solid (0.062 g, 14% yield). (LCMS, ESI pos.) Calculated for C₂₁H₂₉N₃O₄S₂: 452.6 (M+H), Measured: 452.1.

### Example 159

### N¹,N¹-dimethyl-N⁴-(4-methyl-2-(4-methylpiperidin-1-yl)phenyl)benzene-1,4-disulfonamide

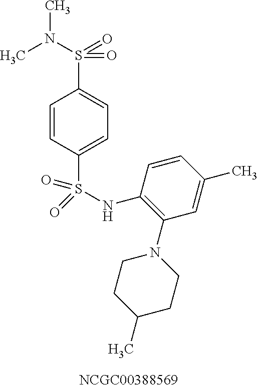

To a solution of 5-methyl-2-(4-methylpiperidin-1-yl)aniline (0.102 g, 0.5 mmol) in Pyridine (1.000 ml) was added 4-(N,N-dimethylsulfamoyl)benzenesulfonyl chloride (0.142 g, 0.500 mmol). The solution was microwaved at 120° C. for 1 hr. The solution was cooled to room temperature. The solution was filtered to give a solid (0.218 g, 48% yield). (LCMS, ESI pos.) Calculated for C₂₁H₂₉N₃O₄S₂: 452.6 (M+H), Measured: 452.1.

### Example 160

### N¹-(5-chloro-2-(4-methylpiperidin-1-yl)phenyl)-N⁴,N⁴-dimethylbenzene-1,4-disulfonamide

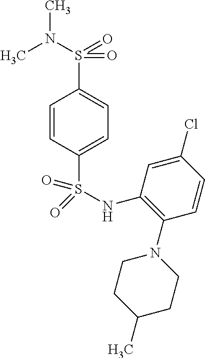 [SMILES: CC1CCN(c2ccc(Cl)cc2NS(=O)(=O)c2ccc(S(=O)(=O)N(C)C)cc2)CC1]

A solution of 5-chloro-2-(4-methylpiperidin-1-yl)aniline (0.112 g, 0.5 mmol) in Pyridine (1.000 ml) was treated with 4-(N,N-dimethylsulfamoyl)benzenesulfonyl chloride (0.142 g, 0.500 mmol). The solution was microwaved at 120 deg for 1 hr. The solution was cooled to room temperature. The solution was filtered to give a solid (0.115 g, 24% yield). (LCMS, ESI pos.) Calculated for C₂₀H₂₆ClN₃O₄S₂: 472.0, 474.0 (M+H), Measured: 472.1, 474.1.

### Example 161

### 4-((2-bromo-3-fluoro-6,6a,7,8,9,10-hexahydro-5H-pyrido[1,2-a]quinoxalin-5-yl)sulfonyl)-N,N-dimethylbenzenesulfonamide

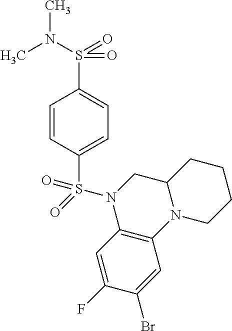 [SMILES: CN(C)S(=O)(=O)c1ccc(S(=O)(=O)N2CC3CCCCN3c3cc(Br)c(F)cc32)cc1]

A solution of 2-bromo-3-fluoro-6,6a,7,8,9,10-hexahydro-5H-pyrido[1,2-a]quinoxaline (0.143 g, 0.5 mmol) in Pyridine (1.000 ml) was treated with 4-(N,N-dimethylsulfamoyl)benzenesulfonyl chloride (0.142 g, 0.500 mmol). The solution was stirred at 120° C. for 1 hour. The crude product was purified by reversed-phase chromatography (4-100% CH₃CN over 15 min) to give desired product (0.013 g, 2% yield). (LCMS, ESI pos.) Calculated for C₂₀H₂₃BrFN₃O₄S₂: 532.4, 534.4 (M+H), Measured: 532.1, 534.1. ¹H NMR (400 MHz, DMSO-d₆) δ 8.02-7.97 (m, 2H), 7.90-7.85 (m, 2H), 7.42 (d, J=10.0 Hz, 1H), 7.24 (d, J=6.8 Hz, 1H), 4.17 (d, J=4.0 Hz, 1H), 4.15-4.08 (m, 1H), 3.81 (d, J=12.5 Hz, 1H), 3.34-3.25 (m, 2H), 3.24-3.18 (m, 2H), 2.67 (s, 6H), 2.15-1.95 (m, 2H), 1.76-1.55 (m, 3H), 1.41-1.24 (m, 1H), 1.14-0.91 (m, 2H).

### Example 162

### N¹-(2-(4-(hydroxymethyl)-4-methylpiperidin-1-yl)phenyl)-N⁴,N⁴-dimethylbenzene-1,4-disulfonamide

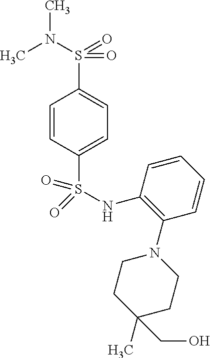 [SMILES: CN(C)S(=O)(=O)c1ccc(S(=O)(=O)Nc2ccccc2N2CCC(C)(CO)CC2)cc1]

A solution of (1-(2-aminophenyl)-3-methylpiperidin-3-yl)methanol (0.110 g, 0.5 mmol) in Pyridine (1.000 ml) was treated with 4-(N,N-dimethylsulfamoyl)benzenesulfonyl chloride (0.142 g, 0.5 mmol). The solution was stirred at 120° C. for 1 hour. The solution was cooled to room temperature. The solution was filtered to give a solid (0.0095 g, 2% yield). (LCMS, ESI pos.) Calculated for C₂₁H₂₉N₃O₅S₂: 468.6 (M+H), Measured: 468.2. ¹H NMR (400 MHz, DMSO-d₆) δ 9.12 (s, 1H), 8.07-8.01 (m, 2H), 7.99-7.92 (m, 2H), 7.30 (dd, J=7.9, 1.6 Hz, 1H), 7.22 (dd, J=7.9, 1.6 Hz, 1H), 7.15 (td, J=7.6, 1.7 Hz, 1H), 7.09 (td, J=7.6, 1.6 Hz, 1H), 4.53 (t, J=5.4 Hz, 1H), 3.22 (dd, J=8.1, 5.2 Hz, 3H), 2.65 (s, 6H), 2.47 (dt, J=11.4, 4.7 Hz, 2H), 1.55 (ddd, J=13.1, 9.3, 3.9 Hz, 2H), 1.25 (dt, J=13.2, 4.2 Hz, 2H), 0.92 (s, 3H).

### Example 163

### N¹-(2-(4-(2-fluoroethyl)piperidin-1-yl)phenyl)-N⁴,N⁴-dimethylbenzene-1,4-disulfonamide

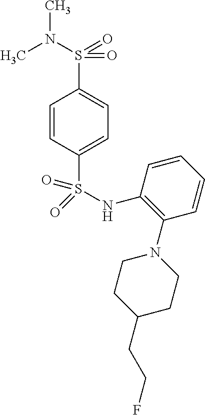 [SMILES: CN(C)S(=O)(=O)c1ccc(S(=O)(=O)Nc2ccccc2N2CCC(CCF)CC2)cc1]

A solution of 2-(4-(2-fluoroethyl)piperidin-1-yl)aniline (0.111 g, 0.5 mmol) in Pyridine (1 ml) was treated with 4-(N,N-dimethylsulfamoyl)benzenesulfonyl chloride (0.142 g, 0.500 mmol). The solution was stirred at 120° C. for 1 hour. The solution was cooled to room temperature. The solution was filtered to give a solid (0.112 g, 24% yield). (LCMS, ESI pos.) Calculated for C₂₁H₂₈FN₃O₄S₂: 470.6 (M+H), Measured: 470.2. ¹H NMR (400 MHz, DMSO-d₆) δ 9.18 (s, 1H), 8.08-8.00 (m, 2H), 8.00-7.93 (m, 2H), 7.33-7.28 (m, 1H), 7.18-7.14 (m, 2H), 7.09 (ddd, J=7.8, 5.4, 3.5 Hz, 1H), 4.61 (t, J=6.1 Hz, 1H), 4.49 (t, J=6.1 Hz, 1H), 2.66 (s, 6H), 2.58 (d, J=11.1 Hz, 1H), 2.49 (dd, J=11.6, 2.4 Hz, 2H), 1.75-1.58 (m, 5H), 1.47 (td, J=7.0, 3.3 Hz, 1H), 1.31 (dd, J=12.0, 3.9 Hz, 2H).

### Example 164

### 4-((6,6a,7,8,9,10-hexahydro-5H-pyrido[1,2-a]quinoxalin-5-yl)sulfonyl)-N,N-dimethylbenzenesulfonamide

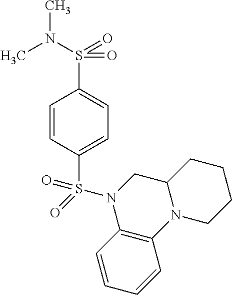 [SMILES: CN(C)S(=O)(=O)c1ccc(S(=O)(=O)N2CC3CCCCN3c3ccccc32)cc1]

A solution of 6,6a,7,8,9,10-hexahydro-5H-pyrido[1,2-a]quinoxaline (0.094 g, 0.5 mmol) in Pyridine (1 ml) was treated with 4-(N,N-dimethylsulfamoyl)benzenesulfonyl chloride (0.142 g, 0.500 mmol). The solution was stirred at 120° C. for 1 hour. The solution was cooled to room temperature. The solution was filtered to give a solid (0.094 g, 22% yield). (LCMS, ESI pos.) Calculated for C₂₀H₂₅N₃O₄S₂: 436.6 (M+H), Measured: 436.0. ¹H NMR (400 MHz, DMSO-d₆) ¹H NMR (400 MHz, DMSO-d₆) δ 7.97-7.92 (m, 2H), 7.77-7.72 (m, 2H), 7.44 (dd, J=8.0, 1.6 Hz, 1H), 7.13 (ddd, J=8.4, 7.2, 1.6 Hz, 1H), 6.92 (dd, J=8.5, 1.3 Hz, 1H), 6.74 (ddd, J=8.0, 7.2, 1.3 Hz, 1H), 4.16 (dd, J=14.4, 4.2 Hz, 1H), 3.78 (d, J=12.4 Hz, 1H), 3.34-3.24 (m, 1H), 2.66 (s, 6H), 2.28-2.18 (m, 1H), 1.97 (td, J=12.5, 2.7 Hz, 1H), 1.72 (d, J=12.2 Hz, 1H), 1.61 (t, J=12.1 Hz, 2H), 1.32 (dddd, J=16.8, 12.7, 8.5, 4.0 Hz, 1H), 1.16-0.93 (m, 2H).

### Example 165

### N¹-(5-fluoro-2-(2-methylpiperidin-1-yl)phenyl)-N⁴,N⁴-dimethylbenzene-1,4-disulfonamide

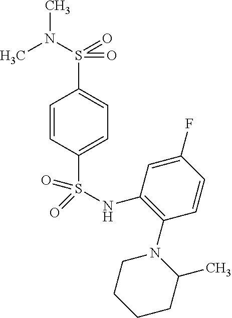

A solution of 5-fluoro-2-(2-methylpiperidin-1-yl)aniline (0.104 g, 0.5 mmol) in Pyridine (1.000 ml) was treated with 4-(N,N-dimethylsulfamoyl)benzenesulfonyl chloride (0.142 g, 0.500 mmol). The solution was stirred at 120° C. for 1 hour. The crude product was purified by reversed-phase chromatography (4-100% CH₃CN over 15 min) to give desired product (0.032 g, 7% yield). (LCMS, ESI pos.) Calculated for C₂₀H₂₆FN₃O₄S₂: 456.6 (M+H), Measured: 456.1. ¹H NMR (400 MHz, DMSO-d6) δ 9.26 (s, 1H), 8.18-8.08 (m, 2H), 8.03-7.90 (m, 2H), 7.41-7.25 (m, 2H), 6.99 (td, J=8.6, 2.9 Hz, 1H), 2.84-2.68 (m, 1H), 2.63 (s, 6H), 2.37 (td, J=11.7, 2.7 Hz, 1H), 1.98-1.87 (m, 1H), 1.78-1.23 (m, 6H), 0.47 (d, J=6.1 Hz, 3H).

### Example 166

### N¹-(5-fluoro-2-(piperidin-1-yl)phenyl)-N⁴,N⁴-dimethylbenzene-1,4-disulfonamide

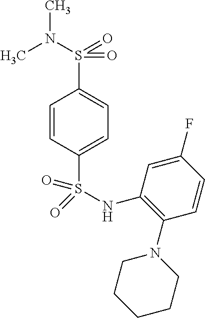 [SMILES: CN(C)S(=O)(=O)c1ccc(S(=O)(=O)Nc2cc(F)ccc2N2CCCCC2)cc1]

A solution of 5-fluoro-2-(piperidin-1-yl)aniline hydrochloride (0.115 g, 0.5 mmol) in Pyridine (1.000 ml) was treated with 4-(N,N-dimethylsulfamoyl)benzenesulfonyl chloride (0.142 g, 0.5 mmol). The solution was stirred at 1200 C for 1 hour. The crude product was purified by reversed-phase chromatography (4-100% CH₃CN over 15 min) to give desired product (0.020 g, 5% yield). (LCMS, ESI pos.) Calculated for C₁₉H₂₄FN₃O₄S₂: 442.5 (M+H), Measured: 442.1. ¹H NMR (400 MHz, DMSO-d6) δ 8.11-8.04 (m, 2H), 8.01-7.93 (m, 2H), 7.21 (ddd, J=21.6, 9.6, 4.5 Hz, 2H), 7.00 (td, J=8.6, 2.9 Hz, 1H), 2.65 (s, 6H), 2.39 (t, J=5.2 Hz, 4H), 1.55 (q, J=5.5 Hz, 4H), 1.45 (d, J=6.7 Hz, 2H).

### Example 167

### 4-((difluoromethyl)sulfonyl)-N-(2-(4-methylpiperidin-1-yl)phenyl)benzenesulfonamide

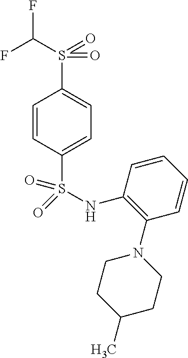 [SMILES: CC1CCN(c2ccccc2NS(=O)(=O)c2ccc(S(=O)(=O)C(F)F)cc2)CC1]

A solution of 2-(4-methylpiperidin-1-yl)aniline (0.095 g, 0.5 mmol) in Pyridine (1 ml) was treated with 4-((difluoromethyl)sulfonyl)benzenesulfonyl chloride (0.145 g, 0.5 mmol). The solution was stirred at 120° C. for 1 hour. The solution was cooled to room temperature. The solution was filtered to give a solid (0.108 g, 24% yield). (LCMS, ESI pos.) Calculated for C₁₉H₂₂F₂N2O₄S₂: 445.5 (M+H), Measured: 445.1. ¹H NMR (400 MHz, DMSO-d6) δ 9.35 (s, 1H), 8.21 (d, J=8.6 Hz, 2H), 8.14 (d, J=8.6 Hz, 2H), 7.43 (s, 1H), 7.30 (dd, J=7.5, 1.7 Hz, 1H), 7.23-7.05 (m, 3H), 2.44 (td, J=11.5, 2.3 Hz, 2H), 1.56-1.45 (m, 2H), 1.37 (ddq, J=10.9, 7.6, 4.6 Hz, 1H), 1.17 (qd, J=11.8, 3.8 Hz, 2H), 0.94 (d, J=6.5 Hz, 3H).

### Example 168

### 4-(ethylsulfonyl)-N-(2-(4-methylpiperidin-1-yl)phenyl)benzenesulfonamide

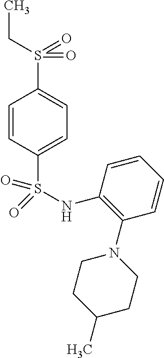 [SMILES: CCS(=O)(=O)c1ccc(S(=O)(=O)Nc2ccccc2N2CCC(C)CC2)cc1]

A solution of 2-(4-methylpiperidin-1-yl)aniline (0.095 g, 0.5 mmol) in Pyridine (1 ml) was treated with 4-(ethylsulfonyl)benzenesulfonyl chloride (0.134 g, 0.5 mmol). The solution was stirred at 120° C. for 1 hour. The solution was cooled to room temperature. The solution was filtered to give a solid (0.103 g, 24% yield). (LCMS, ESI pos.) Calculated for C₂₀H₂₆N₂O₄S₂: 423.6 (M+H), Measured: 423.2. ¹H NMR (400 MHz, DMSO-d6) δ 9.17 (s, 1H), 8.09 (q, J=8.6 Hz, 4H), 7.30 (dd, J=7.7, 1.4 Hz, 1H), 7.18-7.05 (m, 3H), 3.40 (t, J=7.4 Hz, 2H), 2.47 (td, J=11.4, 2.4 Hz, 2H), 1.61-1.49 (m, 2H), 1.48-1.34 (m, 1H), 1.25 (qd, J=11.6, 3.9 Hz, 2H), 1.11 (t, J=7.4 Hz, 3H), 0.96 (d, J=6.4 Hz, 3H).

### Example 169 (a)

### 1-(2-nitrophenyl)-4-(m-tolyl)piperidine

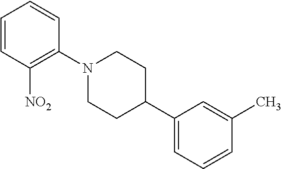 [SMILES: Cc1cccc(C2CCN(c3ccccc3[N+](=O)[O-])CC2)c1]

To a heterogeneous solution of 4-(m-tolyl)piperidine (0.175 g, 1 mmol) and potassium carbonate (0.138 g, 1.000 mmol) (grounded using a mortar and pestle) in DMF (1 ml) was added 1-fluoro-2-nitrobenzene (0.105 ml, 1.000 mmol). The heterogeneous solution was microwaved at 155° C. for 2 hours. The reaction solution was filtered through Celite. The filtrate was concentrated. Used as is for the next step.

### Example 169 (b)

### 2-(4-(m-tolyl)piperidin-1-yl)aniline

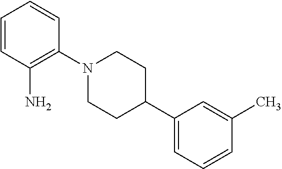 [SMILES: Cc1cccc(C2CCN(c3ccccc3N)CC2)c1]

A solution of 1-(2-nitrophenyl)-4-(m-tolyl)piperidine in MeOH (10 ml) was subjected to H-cube conditions: 0.8 mL/min, 40 bar, 40° C. The solution was concentrated to give the desired product which was used as is for the next step.

### Example 169 (c)

### N¹,N¹-dimethyl-N⁴-(2-(4-(m-tolyl)piperidin-1-yl)phenyl)benzene-1,4-disulfonamide

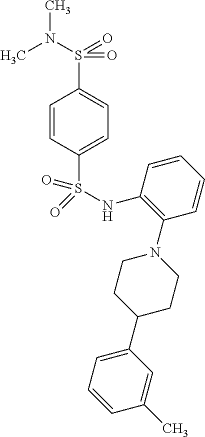 [SMILES: Cc1cccc(C2CCN(c3ccccc3NS(=O)(=O)c3ccc(S(=O)(=O)N(C)C)cc3)CC2)c1]

A solution of 2-(4-(m-tolyl)piperidin-1-yl)aniline (0.266 g, 1.000 mmol) in Pyridine (1 ml) was treated with 4-(N,N-dimethylsulfamoyl)benzenesulfonyl chloride (0.284 g, 1 mmol). The solution was stirred at 120° C. for 1 hour. The solution was cooled to room temperature. The solution was filtered to give a solid (0.010 g, 2% yield). (LCMS, ESI pos.) Calculated for C₂₆H₃₁N₃O₄S₂: 514.7 (M+H), Measured: 514.2. ¹H NMR (400 MHz, DMSO-d6) ¹H NMR (400 MHz, DMSO-d₆) δ 9.26 (s, 1H), 8.06 (d, J=8.4 Hz, 2H), 8.00-7.93 (m, 2H), 7.37 (dd, J=7.9, 1.6 Hz, 1H), 7.31-7.01 (m, 7H), 2.62 (s, 11H), 2.36 (s, 3H), 1.82 (td, J=12.4, 6.3 Hz, 2H), 1.67 (d, J=12.5 Hz, 2H).

### Example 170 (a)

### 1-(2-nitrophenyl)-4-(p-tolyl)piperidine

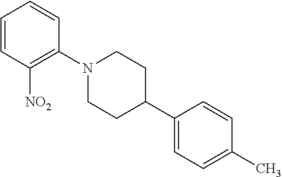 [SMILES: Cc1ccc(C2CCN(c3ccccc3[N+](=O)[O-])CC2)cc1]

To a heterogeneous solution of 4-(p-tolyl)piperidine (0.175 g, 1 mmol) and potassium carbonate (0.138 g, 1 mmol) (grounded using a mortar and pestle) in DMF (1 ml) was added 1-(2-nitrophenyl)-4-(p-tolyl)piperidine. The heterogeneous solution was microwaved at 155° C. for 2 hours. The reaction solution was filtered through Celite. The filtrate was concentrated. Used as is for the next step.

### Example 170 (b)

### 2-(4-(p-tolyl)piperidin-1-yl)aniline

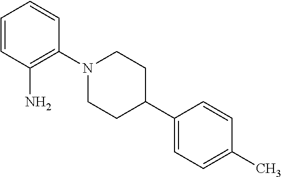 [SMILES: Cc1ccc(C2CCN(c3ccccc3N)CC2)cc1]

A solution of 1-(2-nitrophenyl)-4-(p-tolyl)piperidine in MeOH was subjected to H-cube conditions: 0.8 mL/min, 40 bar, 40° C. The solution was concentrated to give the desired product which was used as is for the next step.

### Example 170 (c)

### N¹,N¹-dimethyl-N⁴-(2-(4-(p-tolyl)piperidin-1-yl)phenyl)benzene-1,4-disulfonamide

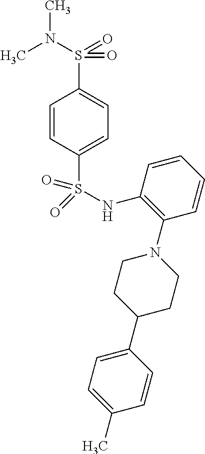 [SMILES: Cc1ccc(C2CCN(c3ccccc3NS(=O)(=O)c3ccc(S(=O)(=O)N(C)C)cc3)CC2)cc1]

A solution of 2-(4-(p-tolyl)piperidin-1-yl)aniline (0.266 g, 1 mmol) in Pyridine (1 ml) was treated with 4-(N,N-dimethylsulfamoyl)benzenesulfonyl chloride (0.284 g, 1.000 mmol). The solution was stirred at 120° C. for 1 hour. The solution was stirred at 120° C. for 1 hour. The crude product was purified by reversed-phase chromatography (4-100% CH₃CN over 15 min) to give desired product (0.004 g, 1% yield). (LCMS, ESI pos.) Calculated for C₂₆H₃₁N₃O₄S₂: 514.7 (M+H), Measured: 514.2.

### Example 171 (a)

### 1-(2-nitrophenyl)-4-phenylpiperidine

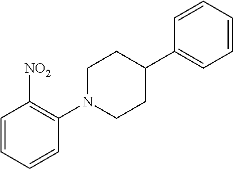 [SMILES: O=[N+]([O-])c1ccccc1N1CCC(c2ccccc2)CC1]

To a heterogeneous solution of 4-phenylpiperidine (0.161 g, 1 mmol) and potassium carbonate (0.138 g, 1.000 mmol) (grounded using a mortar and pestle) in DMF (1 ml) was added 1-fluoro-2-nitrobenzene (0.105 ml, 1.000 mmol). The heterogeneous solution was microwaved at 155° C. for 2 hours. The solution was filtered through Celite. The filtrate was concentrated. The residue was used as is for the next step.

### Example 171 (b)

### 2-(4-phenylpiperidin-1-yl)aniline

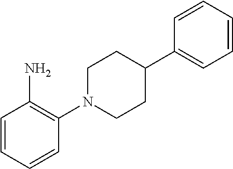 [SMILES: Nc1ccccc1N1CCC(c2ccccc2)CC1]

A solution of 1-(2-nitrophenyl)-4-phenylpiperidine in MeOH (10 ml) was subjected to H-cube conditions: 0.8 mL/min, 40 bar, 40° C. The solution was concentrated. The residue was used as is for the next step.

### Example 171 (c)

### N¹,N¹-dimethyl-N⁴-(2-(4-phenylpiperidin-1-yl)phenyl)benzene-1,4-disulfonamide

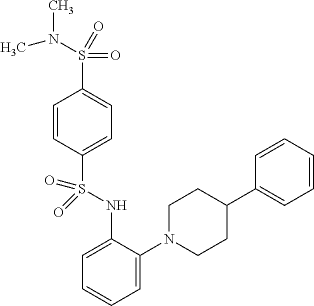 [SMILES: CN(C)S(=O)(=O)c1ccc(S(=O)(=O)Nc2ccccc2N2CCC(c3ccccc3)CC2)cc1]

A solution of 2-(4-phenylpiperidin-1-yl)aniline (0.080 g, 0.317 mmol) in Pyridine (1 ml) was treated with 4-(N,N-dimethylsulfamoyl)benzenesulfonyl chloride (0.09 g, 0.317 mmol). The solution was stirred at 120° C. for 1 hour. The crude product was purified by reversed-phase chromatography (4-100% CH₃CN over 15 min) to give desired product (0.043 g, 9% yield). (LCMS, ESI pos.) Calculated for C₂₅H₂₉N₃O₄S₂: 500.6 (M+H), Measured: 500.2.

### Example 172 (a)

### 4-(3,5-dimethylphenyl)-1-(2-nitrophenyl)piperidine

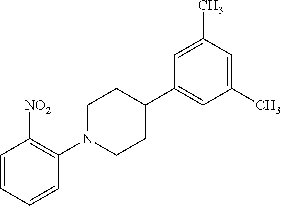 [SMILES: Cc1cc(C)cc(C2CCN(c3ccccc3[N+](=O)[O-])CC2)c1]

To a heterogeneous solution of 4-(3,5-dimethylphenyl)piperidine (0.189 g, 1 mmol) and potassium carbonate (0.138 g, 1.000 mmol) (grounded using a mortar and pestle) in DMF (1 ml) was added 1-fluoro-2-nitrobenzene (0.105 ml, 1.000 mmol). The heterogeneous solution was microwaved at 155° C. for 2 hours. The solution was filtered through Celite. The filtrate was concentrated. The residue was used as is for the next step.

### Example 172 (b)

### 2-(4-(3,5-dimethylphenyl)piperidin-1-yl)aniline

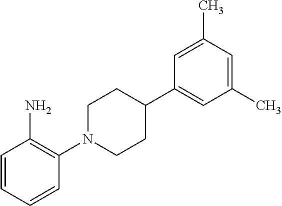 [SMILES: Cc1cc(C)cc(C2CCN(c3ccccc3N)CC2)c1]

A solution of 4-(3,5-dimethylphenyl)-1-(2-nitrophenyl)piperidine in MeOH was subjected to H-cube conditions: 0.8 mL/min, 40 bar, 40° C. The solution was concentrated. The residue was used as is for the next step.

### Example 172 (c)

### N¹-(2-(4-(3,5-dimethylphenyl)piperidin-1-yl)phenyl)-N⁴,N⁴-dimethylbenzene-1,4-disulfonamide

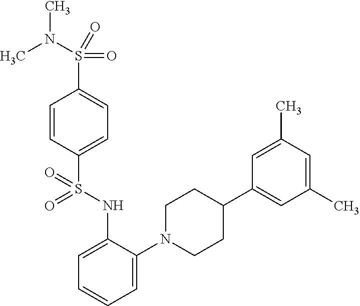

A solution of 2-(4-(3,5-dimethylphenyl)piperidin-1-yl)aniline (0.089 g, 0.317 mmol) in Pyridine (3 ml) was treated with 4-(N,N-dimethylsulfamoyl)benzenesulfonyl chloride (0.09 g, 0.317 mmol). The solution was stirred at 120° C. for 1 hour. The crude product was purified by reversed-phase chromatography (4-100% CH₃CN over 15 min) to give desired product (0.029 g, 6% yield). (LCMS, ESI pos.) Calculated for C₂₇H₃₃N₃O₄S₂: 528.7 (M+H), Measured: 528.0. ¹H NMR (400 MHz, DMSO-d₆) δ 9.26 (s, 1H), 8.07 (d, J=8.2 Hz, 2H), 7.96 (d, J=8.3 Hz, 2H), 7.36 (d, J=7.8 Hz, 1H), 7.27-7.06 (m, 3H), 6.89 (d, J=15.6 Hz, 3H), 2.63 (s, 10H), 2.51-2.43 (m, 1H), 2.31 (s, 6H), 1.82 (ddd, J=20.2, 12.5, 8.1 Hz, 2H), 1.70-1.60 (m, 2H).

### Example 173 (a)

### 8-(2-nitrophenyl)-8-azaspiro[4.5]decane

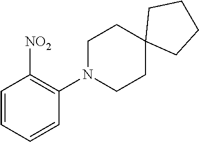 [SMILES: O=[N+]([O-])c1ccccc1N1CCC2(CCCC2)CC1]

To a heterogeneous solution of 8-azaspiro[4.5]decane (0.139 g, 1 mmol) and potassium carbonate (0.138 g, 1.000 mmol) (grounded using a mortar and pestle) in DMF (1 ml) was added 1-fluoro-2-nitrobenzene (0.105 ml, 1.000 mmol). The heterogeneous solution was microwaved at 155° C. for 2 hours. The solution was filtered through Celite. The filtrate was concentrated. The residue was used as is for the next step.

### Example 173 (b)

### 2-(8-azaspiro[4.5]decan-8-yl)aniline

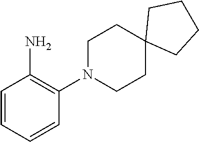 [SMILES: Nc1ccccc1N1CCC2(CCCC2)CC1]

A solution of 8-(2-nitrophenyl)-8-azaspiro[4.5]decane in MeOH was subjected to H-cube conditions: 0.8 mL/min, 40 bar, 40° C. The solution was concentrated. The residue was used as is for the next step.

### Example 173 (c)

### N¹-(2-(8-azaspiro[4.5]decan-8-yl)phenyl)-N⁴,N⁴-dimethylbenzene-1,4-disulfonamide

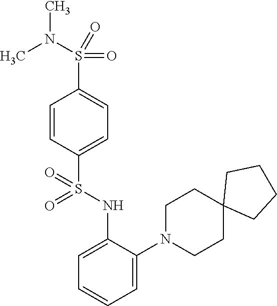 [SMILES: CN(C)S(=O)(=O)c1ccc(S(=O)(=O)Nc2ccccc2N2CCC3(CCCC3)CC2)cc1]

A solution of 2-(8-azaspiro[4.5]decan-8-yl)aniline in Pyridine (3 ml) was treated with 4-(N,N-dimethylsulfamoyl)benzenesulfonyl chloride. The solution was stirred at 120° C. for 1 hour. The solution was cooled to room temperature. The solution was filtered to give a solid (0.045 g, 9% yield). (LCMS, ESI pos.) Calculated for C₂₃H₃₁N₃O₄S₂: 478.6 (M+H), Measured: 478.0. ¹H NMR (400 MHz, DMSO-d₆) δ 9.16 (s, 1H), 8.11-8.01 (m, 2H), 8.00-7.90 (m, 2H), 7.31 (dd, J=7.8, 1.5 Hz, 1H), 7.25-7.04 (m, 3H), 3.37 (s, 3H), 1.68-1.54 (m, 5H), 1.53-1.36 (m, 9H).

### Example 174

### N-(4-(methylsulfonamido)phenyl)-2-(piperidin-1-yl)benzenesulfonamide

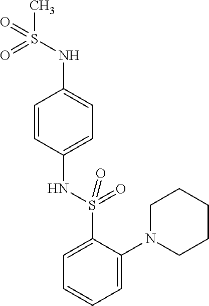 [SMILES: CS(=O)(=O)Nc1ccc(NS(=O)(=O)c2ccccc2N2CCCCC2)cc1]

A solution of N-(4-aminophenyl)methanesulfonamide (0.093 g, 0.5 mmol) in Pyridine (1.000 ml) was treated with 2-(piperidin-1-yl)benzenesulfonyl chloride (0.130 g, 0.500 mmol). The solution was stirred at 120° C. for 1 hour. The crude product was purified by reversed-phase chromatography (4-100% CH₃CN over 15 min) to give desired product (0.097 g, 24% yield). (LCMS, ESI pos.) Calculated for C₁₈H₂₃N₃O₄S₂: 410.5 (M+H), Measured: 410.1.

### Example 175

### 2-(piperidin-1-yl)-N-(4-(N-propylsulfamoyl)phenyl)benzenesulfonamide

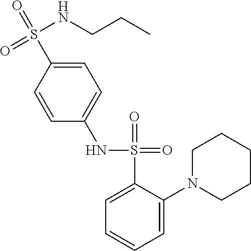 [SMILES: CCCNS(=O)(=O)c1ccc(NS(=O)(=O)c2ccccc2N2CCCCC2)cc1]

A solution of 4-amino-N-propylbenzenesulfonamide (0.107 g, 0.5 mmol) in Pyridine (1.000 ml) was treated with 2-(piperidin-1-yl)benzenesulfonyl chloride (0.130 g, 0.500 mmol). The solution was stirred at 120° C. for 1 hour. The crude product was purified by reversed-phase chromatography (4-100% CH₃CN over 15 min) to give desired product (0.107 g, 24% yield). (LCMS, ESI pos.) Calculated for C₂₀H₂₇N₃O₄S₂: 438.6 (M+H), Measured: 438.1.

### Example 176

### 2-(piperidin-1-yl)-N-(4-(pyridin-3-yl)phenyl)benzenesulfonamide

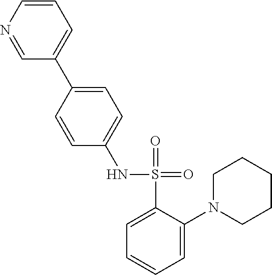 [SMILES: O=S(=O)(Nc1ccc(-c2cccnc2)cc1)c1ccccc1N1CCCCC1]

A solution of 4-(pyridin-3-yl)aniline (0.085 g, 0.5 mmol) in Pyridine (1.000 ml) was treated with 2-(piperidin-1-yl)benzenesulfonyl chloride (0.130 g, 0.500 mmol). The solution was stirred at 120° C. for 1 hour. The crude product was purified by reversed-phase chromatography (4-100% CH₃CN over 15 min) to give desired product (0.125 g, 32% yield). (LCMS, ESI pos.) Calculated for C₂₂H₂₃N₃₀₂S: 394.5 (M+H), Measured: 394.1.

### Example 177

### N,N-dimethyl-4-((2-(piperidin-1-yl)phenyl)sulfonamido)benzenesulfonamide

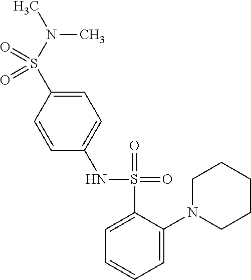 [SMILES: CN(C)S(=O)(=O)c1ccc(NS(=O)(=O)c2ccccc2N2CCCCC2)cc1]

A solution of 4-amino-N,N-dimethylbenzenesulfonamide (0.100 g, 0.5 mmol) in Pyridine (1.000 ml) was treated with 2-(piperidin-1-yl)benzenesulfonyl chloride (0.130 g, 0.500 mmol). The solution was microwaved at 120 deg for 1 hr. The solution was cooled to room temperature. The solution was filtered to give a solid (0.128 g, 30% yield). (LCMS, ESI pos.) Calculated for C₁₉H₂₅N₃O₄S₂: 424.5 (M+H), Measured: 424.1. ¹H NMR (400 MHz, DMSO-d₆) δ 10.26 (s, 1H), 8.00 (dd, J=8.0, 1.6 Hz, 1H), 7.63 (ddd, J=8.3, 7.3, 1.6 Hz, 1H), 7.60-7.56 (m, 2H), 7.46 (dd, J=8.1, 1.2 Hz, 1H), 7.39-7.30 (m, 1H), 7.30-7.21 (m, 2H), 2.79 (t, J=5.2 Hz, 4H), 2.53 (s, 7H), 1.76 (p, J=5.5 Hz, 4H), 1.55 (d, J=6.2 Hz, 2H).

### Example 178

This example demonstrates that transgenic overexpression of ML1 ameliorates muscular dystrophies in mdx mice.

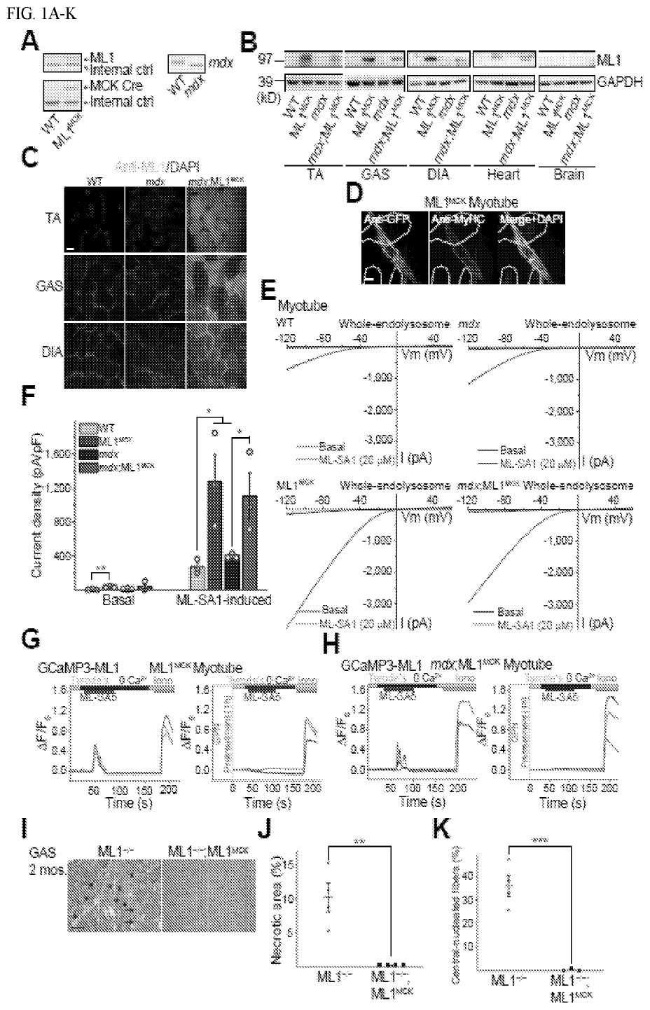

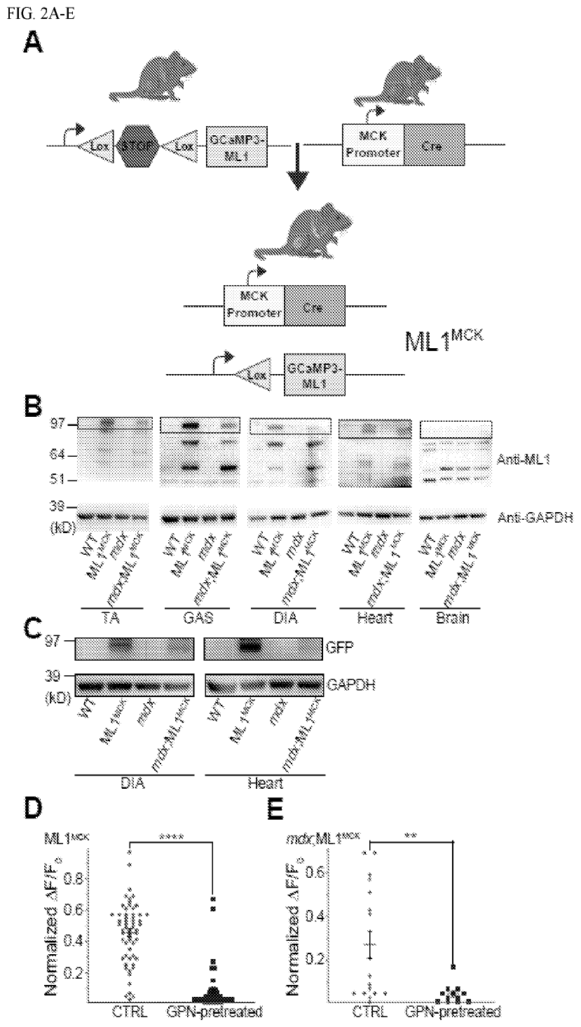

To achieve muscle-specific overexpression of ML1 in vivo, experiments were conducted that crossed a mouse line carrying a GCaMP3-ML1 transgene (ML1 ROSA-lSl) (15) with a Cre line driven by the muscle-specific creatine kinase promoter (MCK-Cre) (16). The resultant ML1 ROSA-lSl;MCK-Cre (abbreviated as ML1^{MCK}) progeny were then crossed with mdx mice with a loss-of-function dystrophin mutation (17) to generate mdx;ML1^{MCK} mice (FIG. 1A, FIG. 2A). Western blotting and immunofluorescence analyses with anti-ML1 antibody revealed ML1 overexpression in both skeletal and cardiac muscle tissues of the mdx;ML1^{MCK} mice; isolated primary myotubes were ML1-immunopositive, whereas non-muscle tissues showed barely detectable immunoreactivity (FIG. 1B-D; FIG. 2B, C).

Whole-lysosomal ML1 currents, activated by TRPML-specific synthetic agonists (ML-SA1 and ML-SA5) (12, 15, 18), were 4-10 times larger in the ML1^{MCK} myotubes than in wild-type (WT) controls (FIG. 1E, F). ML-SAs induced robust glycyl-L-phenylalanine 2-naphthylamide-sensitive lysosomal Ca²⁺ release (15) in ML1^{MCK} myotubes, suggesting that genetically-overexpressed ML1 channels were functionally localized on the late endosomal and lysosomal membranes of muscle cells (FIG. 1G, H; FIG. 2D, E). Introducing ML1^{MCK} into the ML1 knockout (KO) mice resulted in a complete rescue of the dystrophic phenotype, as manifested by the decreases in myofiber necrosis and central nucleation (19) (FIG. 1I-K). Hence, the ML1^{MCK} mice were considered suitable for investigating the in vivo effects of ML1 overexpression on striated muscles.

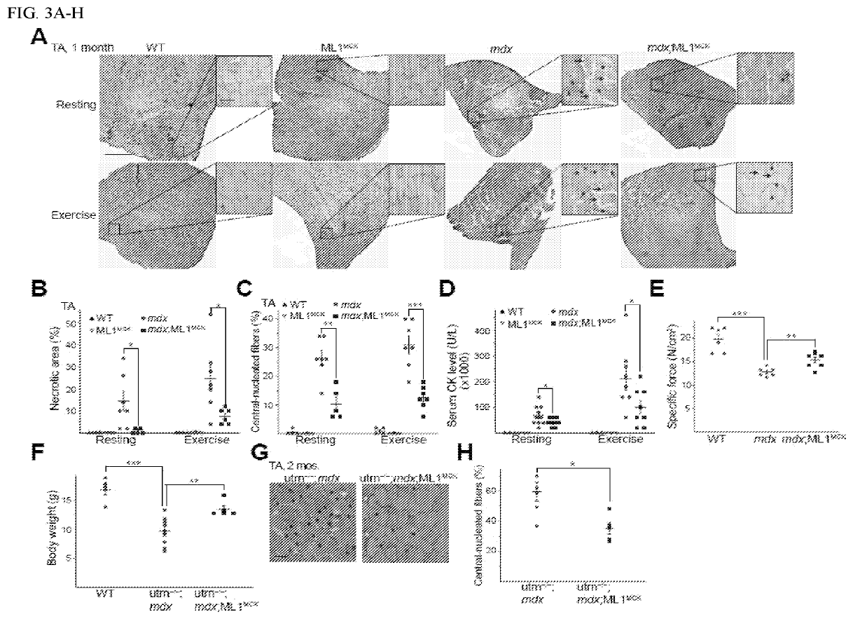

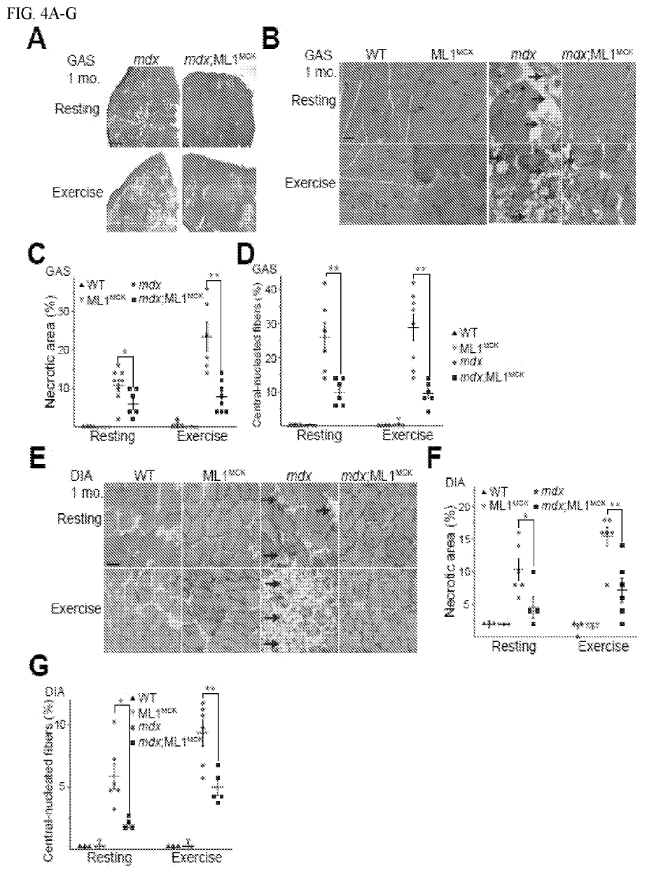

Mdx mice, either homozygous females or hemizygous males, exhibit early-onset muscular dystrophies, as evidenced by myofiber necrosis (myonecrosis) and degeneration/regeneration cycles, which were readily observed by postnatal day 14 (P14) (20). In 1-month-old mdx;ML1^{MCK} mice, heamotoxylin and eosin (H&E) staining revealed that myonecrosis, quantified by the percentage of the necrotic area (i.e., the presence of necrotic myofibers, immune cells, and fibroblasts) in whole cross-sections, was markedly reduced in tibialis anterior (TA) muscles compared with age-matched mdx mice, especially after downhill treadmill exercise (FIG. 3A, B). The number of centrally-nucleated fibers, caused by repeated myocyte degeneration and regeneration, was also significantly reduced in the mdx;ML1^{MCK} muscles (FIG. 3A, C). Similar anti-dystrophic effects were seen in other skeletal muscles, including the gastrocnemius (GAS) and diaphragm (DIA) (FIG. 4A-G). Serum creatine kinase (CK) levels, a diagnostic biomarker of DMD (19), were also reduced by ML1 overexpression (FIG. 3D). Consistent with the histological and biochemical results, physiological assays showed greater specific muscle force in mdx mice following ML1^{MCK} overexpression (FIG. 3E).

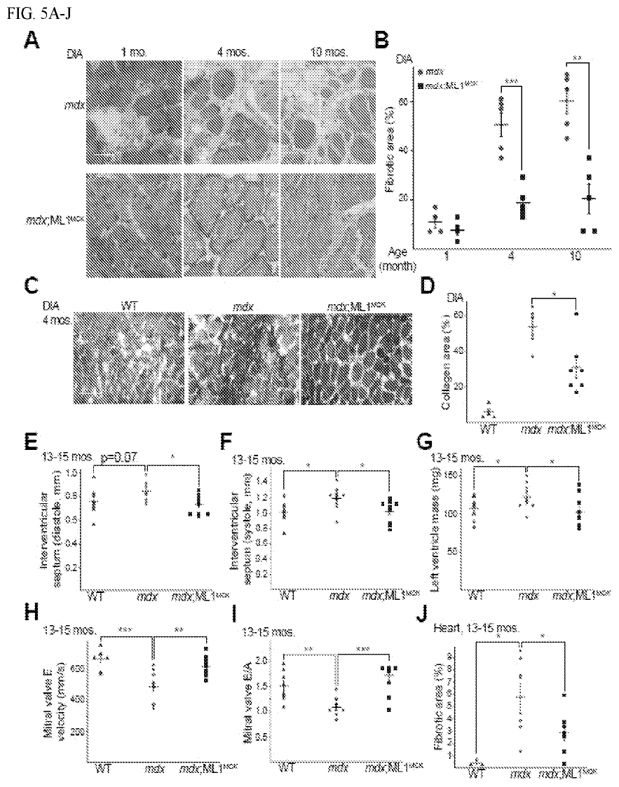

In most skeletal muscles of mdx mice, the dystrophic phenotype did not appear to be progressive, perhaps due to compensatory expression of utrophin, a functional homolog of dystrophin (21). Relative to mdx mice, utrophin^{-/-};mdx (dKO) mice have a much more severe and progressive muscular dystrophy that resembled human DMD (21). Both dystrophy and body weight loss characteristic of the utrophin^{-/-;mdx phenotypes (}21) were improved by ML1^{MCK} overexpression (FIG. 3F-H). Dystrophy of the DIA in mdx mice was also progressive, as seen in human DMD, and respiratory failure is a major cause of death in DMD (6). Consistent with previous studies (6), we observed massive necrosis and subsequent fibrous and adipose tissue replacement (i.e., fibrosis) in mdx DIA muscles (FIG. 5A, B). At all ages examined (1 mo., 4 mos., 10 mos.), fibrosis was reduced significantly by ML1^{MCK} overexpression (FIG. 5A, B). The content of collagen, a major component of fibrous scar tissue (19), was also decreased by ML1^{MCK} overexpression (FIG. 5C, D).

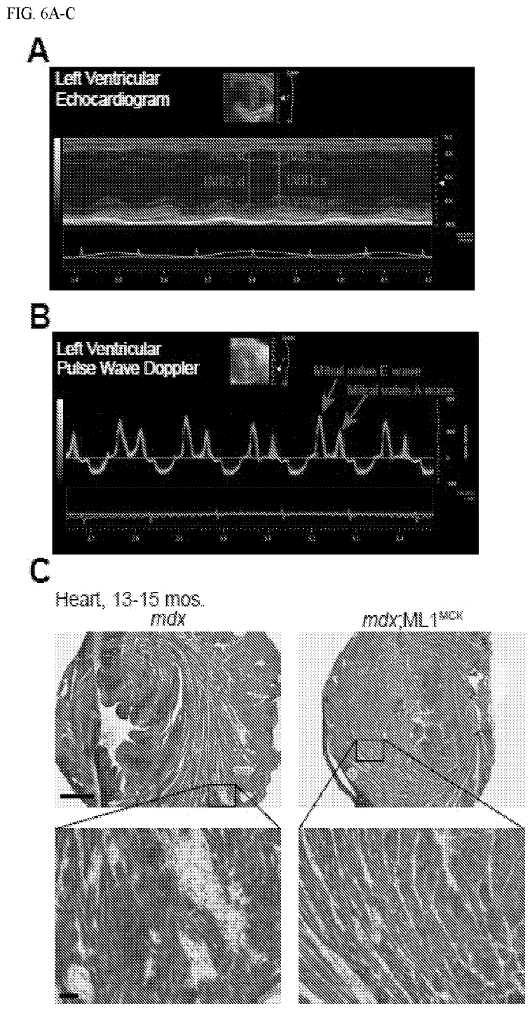

Cardiac failure is another major cause of death in DMD, but cardiomyopathies are observed only in aged (e.g., >10-month-old) mdx mice (22, 23). Echocardiograms in 13-15-month-old WT, mdx, and mdx; ML1^{MCK} male mice were performed (FIG. 6A). Compared with WT mice, mdx mice had thickened interventricular septum and increased left ventricle mass (FIG. 5E-G), both of which are characteristic of dilated cardiomyopathies (22). Both echocardiographic abnormalities were ameliorated by ML1 overexpression (FIG. 5E-G). Experiments also performed Pulse wave Doppler echocardiography to analyze the contractile function of the left ventricle (FIG. 6B). Consistent with previous studies (24), mdx hearts had reduced E wave velocity and E/A ratio (FIG. 5H, I), suggestive of ventricular dysfunction. Both parameters were corrected by ML1^{MCK} overexpression (FIG. 5H, I). Histological analyses showed that cardiac fibrosis in aged (15-month-old) mdx mice was also reduced by ML1^{MCK} expression (FIG. 5J; FIG. 6C). Put together, these results suggest that transgenic overexpression of ML1 is sufficient to attenuate dystrophies of both skeletal and cardiac muscles in DMD-like mouse models.

### Example 179

This example demonstrates that pharmacological activation of ML1 in vivo ameliorates muscular dystrophy in mdx mice.

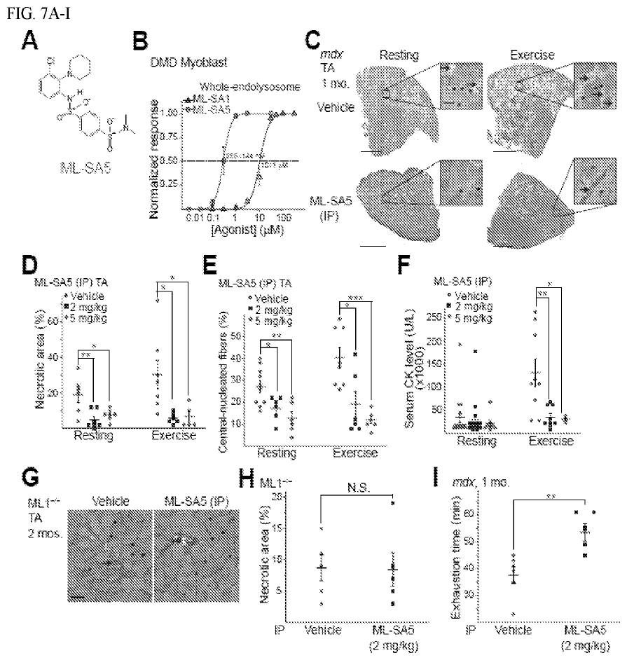

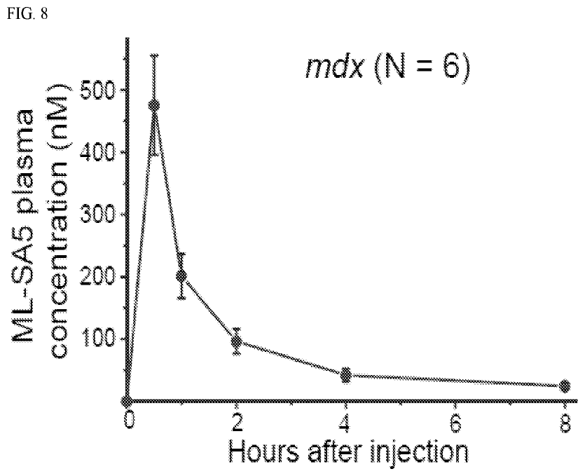

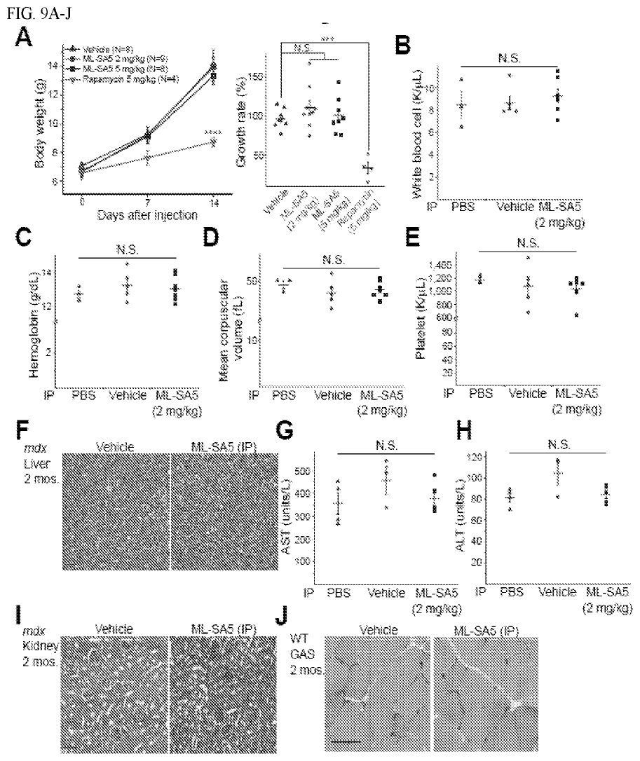

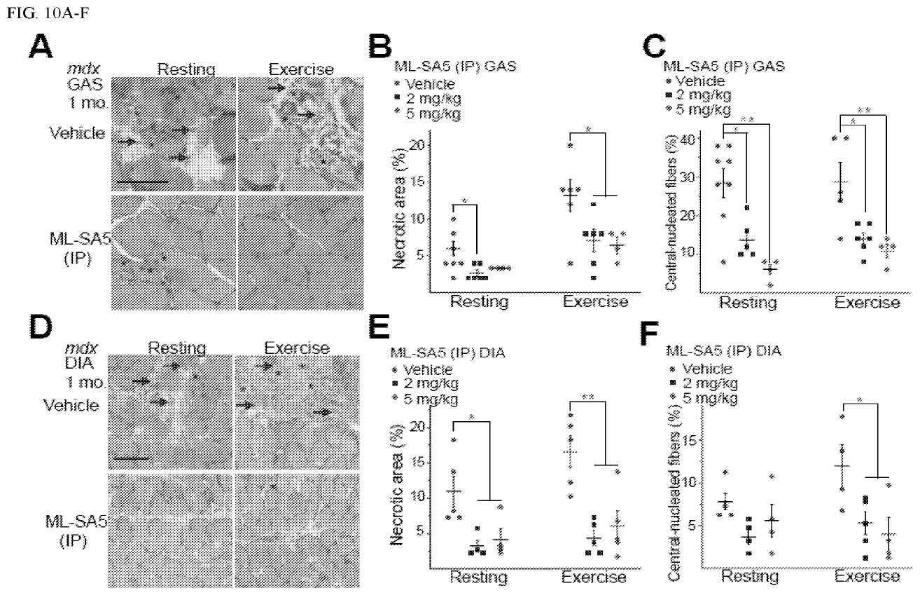

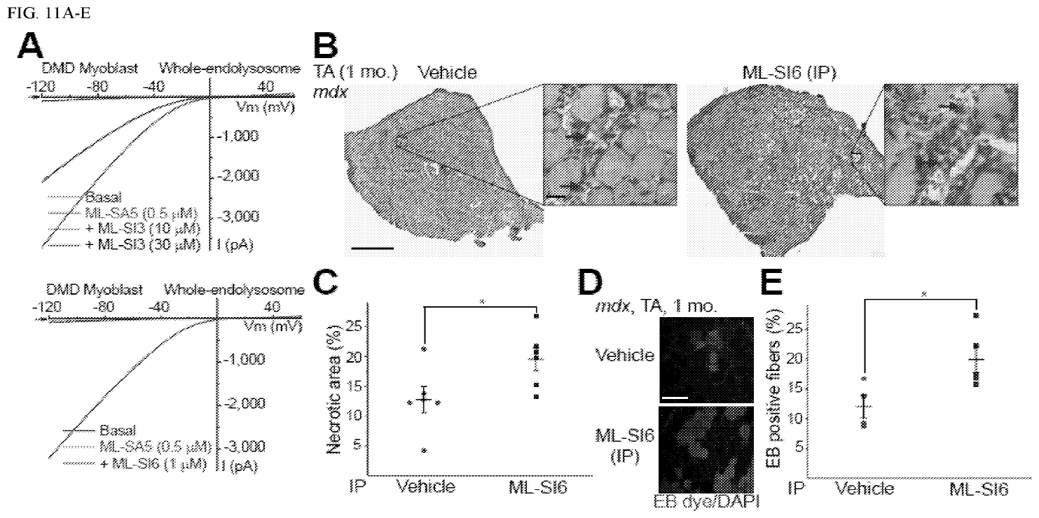

Next, experiments were conducted that tested whether small-molecule ML1 agonists have a muscle protective effect in mdx mice. ML-SA5, a potent ML-SA compound with an in vitro EC₅₀ value in the nM range for endogenous ML1 (FIG. 7A, B), exhibited pharmacokinetic properties suitable for in vivo studies (FIG. 8A). Mdx mice received daily intraperitoneal (i.p.) injection of ML-SA5, starting at P14 and continuing for at least 2 weeks. Like the vehicle [10% DMSO+40% PEG+50% phosphate buffered saline (PBS)]-treated group, no pathological signs were evident following ML-SA5 injection, manifested by normal body weight, complete blood count, liver biochemistry and histology, and kidney histology (FIG. 9A-I), suggesting the minimal toxicity effects. However, at the i.p. dose of 2-5 mg/kg, daily ML-SA5-injection for two weeks decreased TA muscle necrosis by more than 70%, both at rest and after treadmill exercise (FIG. 7C, D). Centrally-nucleated fibers were also decreased in the ML-SA5-injected mdx mice (FIG. 7C, E). Similar rescue effects were also seen in the GAS and DIA muscles (FIG. 10A-F). Consistently, serum CK level was also reduced in mdx mice that received ML1 agonist injection (FIG. 7F). In contrast, i.p.-injection of ML-SI6, a potent ML1 inhibitor (19, 25), worsened the dystrophic phenotype in mdx mice (FIG. 11A-E). Furthermore, in ML1 KO mice that also exhibit a DMD-like phenotype such as necrosis and central nuclei (19), no obvious ML-SA5 rescue effects were seen (FIG. 7G, H). Hence, the actions of ML-SA5 is likely to be mediated through ML1 (i.e., on-target). Finally, at the behavioral level, motor performance in a downhill treadmill test improved markedly following ML-SA injection in mdx mice (FIG. 7I). Therefore, similar to the genetic overexpression studies, pharmacological activation of ML1 in vivo using small molecules was also muscle protective.

### Example 180

This example demonstrates that ML1 facilitates sarcolemma repair, thereby reducing skeletal and cardiac muscle damage in mdx mice.

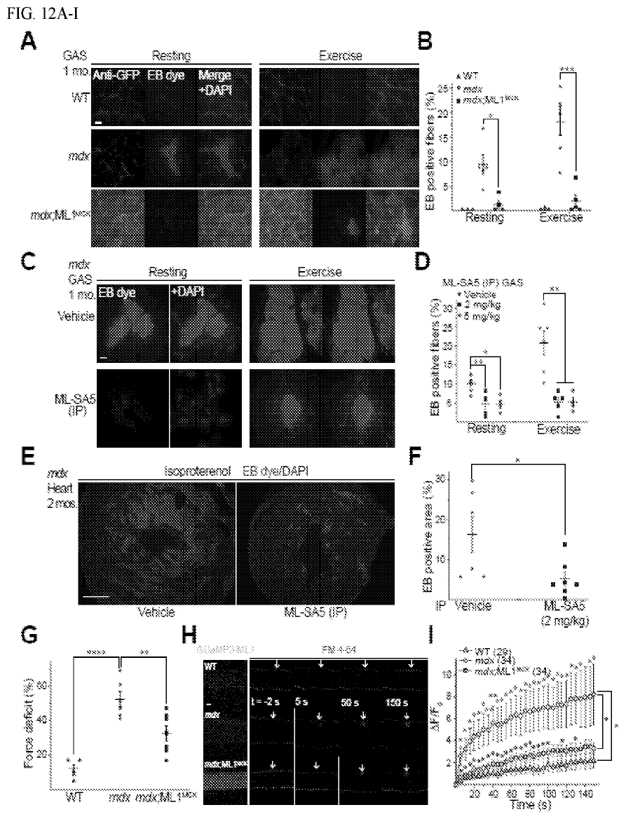

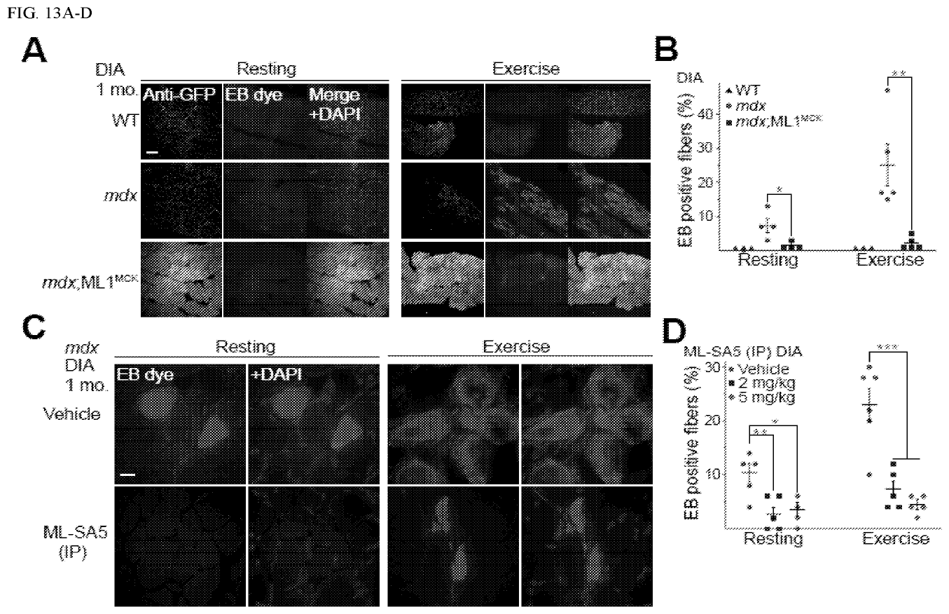

One of the major causes of muscular dystrophy is defective sarcolemma repair, and lysosomal exocytosis is a primary route for resealing damaged membranes (26). Given the essential role of ML1 in lysosomal exocytosis and membrane resealing (19), it is likely that ML1's muscle protective effects are mediated by sarcolemma repair. Skeletal muscle damage was experimentally induced in vivo with a short-term treadmill exercise protocol (27) and confirmed with the entry of the membrane-impermeable Evans Blue (EB) dye (19). The percentage of EB-positive fibers, which never exceeded 2% in WT muscles, reached 9% at rest and 18% after treadmill exercise in mdx muscles (FIG. 12A, B). Notably, mdx;ML1^{MCK} mice had less than 2% of EB-positive muscle fibers, even after treadmill exercise (FIG. 12A, B; FIG. 13A,B). EB uptake was also much reduced in ML-SA-treated mdx muscle (FIG. 12C, D; FIG. 13C, D), but increased in ML-SI-treated mdx mice (FIG. 11D, E). Likewise, when cardiomyocyte damage was induced in young (2-month-old) mdx mice with isoproterenol, EB uptake in the heart tissues was much reduced by ML-SA5 injection (FIG. 12E, F). Collectively, these results suggest that compromised membrane integrity in both skeletal and cardiac muscles can be improved by ML1 upregulation.

Experiments were conducted that also performed an ex vivo muscle damage test, in which in situ force in GAS muscles were measured before and after mechanical stretch by blind experimenters (27). The muscle force deficit seen in mdx mice was markedly reduced with ML1 overexpression (FIG. 12G), showing that ML1 expression has the potential to protect muscles from contraction-induced muscle damage in vivo. To study membrane repair in vitro, experiments used FM4-64 dye to detect membrane disruptions (8) in single myofibers isolated from the flexor digitorum brevis (FDB) muscle (8, 9). Upon laser irradiation, mdx fibers continued to take up FM dye at the injury sites for several minutes, whereas FM dye uptake was transient in ML1-overexpressed mdx muscle (FIG. 12H, I). These results suggest that ML1 activation facilitates membrane repair to reduce muscle damage in mdx mice.

### Example 181

This example demonstrates that upregulation of ML1 in muscle activates transcriptional factor EB (TFEB) and corrects lysosomal insufficiency.

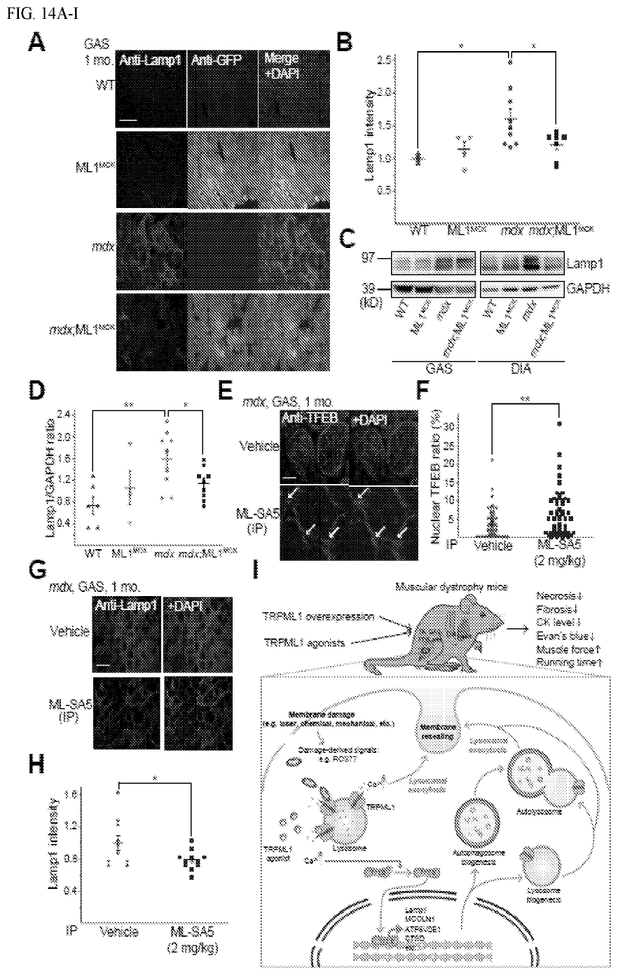

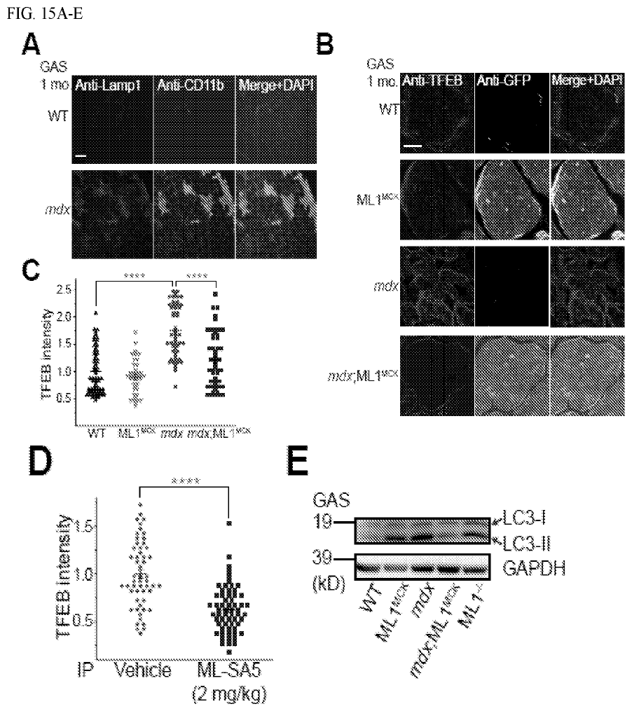

Expression of lysosome-associated membrane protein 1 (Lamp1) is an indicator of lysosome function in vivo, such that Lamp1 upregulation accompanies lysosomal dysfunction or insufficiency (28). Aberrant Lamp1 expression has been reported in several mdx studies (29, 30). We found that Lamp1 levels were increased in mdx compared with WT mice in both GAS and DIA isolated from 1-month-old animals (FIG. 14A-D). In the immunofluorescence analysis, Lamp1 upregulation was apparent in both muscle fibers and surrounding cells (e.g., infiltrated macrophages; FIG. 14A; FIG. 15A), suggesting that inflammation, which is known to be associated with necrosis (31), may also contribute to Lamp1 upregulation. Collectively, these results suggest that there is lysosome insufficiency in the muscle tissues of mdx mice, and that lysosome biogenesis might have been weakly activated to compensate for the deficiency.

ML1 activation has been reported to increase lysosome biogenesis and Lamp1 levels through nuclear translocation of TFEB, a master regulator of lysosomal genes (18, 32). Intriguingly, Lamp1 expression was significantly lower in mdx;ML1^{MCK} mice compared to mdx mice (FIG. 14A-D). This seemingly puzzling result may be explained the fact that ML1 expression boosts lysosome function and decreases necrosis in muscle, obviating the need for compensatory changes via the expression of lysosomal genes. In mdx;ML1^{MCK} mice or ML-SA-injected mdx mice, TFEB nuclear translocation was increased (FIG. 14E, F; FIG. 15B, C), but Lamp1 upregulation and other compensatory lysosomal changes were suppressed (FIG. 14G, H; FIG. 15D, E). Hence, ML1 activation may further boost lysosome function to override lysosome insufficiency, yielding muscle protective effects. Similar scenarios have been reported in several lysosomal storage disorders (LSDs), in which TFEB activation has been found to increase lysosome biogenesis in WT cells while suppressing Lamp1 upregulation in LSD animal models (14, 28).

### Example 182

This example demonstrates that ML1 protects human DMD muscle cells from damage through TFEB and lysosomal exocytosis.

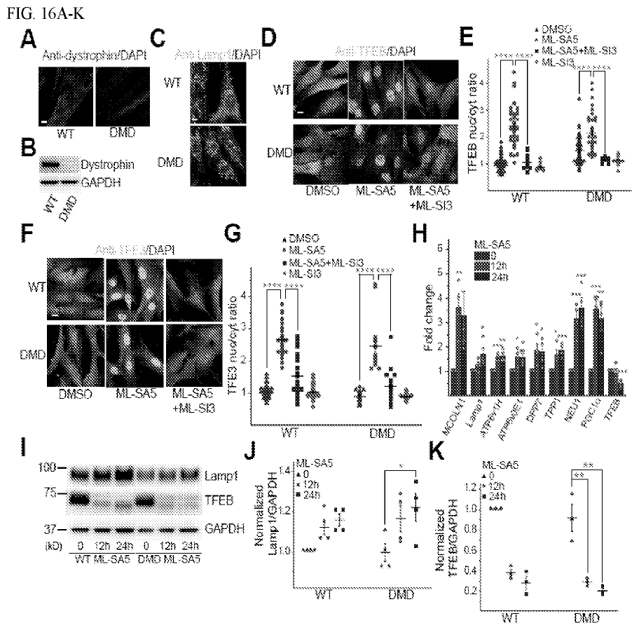

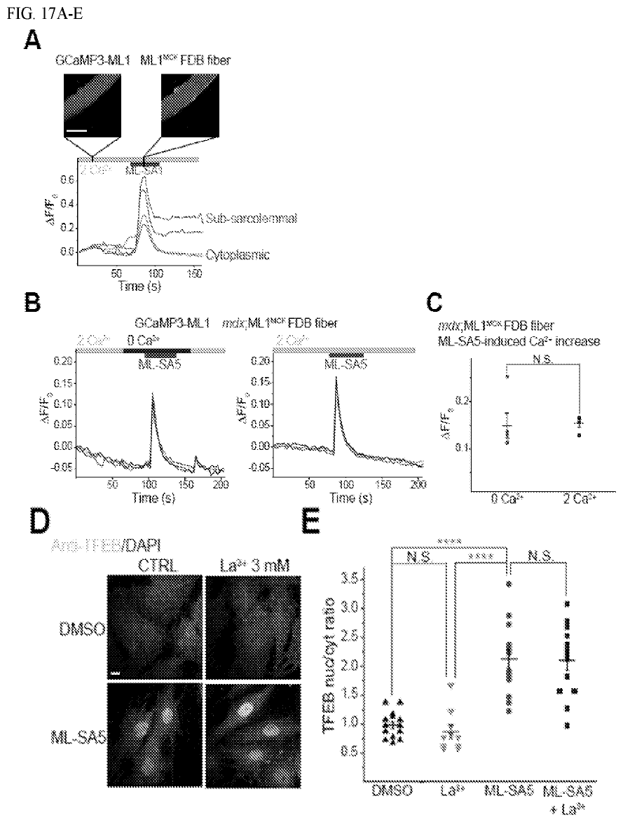

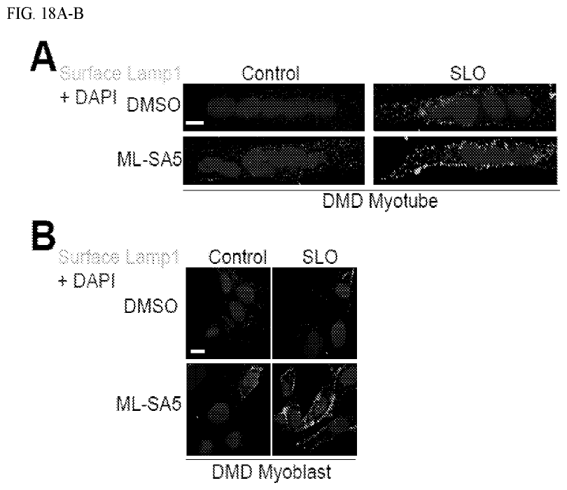

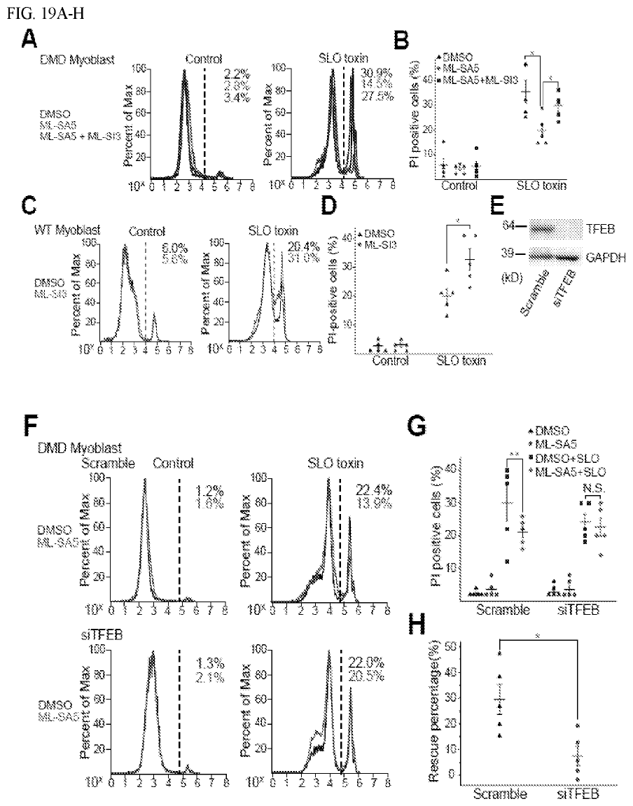

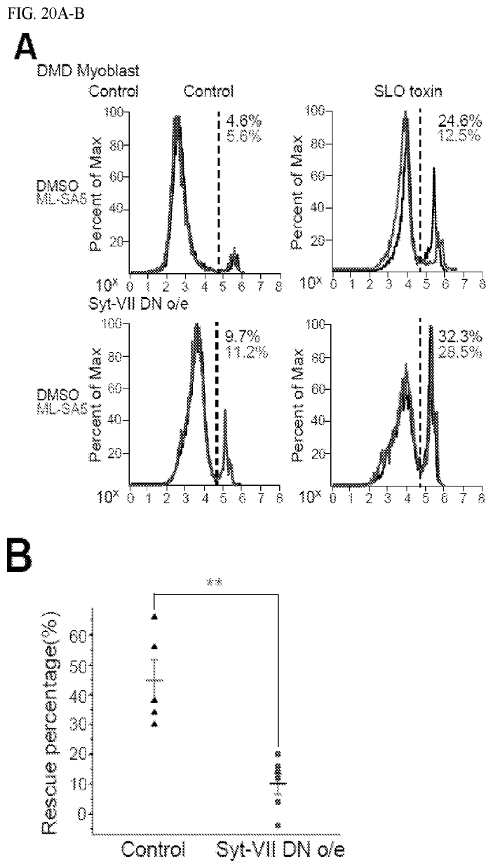

To study the role of ML1 activation in lysosome biogenesis directly, we utilized DMD cells, an immortalized myoblast line developed from a DMD patient's muscle cells (FIG. 16A, B) (33). Only mild Lamp1 upregulation was observed in DMD myoblasts (FIG. 16C), suggesting that the lysosome insufficiency occurring in mdx mice might be caused by extensive in vivo muscle damage. Consistent with the lysosomal patch-clamp studies (FIG. 7B), nanomolar concentrations of ML1 agonists were sufficient to induce striking nuclear translocation of TFEB (FIG. 16D, E) and the related protein TFE3 (FIG. 16F, G). Consistently, expression of TFEB/TFE3 target genes, including those required for lysosome biogenesis and function, were elevated by ML-SA5 treatment (FIG. 16H). Notably, TFEB mRNA and protein expressions were significantly reduced in ML-SA5-treated WT and DMD cells (FIG. 16H-K), suggestive of negative feedback regulation. Hence, ML1 activation may lead to TFEB nuclear translocation, thereby increasing lysosome biogenesis, which in turn reduces lysosome stress and restores lysosome homeostasis.

Whereas increased TFEB activation and lysosome biogenesis may amplify lysosomal trafficking events including lysosomal exocytosis (34), ML1 activation alone can trigger lysosomal exocytosis directly (35). Membrane damage [i.e., chemical injury caused by streptolysin O (SLO) toxin] triggers lysosomal exocytosis and membrane repair (26). In the myofibers, which contain large cytoplasm, ML1-mediated lysosomal Ca²⁺ release may preferentially activate lysosome-localized Ca²⁺ sensors to facilitate lysosomal exocytosis and biogenesis (FIG. 17A-E). Propidium iodide (PI) staining is a common readout for membrane damage and cell viability (36). In DMD myoblasts and differentiated myotubes, both membrane damage and ML-SA treatment induced lysosomal exocytosis (FIG. 18A, B), suggesting that ML-SAs may trigger membrane repair processes by mimicking a yet-to-be-identified damage signal. Consistent with this hypothesis, ML1 agonists and inhibitors decreased and increased, respectively, PI-positive cells following SLO treatment (FIG. 19A-D). The cytoprotective effects of ML-SAs were abolished by knock down of TFEB expression (FIG. 19E-H) or blockage of lysosomal exocytosis with dominant-negative Syt-VII (FIG. 20A, B). Collectively, these results suggest that boosting ML1 activity may promote myocyte survival by reducing membrane damage via a direct and acute mechanism (i.e., lysosomal exocytosis), as well as through an indirect and more sustained mechanism (i.e., lysosome biogenesis; FIG. 14I).

### Example 183

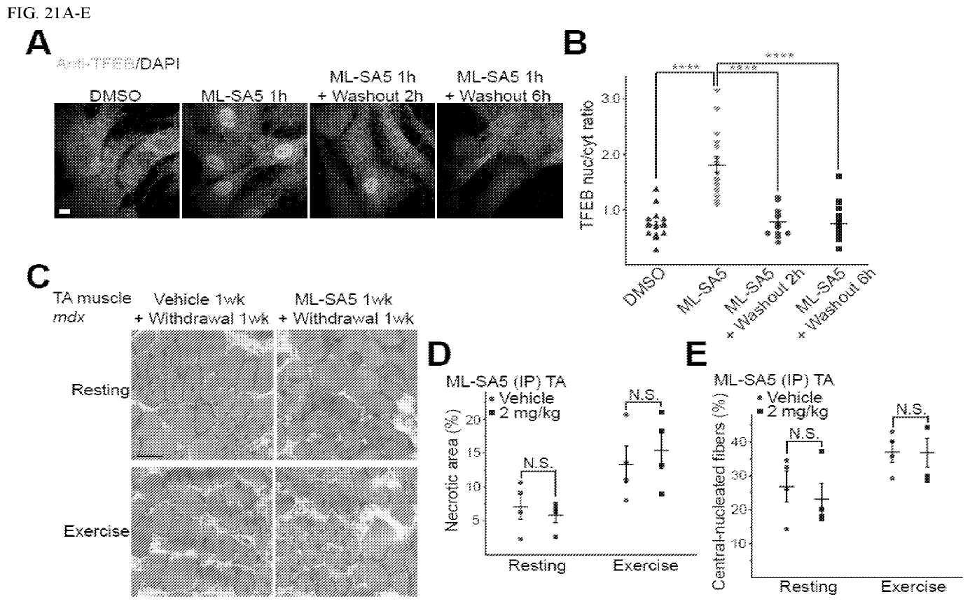

ML1 deficiency causes a DMD-like muscle phenotype in both humans and rodents (19). In the current study, we found that both genetic and pharmacological activation of ML1 had muscle protective effects in mdx mice and human DMD muscle cells. ML1 activation led to TFEB nuclear translocation and increased lysosomal biogenesis, while also increasing lysosomal trafficking (e.g., exocytosis), thereby facilitating sarcolemma repair to reduce muscle damage (FIG. 6I). Hence, this proof-of-concept study has provided strong evidence that small-molecule ML1 agonists can be developed to treat DMD. Upon washout of ML-SA compounds, the muscle protective effects of the drugs disappeared (see FIG. 21), indicating that sustained or periodic activation of lysosome enhancement is required for DMD treatment. Although DMD is not an LSD per se (28), the observed compensatory changes are suggestive of lysosome insufficiency. In both cases, augmenting ML1/TFEB-dependent lysosome biogenesis can alleviate lysosome insufficiency. Since ML1 is a ubiquitously expressed lysosomal channel, there could be “on-target” adverse effects of ML1 activation on non-muscle issues in mdx mice. However, ML1/TFEB activation is most effective in cells/tissues with lysosome dysfunction/insufficiency, and ML1/TFEB-mediated lysosome biogenesis is indeed suppressed by multiple mechanisms in healthy cells (28, 37). Hence, the “on-target”, “over-stimulating” effects of ML1 may be minimal on healthy cells/tissues/animals.

The simplest explanation for the presently observed muscle protective effect of ML1 would be facilitation of lysosomal exocytosis and membrane repair (FIG. 141). Consistent with this hypothesis, blockage of lysosomal exocytosis abolished ML1-dependent muscle protection. TFEB has been shown to be muscle protective in other studies as well (14). In LSDs, ML1 and TFEB form a positive feedback loop that promotes cellular clearance (28). It is possible that the same lysosome program may boost sarcolemma repair to reduce muscle damage in DMD and other muscle diseases. Hence, manipulating lysosome function with small molecules has broad therapeutic potential.

### Example 184

This example provides the materials and methods utilized in Examples 178-183.

Study design. The overall goal of this study is to determine the effect of ML1 upregulation on muscular dystrophy and membrane repair. All data presented here have been replicated in at least four mice or three biological replicates for in vitro experiments, with all histochemical staining data quantified by experimenters who were blind to genotypes/treatments. Based on pilot studies of ML1 upregulation on various pathologic hallmarks in mice, with a power of 0.8 and P<0.05, we calculated a sample size of between 5 and 11 mice per group. Animals were randomly allocated into control and experimental groups.

Mouse lines. We generated muscle cell-specific ML1-overexpressing (ML1 ROSA-lSl;MCK Cre or ML1^{MCK}) mice by crossing ROSA-loxSTOPlox-GCaMP3-ML1 (abbreviated ML1 ROSA-lSl) mice with a muscle cell-specific Cre line (MCK Cre) (15). Mdx (001801) and utrophin^{+/-};mdx (014563) mice were purchased from Jackson Laboratories. ML1−/− mice were kindly provided (19). All mice used in the study were backcrossed to the C₅₇BL/6 genetic background. Mice were used under the University of Michigan's Institutional Animal Care & Use Committee's approval. For cardiac function studies, only hemizygous male mice at the age of 13-15 months were used. For histological and physiological studies of skeletal muscles, both hemizygous male and homozygous female mdx mice were randomly assigned into different groups. The muscle type and age of the mice used for each experiment were indicated in the figures and legends.

Western blotting. After lysing muscle tissues in ice-cold RIPA buffer with 1× protease inhibitor cocktail tablet (Roche), 20-40 μg of total protein aliquots were loaded into 4-12% Bis-Tris or 3-8% Tris-acetate sodium dodecyl sulfate-polyacrylamide gradient gels (Invitrogen) and then transferred to polyvinylidene fluoride membranes via iBlot 2 Gel Transfer Device (Life Technologies). The membranes were blocked in 5% (w/v) skim milk in PBS with 0.05% Tween20 for 1 h, and then incubated overnight in the blocking buffer at 4° C. The primary antibodies used were: anti-ML1 (ACC-081, Alamone lab), anti-green fluorescent protein (GFP) (A6455, Invitrogen), anti-mouse Lamp1 (1D4B, DSHB), anti-human Lamp1 (H₄A3, DSHB), anti-mouse TFEB (A303-673A, Bethyl), anti-human TFEB (4240, Cell Signaling), anti-LC3 (L8918, Sigma), anti-dystrophin (ab15277, Abcam), anti-utrophin (8A4, DSHB), anti-α-dystroglycan (sc-53987, Santa Cruz), and anti-GAPDH (glyceraldehyde 3-phosphate dehydrogenase)(MAB347, Millipore). Peroxidase-conjugated anti-rabbit, anti-mouse, or anti-rat secondary antibodies were applied at room temperature for 1 h, followed by Super-Signal West Pico Chemiluminescence Substrate (Thermo Scientific). Bands were quantitated in ImageJ software.

Immunofluorescence. Muscle tissues, harvested and frozen in 2-methylbutane, pre-chilled in liquid nitrogen, were cryo-sectioned at 12 μm. After being washed with Tris buffered saline (TBS)+0.025% Triton X, sections were blocked with 10% serum and 1% bovine serum albumin in TBS at RT for 2 h. Cell lines and isolated primary cells cultured on coverslips were washed with PBS, fixed in 4% paraformaldehyde, permeabilized in 0.3% Triton X in PBS, and blocked in 1% bovine serum albumin in PBS. Fixed cells and cryosections were then incubated at 4° C. overnight with primary antibodies targeting ML1, GFP, Lamp1, TFEB, dystrophin, or CD11b (M1/70.15.11.5.2, DSHB) at 1:50 or 1:200 solutions. Alexa Fluor secondary antibodies (Invitrogen) were then applied for 1 h in the dark at RT followed by DAPI (4′,6-diamidino-2-phenylindole) counterstaining if necessary. Cells and tissue sections were imaged on a spinning disk confocal imaging system composed of an Olympus IX81 inverted microscope, 10×, 20× and 60× Olympus objectives, a CSU-X1 scanner (Yokogawa), an iXon EM-CCD camera (Andor), and MetaMorph Advanced Imaging acquisition software v.7.7.8.0 (Molecular Devices). Image analysis results were quantified in MetaMorph software.

Primary muscle cell culture. Murine myoblasts were harvested and cultured as previously described (38). Briefly, skeletal muscles were isolated from postnatal day 0-3 pups and dissociated with 0.25% trypsin supplemented with 2 mg/mL collagenase (Sigma) at 37° C. To remove fibroblasts, cells were pre-plated on a standard, non-tissue culture-coated Petri dish for 60-90 min. Muscle cells were then counted, seeded onto collagen-coated glass coverslips, and maintained at 37° C. under 5% CO₂ in F10 medium supplemented with 20% fetal bovine serum (FBS) and penicillin/streptomycin. To induce differentiation, myoblasts were grown to confluence before switching to Dulbecco's modification of Eagle medium (DMEM) containing 5% horse serum and penicillin/streptomycin. Electrophysiology, Ca²⁺ imaging, and immunofluorescence labeling were performed after 3 d of differentiation induction to allow MCK-Cre expression.

Whole-endolysosome electrophysiology. Endolysosomal electrophysiology was performed on isolated endolysosomes from muscle cells treated with 1 μM vacuolin-1 to enlarge late endosomes and lysosomes (39, 40). The bath (internal/cytoplasmic) solution contained 140 mM K-gluconate, 4 mM NaCl, 1 mM EGTA, 2 mM Na₂-ATP, 2 mM MgCl₂, 0.39 mM CaCl₂, 0.2 mM GTP, and 10 mM HEPES [pH adjusted with KOH to 7.2; the free [Ca²⁺]ᵢ was estimated to be ˜100 nM on Maxchelator software (http://maxchelator.stanford.edu/)]. The pipette (luminal) solution consisted of a low-pH Tyrode's solution with 145 mM NaCl, 5 mM KCl, 2 mM CaCl₂), 1 mM MgCl₂, 10 mM HEPES, 10 mM MES, and 10 mM glucose (pH 4.6). A perfusion system was used to ensure efficient solution exchange. Data were collected with an Axopatch 2A patch clamp amplifier, Digidata 1440, and pClamp 10.0 software (Axon Instruments). Currents were digitized at 10 kHz and filtered at 2 kHz. All experiments were conducted at 21-23° C., and all recordings were analyzed in pClamp 10.0 and Origin 8.0 (OriginLab, Northampton, Mass.).

GCaMP3 Ca²⁺ imaging. Ca²⁺ imaging was performed in ML1^{MCK} and mdx;ML1^{MCK} primary myotubes overexpressing GCaMP3-ML1 (15). The fluorescence intensity at 488 nm (F₄₈₈) was recorded with the spinning disk confocal imaging system.

Histochemical staining. Frozen sections were warmed to RT and hydrated in double distilled (MilliQ) H₂O. Tissues were stained with H&E dyes (Fisher) for the nucleus and cytoplasm, respectively, followed by gradient washing in ethanol (70%, 90%, 100%) and xylene (100%). The stained sections were mounted in Permount medium (Fisher). Staining quantification was performed blindly following standardized operating procedures (SOP number: DMD_M.1.2.007). Necrotic myofibers, inflammatory cells and fibroblasts were all counted as necrotic area. Collagen content was assessed with a Masson's Trichrome Kit (Sigma). Area stained with blue color was quantified as fibrotic area.

Muscle force measurement. Mice were anesthetized by i.p. injection of Avertin (tribromoethanol, 250 mg/kg) and placed on a warmed platform to maintain body temperature. GAS muscle contractile properties were measured in situ, as described previously (41). Briefly, after the GAS was isolated from surrounding tissues, the distal tendon was secured to the lever arm of a servomotor (model 6650LR, Cambridge Technology). The knee and foot were clamped to the platform. The muscle was activated by tibial nerve stimulation. With the muscle held at optimal length (L₀), 300-ms trains of stimulus pulses were applied at increasing frequencies until the maximum isometric tetanic force (P₀) was achieved. After the pre-stretch P₀ (preP₀) was recorded, we applied two stretches (12 s apart) of 30% of fiber length (L_{f}), which was estimated by multiplying L₀ by previously determined L_{f}-to-L₀ ratios. After a 1-min rest, post-stretch P₀ (postP₀) was obtained to determine the force deficit. To maintain muscle temperature and moisture, a warm saline drip (37° C.) was applied to the GAS continuously throughout the procedure. After the experiment, mice were euthanized, and muscles were removed and trimmed of their tendons. Physiological cross-sectional areas were determined by dividing muscle mass by the product of L_{f} using a density of mammalian skeletal muscle of 1.06 g/cm³. Specific P₀ (SP₀) refers to P₀ per cross-sectional area and the force deficit was calculated as 1−postP₀/preP₀.

Echocardiography. Echocardiograms were performed by following the recommendations of the American Society of Echocardiography as described previously (42). All echocardiography experiments were performed by one registered echocardiographer who was blind to the mouse genotype. Prior to imaging, 13-15 months old WT, mdx, mdx; ML1^{MCK} mice were weighed and anesthetized with inhaled isoflurane. Imaging was performed using a Vevo 770 Microimaging system (VisualSonics Inc.) equipped with an RMV707B (15-45 MHz) transducer. Left ventricular (LV) wall thickness was measured at end systole and end diastole. Mitral valve E and A wave inflow velocities were sampled at the tips of the mitral valve leaflets from the apical four-chamber view. LV mass (LVM) was calculated using the formula LVM=1.053*[(LVID;d+LVPW;d+IVS;d)³−LVID;d³] with the LV internal diameter (LVID), the LV posterior wall thickness (LVPW), and interventricular septal thickness (IVS) being measured during diastole (d) from the parasternal short-axis view at the tip levels of the mitral valve leaflets.

Injection of small-molecule compounds. At P14, mdx mice were weighed and randomized into treatment groups. ML-SA compounds (dissolved in 10% DMSO, 40% PEG300, and 50% PBS), were administered to mice by i.p. injection. After 14 d of daily injection, the mice were subjected to various behavioral tests or sacrificed for histological and biochemical analyses. For Evan's blue staining in the cardiac muscle and toxicity studies, drug injection was extended for 1 more month until the mice reached 2 mo. old. To induce cardiomyopathy in 2-month-old mdx mice, 0.5 mg/kg P-isoproterenol (Sigma) was injected subcutaneously 1 d before sacrificing (43). ML-SA and ML-SI compounds were identified initially by Ca²⁺-imaging-based high-throughput screening conducted at NIH/NCATS Chemical Genomics Center (pubchem.ncbi.nlm.nih.gov/bioassay/624414), and their potencies were improved by medicinal chemistry. All ML-SA and ML-SI compounds are available upon request under MTA with the University of Michigan.

Treadmill exercise. Mice were trained on an Exer-6M treadmill (Columbus Instruments). Prior to running, they were acclimated to the treadmill chamber for 30 min. To induce damage, P28 mice ran on the treadmill with a 150 downgrade at 12-15 m/min for 30 min 1 d before being sacrificed for analysis (27). To measure exhaustion time, the treadmill was set to a speed of 12-20 m/min.

Uptake of EB dye. Freshly prepared 1% EB dye (Sigma) w/v in PBS was injected i.p. at 10 ml/kg 1 d before tissue collection.

Measurement of CK activity. Venous tail blood was collected before and after treadmill exercise, and serum was separated by centrifugation. Serum CK activity was measured with a Creatine Kinase Activity Assay Kit (Abcam) following the manufacturer's instructions.

Complete blood count and liver biochemistry. Venous tail blood was collected in the EDTA tubes and sent to a lab test for complete blood count at room temperature immediately after collection. For liver biochemistry, serum was separated by centrifugation and stored at −80° C. until sent for analysis.

Myofiber damage assay. After mice were sacrificed, FDB muscles were removed surgically and then digested in 2 mg/mL type I collagenase (Sigma) at 37° C. for 60 min. After trituration and re-suspension in FluoroBrite DMEM solution (Thermo Fisher), single FDB fibers were mounted on a glass bottom dish precoated with Matrigel (Coming) and then stained with 5 g/mL FM 4-64 dye (Thermo Fisher) immediately before imaging. A Leica SP5 inverted confocal microscope system with a multiphoton laser (laser power 2.3 W at wavelength 820 nm) was used to irradiate the fibers and take images. Fluorescence intensity at the injury site was measured by ImageJ software.

Muscle cell line culture. Immortalized human myoblast cell lines, including a WT line (ref. AB1079C₃₈Q) and a DMD line (ref. AB1023 DMD11Q: stop in exon 59), were provided by Dr. Vincent Mouly at the Institut de Myologie in France (44). Myoblasts were grown in KMEM (1 volume of medium-199+4 volumes of DMEM, both from Thermo Fisher) supplemented with 20% FBS, fetuin (25 μg/mL, Sigma), insulin (5 μg/mL, Sigma), basic fibroblast growth factor (0.5 ng/mL, Thermo Fisher), human epidermal growth factor (5 ng/mL, Thermo Fisher), and dexamethasone (0.2 μg/mL, Sigma) in a humidified CO₂ incubator at 37° C. Myoblasts were induced to differentiate into myotubes by switching to medium containing DMEM with 50 μg/mL gentamycin and 10 μg/mL insulin.

RNA extraction and real time quantitative (qRT)-polymerase chain reaction (PCR).

Total RNA was extracted from cultured human myoblasts with TRIzol (Invitrogen) and then purified with a Turbo DNA-free kit (Invitrogen). cDNA was then synthesized with a Superscript III RT kit (Invitrogen). We conducted qRT-PCR with PowerUp SYBR green 2× master mix (Invitrogen) and the following PCR primers (32):

HPRT:
forward (fw):
(SEQ ID NO.: 1)
5′-tggcgtcgtgattagtgatg-3′,
reverse (rev):
(SEQ ID NO.: 2)
5′-aacaccctttccaaatcctca-3′;
PGC1α:
fw:
(SEQ ID NO.: 3)
5′-catgcaaatcacaatcacagg-3′,
rev:
(SEQ ID NO.: 4)
5′-ttgtggcttagctgttgac-3′;
MCOLN1:
fw:
(SEQ ID NO.: 5)
5′-gagtgggtgcgacaagtttc-3′,
rev:
(SEQ ID NO.: 6)
5′-tgttctcttcccggaatgtc-3′;
ATP6V0E1:
fw:
(SEQ ID NO.: 7)
5′-cattgtgatgagcgtgttctgg-3′,
rev:
(SEQ ID NO.: 8)
5′-aactccccggttaggaccctta-3′;
ATP6V1H:
fw:
(SEQ ID NO.: 9)
5′-ggaagtgtcagatgatcccca-3′,
rev:
(SEQ ID NO.: 10)
5′-ccgtttgcctcgtggataat-3′;
CTSF:
fw:
(SEQ ID NO.: 11)
5′-acagaggaggagttccgcacta-3′,
rev:
(SEQ ID NO.: 12)
5′-gcttgcttcatcttgttgcca-3′;
TFEB:
fw:
(SEQ ID NO.: 13)
5′-caaggccaatgacctggac-3′,
rev:
(SEQ ID NO.: 14)
5′-agctccctggactitigcag-3′;
CTSD:
fw:
(SEQ ID NO.: 15)
5′-cttcgacaacctgatgcagc-3′,
rev:
(SEQ ID NO.: 16)
5′-tacttggagtctgtgccacc-3′;
PPP3CA:
fw:
(SEQ ID NO.: 17)
5′-gctgccctgatgaaccaac-3′,
rev:
(SEQ ID NO.: 18)
5′-gcaggtggttctttgaatcgg-3′;
DPP7:
fw:
(SEQ ID NO.: 19)
5′-gattcggaggaacctgagtg-3′,
rev:
(SEQ ID NO.: 20)
5′-cggaagcaggatcttctgg-3′;
CTSB:
fw:
(SEQ ID NO.: 21)
5′-agtggagaatggcacacccta-3′,
rev:
(SEQ ID NO.: 22)
5′-aagagccattgtcacccca-3′;
TPP1:
fw:
(SEQ ID NO.: 23)
5′-gatcccagctctcctcaatac-3′,
rev:
(SEQ ID NO.: 24)
5′-gccatttttgcaccgtgtg-3′;
NEU1:
fw:
(SEQ ID NO.: 25)
5′-tgaagtgtttgcccctggac-3′,
rev:
(SEQ ID NO.: 26)
5′-aggcaccatgatcatcgctg-3′.

Silencing RNA (siRNA) knockdown. TFEB expression was transfected with siRNA oligonucleotides (5′-gaaaggagacgaagguucaacauca-3′(SEQ ID NO.: 27)) and Lipofectamine 2000 (both from Invitrogen). Cells were subjected to biochemical and cell biological analyses 72 h after transfection.

Flow cytometry PI staining. Cells were pretreated with various compounds, digested in accutase, and then washed with Tyrode's solution supplemented with 20% FBS. After counting, cell membranes were damaged with SLO toxin (1 μg/ml, 10 min) at 37° C. His-tagged SLO (carrying a cysteine deletion that eliminates the need for thiol activation (36) was provided by R. Tweeten (University of Oklahoma, Norman, Okla.) and purified as described previously (19). SLO-treated cells were stained with 2 μg/ml PI (Thermo Fisher) for 5 min and analyzed by Attune N×T Acoustic Focusing Cytometer (Life Technologies). Total cell events were measured with Vybrant DyeCycle Violet stain (Invitrogen). At least 10,000 cells were used for each experimental group. Data were analyzed in Attune N×T software.

Lamp1 surface labeling. After 3 d of differentiation, immortalized human myotubes pretreated with ML-SAs were incubated with 0.5-1 μg/mL SLO at 37° C. for 30 min. Non-permeablized cells were labelled with anti-human Lamp1 (H₄A3) antibody, which recognizes a luminal epitope, at 4° C. for 1 h. Cells were then fixed in 2% paraformaldehyde for 30 min, and incubated with Alexa-488 conjugated secondary antibody (Invitrogen) at RT for 1h.

Statistical analysis. Data are presented as the means±standard errors of the mean (s.e.m.). Statistical comparisons were performed with analyses of variance (ANOVAs) and Turkey's post hoc tests or with paired and unpaired Student's t-tests where appropriate. A value <0.05 was considered statistically significant.

### INCORPORATION BY REFERENCE

The entire disclosure of each of the patent documents and scientific articles referred to herein is incorporated by reference for all purposes. The following references are herein incorporated by reference in their entireties:
1. E. Mercuri, F. Muntoni, Muscular dystrophies. Lancet 381, 845-860 (2013).
2. K. E. Davies, K. J. Nowak, Molecular mechanisms of muscular dystrophies: old and new players. Nat Rev Mol Cell Biol 7, 762-773 (2006).
3. F. Rahimov, L. M. Kunkel, The cell biology of disease: cellular and molecular mechanisms underlying muscular dystrophy. J Cell Biol 201, 499-510 (2013).
4. D. G. Allen, N. P. Whitehead, S. C. Froehner, Absence of Dystrophin Disrupts Skeletal Muscle Signaling: Roles of Ca2+, Reactive Oxygen Species, and Nitric Oxide in the Development of Muscular Dystrophy. Physiol Rev 96, 253-305 (2016).
5. K. P. Campbell, Three muscular dystrophies: loss of cytoskeleton-extracellular matrix linkage. Cell 80, 675-679 (1995).
6. H. H. Stedman et al., The mdx mouse diaphragm reproduces the degenerative changes of Duchenne muscular dystrophy. Nature 352, 536-539 (1991).
7. L. Amoasii et al., Single-cut genome editing restores dystrophin expression in a new Mouse model of muscular dystrophy. Sci Transl Med 9, (2017).
8. D. Bansal et al., Defective membrane repair in dysferlin-deficient muscular dystrophy. Nature 423, 168-172 (2003).
9. C. Cai et al., MG53 nucleates assembly of cell membrane repair machinery. Nat Cell Biol 11, 56-64 (2009).
10. P. McNeil, Membrane repair redux: redox of MG53. Nat Cell Biol 11, 7-9 (2009).
11. E. Mills, X. P. Dong, F. Wang, H. Xu, Mechanisms of brain iron transport: insight into neurodegeneration and CNS disorders. Future Med Chem 2, 51-64 (2010).
12. D. Shen et al., Lipid storage disorders block lysosomal trafficking by inhibiting a TRP channel and lysosomal calcium release. Nat Commun 3, 731 (2012).
13. X. Cheng et al., The intracellular Ca(2)(+) channel MCOLN1 is required for sarcolemma repair to prevent muscular dystrophy. Nat Med 20, 1187-1192 (2014).
14. C. Spampanato et al., Transcription factor EB (TFEB) is a new therapeutic target for Pompe disease. FMBO Mol Med 5, 691-706 (2013).
15. N. Sahoo et al., Gastric Acid Secretion from Parietal Cells Is Mediated by a Ca(2+) Efflux Channel in the Tubulovesicle. Dev Cell 41, 262-273 e266 (2017).
16. J. C. Bruning et al., A muscle-specific insulin receptor knockout exhibits features of the metabolic syndrome of NIDDM without altering glucose tolerance. Molecular cell 2, 559-569 (1998).
17. M. S. Clarke, R. Khakee, P. L. McNeil, Loss of cytoplasmic basic fibroblast growth factor from physiologically wounded myofibers of normal and dystrophic muscle. J Cell Sci 106 (Pt 1), 121-133 (1993).
18. X. Zhang et al., MCOLN1 is a ROS sensor in lysosomes that regulates autophagy. Nat Commun 7, 12109 (2016).
19. X. Cheng et al., The intracellular Ca(2+) channel MCOLN1 is required for sarcolemma repair to prevent muscular dystrophy. Nat Med 20, 1187-1192 (2014).
20. V. Straub, J. A. Rafael, J. S. Chamberlain, K. P. Campbell, Animal models for muscular dystrophy show different patterns of sarcolemmal disruption. J Cell Biol 139, 375-385 (1997).
21. R. M. Grady et al., Skeletal and cardiac myopathies in mice lacking utrophin and dystrophin: a model for Duchenne muscular dystrophy. Cell 90, 729-738 (1997).
22. J. G. Quinlan et al., Evolution of the mdx mouse cardiomyopathy: physiological and morphological findings. Neuromuscul Disord 14, 491-496 (2004).
23. D. Danilowicz, M. Rutkowski, D. Myung, D. Schively, Echocardiography in duchenne muscular dystrophy. Muscle Nerve 3, 298-303 (1980).
24. C. M. Adamo et al., Sildenafil reverses cardiac dysfunction in the <em>mdx</em> mouse model of Duchenne muscular dystrophy. Proceedings of the National Academy of Sciences 107, 19079 (2010).
25. W. Wang et al., Up-regulation of lysosomal TRPML1 channels is essential for lysosomal adaptation to nutrient starvation. Proc Natl Acad Sci USA 112, E1373-1381 (2015).
26. X. Cheng, X. Zhang, L. Yu, H. Xu, Calcium signaling in membrane repair. Semin Cell Dev Biol 45, 24-31 (2015).
27. H. Radley-Crabb et al., A single 30 min treadmill exercise session is suitable for ‘proof-of concept studies’ in adult mdx mice: a comparison of the early consequences of two different treadmill protocols. Neuromuscul Disord 22, 170-182 (2012).
28. D. Xu H. & Ren, Lysosomal Physiology. Annual Review in Physiology 74, (2014).
29. R. Pal et al., Src-dependent impairment of autophagy by oxidative stress in a mouse model of Duchenne muscular dystrophy. Nat Commun 5, 4425 (2014).
30. S. Duguez et al., Dystrophin deficiency leads to disturbance of LAMP1-vesicle-associated protein secretion. Cellular and molecular life sciences: CMLS 70, 2159-2174 (2013).
31. J. G. Tidball, Inflammatory processes in muscle injury and repair. Am J Physiol Regul Integr Comp Physiol 288, R345-353 (2005).
32. D. L. Medina et al., Lysosomal calcium signalling regulates autophagy through calcineurin and TFEB. Nat Cell Biol 17, 288-299 (2015).
33. A. Sarathy et al., SU9516 Increases alpha7beta1 Integrin and Ameliorates Disease Progression in the mdx Mouse Model of Duchenne Muscular Dystrophy. Mol Ther 25, 1395-1407 (2017).
34. D. L. Medina et al., Transcriptional activation of lysosomal exocytosis promotes cellular clearance. Dev Cell 21, 421-430 (2011).
35. M. Samie et al., A TRP channel in the lysosome regulates large particle phagocytosis via focal exocytosis. Dev Cell 26, 511-524 (2013).
36. V. Idone et al., Repair of injured plasma membrane by rapid Ca2+-dependent endocytosis. J Cell Biol 180, 905-914 (2008).
37. P. Li, M. Gu, H. Xu, Lysosomal Ion Channels as Decoders of Cellular Signals. Trends Biochem Sci 44, 110-124 (2019).
38. M. L. Springer, T. A. Rando, H. M. Blau, Gene delivery to muscle. Curr Protoc Hum Genet Chapter 13, Unit 13 14 (2002).
39. X. P. Dong et al., The type IV mucolipidosis-associated protein TRPML1 is an endolysosomal iron release channel. Nature 455, 992-996 (2008).
40. X. P. Dong et al., PI(3,5)P(2) Controls Membrane Traffic by Direct Activation of Mucolipin Ca Release Channels in the Endolysosome. Nat Commun 1, (2010).
41. L. M. Larkin et al., Skeletal muscle weakness due to deficiency of CuZn-superoxide dismutase is associated with loss of functional innervation. Am J Physiol Regul Integr Comp Physiol 301, R1400-1407 (2011).
42. M. O. Boluyt, K. Converso, H. S. Hwang, A. Mikkor, M. W. Russell, Echocardiographic assessment of age-associated changes in systolic and diastolic function of the female F344 rat heart. J Appl Physiol (1985) 96, 822-828 (2004).
43. Y. Yue, J. W. Skimming, M. Liu, T. Strawn, D. Duan, Full-length dystrophin expression in half of the heart cells ameliorates β-isoproterenol-induced cardiomyopathy in mdx mice. Human Molecular Genetics 13, 1669-1675 (2004).
44. K. Mamchaoui et al., Immortalized pathological human myoblasts: towards a universal tool for the study of neuromuscular disorders. Skeletal Muscle 1, 34 (2011).
45. A. G. Garrity et al., The endoplasmic reticulum, not the pH gradient, drives calcium refilling of lysosomes. Elife 5, (2016).

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A compound encompassed within one of the following formulas: 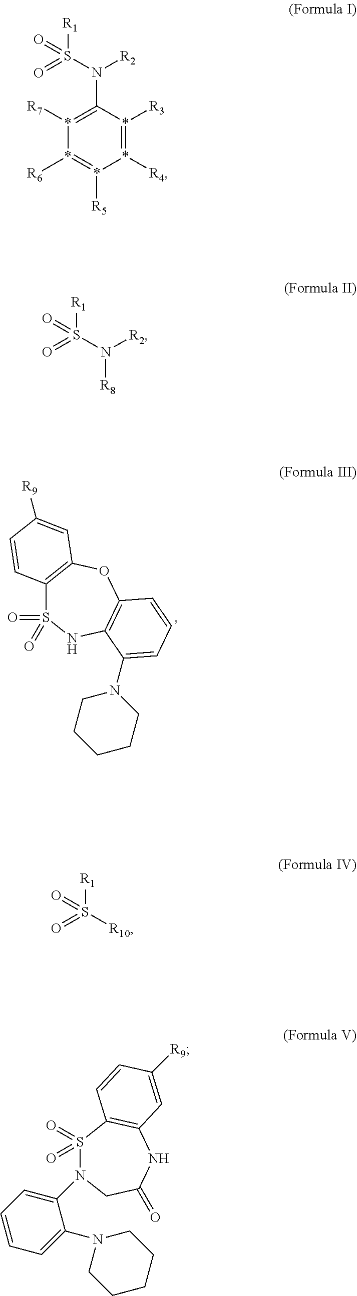
including pharmaceutically acceptable salts, solvates, and/or prodrugs thereof,
wherein the particular chemical moiety for R1, R2, R3, R4, R5, R6, R7 R8, R9, R10, and * independently include any chemical moiety that permits the resulting compound to have one or more of the following properties:
a capability to improve aberrant membrane repair capability related to diminished ML1 activity;
a capability to improve aberrant lysosomal exocytosis activity related to diminished ML1 activity;
a capability to improve aberrant lysosomal Ca²⁺ release channel activity related to diminished ML1 activity (required for lysosomal exocytosis);
a capability to repair damaged sarcolemma related to diminished ML1 activity;
a capability to improve aberrant Ca²⁺ dependent delivery of lysosomal membranes to damaged sarcolemma related to diminished ML1 activity;
a capability to improve aberrant lysosomal activity related to diminished ML1 activity;
a capability to repair muscle damage related to diminished ML1 activity;
a capability to ameliorate myofiber necrosis related to diminished ML1 activity;
a capability to ameliorate central nucleation related to diminished ML1 activity;
a capability to ameliorate fibrosis related to diminished ML1 activity;
a capability to ameliorate elevated serum creatine kinase (CK) levels related to diminished ML1 activity;
a capability to ameliorate reduced muscle force related to diminished ML1 activity;
a capability to ameliorate impaired motor ability related to diminished ML1 activity; and
a capability to restore lysosome function via activation of transcriptional factor EB (TFEB).

2. The compound of claim 1, wherein each “*” is independently either carbon or nitrogen.

3. The compound of claim 1, wherein at least one “*” is nitrogen, and the remainder are carbon.

4. The compound of claim 1, wherein each “*” is carbon.

5. The compound of claim 1, wherein R1 is selected from hydrogen,

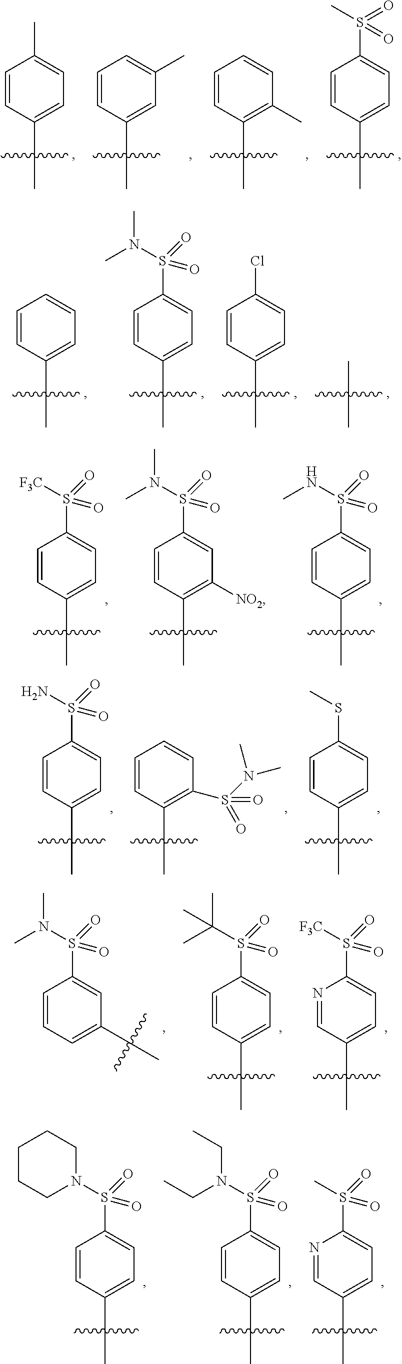

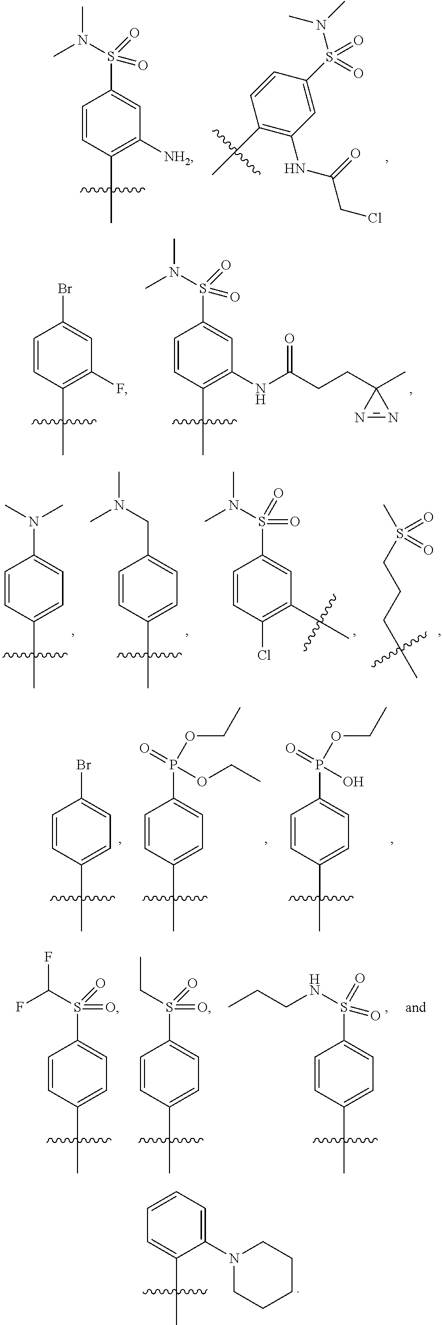

6. The compound of claim 1, wherein R2 is selected from hydrogen,

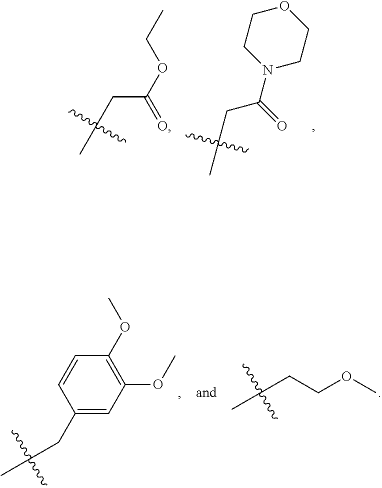

7. The compound of claim 1, wherein R3, R4, R5, R6 and R7 are each independently selected from hydrogen, Chlorine, Fluorine, CH₃,

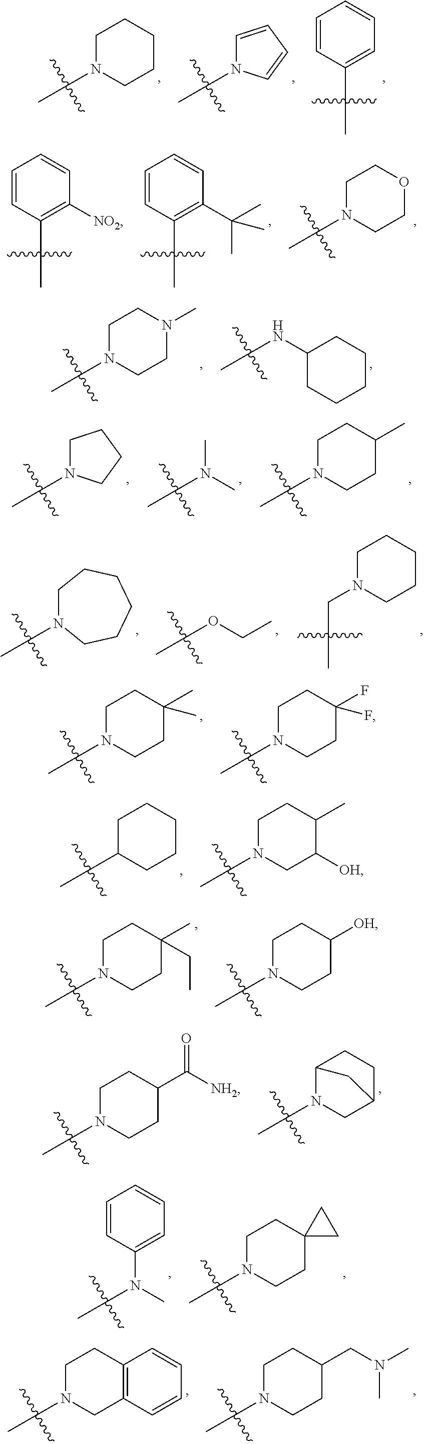

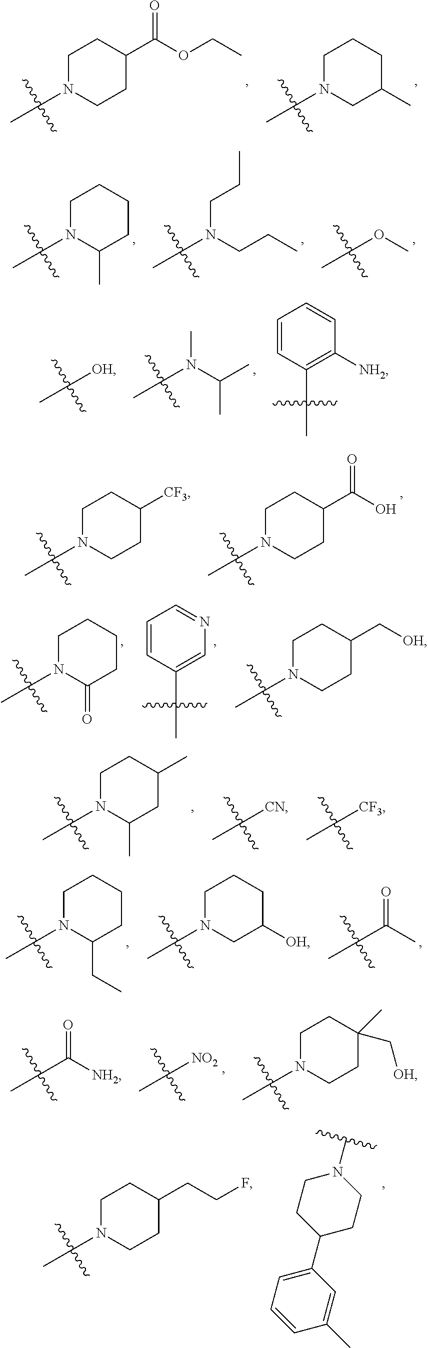

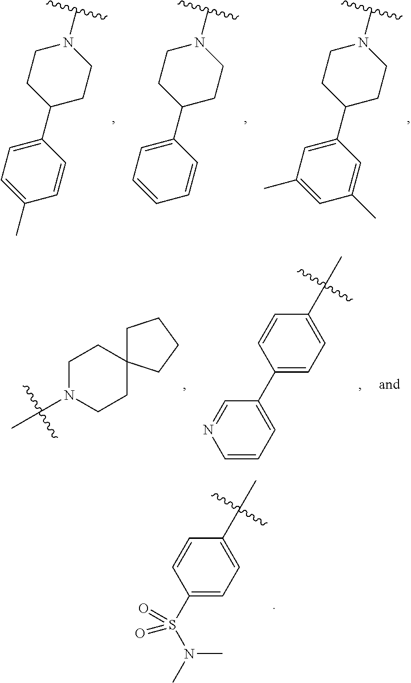 [SMILES: *N1CCC(c2ccc(C)cc2)CC1 ; *N1CCC(c2ccccc2)CC1 ; *N1CCC(c2cc(C)cc(C)c2)CC1 ; *N1CCC2(CCCC2)CC1 ; *c1ccc(-c2cccnc2)cc1 ; *c1ccc(S(=O)(=O)N(C)C)cc1]

8. The compound of claim 7, wherein at least two of R3, R4, R5, R6 and R7 are hydrogen.

9. The compound of claim 7, wherein at least three of R3, R4, R5, R6 and R7 are hydrogen.

10. The compound of claim 7, wherein at least four of R3, R4, R5, R6 and R7 are hydrogen.

11. The compound of claim 1, wherein R8 is selected from hydrogen,

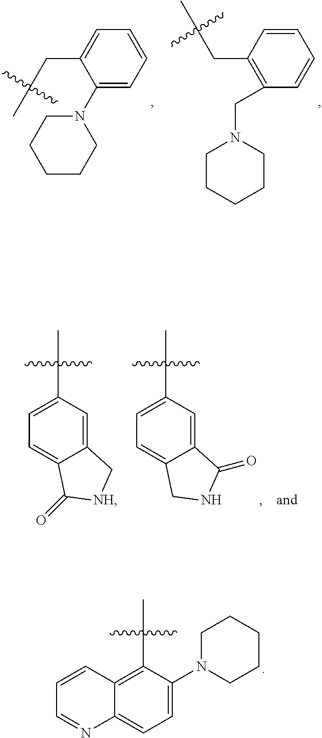

12. The compound of claim 1, wherein R9 is selected from hydrogen, 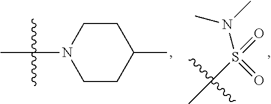
Bromine,

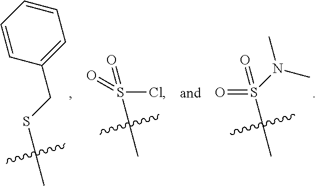

13. The compound of claim 1, wherein R10 is selected from hydrogen,

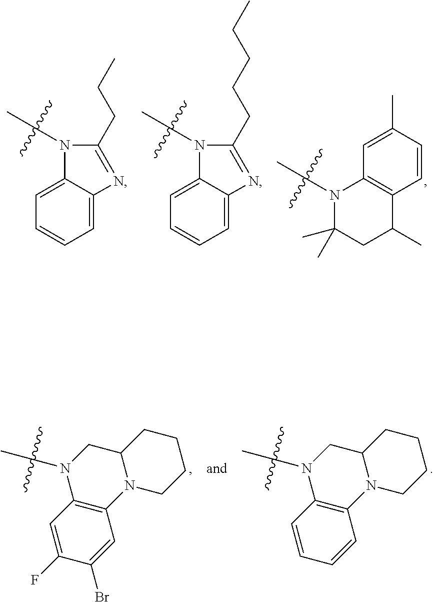 [SMILES: *n1c(CCC)nc2ccccc21 ; *n1c(CCCCC)nc2ccccc21 ; *N1c2cc(C)ccc2C(C)CC1(C)C ; *N1CC2CCCCN2c2cc(Br)c(F)cc21 ; *N1CC2CCCCN2c2ccccc21]

14. The compound of claim 1, wherein said compound is selected from the group consisting of:

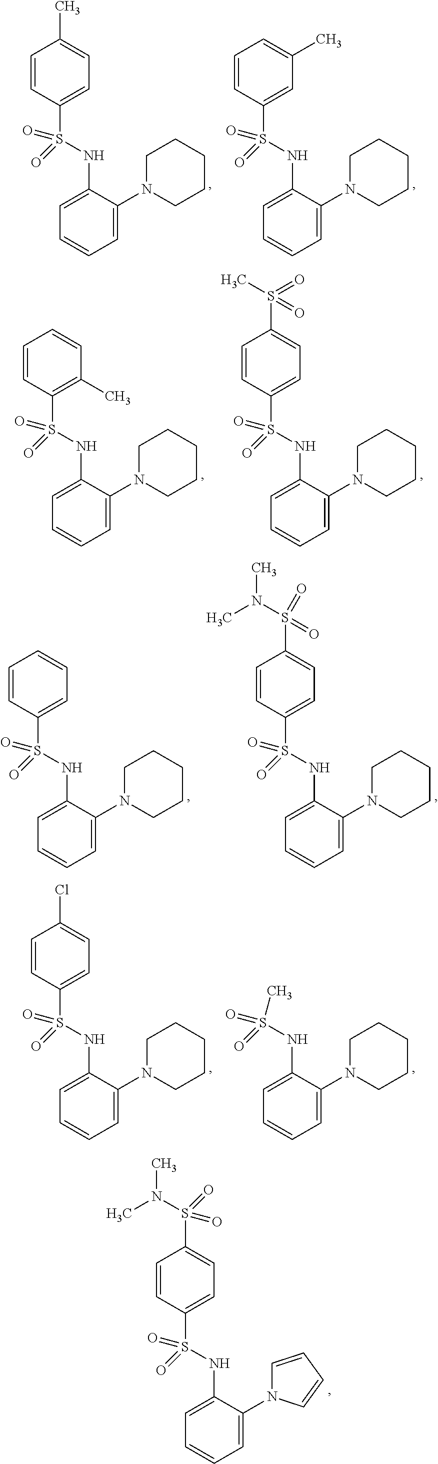

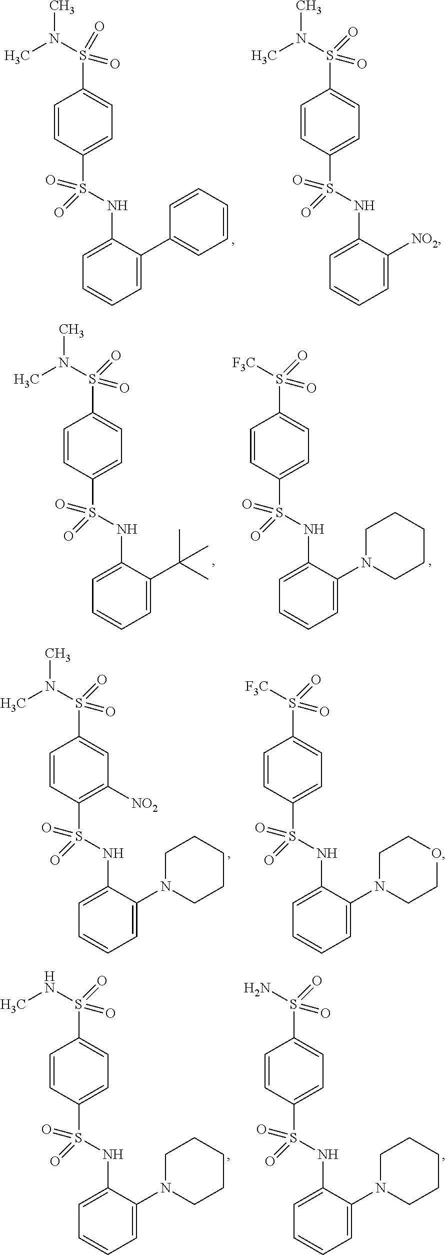

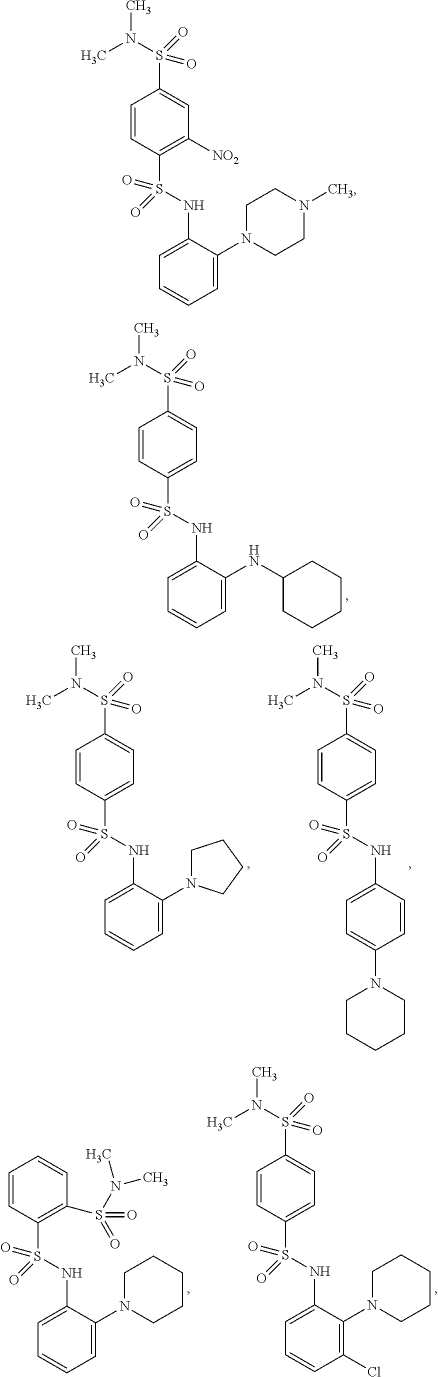

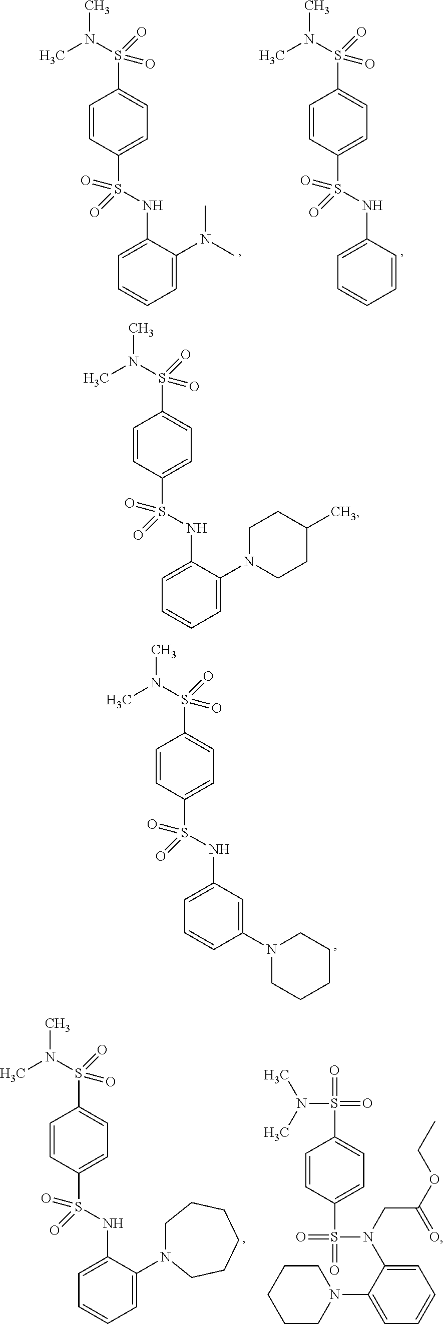

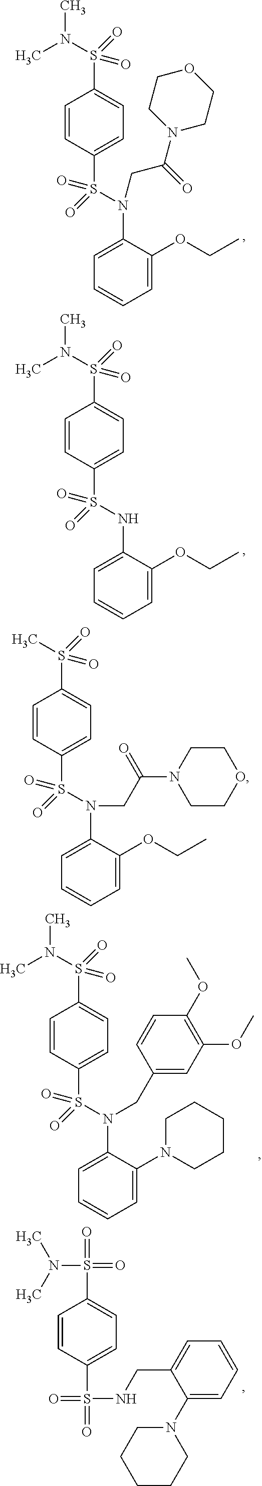

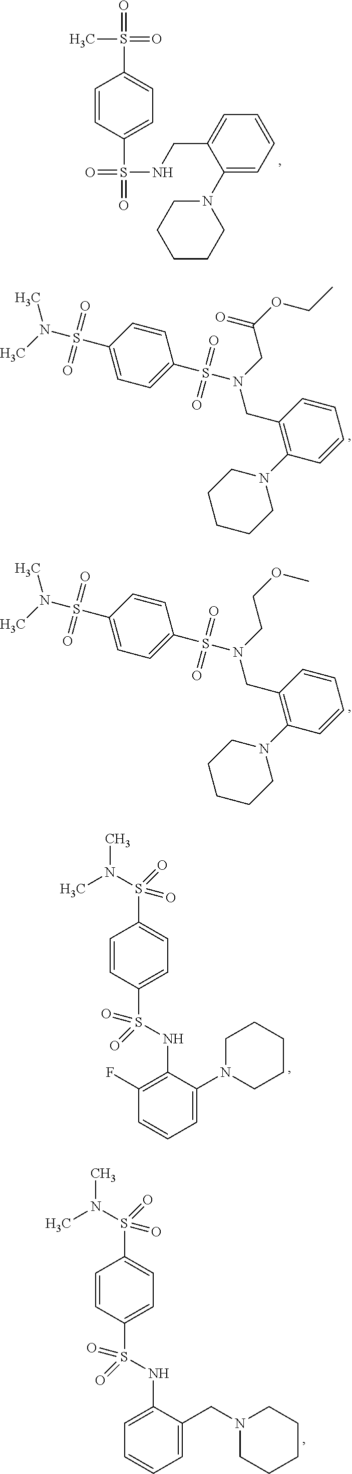 [SMILES: CS(=O)(=O)c1ccc(S(=O)(=O)NCc2ccccc2N2CCCCC2)cc1 ; CCOC(=O)CN(Cc1ccccc1N1CCCCC1)S(=O)(=O)c1ccc(S(=O)(=O)N(C)C)cc1 ; COCCN(Cc1ccccc1N1CCCCC1)S(=O)(=O)c1ccc(S(=O)(=O)N(C)C)cc1 ; CN(C)S(=O)(=O)c1ccc(S(=O)(=O)Nc2c(F)cccc2N2CCCCC2)cc1 ; CN(C)S(=O)(=O)c1ccc(S(=O)(=O)Nc2ccccc2CN2CCCCC2)cc1]

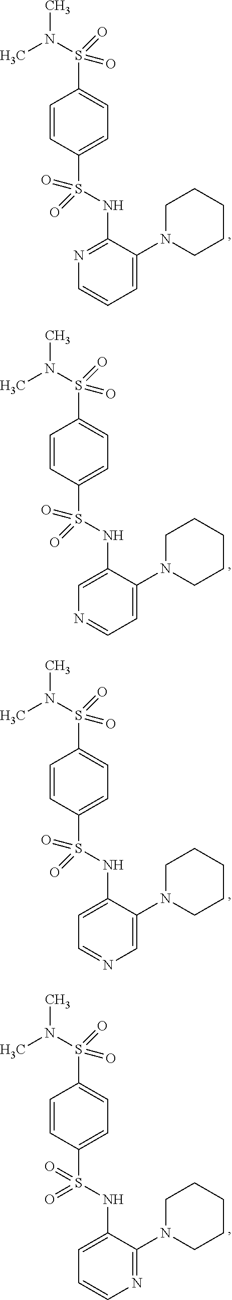 [SMILES: CN(C)S(=O)(=O)c1ccc(S(=O)(=O)Nc2ncccc2N2CCCCC2)cc1 ; CN(C)S(=O)(=O)c1ccc(S(=O)(=O)Nc2cnccc2N2CCCCC2)cc1 ; CN(C)S(=O)(=O)c1ccc(S(=O)(=O)Nc2ccncc2N2CCCCC2)cc1 ; CN(C)S(=O)(=O)c1ccc(S(=O)(=O)Nc2cccnc2N2CCCCC2)cc1]

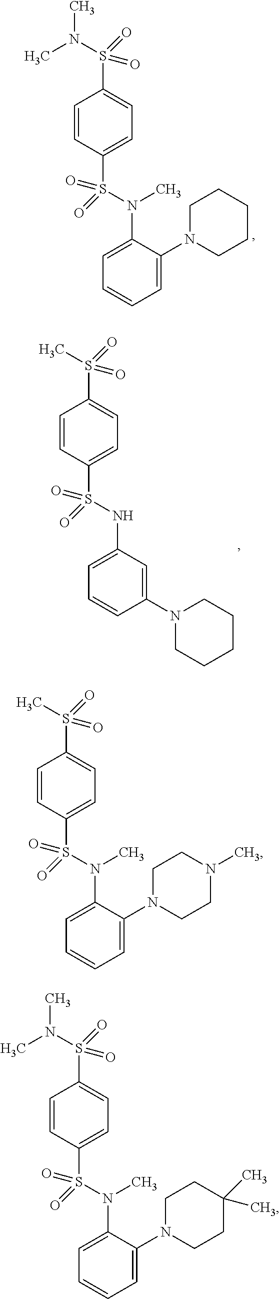

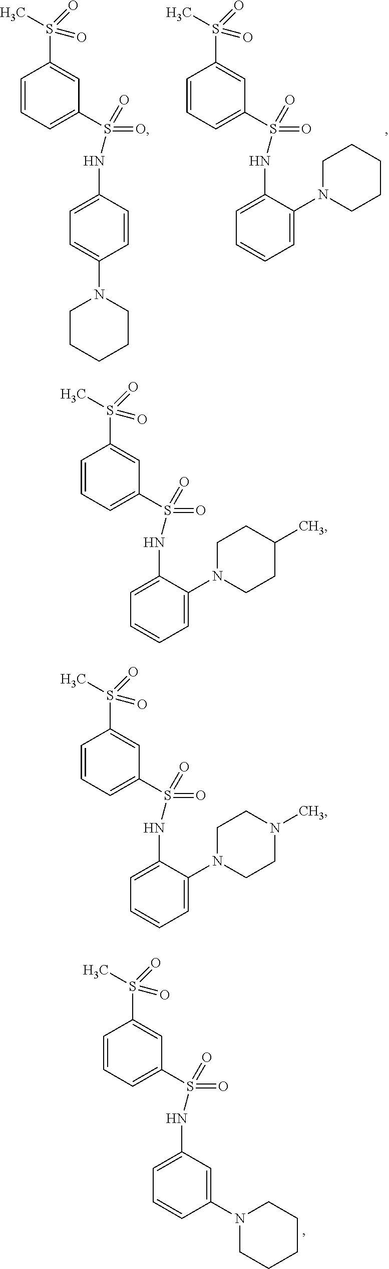

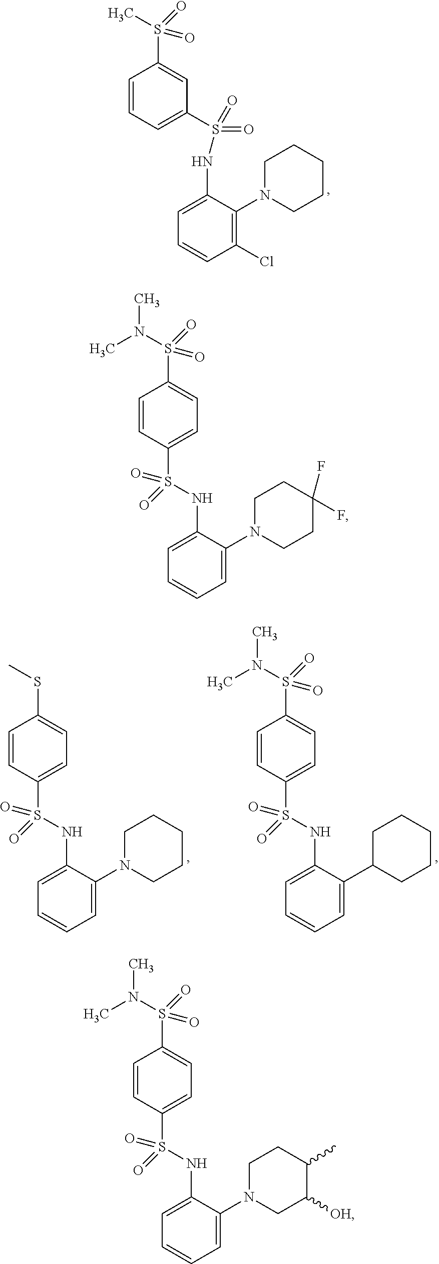 [SMILES: CS(=O)(=O)c1cccc(S(=O)(=O)Nc2cccc(Cl)c2N2CCCCC2)c1 ; CN(C)S(=O)(=O)c1ccc(S(=O)(=O)Nc2ccccc2N2CCC(F)(F)CC2)cc1 ; CSc1ccc(S(=O)(=O)Nc2ccccc2N2CCCCC2)cc1 ; CN(C)S(=O)(=O)c1ccc(S(=O)(=O)Nc2ccccc2C2CCCCC2)cc1 ; CC1CCN(c2ccccc2NS(=O)(=O)c2ccc(S(=O)(=O)N(C)C)cc2)CC1O]

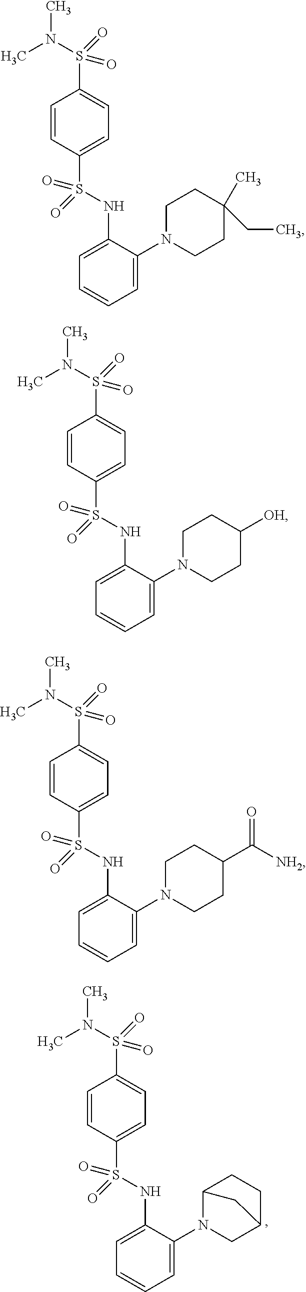

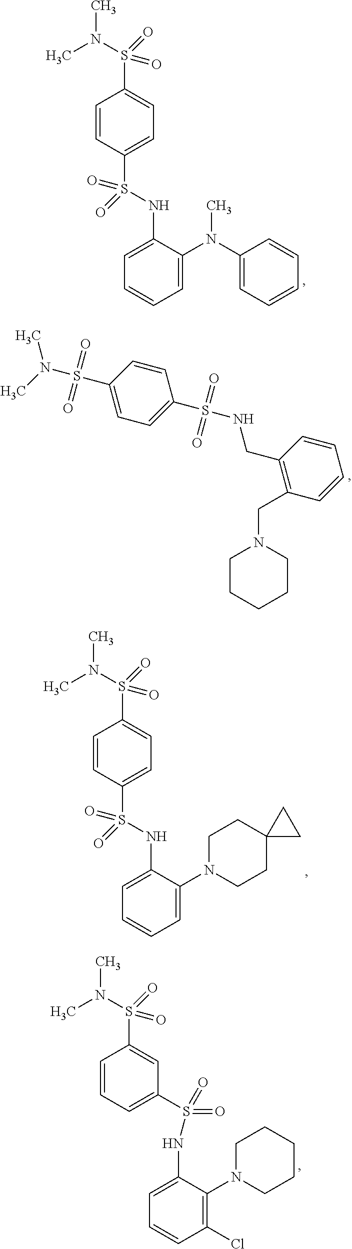 [SMILES: CN(c1ccccc1)c1ccccc1NS(=O)(=O)c1ccc(S(=O)(=O)N(C)C)cc1 ; CN(C)S(=O)(=O)c1ccc(S(=O)(=O)NCc2ccccc2CN2CCCCC2)cc1 ; CN(C)S(=O)(=O)c1ccc(S(=O)(=O)Nc2ccccc2N2CCC3(CC2)CC3)cc1 ; CN(C)S(=O)(=O)c1cccc(S(=O)(=O)Nc2cccc(Cl)c2N2CCCCC2)c1]

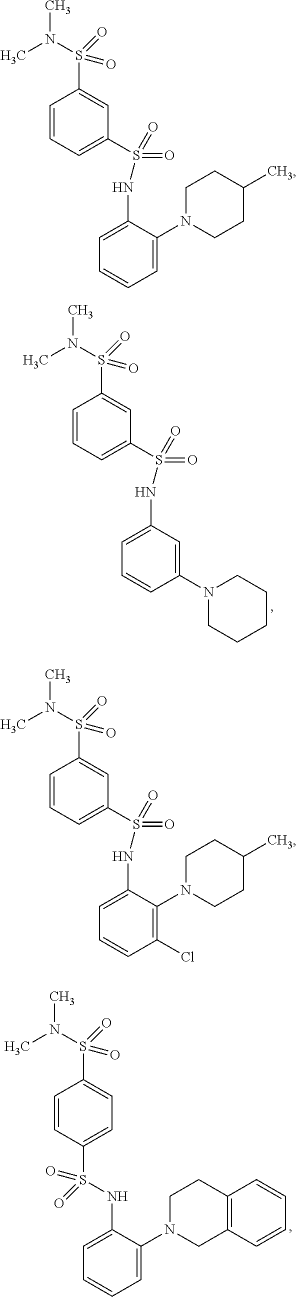 [SMILES: CC1CCN(c2ccccc2NS(=O)(=O)c2cccc(S(=O)(=O)N(C)C)c2)CC1 ; CN(C)S(=O)(=O)c1cccc(S(=O)(=O)Nc2cccc(N3CCCCC3)c2)c1 ; CC1CCN(c2c(Cl)cccc2NS(=O)(=O)c2cccc(S(=O)(=O)N(C)C)c2)CC1 ; CN(C)S(=O)(=O)c1ccc(S(=O)(=O)Nc2ccccc2N2CCc3ccccc3C2)cc1]

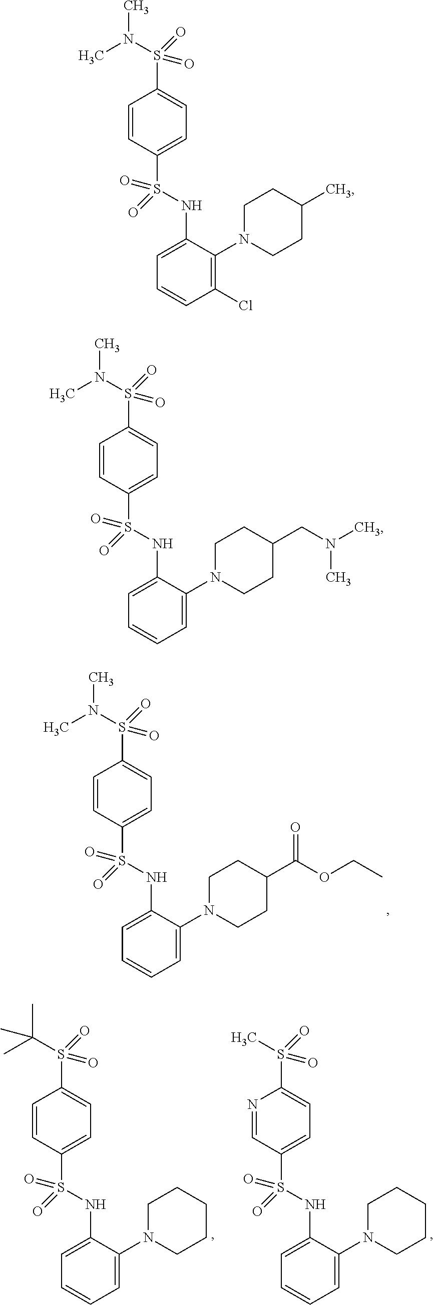

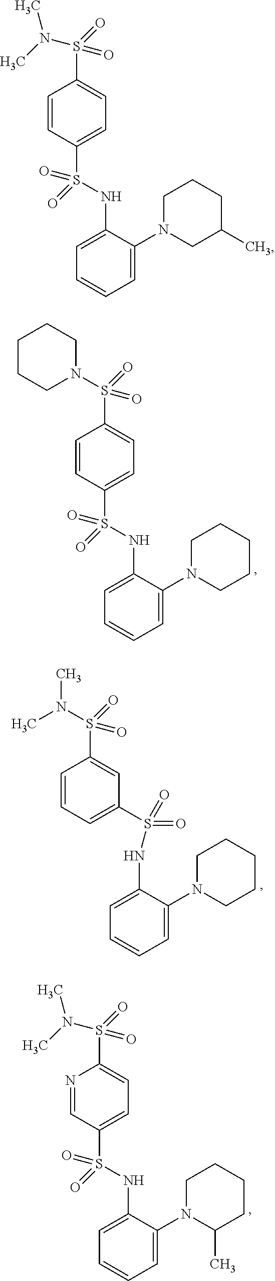 [SMILES: CC1CCCN(c2ccccc2NS(=O)(=O)c2ccc(S(=O)(=O)N(C)C)cc2)C1 ; O=S(=O)(Nc1ccccc1N1CCCCC1)c1ccc(S(=O)(=O)N2CCCCC2)cc1 ; CN(C)S(=O)(=O)c1cccc(S(=O)(=O)Nc2ccccc2N2CCCCC2)c1 ; CC1CCCCN1c1ccccc1NS(=O)(=O)c1ccc(S(=O)(=O)N(C)C)nc1]

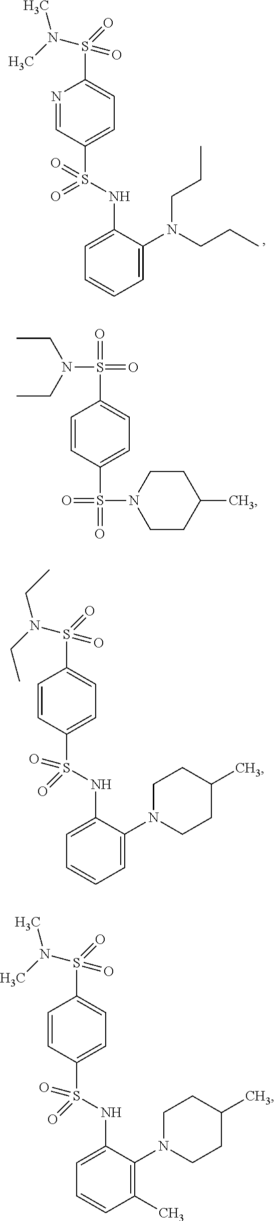

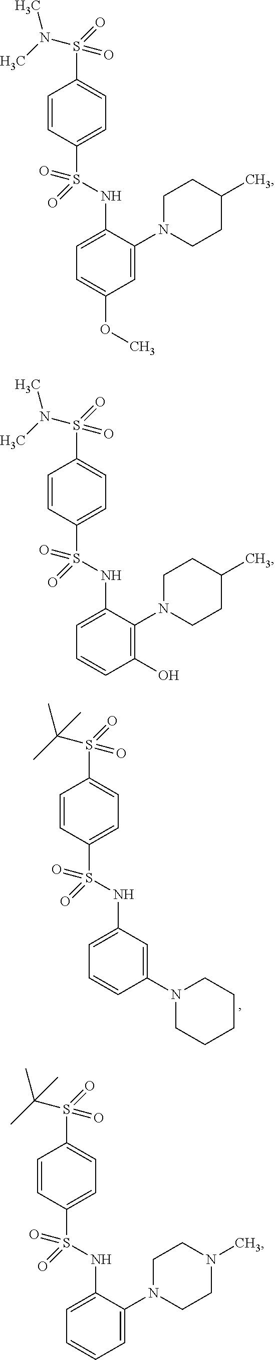

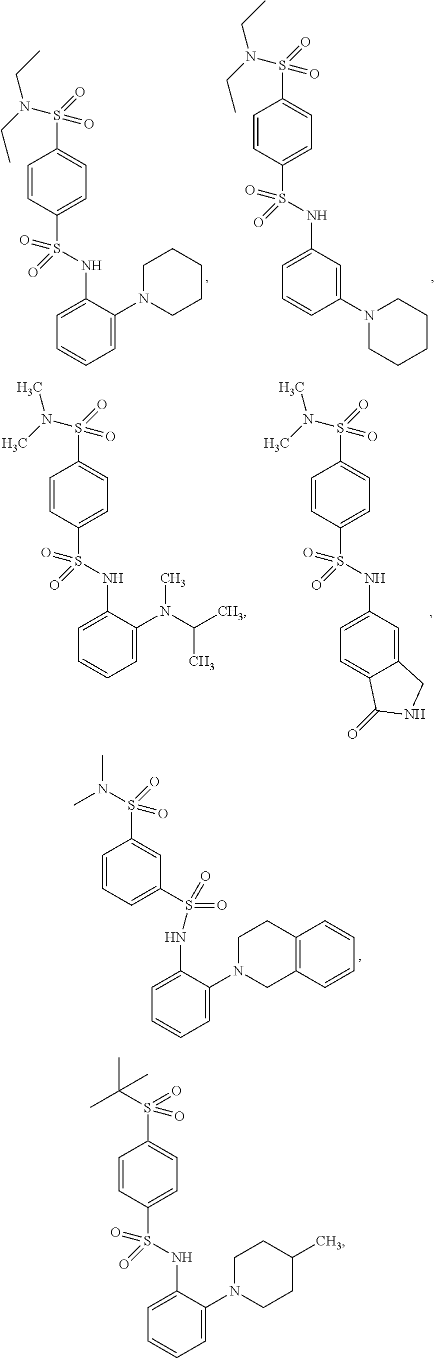

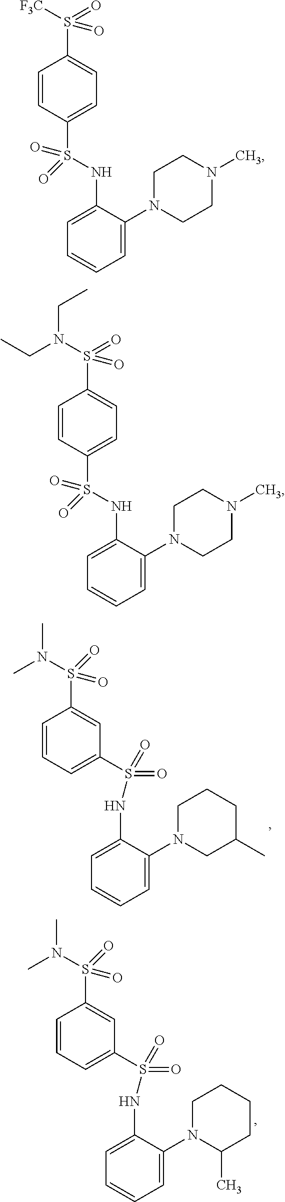

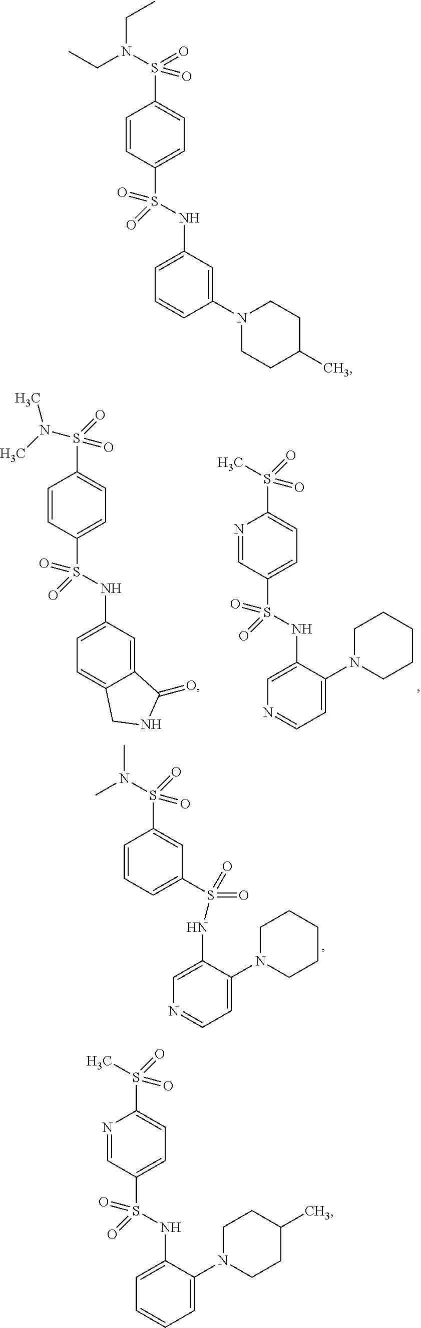

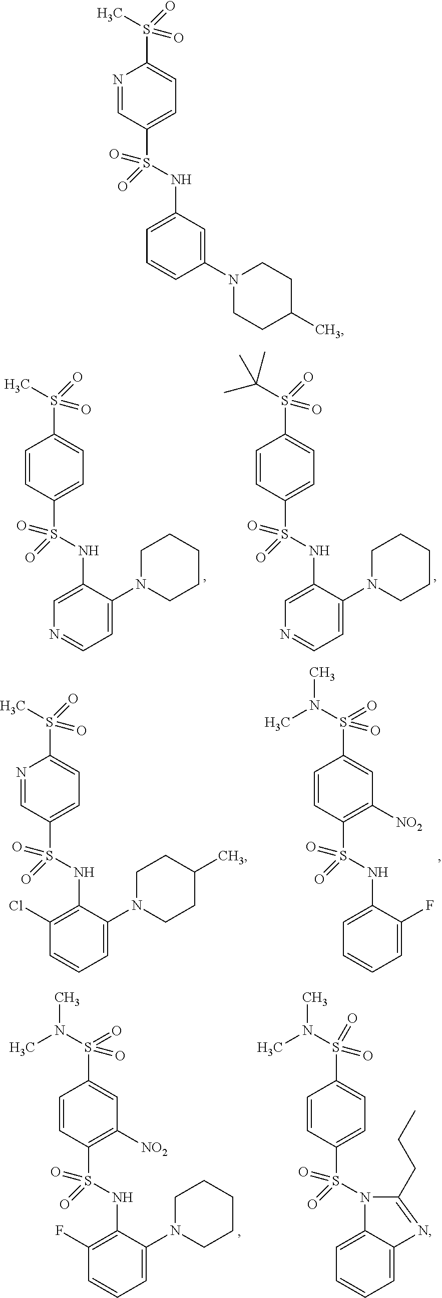

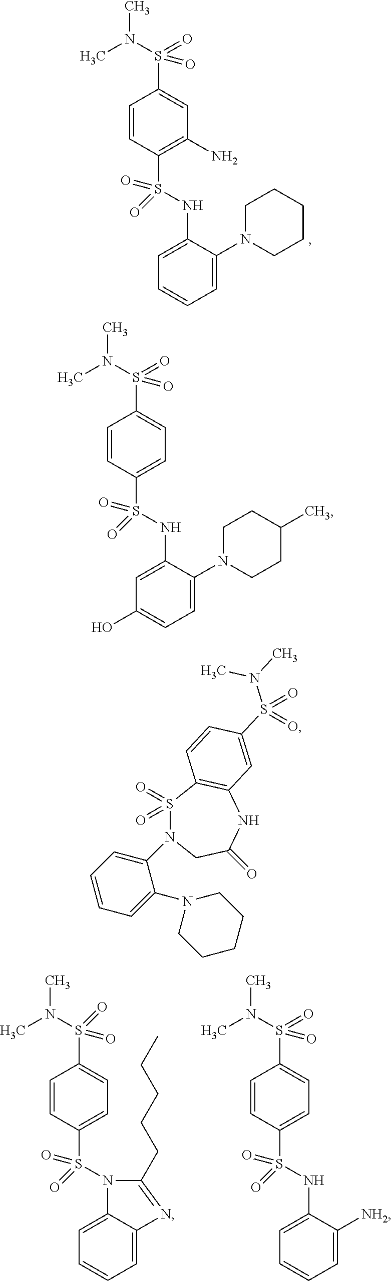

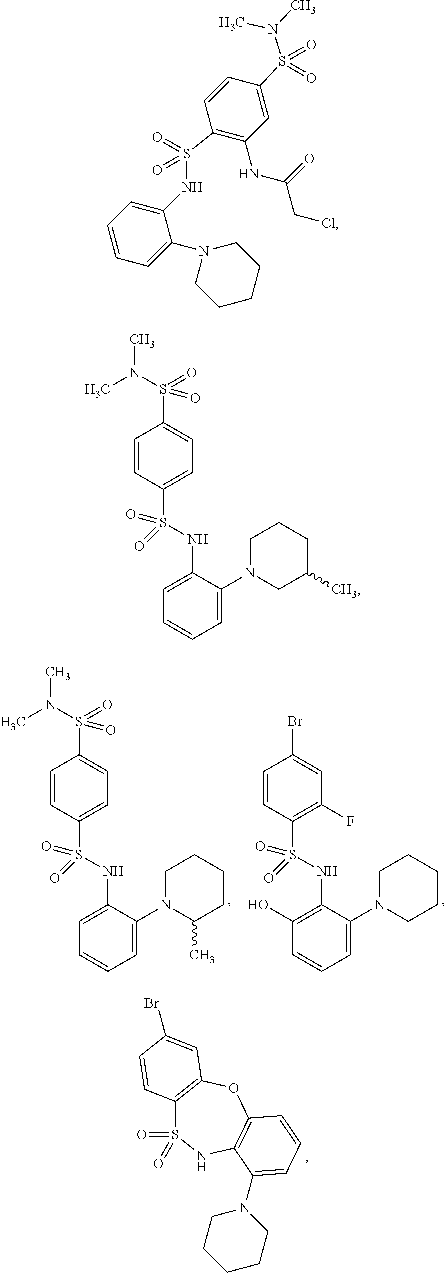

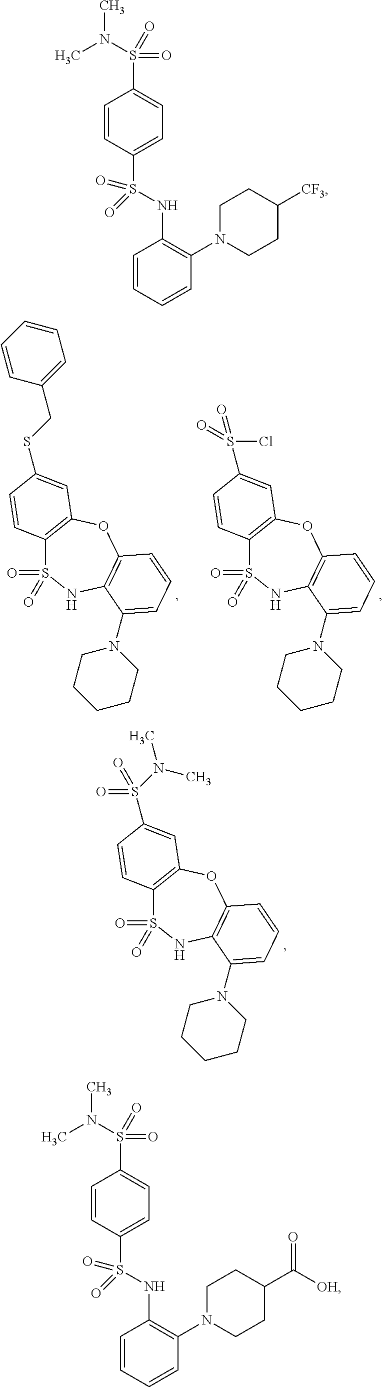

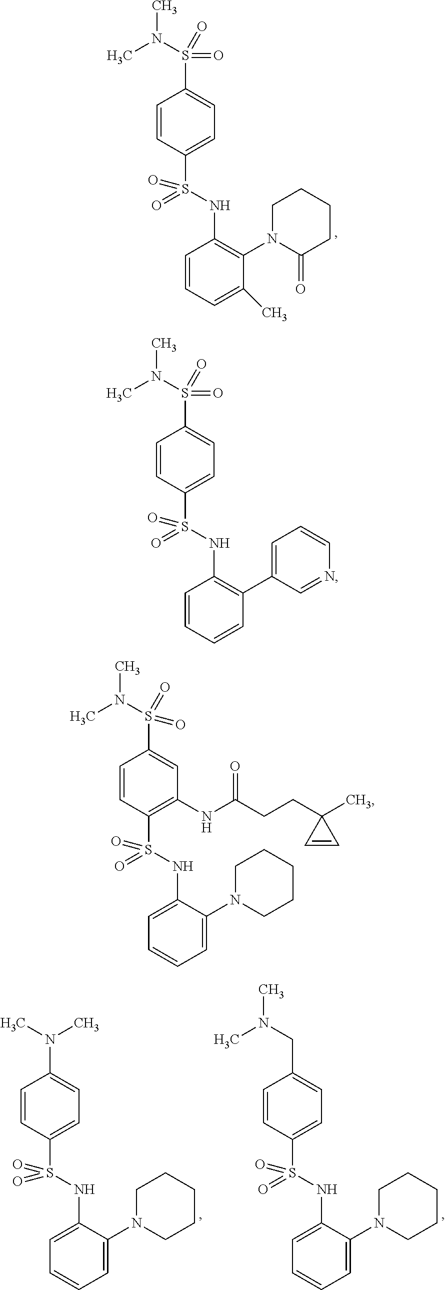 [SMILES: Cc1cccc(NS(=O)(=O)c2ccc(S(=O)(=O)N(C)C)cc2)c1N1CCCCC1=O ; CN(C)S(=O)(=O)c1ccc(S(=O)(=O)Nc2ccccc2-c2cccnc2)cc1 ; CN(C)S(=O)(=O)c1ccc(S(=O)(=O)Nc2ccccc2N2CCCCC2)c(NC(=O)CCC2(C)C=C2)c1 ; CN(C)c1ccc(S(=O)(=O)Nc2ccccc2N2CCCCC2)cc1 ; CN(C)Cc1ccc(S(=O)(=O)Nc2ccccc2N2CCCCC2)cc1]

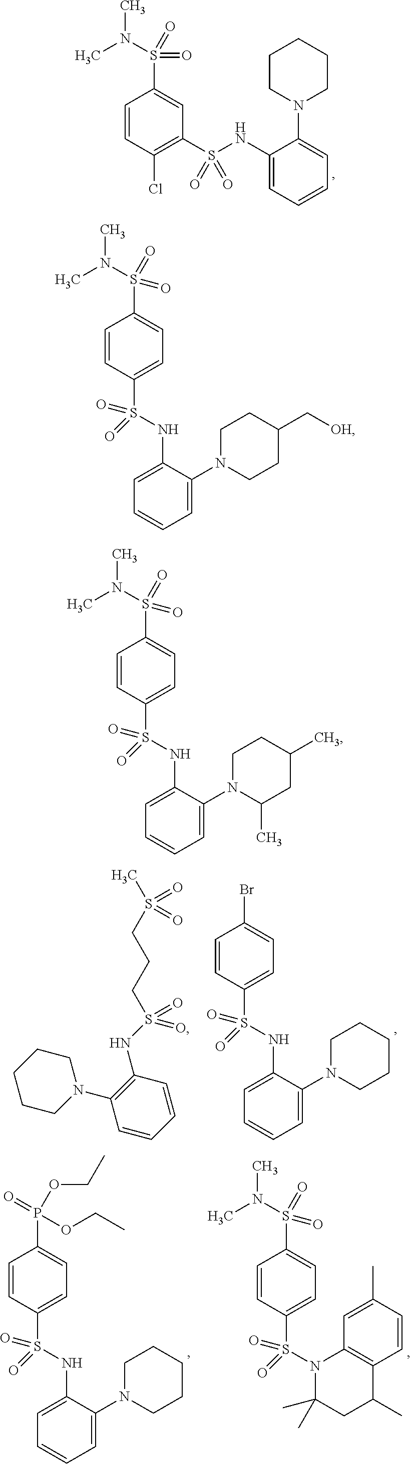

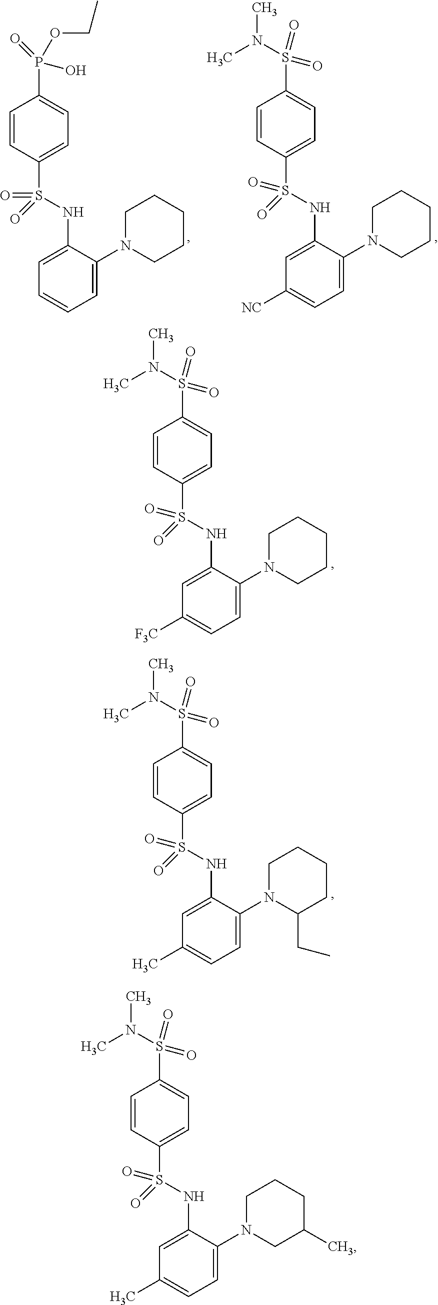

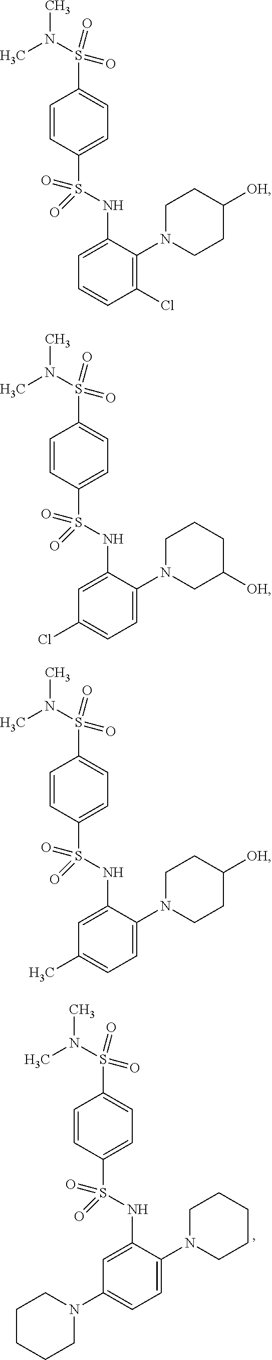

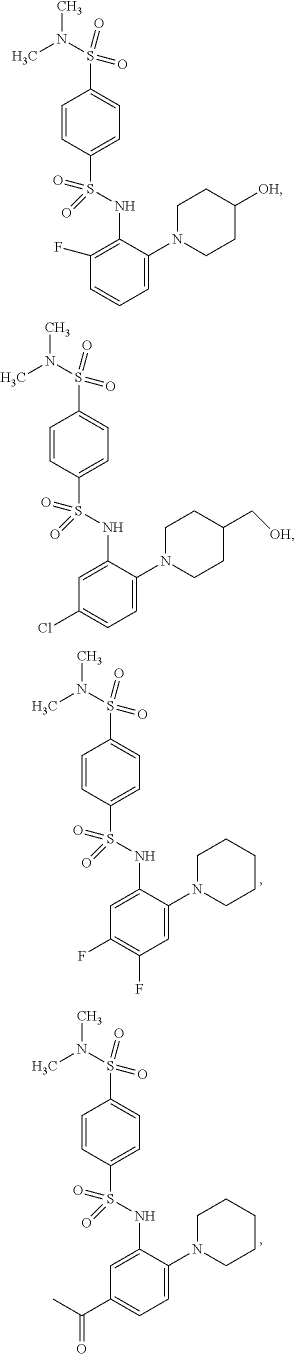

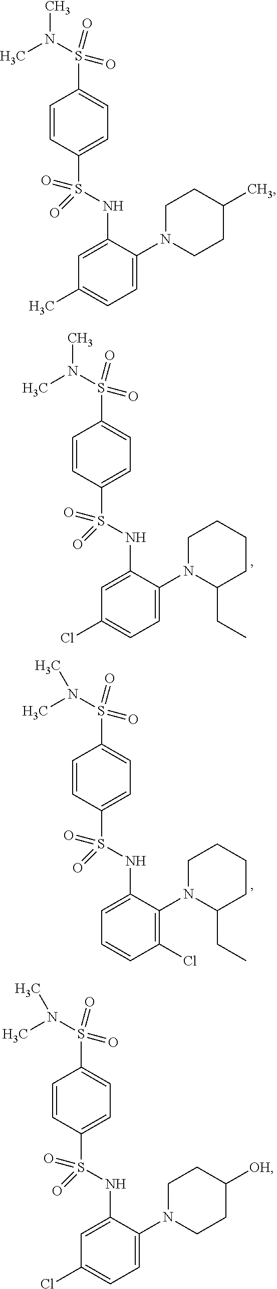

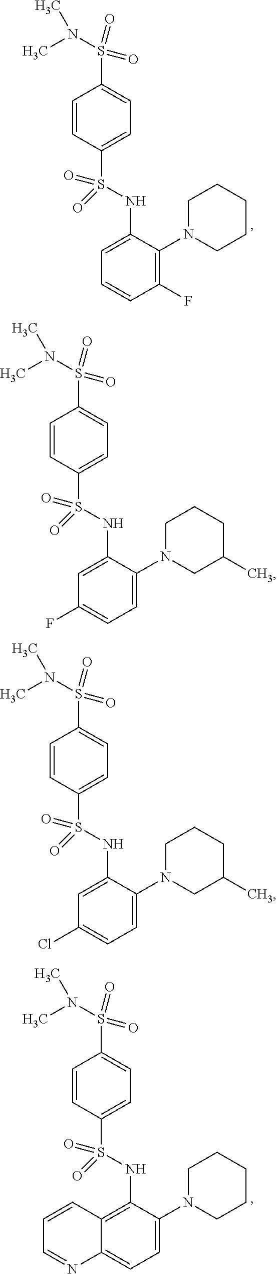

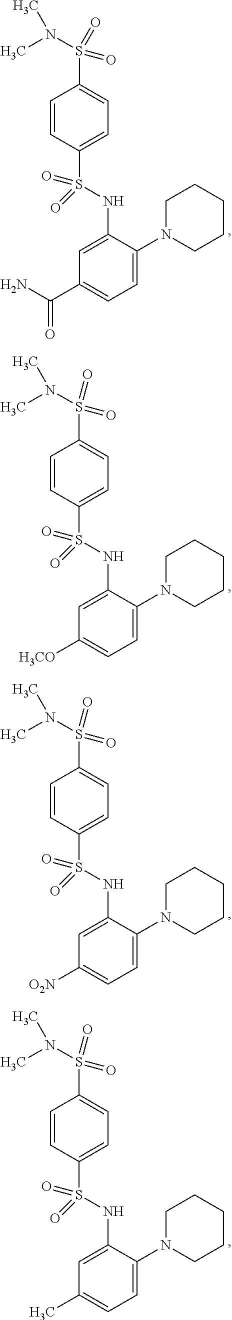

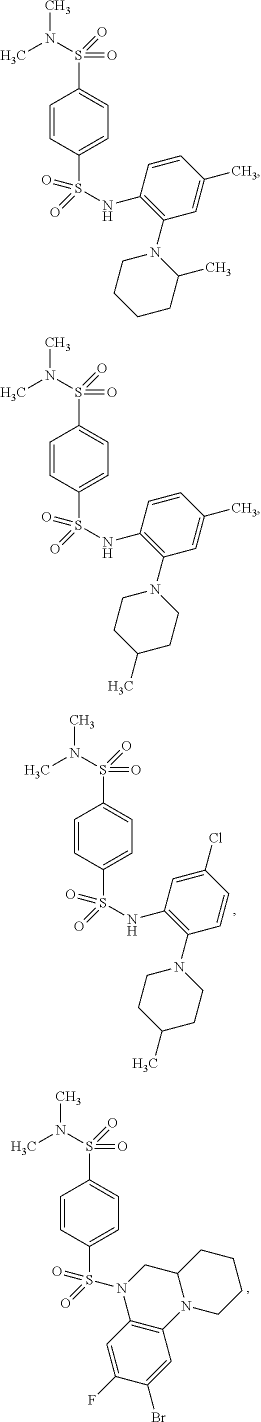 [SMILES: Cc1ccc(NS(=O)(=O)c2ccc(S(=O)(=O)N(C)C)cc2)c(N2CCCCC2C)c1 ; Cc1ccc(NS(=O)(=O)c2ccc(S(=O)(=O)N(C)C)cc2)c(N2CCC(C)CC2)c1 ; CC1CCN(c2ccc(Cl)cc2NS(=O)(=O)c2ccc(S(=O)(=O)N(C)C)cc2)CC1 ; CN(C)S(=O)(=O)c1ccc(S(=O)(=O)N2CC3CCCCN3c3cc(Br)c(F)cc32)cc1]

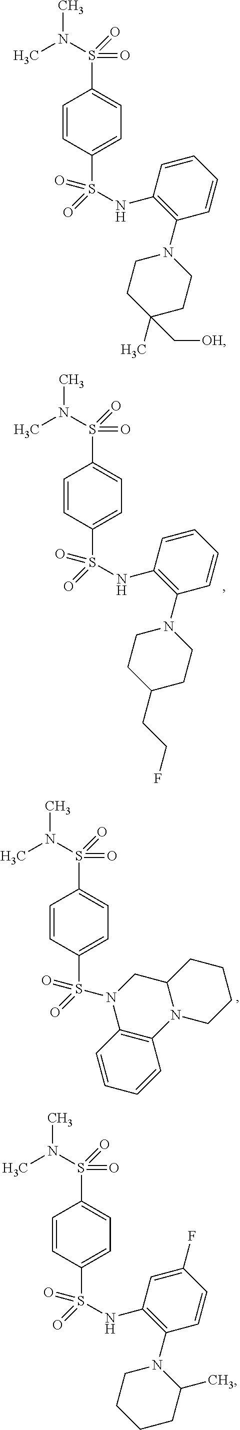 [SMILES: CN(C)S(=O)(=O)c1ccc(S(=O)(=O)Nc2ccccc2N2CCC(C)(CO)CC2)cc1 ; CN(C)S(=O)(=O)c1ccc(S(=O)(=O)Nc2ccccc2N2CCC(CCF)CC2)cc1 ; CN(C)S(=O)(=O)c1ccc(S(=O)(=O)N2CC3CCCCN3c3ccccc32)cc1 ; CC1CCCCN1c1ccc(F)cc1NS(=O)(=O)c1ccc(S(=O)(=O)N(C)C)cc1]

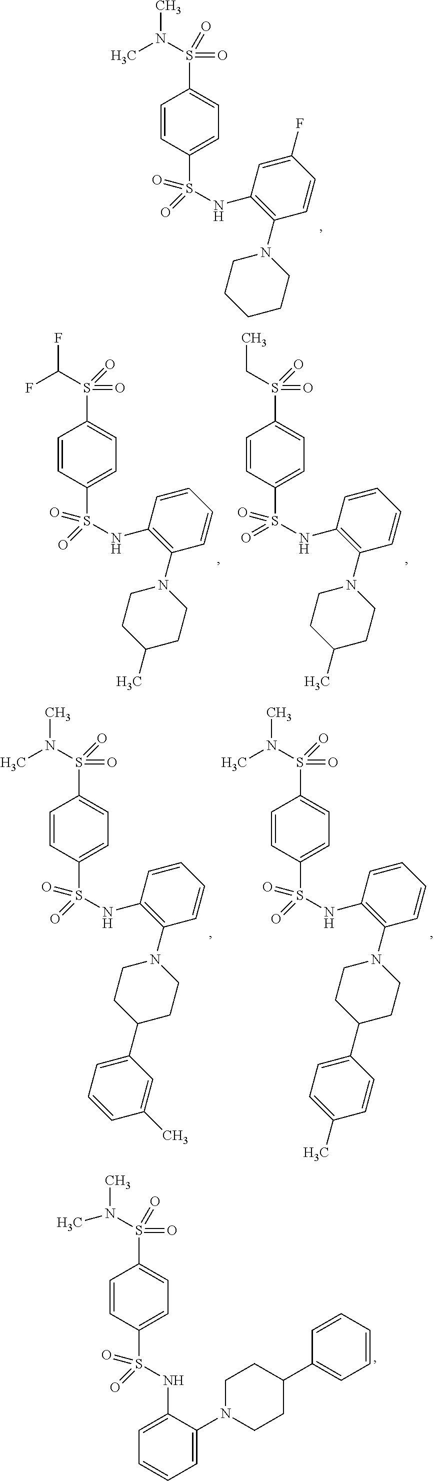

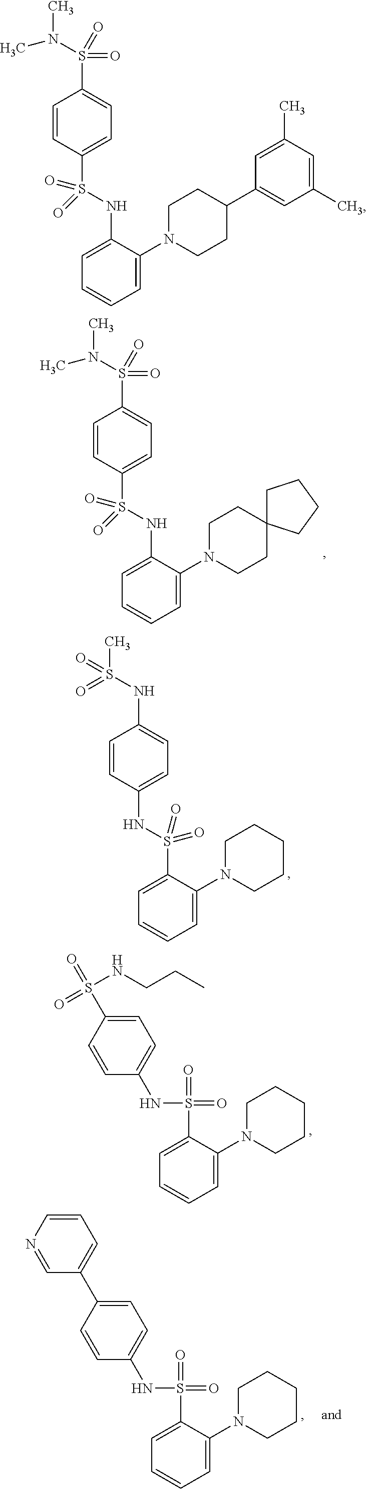

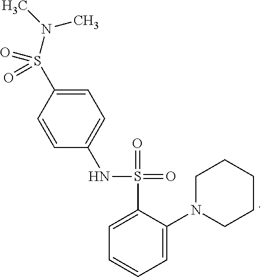 [SMILES: CN(C)S(=O)(=O)c1ccc(NS(=O)(=O)c2ccccc2N2CCCCC2)cc1]

15. A compound as provided in Examples 1-177.

16. A pharmaceutical composition comprising a compound of claim 1 or 15.

17. A method of treating, ameliorating, or preventing a disorder related to diminished ML1 activity in a patient comprising administering to said patient a therapeutically effective amount of the pharmaceutical composition of claim 16.

18. The method of claim 17, wherein the disorder related to diminished ML1 activity is DMD and/or another muscular dystrophy disorder related to ML1 activity (e.g., Becker's Muscular Dystrophy (BMD), Limb Girdle Muscular Dystrophy (LGMD), distal muscular dystrophy, facioscapulohumeral dystrophy, myotonic muscular dystrophy, Emery-Dreifuss muscular dystrophy, oculopharyngeal muscular dystrophy, and Congenital Muscular Dystrophy (CMD) (e.g., Laminin-α2-deficient CMD (MDC1A), Ullrich CMG (UCMDs 1, 2 and 3), Walker-Warburg syndrome (WWS), Muscle-eye-brain disease (MEB), Fukuyama CMD (FCMD), CMD plus secondary laminin deficiency 1 (MDC1B), CMD plus secondary laminin deficiency 2 (MDC1C), CMD with mental retardation and pachygyria (MDC1D), and Rigid spine with muscular dystrophy Type 1 (RSMD1)).

19. The method of claim 18, wherein said patient is a human patient.

20. A method of treating, ameliorating, or preventing a symptom associated with a disorder related to diminished ML1 activity in a patient comprising administering to said patient a therapeutically effective amount of the pharmaceutical composition of claim 16.

21. The method of claim 20, wherein the symptom is one or more symptoms selected from aberrant membrane repair capability related to diminished ML1 activity; aberrant lysosomal exocytosis activity related to diminished ML1 activity; aberrant lysosomal Ca²⁺ release channel activity related to diminished ML1 activity (required for lysosomal exocytosis); damaged sarcolemma related to diminished ML1 activity; aberrant Ca²⁺ dependent delivery of lysosomal membranes to damaged sarcolemma related to diminished ML1 activity; aberrant lysosomal activity related to diminished ML1 activity; muscle damage related to diminished ML1 activity; myofiber necrosis related to diminished ML1 activity; central nucleation related to diminished ML1 activity; fibrosis related to diminished ML1 activity; elevated serum creatine kinase (CK) levels related to diminished ML1 activity; reduced muscle force related to diminished ML1 activity; impaired motor ability related to diminished ML1 activity.

22. The method of claim 20, wherein the patient is a human patient suspect of having or having DMD and/or a muscular dystrophy disorder related to ML1 activity (e.g., Becker's Muscular Dystrophy (BMD), Limb Girdle Muscular Dystrophy (LGMD), distal muscular dystrophy, facioscapulohumeral dystrophy, myotonic muscular dystrophy, Emery-Dreifuss muscular dystrophy, oculopharyngeal muscular dystrophy, and Congenital Muscular Dystrophy (CMD) (e.g., Laminin-α2-deficient CMD (MDC1A), Ullrich CMG (UCMDs 1, 2 and 3), Walker-Warburg syndrome (WWS), Muscle-eye-brain disease (MEB), Fukuyama CMD (FCMD), CMD plus secondary laminin deficiency 1 (MDC1B), CMD plus secondary laminin deficiency 2 (MDC1C), CMD with mental retardation and pachygyria (MDC1D), and Rigid spine with muscular dystrophy Type 1 (RSMD1)).

23. A kit comprising a compound of claim 1 or claim 15 and instructions for administering said compound to a patient having a disorder related to diminished ML1 activity.

24. The kit of claim 23, wherein the disorder related to disorder related to diminished ML1 activity is DMD and/or a muscular dystrophy disorder related to ML1 activity (e.g., Becker's Muscular Dystrophy (BMD), Limb Girdle Muscular Dystrophy (LGMD), distal muscular dystrophy, facioscapulohumeral dystrophy, myotonic muscular dystrophy, Emery-Dreifuss muscular dystrophy, oculopharyngeal muscular dystrophy, and Congenital Muscular Dystrophy (CMD) (e.g., Laminin-α2-deficient CMD (MDC1A), Ullrich CMG (UCMDs 1, 2 and 3), Walker-Warburg syndrome (WWS), Muscle-eye-brain disease (MEB), Fukuyama CMD (FCMD), CMD plus secondary laminin deficiency 1 (MDC1B), CMD plus secondary laminin deficiency 2 (MDC1C), CMD with mental retardation and pachygyria (MDC1D), and Rigid spine with muscular dystrophy Type 1 (RSMD1)).
